SEQUENCE LISTING (1021 sequences)

SEQ ID NO: 1 - protein, 14 aa, Homo sapiens
  misc_feature 2, 4, 5, 8, 11, 13: Xaa at positions 2, 4, 5, 8, 11 and 13 is any amino acid
YXCXXGPXTWXCXP

SEQ ID NO: 2 - protein, 14 aa, Homo sapiens
  misc_feature 2, 4, 5, 8, 11, 13: Xaa at positions 2, 4, 5, 8, 11 and 13 is any amino acid
YXCXXGPXTWXCXP

SEQ ID NO: 3 - protein, 14 aa, Homo sapiens
  misc_feature 2, 4, 5, 8, 11, 13: Xaa at positions 2, 4, 5, 8, 11 and 13 is any amino acid
YXCXXGPXTWXCXP

SEQ ID NO: 4 - protein, 14 aa, Homo sapiens
  misc_feature 2, 4, 5, 8, 11, 13: Xaa at positions 2, 4, 5, 8, 11 and 13 is any amino acid
YXCXXGPXTWXCXP

SEQ ID NO: 5 - protein, 20 aa, Homo sapiens
GGTYSCHFGPLTWVCKPQGG

SEQ ID NO: 6 - protein, 20 aa, Homo sapiens
GGDYHCRMGPLTWVCKPLGG

SEQ ID NO: 7 - protein, 20 aa, Homo sapiens
GGVYACRMGPITWVCSPLGG

SEQ ID NO: 8 - protein, 20 aa, Homo sapiens
VGNYMCHFGPITWVCRPGGG

SEQ ID NO: 9 - protein, 20 aa, Homo sapiens
GGLYLCRFGPVTWDCGYKGG

SEQ ID NO: 10 - protein, 20 aa, Homo sapiens
GGTYSCHFGPLTWVCKPQGG

SEQ ID NO: 11 - protein, 20 aa, Homo sapiens
GGTYSCHFGPLTWVCKPQGG

SEQ ID NO: 12 - protein, 23 aa, Homo sapiens
GGTYSCHFGPLTWVCKPQGGSSK

SEQ ID NO: 13 - protein, 23 aa, Homo sapiens
GGTYSCHFGPLTWVCKPQGGSSK

SEQ ID NO: 14 - protein, 23 aa, Homo sapiens
GGTYSCHFGPLTWVCKPQGGSSK

SEQ ID NO: 15 - protein, 22 aa, Homo sapiens
GGTYSCHFGPLTWVCKPQGGSS

SEQ ID NO: 16 - protein, 23 aa, Homo sapiens
GGTYSCHFGPLTWVCKPQGGSSK

SEQ ID NO: 17 - protein, 10 aa, Homo sapiens
  misc_feature 2, 3, 6, 9: Xaa at positions 2, 3, 6, and 9 is any amino acid
CXXGPXTWXC

SEQ ID NO: 18 - protein, 19 aa, Homo sapiens
GGTYSCHGPLTWVCKPQGG

SEQ ID NO: 19 - protein, 19 aa, Homo sapiens
VGNYMAHMGPITWVCRPGG

SEQ ID NO: 20 - protein, 18 aa, Homo sapiens
GGPHHVYACRMGPLTWIC

SEQ ID NO: 21 - protein, 18 aa, Homo sapiens
GGTYSCHFGPLTWVCKPQ

SEQ ID NO: 22 - protein, 20 aa, Homo sapiens
GGLYACHMGPMTWVCQPLRG

SEQ ID NO: 23 - protein, 22 aa, Homo sapiens
TIAQYICYMGPETWECRPSPKA

SEQ ID NO: 24 - protein, 13 aa, Homo sapiens
YSCHFGPLTWVCK

SEQ ID NO: 25 - protein, 11 aa, Homo sapiens
YCHFGPLTWVC

SEQ ID NO: 26 - protein, 10 aa, Homo sapiens
  misc_feature 1, 2, 3, 6, 9, 10: Xaa at positions 1, 2, 3, 6, 9 and 10 is any amino acid
XXXGPXTWXX

SEQ ID NO: 27 - protein, 12 aa, Homo sapiens
  Misc_feature 2, 3, 4, 5, 8, 11, 12: Xaa at positions 2-5, 8 and 11-12 is any amino acid
YXXXXGPXTWXX

SEQ ID NO: 28 - protein, 14 aa, Homo sapiens
  misc_feature 1, 3, 4, 5, 6, 9, 10, 11, 12, 13, 14: Xaa at positions 1, 3-6 and 9-14 is any amino acid
XYXXXXGPXXXXXX

SEQ ID NO: 29 - protein, 18 aa, Homo sapiens
  misc_feature 1, 3, 4, 6, 9, 12, 14, 15, 16: Xaa at positions 1, 3, 5, 6, 9, 12 and 14-16 is any amino acid
XYXCXXGPXTWXCXXXLL

SEQ ID NO: 30 - protein, 20 aa, Homo sapiens
GGLYLCRFGPVTWDCGYKGG

SEQ ID NO: 31 - protein, 20 aa, Homo sapiens
GGTYSCHFGPLTWVCKPQGG

SEQ ID NO: 32 - protein, 20 aa, Homo sapiens
GGDYHCRMGPLTWVCKPLGG

SEQ ID NO: 33 - protein, 20 aa, Homo sapiens
VGNYMCHFGPITWVCRPGGG

SEQ ID NO: 34 - protein, 20 aa, Homo sapiens
GGVYACRMGPITWVCSPLGG

SEQ ID NO: 35 - protein, 19 aa, Homo sapiens
VGNYMAHMGPITWVCRPGG

SEQ ID NO: 36 - protein, 18 aa, Homo sapiens
GGTYSCHFGPLTWVCKPQ

SEQ ID NO: 37 - protein, 20 aa, Homo sapiens
GGLYACHMGPMTWVCQPLRG

SEQ ID NO: 38 - protein, 22 aa, Homo sapiens
TIAQYICYMGPETWECRPSPKA

SEQ ID NO: 39 - protein, 13 aa, Homo sapiens
YSCHFGPLTWVCK

SEQ ID NO: 40 - protein, 11 aa, Homo sapiens
YCHFGPLTWVC

SEQ ID NO: 41 - protein, 12 aa, Homo sapiens
SCHFGPLTWVCK

SEQ ID NO: 42 - protein, 12 aa, Homo sapiens
  misc_feature 2-5, 8 and 11-12: Xaa at positions 2-5, 8 and 11-12 is any amino acid
AXXXXGPXTWXX

SEQ ID NO: 43 - protein, 10 aa, Homo sapiens
  Xaa at positions 1-4, 6 and 8-10 is any amino acid
XXXXQXYXXX

SEQ ID NO: 44 - protein, 8 aa, Homo sapiens
  misc_feature 1-2, 4 and 6-8: Xaa at positions 1-2, 4 and 6-8 is any amino acid
XXQXYXXX

SEQ ID NO: 45 - protein, 8 aa, Homo sapiens
  misc_feature 1-2, 4 and 6-8: Xaa at positions 1-2, 4 and 6-8 is any amino acid
XXQXYXXX

SEQ ID NO: 46 - protein, 8 aa, Homo sapiens
  misc_feature 1-2, 4 and 6-8: Xaa at positions 1-2, 4 and 6-8 is any amino acid
XXQXYXXX

SEQ ID NO: 47 - protein, 9 aa, Homo sapiens
  misc_feature 1-3, 5 and 7-9: Xaa at positions 1-3, 5 and 7-9 is any amino acid
XXXQXYXXX

SEQ ID NO: 48 - protein, 21 aa, Homo sapiens
  misc_feature 1-14, 16 and 18-20: Xaa at positions 1-14, 16 and 18-20 is any amino acid
XXXXXXXXXXXXXXQXYXXXL

SEQ ID NO: 49 - protein, 11 aa, Homo sapiens
  m isc_feature 1 and 3-11: Xaa at positions 1 and 3-11 is any amino acid
XNXXXXXXXXX

SEQ ID NO: 50 - protein, 21 aa, Homo sapiens
TANVSSFEWTPYYWQPYALPL

SEQ ID NO: 51 - protein, 18 aa, Homo sapiens
SWTDYGYWQPYALPISGL

SEQ ID NO: 52 - protein, 21 aa, Homo sapiens
ETPFTWEESNAYYWQPYALPL

SEQ ID NO: 53 - protein, 21 aa, Homo sapiens
ENTYSPNWADSMYWQPYALPL

SEQ ID NO: 54 - protein, 21 aa, Homo sapiens
SVGEDHNFWTSEYWQPYALPL

SEQ ID NO: 55 - protein, 21 aa, Homo sapiens
DGYDRWRQSGERYWQPYALPL

SEQ ID NO: 56 - protein, 11 aa, Homo sapiens
FEWTPGYWQPY

SEQ ID NO: 57 - protein, 11 aa, Homo sapiens
FEWTPGYWQHY

SEQ ID NO: 58 - protein, 11 aa, Homo sapiens
  misc_feature 10: Xaa at position 10 is any amino acid
FEWTPGWYQXY

SEQ ID NO: 59 - protein, 11 aa, Homo sapiens
  misc_feature 10: Xaa at position 10 is any amino acid
FEWTPGWYQXY

SEQ ID NO: 60 - protein, 13 aa, Homo sapiens
  misc_feature 12: Xaa at position 12 is any amino acid
FEVFFPGWPYQXY

SEQ ID NO: 61 - protein, 11 aa, Homo sapiens
  misc_feature 10: Xaa at position 10 is any amino acid
FAWTPGYWQXY

SEQ ID NO: 62 - protein, 11 aa, Homo sapiens
  misc_feature 10: Xaa at position 10 is any amino acid
FEWAPGYWQXY

SEQ ID NO: 63 - protein, 11 aa, Homo sapiens
  misc_feature 10: Xaa at position 10 is any amino acid
FEWVPGYWQXY

SEQ ID NO: 64 - protein, 11 aa, Homo sapiens
  misc_feature 10: Xaa at position 10 is any amino acid
FEWTPGYWQXY

SEQ ID NO: 65 - protein, 11 aa, Homo sapiens
  misc_feature 10: Xaa at position 10 is any amino acid
FEWTPGYWQXY

SEQ ID NO: 66 - protein, 11 aa, Homo sapiens
  misc_feature 10: Xaa at position 10 is any amino acid
FEWTPAWYQXY

SEQ ID NO: 67 - protein, 13 aa, Homo sapiens
  misc_feature 12: Xaa at position 12 is any amino acid
FEWTPSARWYQXY

SEQ ID NO: 68 - protein, 11 aa, Homo sapiens
FEWTPGYYQPY

SEQ ID NO: 69 - protein, 11 aa, Homo sapiens
FEWTPGWWQPY

SEQ ID NO: 70 - protein, 11 aa, Homo sapiens
FEWTPNYWQPY

SEQ ID NO: 71 - protein, 12 aa, Homo sapiens
  misc_feature 11: Xaa at position 11 is any amino acid
FEVFFPVYWQXY

SEQ ID NO: 72 - protein, 13 aa, Homo sapiens
  misc_feature 12: Xaa at position 12 is any amino acid
FEWTPECGYWQXY

SEQ ID NO: 73 - protein, 12 aa, Homo sapiens
  misc_feature 11: Xaa at position 11 is any amino acid
FEWTPAIYWQXY

SEQ ID NO: 74 - protein, 14 aa, Homo sapiens
  misc_feature 13: Xaa at position 13 is any amino acid
FEVFFSARGYWQXY

SEQ ID NO: 75 - protein, 11 aa, Homo sapiens
FEWTPGYWQPY

SEQ ID NO: 76 - protein, 11 aa, Homo sapiens
FEWTPGYWQHY

SEQ ID NO: 77 - protein, 11 aa, Homo sapiens
  misc_feature 10: Xaa at position 10 is any amino acid
FEWTPGWYQXY

SEQ ID NO: 78 - protein, 11 aa, Homo sapiens
  misc_feature 10: Xaa at position 10 is any amino acid
FEWTPGWYQXY

SEQ ID NO: 79 - protein, 11 aa, Homo sapiens
  misc_feature 10: Xaa at position 10 is any amino acid
FEWTPGWYQXY

SEQ ID NO: 80 - protein, 11 aa, Homo sapiens
  misc_feature 10: Xaa at position 10 is any amino acid
FAWTPGYWQXY

SEQ ID NO: 81 - protein, 11 aa, Homo sapiens
  misc_feature 10: Xaa at position 10 is any amino acid
FEWAPGYWQXY

SEQ ID NO: 82 - protein, 11 aa, Homo sapiens
  misc_feature 10: Xaa at position 10 is any amino acid
FEWVPGYWQXY

SEQ ID NO: 83 - protein, 11 aa, Homo sapiens
  misc_feature 10: Xaa at position 10 is any amino acid
FEWTPGYWQXY

SEQ ID NO: 84 - protein, 12 aa, Homo sapiens
  misc_feature 11: Xaa at position 11 is any amino acid
AFEWTPGYWQXY

SEQ ID NO: 85 - protein, 11 aa, Homo sapiens
  misc_feature 10: Xaa at position 10 is any amino acid
FEWTPAWYQXY

SEQ ID NO: 86 - protein, 13 aa, Homo sapiens
  misc_feature 12: Xaa at position 12 is any amino acid
FEWTPSARWYQXY

SEQ ID NO: 87 - protein, 11 aa, Homo sapiens
FEWTPGYYQPY

SEQ ID NO: 88 - protein, 11 aa, Homo sapiens
FEWTPGWWQPY

SEQ ID NO: 89 - protein, 11 aa, Homo sapiens
FEWTPNYWQPY

SEQ ID NO: 90 - protein, 11 aa, Homo sapiens
  misc_feature 10: Xaa at position 10 is any amino acid
FEWTPVYWQXY

SEQ ID NO: 91 - protein, 13 aa, Homo sapiens
  misc_feature 12: Xaa at position 12 is any amino acid
FEWTPECGYWQXY

SEQ ID NO: 92 - protein, 12 aa, Homo sapiens
  misc_feature 11: Xaa at position 11 is any amino acid
FEWTPAIYWQXY

SEQ ID NO: 93 - protein, 13 aa, Homo sapiens
  misc_feature 12: Xaa at position 12 is any amino acid
FEWTSARGYWQXY

SEQ ID NO: 94 - protein, 15 aa, Homo sapiens
FEWTPGYWQPYALPL

SEQ ID NO: 95 - protein, 10 aa, Homo sapiens
  misc_feature 9: Xaa at position 9 is any amino acid
EWTPGYYQXY

SEQ ID NO: 96 - protein, 11 aa, Homo sapiens
  misc_feature 10: Xaa at position 10 is any amino acid
YEWTPGYYQXY

SEQ ID NO: 97 - protein, 11 aa, Homo sapiens
  misc_feature 10: Xaa at position 10 is any amino acid
FEWVPGYYQXY

SEQ ID NO: 98 - protein, 11 aa, Homo sapiens
  misc_feature 10: Xaa at position 10 is any amino acid
FEWTPSYYQXY

SEQ ID NO: 99 - protein, 11 aa, Homo sapiens
  misc_feature 10: Xaa at position 10 is any amino acid
FEWTPNYYQXY

SEQ ID NO: 100 - protein, 4 aa, Homo sapiens
TKPR

SEQ ID NO: 101 - protein, 5 aa, Homo sapiens
RKSSK

SEQ ID NO: 102 - protein, 5 aa, Homo sapiens
RKQDK

SEQ ID NO: 103 - protein, 6 aa, Homo sapiens
NRKQDK

SEQ ID NO: 104 - protein, 6 aa, Homo sapiens
RKQDKR

SEQ ID NO: 105 - protein, 9 aa, Homo sapiens
ENRKQDKRF

SEQ ID NO: 106 - protein, 6 aa, Homo sapiens
VTKFYF

SEQ ID NO: 107 - protein, 5 aa, Homo sapiens
VTKFY

SEQ ID NO: 108 - protein, 5 aa, Homo sapiens
VTDFY

SEQ ID NO: 109 - protein, 11 aa, Homo sapiens
SHLYWQPYSVQ

SEQ ID NO: 110 - protein, 12 aa, Homo sapiens
TLVYWQPYSLQT

SEQ ID NO: 111 - protein, 12 aa, Homo sapiens
RGDYWQPYSVQS

SEQ ID NO: 112 - protein, 12 aa, Homo sapiens
VHVYWQPYSVQT

SEQ ID NO: 113 - protein, 12 aa, Homo sapiens
RLVYWQPYSVQT

SEQ ID NO: 114 - protein, 12 aa, Homo sapiens
SRVWFQPYSLQS

SEQ ID NO: 115 - protein, 12 aa, Homo sapiens
NMVYWQPYSIQT

SEQ ID NO: 116 - protein, 12 aa, Homo sapiens
SVVFWQPYSVQT

SEQ ID NO: 117 - protein, 12 aa, Homo sapiens
TFVYWQPYALPL

SEQ ID NO: 118 - protein, 12 aa, Homo sapiens
TLVYWQPYSIQR

SEQ ID NO: 119 - protein, 12 aa, Homo sapiens
RLVYWQPYSVQR

SEQ ID NO: 120 - protein, 12 aa, Homo sapiens
SPVFWQPYSIQI

SEQ ID NO: 121 - protein, 12 aa, Homo sapiens
WIEWWQPYSVQS

SEQ ID NO: 122 - protein, 12 aa, Homo sapiens
SLIYWQPYSLQM

SEQ ID NO: 123 - protein, 12 aa, Homo sapiens
TRLYWQPYSVQR

SEQ ID NO: 124 - protein, 12 aa, Homo sapiens
RCDYWQPYSVQT

SEQ ID NO: 125 - protein, 12 aa, Homo sapiens
MRVFWQPYSVQN

SEQ ID NO: 126 - protein, 12 aa, Homo sapiens
KIVYWQPYSVQT

SEQ ID NO: 127 - protein, 12 aa, Homo sapiens
RHLYWQPYSVQR

SEQ ID NO: 128 - protein, 12 aa, Homo sapiens
ALVWWQPYSEQI

SEQ ID NO: 129 - protein, 12 aa, Homo sapiens
SRVWFQPYSLQS

SEQ ID NO: 130 - protein, 10 aa, Homo sapiens
WEQPYALPLE

SEQ ID NO: 131 - protein, 12 aa, Homo sapiens
QLVWWQPYSVQR

SEQ ID NO: 132 - protein, 12 aa, Homo sapiens
DLRYWQPYSVQV

SEQ ID NO: 133 - protein, 12 aa, Homo sapiens
ELVWWQPYSLQL

SEQ ID NO: 134 - protein, 12 aa, Homo sapiens
DLVWWQPYSVQW

SEQ ID NO: 135 - protein, 12 aa, Homo sapiens
NGNYWQPYSFQV

SEQ ID NO: 136 - protein, 12 aa, Homo sapiens
ELVYWQPYSIQR

SEQ ID NO: 137 - protein, 13 aa, Homo sapiens
ELMYAIQPYSVQE

SEQ ID NO: 138 - protein, 12 aa, Homo sapiens
NLLYWQPYSMQD

SEQ ID NO: 139 - protein, 12 aa, Homo sapiens
GYEWYQPYSVQR

SEQ ID NO: 140 - protein, 12 aa, Homo sapiens
SRVWYQPYSVQR

SEQ ID NO: 141 - protein, 12 aa, Homo sapiens
LSEQYQPYSVQR

SEQ ID NO: 142 - protein, 12 aa, Homo sapiens
GGGWWQPYSVQR

SEQ ID NO: 143 - protein, 12 aa, Homo sapiens
VGRWYQPYSVQR

SEQ ID NO: 144 - protein, 12 aa, Homo sapiens
VHVYWQPYSVQR

SEQ ID NO: 145 - protein, 12 aa, Homo sapiens
QARWYQPYSVQR

SEQ ID NO: 146 - protein, 12 aa, Homo sapiens
VHVYWQPYSVQT

SEQ ID NO: 147 - protein, 12 aa, Homo sapiens
RSVYWQPYSVQR

SEQ ID NO: 148 - protein, 12 aa, Homo sapiens
TRVWFQPYSVQR

SEQ ID NO: 149 - protein, 12 aa, Homo sapiens
GRIWFQPYSVQR

SEQ ID NO: 150 - protein, 12 aa, Homo sapiens
GRVWFQPYSVQR

SEQ ID NO: 151 - protein, 12 aa, Homo sapiens
ARTWYQPYSVQR

SEQ ID NO: 152 - protein, 12 aa, Homo sapiens
ARVWWQPYSVQM

SEQ ID NO: 153 - protein, 12 aa, Homo sapiens
RLMFYQPYSVQR

SEQ ID NO: 154 - protein, 12 aa, Homo sapiens
ESMWYQPYSVQR

SEQ ID NO: 155 - protein, 12 aa, Homo sapiens
HFGWWQPYSVHM

SEQ ID NO: 156 - protein, 12 aa, Homo sapiens
ARFWWQPYSVQR

SEQ ID NO: 157 - protein, 12 aa, Homo sapiens
RLVYWQPYAPIY

SEQ ID NO: 158 - protein, 12 aa, Homo sapiens
RLVYWQPYSYQT

SEQ ID NO: 159 - protein, 12 aa, Homo sapiens
RLVYWQPYSLPI

SEQ ID NO: 160 - protein, 12 aa, Homo sapiens
RLVYWQPYSVQA

SEQ ID NO: 161 - protein, 12 aa, Homo sapiens
SRVWYQPYAKGL

SEQ ID NO: 162 - protein, 12 aa, Homo sapiens
SRVWYQPYAQGL

SEQ ID NO: 163 - protein, 12 aa, Homo sapiens
SRVWYQPYAMPL

SEQ ID NO: 164 - protein, 12 aa, Homo sapiens
SRVWYQPYSVQA

SEQ ID NO: 165 - protein, 12 aa, Homo sapiens
SRVWYQPYSLGL

SEQ ID NO: 166 - protein, 12 aa, Homo sapiens
SRVWYQPYAREL

SEQ ID NO: 167 - protein, 12 aa, Homo sapiens
SRVWYQPYSRQP

SEQ ID NO: 168 - protein, 12 aa, Homo sapiens
SRVWYQPYFVQP

SEQ ID NO: 169 - protein, 12 aa, Homo sapiens
EYEWYQPYALPL

SEQ ID NO: 170 - protein, 12 aa, Homo sapiens
IPEYWQPYALPL

SEQ ID NO: 171 - protein, 12 aa, Homo sapiens
SRIWWQPYALPL

SEQ ID NO: 172 - protein, 12 aa, Homo sapiens
DPLFWQPYALPL

SEQ ID NO: 173 - protein, 12 aa, Homo sapiens
SRQWVQPYALPL

SEQ ID NO: 174 - protein, 12 aa, Homo sapiens
IRSWWQPYALPL

SEQ ID NO: 175 - protein, 11 aa, Homo sapiens
RGYWQPYALPL

SEQ ID NO: 176 - protein, 12 aa, Homo sapiens
RLLWVQPYALPL

SEQ ID NO: 177 - protein, 12 aa, Homo sapiens
EYRWFQPYALPL

SEQ ID NO: 178 - protein, 12 aa, Homo sapiens
DAYWVQPYALPL

SEQ ID NO: 179 - protein, 12 aa, Homo sapiens
WSGYFQPYALPL

SEQ ID NO: 180 - protein, 12 aa, Homo sapiens
NIEFWQPYALPL

SEQ ID NO: 181 - protein, 12 aa, Homo sapiens
TRDWVQPYALPL

SEQ ID NO: 182 - protein, 12 aa, Homo sapiens
DSSWYQPYALPL

SEQ ID NO: 183 - protein, 12 aa, Homo sapiens
IGNWYQPYALPL

SEQ ID NO: 184 - protein, 12 aa, Homo sapiens
NLRWDQPYALPL

SEQ ID NO: 185 - protein, 12 aa, Homo sapiens
LPEFWQPYALPL

SEQ ID NO: 186 - protein, 12 aa, Homo sapiens
DSYWWQPYALPL

SEQ ID NO: 187 - protein, 12 aa, Homo sapiens
RSQYYQPYALPL

SEQ ID NO: 188 - protein, 12 aa, Homo sapiens
ARFWLQPYALPL

SEQ ID NO: 189 - protein, 12 aa, Homo sapiens
NSYFWQPYALPL

SEQ ID NO: 190 - protein, 12 aa, Homo sapiens
RFMYWQPYSVQR

SEQ ID NO: 191 - protein, 12 aa, Homo sapiens
AHLFWQPYSVQR

SEQ ID NO: 192 - protein, 9 aa, Homo sapiens
WWQPYALPL

SEQ ID NO: 193 - protein, 9 aa, Homo sapiens
YYQPYALPL

SEQ ID NO: 194 - protein, 9 aa, Homo sapiens
YFQPYALGL

SEQ ID NO: 195 - protein, 10 aa, Homo sapiens
YWYQPYALPL

SEQ ID NO: 196 - protein, 10 aa, Homo sapiens
RWWQPYATPL

SEQ ID NO: 197 - protein, 10 aa, Homo sapiens
GWYQPYALGF

SEQ ID NO: 198 - protein, 10 aa, Homo sapiens
YWYQPYALGL

SEQ ID NO: 199 - protein, 10 aa, Homo sapiens
IWYQPYAMPL

SEQ ID NO: 200 - protein, 10 aa, Homo sapiens
SNMQPYQRLS

SEQ ID NO: 201 - protein, 20 aa, Homo sapiens
TFVYWQPYAVGLPAAETACN

SEQ ID NO: 202 - protein, 20 aa, Homo sapiens
TFVYWQPYSVQMTITGKVTM

SEQ ID NO: 203 - protein, 20 aa, Homo sapiens
  misc_feature 12-13 and 16: Xaa at positions 12-13 and 16 is any amino acid
TFVYWQPYSSHXXVPXGFPL

SEQ ID NO: 204 - protein, 20 aa, Homo sapiens
TFVYWQPYYGNPQWAIHVRH

SEQ ID NO: 205 - protein, 20 aa, Homo sapiens
TFVYWQPYVLLELPEGAVRA

SEQ ID NO: 206 - protein, 20 aa, Homo sapiens
TFVYWQPYVDYVWPIPIAQV

SEQ ID NO: 207 - protein, 11 aa, Homo sapiens
GWYQPYVDGWR

SEQ ID NO: 208 - protein, 12 aa, Homo sapiens
RWEQPYVKDGWS

SEQ ID NO: 209 - protein, 12 aa, Homo sapiens
EWYQPYALGWAR

SEQ ID NO: 210 - protein, 10 aa, Homo sapiens
GWWQPYARGL

SEQ ID NO: 211 - protein, 12 aa, Homo sapiens
LFEQPYAKALGL

SEQ ID NO: 212 - protein, 12 aa, Homo sapiens
GWEQPYARGLAG

SEQ ID NO: 213 - protein, 12 aa, Homo sapiens
AWVQPYATPLDE

SEQ ID NO: 214 - protein, 12 aa, Homo sapiens
MWYQPYSSQPAE

SEQ ID NO: 215 - protein, 12 aa, Homo sapiens
GWTQPYSQQGEV

SEQ ID NO: 216 - protein, 12 aa, Homo sapiens
DWFQPYSIQSDE

SEQ ID NO: 217 - protein, 11 aa, Homo sapiens
PWIQPYARGFG

SEQ ID NO: 218 - protein, 12 aa, Homo sapiens
RPLYWQPYSVQV

SEQ ID NO: 219 - protein, 12 aa, Homo sapiens
TLIYWQPYSVQI

SEQ ID NO: 220 - protein, 12 aa, Homo sapiens
RFDYWQPYSDQT

SEQ ID NO: 221 - protein, 12 aa, Homo sapiens
WHQFVQPYALPL

SEQ ID NO: 222 - protein, 12 aa, Homo sapiens
WHQFVQPYALPL

SEQ ID NO: 223 - protein, 17 aa, Homo sapiens
EWDSVYWQPYSVQTLLR

SEQ ID NO: 224 - protein, 17 aa, Homo sapiens
WEQNVYWQPYSVQSFAD

SEQ ID NO: 225 - protein, 16 aa, Homo sapiens
SDVVYWQPYSVQSLEM

SEQ ID NO: 226 - protein, 17 aa, Homo sapiens
YYDGVYWQPYSVQVMPA

SEQ ID NO: 227 - protein, 12 aa, Homo sapiens
SDIWYQPYALPL

SEQ ID NO: 228 - protein, 12 aa, Homo sapiens
QRIWWQPYALPL

SEQ ID NO: 229 - protein, 12 aa, Homo sapiens
SRIWWQPYALPL

SEQ ID NO: 230 - protein, 12 aa, Homo sapiens
RSLYWQPYALPL

SEQ ID NO: 231 - protein, 12 aa, Homo sapiens
TIIWEQPYALPL

SEQ ID NO: 232 - protein, 12 aa, Homo sapiens
WETWYQPYALPL

SEQ ID NO: 233 - protein, 12 aa, Homo sapiens
SYDWEQPYALPL

SEQ ID NO: 234 - protein, 12 aa, Homo sapiens
SRIWCQPYALPL

SEQ ID NO: 235 - protein, 12 aa, Homo sapiens
EIMFWQPYALPL

SEQ ID NO: 236 - protein, 12 aa, Homo sapiens
DYVWQQPYALPL

SEQ ID NO: 237 - protein, 15 aa, Homo sapiens
MDLLVQWYQPYALPL

SEQ ID NO: 238 - protein, 15 aa, Homo sapiens
GSKVILWYQPYALPL

SEQ ID NO: 239 - protein, 15 aa, Homo sapiens
RQGANIWYQPYALPL

SEQ ID NO: 240 - protein, 15 aa, Homo sapiens
GGGDEPWYQPYALPL

SEQ ID NO: 241 - protein, 15 aa, Homo sapiens
SQLERTWYQPYALPL

SEQ ID NO: 242 - protein, 15 aa, Homo sapiens
ETWVREWYQPYALPL

SEQ ID NO: 243 - protein, 15 aa, Homo sapiens
KKGSTQWYQPYALPL

SEQ ID NO: 244 - protein, 15 aa, Homo sapiens
LQARMNWYQPYALPL

SEQ ID NO: 245 - protein, 15 aa, Homo sapiens
EPRSQKWYQPYALPL

SEQ ID NO: 246 - protein, 15 aa, Homo sapiens
VKQKWRWYQPYALPL

SEQ ID NO: 247 - protein, 15 aa, Homo sapiens
LRRHDVWYQPYALPL

SEQ ID NO: 248 - protein, 15 aa, Homo sapiens
RSTASIWYQPYALPL

SEQ ID NO: 249 - protein, 15 aa, Homo sapiens
ESKEDQWYQPYALPL

SEQ ID NO: 250 - protein, 15 aa, Homo sapiens
EGLTMKWYQPYALPL

SEQ ID NO: 251 - protein, 15 aa, Homo sapiens
EGSREGWYQPYALPL

SEQ ID NO: 252 - protein, 12 aa, Homo sapiens
VIEWWQPYALPL

SEQ ID NO: 253 - protein, 12 aa, Homo sapiens
VWYWEQPYALPL

SEQ ID NO: 254 - protein, 12 aa, Homo sapiens
ASEWWQPYALPL

SEQ ID NO: 255 - protein, 12 aa, Homo sapiens
FYEWWQPYALPL

SEQ ID NO: 256 - protein, 12 aa, Homo sapiens
EGWWVQPYALPL

SEQ ID NO: 257 - protein, 12 aa, Homo sapiens
WGEWLQPYALPL

SEQ ID NO: 258 - protein, 12 aa, Homo sapiens
DYVWEQPYALPL

SEQ ID NO: 259 - protein, 12 aa, Homo sapiens
AHTWWQPYALPL

SEQ ID NO: 260 - protein, 12 aa, Homo sapiens
FIEWFQPYALPL

SEQ ID NO: 261 - protein, 12 aa, Homo sapiens
WLAWEQPYALPL

SEQ ID NO: 262 - protein, 12 aa, Homo sapiens
VMEWWQPYALPL

SEQ ID NO: 263 - protein, 11 aa, Homo sapiens
ERMWQPYALPL

SEQ ID NO: 264 - protein, 12 aa, Homo sapiens
  misc_feature 2, 3, 5, and 6: Xaa at positions 2, 3, 5, and 6 is any amino acid
NXXWXXPYALPL

SEQ ID NO: 265 - protein, 12 aa, Homo sapiens
WGNWYQPYALPL

SEQ ID NO: 266 - protein, 12 aa, Homo sapiens
TLYWEQPYALPL

SEQ ID NO: 267 - protein, 12 aa, Homo sapiens
VWRWEQPYALPL

SEQ ID NO: 268 - protein, 11 aa, Homo sapiens
LLWTQPYALPL

SEQ ID NO: 269 - protein, 12 aa, Homo sapiens
  misc_feature 5, 6: Xaa at positions 5 and 6 is any amino acid
SRIWXXPYALPL

SEQ ID NO: 270 - protein, 12 aa, Homo sapiens
SDIWYQPYALPL

SEQ ID NO: 271 - protein, 12 aa, Homo sapiens
  misc_feature 5, 6: Xaa at positions 5 and 6 is any amino acid
WGYYXXPYALPL

SEQ ID NO: 272 - protein, 12 aa, Homo sapiens
TSGWYQPYALPL

SEQ ID NO: 273 - protein, 12 aa, Homo sapiens
  misc_feature 5, 6: Xaa at positions 5 and 6 is any amino acid
VHPYXXPYALPL

SEQ ID NO: 274 - protein, 12 aa, Homo sapiens
EHSYFQPYALPL

SEQ ID NO: 275 - protein, 12 aa, Homo sapiens
  misc_feature 1, 2: Xaa at positions 1 and 2 is any amino acid
XXIWYQPYALPL

SEQ ID NO: 276 - protein, 12 aa, Homo sapiens
AQLHSQPYALPL

SEQ ID NO: 277 - protein, 12 aa, Homo sapiens
WANWFQPYALPL

SEQ ID NO: 278 - protein, 12 aa, Homo sapiens
SRLYSQPYALPL

SEQ ID NO: 279 - protein, 12 aa, Homo sapiens
GVTFSQPYALPL

SEQ ID NO: 280 - protein, 12 aa, Homo sapiens
SIVWSQPYALPL

SEQ ID NO: 281 - protein, 12 aa, Homo sapiens
SRDLVQPYALPL

SEQ ID NO: 282 - protein, 17 aa, Homo sapiens
HWGHVYWQPYSVQDDLG

SEQ ID NO: 283 - protein, 17 aa, Homo sapiens
SWHSVYWQPYSVQSVPE

SEQ ID NO: 284 - protein, 17 aa, Homo sapiens
WRDSVYWQPYSVQPESA

SEQ ID NO: 285 - protein, 17 aa, Homo sapiens
TWDAVYWQPYSVQKWLD

SEQ ID NO: 286 - protein, 17 aa, Homo sapiens
TPPWVYWQPYSVQSLDP

SEQ ID NO: 287 - protein, 17 aa, Homo sapiens
YWSSVYWQPYSVQSVHS

SEQ ID NO: 288 - protein, 10 aa, Homo sapiens
YWYQPYALGL

SEQ ID NO: 289 - protein, 10 aa, Homo sapiens
YWYQPYALPL

SEQ ID NO: 290 - protein, 10 aa, Homo sapiens
EWIQPYATGL

SEQ ID NO: 291 - protein, 10 aa, Homo sapiens
NWEQPYAKPL

SEQ ID NO: 292 - protein, 10 aa, Homo sapiens
AFYQPYALPL

SEQ ID NO: 293 - protein, 10 aa, Homo sapiens
FLYQPYALPL

SEQ ID NO: 294 - protein, 10 aa, Homo sapiens
VCKQPYLEWC

SEQ ID NO: 295 - protein, 21 aa, Homo sapiens
ETPFTWEESNAYYWQPYALPL

SEQ ID NO: 296 - protein, 21 aa, Homo sapiens
QGWLTWQDSVDMYWQPYALPL

SEQ ID NO: 297 - protein, 21 aa, Homo sapiens
FSEAGYTWPENTYWQPYALPL

SEQ ID NO: 298 - protein, 21 aa, Homo sapiens
TESPGGLDWAKIYWQPYALPL

SEQ ID NO: 299 - protein, 21 aa, Homo sapiens
DGYDRWRQSGERYWQPYALPL

SEQ ID NO: 300 - protein, 21 aa, Homo sapiens
TANVSSFEWTPGYWQPYALPL

SEQ ID NO: 301 - protein, 21 aa, Homo sapiens
SVGEDHNFWTSEYWQPYALPL

SEQ ID NO: 302 - protein, 21 aa, Homo sapiens
MNDQTSEVSTFPYWQPYALPL

SEQ ID NO: 303 - protein, 21 aa, Homo sapiens
SWSEAFEQPRNLYWQPYALPL

SEQ ID NO: 304 - protein, 21 aa, Homo sapiens
QYAEPSALNDWGYWQPYALPL

SEQ ID NO: 305 - protein, 21 aa, Homo sapiens
NGDWATADWSNYYWQPYALPL

SEQ ID NO: 306 - protein, 15 aa, Homo sapiens
THDEHIYWQPYALPL

SEQ ID NO: 307 - protein, 21 aa, Homo sapiens
MLEKTYTTWTPGYWQPYALPL

SEQ ID NO: 308 - protein, 20 aa, Homo sapiens
WSDPLTRDADLYWQPYALPL

SEQ ID NO: 309 - protein, 21 aa, Homo sapiens
SDAFTTQDSQAMYWQPYALPL

SEQ ID NO: 310 - protein, 21 aa, Homo sapiens
GDDAAWRTDSLTYWQPYALPL

SEQ ID NO: 311 - protein, 21 aa, Homo sapiens
AIIRQLYRWSEMYWQPYALPL

SEQ ID NO: 312 - protein, 21 aa, Homo sapiens
ENTYSPNWADSMYWQPYALPL

SEQ ID NO: 313 - protein, 21 aa, Homo sapiens
MNDQTSEVSTFPYWQPYALPL

SEQ ID NO: 314 - protein, 21 aa, Homo sapiens
SVGEDHNFWTSEYWQPYALPL

SEQ ID NO: 315 - protein, 21 aa, Homo sapiens
QTPFTWEESNAYYWQPYALPL

SEQ ID NO: 316 - protein, 21 aa, Homo sapiens
ENPFTWQESNAYYWQPYALPL

SEQ ID NO: 317 - protein, 21 aa, Homo sapiens
VTPFTWEDSNVFYWQPYALPL

SEQ ID NO: 318 - protein, 21 aa, Homo sapiens
QIPFTWEQSNAYYWQPYALPL

SEQ ID NO: 319 - protein, 21 aa, Homo sapiens
QAPLTWQESAAYYWQPYALPL

SEQ ID NO: 320 - protein, 21 aa, Homo sapiens
EPTFTWEESKATYWQPYALPL

SEQ ID NO: 321 - protein, 21 aa, Homo sapiens
TTTLTWEESNAYYWQPYALPL

SEQ ID NO: 322 - protein, 21 aa, Homo sapiens
ESPLTWEESSALYWQPYALPL

SEQ ID NO: 323 - protein, 21 aa, Homo sapiens
ETPLTWEESNAYYWQPYALPL

SEQ ID NO: 324 - protein, 21 aa, Homo sapiens
EATFTWAESNAYYWQPYALPL

SEQ ID NO: 325 - protein, 21 aa, Homo sapiens
EALFTWKESTAYYWQPYALPL

SEQ ID NO: 326 - protein, 20 aa, Homo sapiens
STPTWEESNAYYWQPYALPL

SEQ ID NO: 327 - protein, 21 aa, Homo sapiens
ETPFTWEESNAYYWQPYALPL

SEQ ID NO: 328 - protein, 21 aa, Homo sapiens
KAPFTWEESQAYYWQPYALPL

SEQ ID NO: 329 - protein, 21 aa, Homo sapiens
STSFTWEESNAYYWQPYALPL

SEQ ID NO: 330 - protein, 21 aa, Homo sapiens
DSTFTWEESNAYYWQPYALPL

SEQ ID NO: 331 - protein, 21 aa, Homo sapiens
YIPFTWEESNAYYWQPYALPL

SEQ ID NO: 332 - protein, 21 aa, Homo sapiens
QTAFTWEESNAYYWQPYALPL

SEQ ID NO: 333 - protein, 21 aa, Homo sapiens
ETLFTWEESNATYWQPYALPL

SEQ ID NO: 334 - protein, 21 aa, Homo sapiens
VSSFTWEESNAYYWQPYALPL

SEQ ID NO: 335 - protein, 7 aa, Homo sapiens
QPYALPL

SEQ ID NO: 336 - protein, 9 aa, Homo sapiens
  misc_feature 4: Xaa at position 4 is any amino acid
PYQXYALPL

SEQ ID NO: 337 - protein, 21 aa, Homo sapiens
TANVSSFEWTPGYWQPYALPL

SEQ ID NO: 338 - protein, 15 aa, Homo sapiens
FEWTPGYWQPYALPL

SEQ ID NO: 339 - protein, 15 aa, Homo sapiens
  misc_feature 10: Xaa at position 10 is any amino acid
FEWTPGYWQXYALPL

SEQ ID NO: 340 - protein, 15 aa, Homo sapiens
  misc_feature 10: Xaa at position 10 is any amino acid
FEWTPGYYQXYALPL

SEQ ID NO: 341 - protein, 21 aa, Homo sapiens
ETPFTWEESNAYYWQPYALPL

SEQ ID NO: 342 - protein, 18 aa, Homo sapiens
  misc_feature 13: Xaa at position 13 is any amino acid
FTWEESNAYYWQXYALPL

SEQ ID NO: 343 - protein, 16 aa, Homo sapiens
ADVLYWQPYAPVTLWV

SEQ ID NO: 344 - protein, 17 aa, Homo sapiens
GDVAEYWQPYALPLTSL

SEQ ID NO: 345 - protein, 18 aa, Homo sapiens
SWTDYGYWQPYALPISGL

SEQ ID NO: 346 - protein, 15 aa, Homo sapiens
FEWTPGYWQPYALPL

SEQ ID NO: 347 - protein, 15 aa, Homo sapiens
  misc_feature 10: Xaa at position 10 is any amino acid
FEWTPGYWQXYALPL

SEQ ID NO: 348 - protein, 15 aa, Homo sapiens
FEWTPGWYQPYALPL

SEQ ID NO: 349 - protein, 15 aa, Homo sapiens
  misc_feature 10: Xaa at position 10 is any amino acid
FEWTPGWYQXYALPL

SEQ ID NO: 350 - protein, 15 aa, Homo sapiens
FEWTPGYYQPYALPL

SEQ ID NO: 351 - protein, 15 aa, Homo sapiens
  misc_feature 10: Xaa at position 10 is any amino acid
FEWTPGYYQXYALPL

SEQ ID NO: 352 - protein, 21 aa, Homo sapiens
TANVSSFEWTPGYWQPYALPL

SEQ ID NO: 353 - protein, 18 aa, Homo sapiens
SWTDYGYWQPYALPISGL

SEQ ID NO: 354 - protein, 21 aa, Homo sapiens
ETPFTWEESNAWAIQPYALPL

SEQ ID NO: 355 - protein, 21 aa, Homo sapiens
ENTYSPNWADSMYWQPYALPL

SEQ ID NO: 356 - protein, 21 aa, Homo sapiens
SVGEDHNFWTSEYWQPYALPL

SEQ ID NO: 357 - protein, 21 aa, Homo sapiens
DGYDRWRQSGERYWQPYALPL

SEQ ID NO: 358 - protein, 15 aa, Homo sapiens
FEWTPGYWQPYALPL

SEQ ID NO: 359 - protein, 11 aa, Homo sapiens
FEWTPGYWQPY

SEQ ID NO: 360 - protein, 11 aa, Homo sapiens
  misc_feature 10: Xaa at position 10 is any amino acid
FEWTPGYWQXY

SEQ ID NO: 361 - protein, 10 aa, Homo sapiens
EWTPGYWQPY

SEQ ID NO: 362 - protein, 11 aa, Homo sapiens
  misc_feature 10: Xaa at position 10 is any amino acid
FEWTPGWYQXY

SEQ ID NO: 363 - protein, 11 aa, Homo sapiens
  misc_feature 10: Xaa at position 10 is any amino acid
AEWTPGYWQXY

SEQ ID NO: 364 - protein, 11 aa, Homo sapiens
  misc_feature 10: Xaa at position 10 is any amino acid
FAWTPGYWQXY

SEQ ID NO: 365 - protein, 11 aa, Homo sapiens
  misc_feature 10: Xaa at position 10 is any amino acid
FEATPGYWQXY

SEQ ID NO: 366 - protein, 11 aa, Homo sapiens
  misc_feature 10: Xaa at position 10 is any amino acid
FEWAPGYWQXY

SEQ ID NO: 367 - protein, 11 aa, Homo sapiens
  misc_feature 10: Xaa at position 10 is any amino acid
FEWTAGYWQXY

SEQ ID NO: 368 - protein, 11 aa, Homo sapiens
  misc_feature 10: Xaa at position 10 is any amino acid
FEWTPAYWQXY

SEQ ID NO: 369 - protein, 11 aa, Homo sapiens
  misc_feature 10: Xaa at position 10 is any amino acid
FEWTPGAWQXY

SEQ ID NO: 370 - protein, 11 aa, Homo sapiens
  misc_feature 10: Xaa at position 10 is any amino acid
FEWTPGYAQXY

SEQ ID NO: 371 - protein, 11 aa, Homo sapiens
  misc_feature 10: Xaa at position 10 is any amino acid
FEWTPGYWQXA

SEQ ID NO: 372 - protein, 11 aa, Homo sapiens
  misc_feature 10: Xaa at position 10 is any amino acid
FEWTGGYWQXY

SEQ ID NO: 373 - protein, 11 aa, Homo sapiens
  misc_feature 10: Xaa at position 10 is any amino acid
FEWTPGYWQXY

SEQ ID NO: 374 - protein, 11 aa, Homo sapiens
  misc_feature 10: Xaa at positions 5 and 10 is any amino acid
FEWTXGYWQXY

SEQ ID NO: 375 - protein, 14 aa, Homo sapiens
  misc_feature 13: Xaa at position 13 is any amino acid
FEVFFPECGYWQXY

SEQ ID NO: 376 - protein, 12 aa, Homo sapiens
  misc_feature 11: Xaa at position 11 is any amino acid
FEWTPAIYWQXY

SEQ ID NO: 377 - protein, 13 aa, Homo sapiens
  misc_feature 12: Xaa at position 12 is any amino acid
FEWTPSARWYQXY

SEQ ID NO: 378 - protein, 13 aa, Homo sapiens
  misc_feature 12: Xaa at position 12 is any amino acid
FEWTSARGYWQXY

SEQ ID NO: 379 - protein, 11 aa, Homo sapiens
  misc_feature 10: Xaa at position 10 is any amino acid
FEWTPNYWQXY

SEQ ID NO: 380 - protein, 11 aa, Homo sapiens
  misc_feature 10: Xaa at position 10 is any amino acid
FEWTPVYWQXY

SEQ ID NO: 381 - protein, 11 aa, Homo sapiens
  misc_feature 10: Xaa at position 10 is any amino acid
FEWTVPYWQXY

SEQ ID NO: 382 - protein, 12 aa, Homo sapiens
  misc_feature 11: Xaa at position 11 is any amino acid
FEWTPGVVYQXY

SEQ ID NO: 383 - protein, 12 aa, Homo sapiens
  misc_feature 11: Xaa at position 11 is any amino acid
FEVFFPGYWQXY

SEQ ID NO: 384 - protein, 11 aa, Homo sapiens
  misc_feature 10: Xaa at position 10 is any amino acid
EVFFPGYYQXY

SEQ ID NO: 385 - protein, 11 aa, Homo sapiens
  misc_feature 10: Xaa at position 10 is any amino acid
YEWTPGYYQXY

SEQ ID NO: 386 - protein, 11 aa, Homo sapiens
  misc_feature 10: Xaa at position 10 is any amino acid
FEWVPGYYQXY

SEQ ID NO: 387 - protein, 12 aa, Homo sapiens
  misc_feature 10: Xaa at position 10 is any amino acid
FEVFFPGYYQXY

SEQ ID NO: 388 - protein, 12 aa, Homo sapiens
  misc_feature 10: Xaa at position 10 is any amino acid
FEVFFPSYYQXY

SEQ ID NO: 389 - protein, 11 aa, Homo sapiens
  misc_feature 10: Xaa at position 10 is any amino acid
FEWTPNYYQXY

SEQ ID NO: 390 - protein, 12 aa, Homo sapiens
  misc_feature 5: Xaa at position 5 is any amino acid
SHLYXQPYSVQM

SEQ ID NO: 391 - protein, 13 aa, Homo sapiens
  misc_feature 5: Xaa at position 5 is any amino acid
TLVYXLDPYSLQT

SEQ ID NO: 392 - protein, 12 aa, Homo sapiens
  misc_feature 5: Xaa at position 5 is any amino acid
RGDYXQPYSVQS

SEQ ID NO: 393 - protein, 12 aa, Homo sapiens
  misc_feature 5: Xaa at position 5 is any amino acid
NMVYXQPYSIQT

SEQ ID NO: 394 - protein, 9 aa, Homo sapiens
VYWQPYSVQ

SEQ ID NO: 395 - protein, 9 aa, Homo sapiens
  misc_feature 3: Xaa at position 3 is any amino acid
VYXQPYSVQ

SEQ ID NO: 396 - protein, 12 aa, Homo sapiens
  misc_feature 7: Xaa at position 7 is any amino acid
TFVYWQXYALPL

SEQ ID NO: 397 - protein, 10 aa, Homo sapiens
  misc_feature 10: Xaa at position 10 is any amino acid
FEWTPGYYQX

SEQ ID NO: 398 - protein, 11 aa, Homo sapiens
  misc_feature 11: Xaa at position 11 is any amino acid
XFEWTPGYYQX

SEQ ID NO: 399 - protein, 10 aa, Homo sapiens
  misc_feature 9: Xaa at position 9 is any amino acid
FEWTPGYQXY

SEQ ID NO: 400 - protein, 10 aa, Homo sapiens
  misc_feature 9: Xaa at position 9 is any amino acid
FEWTPGYQXY

SEQ ID NO: 401 - protein, 10 aa, Homo sapiens
  misc_feature 9: Xaa at position 9 is any amino acid
FEWTPGYQXY

SEQ ID NO: 402 - protein, 10 aa, Homo sapiens
  misc_feature 9: Xaa at position 9 is any amino acid
FEWTPGYQXY

SEQ ID NO: 403 - protein, 10 aa, Homo sapiens
  misc_feature 9: Xaa at position 9 is any amino acid
FETPGYYQXY

SEQ ID NO: 404 - protein, 10 aa, Homo sapiens
  misc_feature 9: Xaa at position 9 is any amino acid
FETPGYYQXY

SEQ ID NO: 405 - protein, 10 aa, Homo sapiens
  misc_feature 9: Xaa at position 9 is any amino acid
EWTPGYYQXY

SEQ ID NO: 406 - protein, 10 aa, Homo sapiens
  misc_feature 9: Xaa at position 9 is any amino acid
EWTPGYYQXY

SEQ ID NO: 407 - protein, 9 aa, Homo sapiens
VYWQPYSVQ

SEQ ID NO: 408 - protein, 12 aa, Homo sapiens
RLVYWQPYSVQR

SEQ ID NO: 409 - protein, 11 aa, Homo sapiens
RLVYQPYSVQR

SEQ ID NO: 410 - protein, 12 aa, Homo sapiens
RLDYWQPYSVQR

SEQ ID NO: 411 - protein, 12 aa, Homo sapiens
RLVWFQPYSVQR

SEQ ID NO: 412 - protein, 12 aa, Homo sapiens
RLVYWQPYSIQR

SEQ ID NO: 413 - protein, 11 aa, Homo sapiens
DNSSWYDSFLL

SEQ ID NO: 414 - protein, 11 aa, Homo sapiens
DNTAWYESFLA

SEQ ID NO: 415 - protein, 11 aa, Homo sapiens
DNTAWYENFLL

SEQ ID NO: 416 - protein, 17 aa, Homo sapiens
PAREDNTAWYDSFLIWC

SEQ ID NO: 417 - protein, 17 aa, Homo sapiens
TSEYDNTTWYEKFLASQ

SEQ ID NO: 418 - protein, 17 aa, Homo sapiens
SQIPDNTAWYQSFLLHG

SEQ ID NO: 419 - protein, 17 aa, Homo sapiens
SPFIDNTAWYENFLLTY

SEQ ID NO: 420 - protein, 17 aa, Homo sapiens
EQIYDNTAWYDHFLLSY

SEQ ID NO: 421 - protein, 17 aa, Homo sapiens
TPFIDNTAWYENFLLTY

SEQ ID NO: 422 - protein, 17 aa, Homo sapiens
TYTYDNTAWYERFLMSY

SEQ ID NO: 423 - protein, 17 aa, Homo sapiens
TMTQDNTAWYENFLLSY

SEQ ID NO: 424 - protein, 17 aa, Homo sapiens
TIDNTAWYANLVQTYPQ

SEQ ID NO: 425 - protein, 17 aa, Homo sapiens
TIDNTAWYERFLAQYPD

SEQ ID NO: 426 - protein, 17 aa, Homo sapiens
HIDNTAWYENFLLTYTP

SEQ ID NO: 427 - protein, 17 aa, Homo sapiens
SQDNTAWYENFLLSYKA

SEQ ID NO: 428 - protein, 17 aa, Homo sapiens
QIDNTAWYERFLLQYNA

SEQ ID NO: 429 - protein, 17 aa, Homo sapiens
NQDNTAWYESFLLQYNT

SEQ ID NO: 430 - protein, 17 aa, Homo sapiens
TIDNTAWYENFLLNHNL

SEQ ID NO: 431 - protein, 17 aa, Homo sapiens
HYDNTAWYERFLQQGWH

SEQ ID NO: 432 - protein, 21 aa, Homo sapiens
ETPFTWEESNAYYWQPYALPL

SEQ ID NO: 433 - protein, 21 aa, Homo sapiens
YIPFTWEESNAYYWQPYALPL

SEQ ID NO: 434 - protein, 21 aa, Homo sapiens
DGYDRWRQSGERYWQPYALPL

SEQ ID NO: 435 - protein, 10 aa, Homo sapiens
  misc_feature 5: Xaa at position 5 is any amino acid
YIYQXYALPL

SEQ ID NO: 436 - protein, 21 aa, Homo sapiens
TANVSSFEWTPGYWQPYALPL

SEQ ID NO: 437 - protein, 15 aa, Homo sapiens
  misc_feature 10: Xaa at position 10 is any amino acid
FEWTPGYWQXYALPL

SEQ ID NO: 438 - protein, 17 aa, Homo sapiens
FEWTPGYWQPYALPLSD

SEQ ID NO: 439 - protein, 15 aa, Homo sapiens
  misc_feature 10: Xaa at position 10 is any amino acid
FEWTPGYYQXYALPL

SEQ ID NO: 440 - protein, 11 aa, Homo sapiens
  misc_feature 10: Xaa at position 10 is any amino acid
FEWTPGYWQXY

SEQ ID NO: 441 - protein, 12 aa, Homo sapiens
  misc_feature 10: Xaa at position 11 is any amino acid
FEVVTPGYWQXY

SEQ ID NO: 442 - protein, 11 aa, Homo sapiens
  misc_feature 10: Xaa at position 10 is any amino acid
FEWTPGWYQXY

SEQ ID NO: 443 - protein, 11 aa, Homo sapiens
  misc_feature 10: Xaa at position 10 is any amino acid
FEWTPGYYQXY

SEQ ID NO: 444 - protein, 10 aa, Homo sapiens
  misc_feature 9: Xaa at position 9 is any amino acid
FEWTPYWQXY

SEQ ID NO: 445 - protein, 10 aa, Homo sapiens
  misc_feature 9: Xaa at position 9 is any amino acid
FEWTPWYQXY

SEQ ID NO: 446 - protein, 10 aa, Homo sapiens
  misc_feature 9: Xaa at position 9 is any amino acid
FEWTPYYQXY

SEQ ID NO: 447 - protein, 15 aa, Homo sapiens
  misc_feature 10: Xaa at position 10 is any amino acid
FEWTPGYYQXYALPL

SEQ ID NO: 448 - protein, 15 aa, Homo sapiens
  misc_feature 10: Xaa at position 10 is any amino acid
FEWTPGYWQXYALPL

SEQ ID NO: 449 - protein, 15 aa, Homo sapiens
  misc_feature 10: Xaa at position 10 is any amino acid
FEWTPGWYQXYALPL

SEQ ID NO: 450 - protein, 21 aa, Homo sapiens
TANVSSFEWTPGYWQPYALPL

SEQ ID NO: 451 - protein, 11 aa, Homo sapiens
  misc_feature 10: Xaa at position 10 is any amino acid
FEWTPGYWQXY

SEQ ID NO: 452 - protein, 11 aa, Homo sapiens
  misc_feature 10: Xaa at position 10 is any amino acid
FEWTPGWYQXY

SEQ ID NO: 453 - protein, 11 aa, Homo sapiens
  misc_feature 10: Xaa at position 10 is any amino acid
FEWTPGYYQXY

SEQ ID NO: 454 - protein, 11 aa, Homo sapiens
  misc_feature
  Xaa at position 10 is any amino acid
FEWTPAYWQXY

SEQ ID NO: 455 - protein, 11 aa, Homo sapiens
  misc_feature
  Xaa at position 10 is any amino acid
FEWTPAWYQXY

SEQ ID NO: 456 - protein, 11 aa, Homo sapiens
  misc_feature
  Xaa at position 10 is any amino acid
FEWTPAYYQXY

SEQ ID NO: 457 - protein, 13 aa, Homo sapiens
EGPTLRQWLAARA

SEQ ID NO: 458 - protein, 14 aa, Homo sapiens
IEGPTLRQWLAAKA

SEQ ID NO: 459 - protein, 14 aa, Homo sapiens
IEGPTLREWLAARA

SEQ ID NO: 460 - protein, 14 aa, Homo sapiens
IEGPTLRQWLAARA

SEQ ID NO: 461 - protein, 14 aa, Homo sapiens
IEGPTLRQWLAAKA

SEQ ID NO: 462 - protein, 14 aa, Homo sapiens
IEGPTLRQCLAARA

SEQ ID NO: 463 - protein, 14 aa, Homo sapiens
IEGPTLRQWLAARA

SEQ ID NO: 464 - protein, 14 aa, Homo sapiens
IEGPTLRQWLAARA

SEQ ID NO: 465 - protein, 14 aa, Homo sapiens
IEGPTLRQCLAARA

SEQ ID NO: 466 - protein, 14 aa, Homo sapiens
IEGPTLRQCLAARA

SEQ ID NO: 467 - protein, 14 aa, Homo sapiens
IEGPTLRQWLAARA

SEQ ID NO: 468 - protein, 9 aa, Homo sapiens
  misc_feature 6, 7, 8: Xaa at positions 6, 7, and 8 is any amino acid
VRDQIXXXL

SEQ ID NO: 469 - protein, 6 aa, Homo sapiens
TLREWL

SEQ ID NO: 470 - protein, 10 aa, Homo sapiens
GRVRDQVAGW

SEQ ID NO: 471 - protein, 10 aa, Homo sapiens
GRVKDQIAQL

SEQ ID NO: 472 - protein, 10 aa, Homo sapiens
GVRDQVSWAL

SEQ ID NO: 473 - protein, 10 aa, Homo sapiens
ESVREQVMKY

SEQ ID NO: 474 - protein, 10 aa, Homo sapiens
SVRSQISASL

SEQ ID NO: 475 - protein, 10 aa, Homo sapiens
GVRETVYRHM

SEQ ID NO: 476 - protein, 11 aa, Homo sapiens
GVREVIVMHML

SEQ ID NO: 477 - protein, 11 aa, Homo sapiens
GRVRDQIWAAL

SEQ ID NO: 478 - protein, 11 aa, Homo sapiens
AGVRDQILIWL

SEQ ID NO: 479 - protein, 11 aa, Homo sapiens
GRVRDQIMLSL

SEQ ID NO: 480 - protein, 11 aa, Homo sapiens
  misc_feature 8, 9, 10: Xaa at positions 8, 9, and 10 is any amino acid
GRVRDQIXXXL

SEQ ID NO: 481 - protein, 10 aa, Homo sapiens
CTLRQWLQGC

SEQ ID NO: 482 - protein, 10 aa, Homo sapiens
CTLQEFLEGC

SEQ ID NO: 483 - protein, 10 aa, Homo sapiens
CTRTEWLHGC

SEQ ID NO: 484 - protein, 12 aa, Homo sapiens
CTLREWLHGGFC

SEQ ID NO: 485 - protein, 12 aa, Homo sapiens
CTLREWVFAGLC

SEQ ID NO: 486 - protein, 13 aa, Homo sapiens
CTLRQWLILLGMC

SEQ ID NO: 487 - protein, 14 aa, Homo sapiens
CTLAEFLASGVEQC

SEQ ID NO: 488 - protein, 14 aa, Homo sapiens
CSLQEFLSHGGYVC

SEQ ID NO: 489 - protein, 14 aa, Homo sapiens
CTLREFLDPTTAVC

SEQ ID NO: 490 - protein, 14 aa, Homo sapiens
CTLKEWLVSHEVWC

SEQ ID NO: 491 - protein, 10 aa, Homo sapiens
  misc_feature 8, 9: Xaa at positions 8 and 9 is any amino acid
CTLREWLXXC

SEQ ID NO: 492 - protein, 11 aa, Homo sapiens
  misc_feature 8, 9, 10: Xaa at positions 8, 9, and 10 is any amino acid
CTLREWLXXXC

SEQ ID NO: 493 - protein, 12 aa, Homo sapiens
  misc_feature 8, 9, 10: Xaa at positions 8, 9, 10, and 11 is any amino acid
CTLREWLXXXXC

SEQ ID NO: 494 - protein, 13 aa, Homo sapiens
  misc_feature 8, 9, 10, 11, 12: Xaa at positions 8, 9, 10, 11, and 12 is any amino acid
CTLREWLXXXXXC

SEQ ID NO: 495 - protein, 14 aa, Homo sapiens
  misc_feature 8, 9, 10, 11, 12, 13: Xaa at positions 8, 9, 10, 11, 12, and 13 is any amino acid
CTLREWLXXXXXXC

SEQ ID NO: 496 - protein, 10 aa, Homo sapiens
REGPTLRQWM

SEQ ID NO: 497 - protein, 10 aa, Homo sapiens
EGPTLRQWLA

SEQ ID NO: 498 - protein, 10 aa, Homo sapiens
ERGPFWAKAC

SEQ ID NO: 499 - protein, 10 aa, Homo sapiens
REGPRCVMWM

SEQ ID NO: 500 - protein, 14 aa, Homo sapiens
CGTEGPTLSTWLDC

SEQ ID NO: 501 - protein, 14 aa, Homo sapiens
CEQDGPTLLEWLKC

SEQ ID NO: 502 - protein, 14 aa, Homo sapiens
CELVGPSLMSWLTC

SEQ ID NO: 503 - protein, 14 aa, Homo sapiens
CLTGPFVTQWLYEC

SEQ ID NO: 504 - protein, 14 aa, Homo sapiens
CRAGPTLLEWLTLC

SEQ ID NO: 505 - protein, 14 aa, Homo sapiens
CADGPTLREWISFC

SEQ ID NO: 506 - protein, 13 aa, Homo sapiens
  misc_feature 2, 12: Xaa at positions 2 and 12 is any amino acid
CXEGPTLREWLXC

SEQ ID NO: 507 - protein, 14 aa, Homo sapiens
  misc_feature 2, 12, 13: Xaa at positions 2, 12 and 13 is any amino acid
CXEGPTLREWLXXC

SEQ ID NO: 508 - protein, 14 aa, Homo sapiens
  misc_feature 2, 3, 13: Xaa at positions 2, 3, and 13 is any amino acid
CXXEGPTLREWLXC

SEQ ID NO: 509 - protein, 15 aa, Homo sapiens
  misc_feature 2, 3, 13, 14: Xaa at positions 2, 3, 13, and 14 is any amino acid
CXXEGPTLREWLXXC

SEQ ID NO: 510 - protein, 13 aa, Homo sapiens
  misc_feature 2, 12: Xaa at positions 2 and 12 is any amino acid
CXEGPTLREWLXC

SEQ ID NO: 511 - protein, 16 aa, Homo sapiens
GGCTLREWLHGGFCGG

SEQ ID NO: 512 - protein, 18 aa, Homo sapiens
GGCADGPTLREWISFCGG

SEQ ID NO: 513 - protein, 19 aa, Homo sapiens
GNADGPTLRQWLEGRRPKN

SEQ ID NO: 514 - protein, 19 aa, Homo sapiens
LAIEGPTLRQWLHGNGRDT

SEQ ID NO: 515 - protein, 19 aa, Homo sapiens
HGRVGPTLREWKTQVATKK

SEQ ID NO: 516 - protein, 18 aa, Homo sapiens
TIKGPTLRQWLKSREHTS

SEQ ID NO: 517 - protein, 18 aa, Homo sapiens
ISDGPTLKEWLSVTRGAS

SEQ ID NO: 518 - protein, 18 aa, Homo sapiens
SIEGPTLREWLTSRTPHS

SEQ ID NO: 519 - protein, 5 aa, Homo sapiens
EEDCK

SEQ ID NO: 520 - protein, 5 aa, Homo sapiens
EEDCK

SEQ ID NO: 521 - protein, 5 aa, Homo sapiens
EEDCK

SEQ ID NO: 522 - protein, 5 aa, Homo sapiens
  misc_feature 4: Xaa at position 4 is any amino acid
EEDXK

SEQ ID NO: 523 - protein, 5 aa, Homo sapiens
EEDCK

SEQ ID NO: 524 - protein, 5 aa, Homo sapiens
  misc_feature 4: Xaa at position 4 is any amino acid
EEDXK

SEQ ID NO: 525 - protein, 11 aa, Homo sapiens
YCFTASENHCY

SEQ ID NO: 526 - protein, 11 aa, Homo sapiens
YCFTNSENHCY

SEQ ID NO: 527 - protein, 11 aa, Homo sapiens
YCFTRSENHCY

SEQ ID NO: 528 - protein, 9 aa, Homo sapiens
FCASENHCY

SEQ ID NO: 529 - protein, 9 aa, Homo sapiens
YCASENHCY

SEQ ID NO: 530 - protein, 9 aa, Homo sapiens
FCNSENHCY

SEQ ID NO: 531 - protein, 9 aa, Homo sapiens
FCNSENRCY

SEQ ID NO: 532 - protein, 10 aa, Homo sapiens
FCNSVENRCY

SEQ ID NO: 533 - protein, 11 aa, Homo sapiens
YCSQSVSNDCF

SEQ ID NO: 534 - protein, 9 aa, Homo sapiens
FCVSNDRCY

SEQ ID NO: 535 - protein, 11 aa, Homo sapiens
YCRKELGQVCY

SEQ ID NO: 536 - protein, 9 aa, Homo sapiens
YCKEPGQCY

SEQ ID NO: 537 - protein, 9 aa, Homo sapiens
YCRKEMGCY

SEQ ID NO: 538 - protein, 9 aa, Homo sapiens
FCRKEMGCY

SEQ ID NO: 539 - protein, 9 aa, Homo sapiens
YCWSQNLCY

SEQ ID NO: 540 - protein, 10 aa, Homo sapiens
YCELSQYLCY

SEQ ID NO: 541 - protein, 9 aa, Homo sapiens
YCWSQNYCY

SEQ ID NO: 542 - protein, 9 aa, Homo sapiens
YCWSQYLCY

SEQ ID NO: 543 - protein, 15 aa, Homo sapiens
DFLPHYKNTSLGHRP

SEQ ID NO: 544 - protein, 7 aa, Homo sapiens
  misc_feature 2, 7: Xaa at positions 2 and 7 is any amino acid
RXETXWX

SEQ ID NO: 545 - protein, 7 aa, Homo sapiens
  misc_feature 2, 5: Xaa at positions 2 and 5 is any amino acid
RXETXWX

SEQ ID NO: 546 - protein, 5 aa, Homo sapiens
  misc_feature 5: Xaa at position 5 is any amino acid
RGDGX

SEQ ID NO: 547 - protein, 7 aa, Homo sapiens
  misc_feature 6: Xaa at position 6 is any amino acid
CRGDGXC

SEQ ID NO: 548 - protein, 9 aa, Homo sapiens
  misc_feature 2, 3, 7, 8: Xaa at positions 2, 3, 7 and 8 is any amino acid
CXXRLDXXC

SEQ ID NO: 549 - protein, 9 aa, Homo sapiens
CARRLDAPC

SEQ ID NO: 550 - protein, 9 aa, Homo sapiens
CPSRLDSPC

SEQ ID NO: 551 - protein, 9 aa, Homo sapiens
  misc_feature 1, 2, 3, 7, 8, 9: Xaa at positions 1, 2, 3, 7, 8, and 9 is any amino acid
XXXRGDXXX

SEQ ID NO: 552 - protein, 9 aa, Homo sapiens
  misc_feature 2, 8: Xaa at positions 2 and 8 is any amino acid
CXCRGDCXC

SEQ ID NO: 553 - protein, 9 aa, Homo sapiens
CDCRGDCFC

SEQ ID NO: 554 - protein, 9 aa, Homo sapiens
CDCRGDCLC

SEQ ID NO: 555 - protein, 9 aa, Homo sapiens
CLCRGDCIC

SEQ ID NO: 556 - protein, 8 aa, Homo sapiens
  misc_feature 1, 2, 5, 6, 7, 8: Xaa at positions 1, 2, 5, 6, 7, and 8 is any amino acid
XXDDXXXX

SEQ ID NO: 557 - protein, 10 aa, Homo sapiens
  misc_feature 1, 2, 3, 6, 7, 8, 9, 10: Xaa at positions 1, 2, 3, and 6, 7, 8, 9, and 10 is any amino acid
XXXDDXXXXX

SEQ ID NO: 558 - protein, 7 aa, Homo sapiens
CWDDGWL

SEQ ID NO: 559 - protein, 9 aa, Homo sapiens
CWDDLWWLC

SEQ ID NO: 560 - protein, 8 aa, Homo sapiens
CWDDGLMC

SEQ ID NO: 561 - protein, 8 aa, Homo sapiens
CWDDGWMC

SEQ ID NO: 562 - protein, 9 aa, Homo sapiens
CSWDDGWLC

SEQ ID NO: 563 - protein, 9 aa, Homo sapiens
CPDDLWWLC

SEQ ID NO: 564 - protein, 13 aa, Homo sapiens
CGRECPRLCQSSC

SEQ ID NO: 565 - protein, 13 aa, Homo sapiens
CNGRCVSGCAGRC

SEQ ID NO: 566 - protein, 8 aa, Homo sapiens
CLSGSLSC

SEQ ID NO: 567 - protein, 6 aa, Homo sapiens
NGRAHA

SEQ ID NO: 568 - protein, 5 aa, Homo sapiens
CNGRC

SEQ ID NO: 569 - protein, 9 aa, Homo sapiens
CDCRGDCFC

SEQ ID NO: 570 - protein, 7 aa, Homo sapiens
CGSLVRC

SEQ ID NO: 571 - protein, 5 aa, Homo sapiens
  misc_feature 3, 4: Xaa at positions 3 and 4 is any amino acid
DLXXL

SEQ ID NO: 572 - protein, 12 aa, Homo sapiens
RTDLDSLRTYTL

SEQ ID NO: 573 - protein, 10 aa, Homo sapiens
RTDLDSLRTY

SEQ ID NO: 574 - protein, 9 aa, Homo sapiens
RTDLDSLRT

SEQ ID NO: 575 - protein, 8 aa, Homo sapiens
RTDLDSLR

SEQ ID NO: 576 - protein, 12 aa, Homo sapiens
GDLDLLKLRLTL

SEQ ID NO: 577 - protein, 12 aa, Homo sapiens
GDLHSLRQLLSR

SEQ ID NO: 578 - protein, 12 aa, Homo sapiens
RDDLHMLRLQLW

SEQ ID NO: 579 - protein, 12 aa, Homo sapiens
SSDLHALKKRYG

SEQ ID NO: 580 - protein, 12 aa, Homo sapiens
RGDLKQLSELTW

SEQ ID NO: 581 - protein, 12 aa, Homo sapiens
RGDLAALSAPPV

SEQ ID NO: 582 - protein, 12 aa, Homo sapiens
DITWDQLWDLMK

SEQ ID NO: 583 - protein, 12 aa, Homo sapiens
DITWDELWKIMN

SEQ ID NO: 584 - protein, 12 aa, Homo sapiens
DYTWFELWDMMQ

SEQ ID NO: 585 - protein, 12 aa, Homo sapiens
QITWAQLWNMMK

SEQ ID NO: 586 - protein, 12 aa, Homo sapiens
DMTWHDLWTLMS

SEQ ID NO: 587 - protein, 12 aa, Homo sapiens
DYSWHDLWEMMS

SEQ ID NO: 588 - protein, 12 aa, Homo sapiens
EITWDQLWEVMN

SEQ ID NO: 589 - protein, 12 aa, Homo sapiens
HVSWEQLWDIMN

SEQ ID NO: 590 - protein, 12 aa, Homo sapiens
HITWDQLWRIMT

SEQ ID NO: 591 - protein, 13 aa, Homo sapiens
RNMSWLELWEHMK

SEQ ID NO: 592 - protein, 18 aa, Homo sapiens
AEWTWDQLWHVMNPAESQ

SEQ ID NO: 593 - protein, 14 aa, Homo sapiens
HRAEWLALWEQMSP

SEQ ID NO: 594 - protein, 14 aa, Homo sapiens
KKEDWLALWRIMSV

SEQ ID NO: 595 - protein, 11 aa, Homo sapiens
ITWDQLWDLMK

SEQ ID NO: 596 - protein, 12 aa, Homo sapiens
DITWDQLWDLMK

SEQ ID NO: 597 - protein, 12 aa, Homo sapiens
DITWDQLWDLMK

SEQ ID NO: 598 - protein, 12 aa, Homo sapiens
DITWDQLWDLMK

SEQ ID NO: 599 - protein, 16 aa, Homo sapiens
CQNRYTDLVAIQNKNE

SEQ ID NO: 600 - protein, 17 aa, Homo sapiens
AENWADNEPNNKRNNED

SEQ ID NO: 601 - protein, 19 aa, Homo sapiens
RKNNKTWTWVGTKKALTNE

SEQ ID NO: 602 - protein, 13 aa, Homo sapiens
KKALTNEAENWAD

SEQ ID NO: 603 - protein, 16 aa, Homo sapiens
  misc_feature 3, 15: Xaa at positions 3 and 15 is any amino acid
CQXRYTDLVAIQNKXE

SEQ ID NO: 604 - protein, 19 aa, Homo sapiens
  misc_feature 3, 5, 6, 13, 15: Xaa at positions 3, 5, 6, 13 and 15 is any amino acid
RKXNXXWTWVGTXKXLTEE

SEQ ID NO: 605 - protein, 17 aa, Homo sapiens
  misc_feature 13, 15: Xaa at positions 13 and 15 is any amino acid
AENWADGEPNNKXNXED

SEQ ID NO: 606 - protein, 16 aa, Homo sapiens
  misc_feature 2, 3, 4, 7, 15: Xaa at positions 2, 3, 4, 7, and 15 is any amino acid
CXXXYTXLVAIQNKXE

SEQ ID NO: 607 - protein, 19 aa, Homo sapiens
  misc_feature 3-6, 8, 13, 15 and 18: Xaa at positions 3-6, 8, 13, 15 and 18 is any amino acid
RKXXXXWXWVGTXKXLTXE

SEQ ID NO: 608 - protein, 16 aa, Homo sapiens
  misc_feature 2, 5, 6, 7, 12, 13 and 14: Xaa at positions 2, 5, 6, 7, 12, 13 and 14 is any amino acid
AXNWXXXEPNNXXXED

SEQ ID NO: 609 - protein, 13 aa, Homo sapiens
  misc_feature 1, 3, 6, 9, 12, and 13: Xaa at positions 1, 3, 6, 9, 12, and 13 is any amino acid
XKXKTXEAXNWXX

SEQ ID NO: 610 - protein, 33 aa, Homo sapiens
GFFALIPKIISSPLFKTLLSAVGSALSSSGGQQ

SEQ ID NO: 611 - protein, 33 aa, Homo sapiens
GFFALIPKIISSPLFKTLLSAVGSALSSSGGQE

SEQ ID NO: 612 - protein, 22 aa, Homo sapiens
GFFALIPKIISSPLFKTLLSAV

SEQ ID NO: 613 - protein, 22 aa, Homo sapiens
GFFALIPKIISSPLFKTLLSAV

SEQ ID NO: 614 - protein, 23 aa, Homo sapiens
KGFFALIPKIISSPLFKTLLSAV

SEQ ID NO: 615 - protein, 24 aa, Homo sapiens
KKGFFALIPKIISSPLFKTLLSAV

SEQ ID NO: 616 - protein, 24 aa, Homo sapiens
KKGFFALIPKIISSPLFKTLLSAV

SEQ ID NO: 617 - protein, 11 aa, Homo sapiens
GFFALIPKIIS

SEQ ID NO: 618 - protein, 26 aa, Homo sapiens
GIGAVLKVLTTGLPALISWIKRKRQQ

SEQ ID NO: 619 - protein, 26 aa, Homo sapiens
GIGAVLKVLTTGLPALISWIKRKRQQ

SEQ ID NO: 620 - protein, 26 aa, Homo sapiens
GIGAVLKVLTTGLPALISWIKRKRQQ

SEQ ID NO: 621 - protein, 22 aa, Homo sapiens
GIGAVLKVLTTGLPALISWIKR

SEQ ID NO: 622 - protein, 19 aa, Homo sapiens
AVLKVLTTGLPALISWIKR

SEQ ID NO: 623 - protein, 12 aa, Homo sapiens
KLLLLLKLLLLK

SEQ ID NO: 624 - protein, 12 aa, Homo sapiens
KLLLKLLLKLLK

SEQ ID NO: 625 - protein, 12 aa, Homo sapiens
KLLLKLKLKLLK

SEQ ID NO: 626 - protein, 12 aa, Homo sapiens
KKLLKLKLKLKK

SEQ ID NO: 627 - protein, 12 aa, Homo sapiens
KLLLKLLLKLLK

SEQ ID NO: 628 - protein, 12 aa, Homo sapiens
KLLLKLKLKLLK

SEQ ID NO: 629 - protein, 6 aa, Homo sapiens
KLLLLK

SEQ ID NO: 630 - protein, 8 aa, Homo sapiens
KLLLKLLK

SEQ ID NO: 631 - protein, 12 aa, Homo sapiens
KLLLKLKLKLLK

SEQ ID NO: 632 - protein, 12 aa, Homo sapiens
KLLLKLKLKLLK

SEQ ID NO: 633 - protein, 12 aa, Homo sapiens
KLLLKLKLKLLK

SEQ ID NO: 634 - protein, 12 aa, Homo sapiens
KAAAKAAAKAAK

SEQ ID NO: 635 - protein, 12 aa, Homo sapiens
KVVVKVVVKVVK

SEQ ID NO: 636 - protein, 12 aa, Homo sapiens
KVVVKVKVKVVK

SEQ ID NO: 637 - protein, 11 aa, Homo sapiens
KVVVKVKVKVK

SEQ ID NO: 638 - protein, 12 aa, Homo sapiens
KVVVKVKVKVVK

SEQ ID NO: 639 - protein, 6 aa, Homo sapiens
KLILKL

SEQ ID NO: 640 - protein, 6 aa, Homo sapiens
KVLHLL

SEQ ID NO: 641 - protein, 6 aa, Homo sapiens
LKLRLL

SEQ ID NO: 642 - protein, 6 aa, Homo sapiens
KPLHLL

SEQ ID NO: 643 - protein, 8 aa, Homo sapiens
KLILKLVR

SEQ ID NO: 644 - protein, 8 aa, Homo sapiens
KVFHLLHL

SEQ ID NO: 645 - protein, 8 aa, Homo sapiens
HKFRILKL

SEQ ID NO: 646 - protein, 8 aa, Homo sapiens
KPFHILHL

SEQ ID NO: 647 - protein, 12 aa, Homo sapiens
KIIIKIKIKIIK

SEQ ID NO: 648 - protein, 12 aa, Homo sapiens
KIIIKIKIKIIK

SEQ ID NO: 649 - protein, 12 aa, Homo sapiens
KIIIKIKIKIIK

SEQ ID NO: 650 - protein, 12 aa, Homo sapiens
KIPIKIKIKIPK

SEQ ID NO: 651 - protein, 12 aa, Homo sapiens
KIPIKIKIKIVK

SEQ ID NO: 652 - protein, 12 aa, Homo sapiens
RIIIRIRIRIIR

SEQ ID NO: 653 - protein, 12 aa, Homo sapiens
RIIIRIRIRIIR

SEQ ID NO: 654 - protein, 12 aa, Homo sapiens
RIIIRIRIRIIR

SEQ ID NO: 655 - protein, 12 aa, Homo sapiens
RIVIRIRIRLIR

SEQ ID NO: 656 - protein, 12 aa, Homo sapiens
RIIVRIRLRIIR

SEQ ID NO: 657 - protein, 12 aa, Homo sapiens
RIGIRLRVRIIR

SEQ ID NO: 658 - protein, 12 aa, Homo sapiens
KIVIRIRIRLIR

SEQ ID NO: 659 - protein, 12 aa, Homo sapiens
RIAVKWRLRFIK

SEQ ID NO: 660 - protein, 12 aa, Homo sapiens
KIGWKLRVRIIR

SEQ ID NO: 661 - protein, 12 aa, Homo sapiens
KKIGWLIIRVRR

SEQ ID NO: 662 - protein, 14 aa, Homo sapiens
RIVIRIRIRLIRIR

SEQ ID NO: 663 - protein, 14 aa, Homo sapiens
RIIVRIRLRIIRVR

SEQ ID NO: 664 - protein, 14 aa, Homo sapiens
RIGIRLRVRIIRRV

SEQ ID NO: 665 - protein, 16 aa, Homo sapiens
KIVIRIRARLIRIRIR

SEQ ID NO: 666 - protein, 16 aa, Homo sapiens
RIIVKIRLRIIKKIRL

SEQ ID NO: 667 - protein, 16 aa, Homo sapiens
KIGIKARVRIIRVKII

SEQ ID NO: 668 - protein, 16 aa, Homo sapiens
RIIVHIRLRIIHHIRL

SEQ ID NO: 669 - protein, 16 aa, Homo sapiens
HIGIKAHVRIIRVHII

SEQ ID NO: 670 - protein, 16 aa, Homo sapiens
RIYVKIHLRYIKKIRL

SEQ ID NO: 671 - protein, 16 aa, Homo sapiens
KIGHKARVHIIRYKII

SEQ ID NO: 672 - protein, 16 aa, Homo sapiens
RIYVKPHPRYIKKIRL

SEQ ID NO: 673 - protein, 16 aa, Homo sapiens
KPGHKARPHIIRYKII

SEQ ID NO: 674 - protein, 19 aa, Homo sapiens
KIVIRIRIRLIRIRIRKIV

SEQ ID NO: 675 - protein, 19 aa, Homo sapiens
RIIVKIRLRIIKKIRLIKK

SEQ ID NO: 676 - protein, 19 aa, Homo sapiens
KIGWKLRVRIIRVKIGRLR

SEQ ID NO: 677 - protein, 25 aa, Homo sapiens
KIVIRIRIRLIRIRIRKIVKVKRIR

SEQ ID NO: 678 - protein, 26 aa, Homo sapiens
RFAVKIRLRIIKKIRLIKKIRKRVIK

SEQ ID NO: 679 - protein, 30 aa, Homo sapiens
KAGWKLRVRIIRVKIGRLRKIGWKKRVRIK

SEQ ID NO: 680 - protein, 16 aa, Homo sapiens
RIYVKPHPRYIKKIRL

SEQ ID NO: 681 - protein, 16 aa, Homo sapiens
KPGHKARPHIIRYKII

SEQ ID NO: 682 - protein, 19 aa, Homo sapiens
KIVIRIRIRLIRIRIRKIV

SEQ ID NO: 683 - protein, 19 aa, Homo sapiens
RIIVKIRLRIIKKIRLIKK

SEQ ID NO: 684 - protein, 16 aa, Homo sapiens
RIYVSKISIYIKKIRL

SEQ ID NO: 685 - protein, 19 aa, Homo sapiens
KIVIFTRIRLTSIRIRSIV

SEQ ID NO: 686 - protein, 16 aa, Homo sapiens
KPIHKARPTIIRYKMI

SEQ ID NO: 687 - protein, 25 aa, Homo sapiens
CKGFFALIPKIISSPLFKTLLSAVC

SEQ ID NO: 688 - protein, 26 aa, Homo sapiens
CKKGFFALIPKIISSPLFKTLLSAVC

SEQ ID NO: 689 - protein, 27 aa, Homo sapiens
CKKKGFFALIPKIISSPLFKTLLSAVC

SEQ ID NO: 690 - protein, 16 aa, Homo sapiens
CRIVIRIRIRLIRIRC

SEQ ID NO: 691 - protein, 18 aa, Homo sapiens
CKPGHKARPHIIRYKIIC

SEQ ID NO: 692 - protein, 28 aa, Homo sapiens
CRFAVKIRLRIIKKIRLIKKIRKRVIKC

SEQ ID NO: 693 - protein, 13 aa, Homo sapiens
KLLLKLLLKLLKC

SEQ ID NO: 694 - protein, 12 aa, Homo sapiens
KLLLKLLLKLLK

SEQ ID NO: 695 - protein, 13 aa, Homo sapiens
KLLLKLKLKLLKC

SEQ ID NO: 696 - protein, 12 aa, Homo sapiens
KLLLKLLLKLLK

SEQ ID NO: 697 - protein, 28 aa, Homo sapiens
HSDAVFYDNYTRLRKQMAVKKYLNSILN

SEQ ID NO: 698 - protein, 28 aa, Homo sapiens
HSDAVFYDNYTRLRKQMAVKKYLNSILN

SEQ ID NO: 699 - protein, 4 aa, Homo sapiens
  misc_feature 1, 2, 3, and 4: Xaa at positions 1, 2, 3, and 4 is any amino acid
XXXX

SEQ ID NO: 700 - protein, 5 aa, Homo sapiens
  misc_feature 1, 3: Xaa at positions 1 and 3 is any amino acid
XSXLN

SEQ ID NO: 701 - protein, 4 aa, Homo sapiens
KKYL

SEQ ID NO: 702 - protein, 5 aa, Homo sapiens
NSILN

SEQ ID NO: 703 - protein, 4 aa, Homo sapiens
KKYL

SEQ ID NO: 704 - protein, 4 aa, Homo sapiens
KKYA

SEQ ID NO: 705 - protein, 6 aa, Homo sapiens
AVKKYL

SEQ ID NO: 706 - protein, 5 aa, Homo sapiens
NSILN

SEQ ID NO: 707 - protein, 4 aa, Homo sapiens
KKYV

SEQ ID NO: 708 - protein, 4 aa, Homo sapiens
SILN

SEQ ID NO: 709 - protein, 5 aa, Homo sapiens
KKYLL

SEQ ID NO: 710 - protein, 5 aa, Homo sapiens
NSYLN

SEQ ID NO: 711 - protein, 5 aa, Homo sapiens
NSIYN

SEQ ID NO: 712 - protein, 11 aa, Homo sapiens
KKYLPPNSILN

SEQ ID NO: 713 - protein, 5 aa, Homo sapiens
LKKYL

SEQ ID NO: 714 - protein, 4 aa, Homo sapiens
KKYL

SEQ ID NO: 715 - protein, 4 aa, Homo sapiens
KKYL

SEQ ID NO: 716 - protein, 5 aa, Homo sapiens
VKKYL

SEQ ID NO: 717 - protein, 6 aa, Homo sapiens
LNSILN

SEQ ID NO: 718 - protein, 7 aa, Homo sapiens
YLNSILN

SEQ ID NO: 719 - protein, 5 aa, Homo sapiens
KKYLN

SEQ ID NO: 720 - protein, 6 aa, Homo sapiens
KKYLNS

SEQ ID NO: 721 - protein, 7 aa, Homo sapiens
KKYLNSI

SEQ ID NO: 722 - protein, 8 aa, Homo sapiens
KKYLNSIL

SEQ ID NO: 723 - protein, 4 aa, Homo sapiens
KKYL

SEQ ID NO: 724 - protein, 5 aa, Homo sapiens
KKYDA

SEQ ID NO: 725 - protein, 6 aa, Homo sapiens
AVKKYL

SEQ ID NO: 726 - protein, 5 aa, Homo sapiens
NSILN

SEQ ID NO: 727 - protein, 4 aa, Homo sapiens
KKYV

SEQ ID NO: 728 - protein, 4 aa, Homo sapiens
SILN

SEQ ID NO: 729 - protein, 5 aa, Homo sapiens
NSYLN

SEQ ID NO: 730 - protein, 5 aa, Homo sapiens
NSIYN

SEQ ID NO: 731 - protein, 5 aa, Homo sapiens
KKYLL

SEQ ID NO: 732 - protein, 11 aa, Homo sapiens
KKYLPPNSILN

SEQ ID NO: 733 - protein, 4 aa, Homo sapiens
KKYL

SEQ ID NO: 734 - protein, 5 aa, Homo sapiens
KKYDA

SEQ ID NO: 735 - protein, 6 aa, Homo sapiens
AVKKYL

SEQ ID NO: 736 - protein, 5 aa, Homo sapiens
NSILN

SEQ ID NO: 737 - protein, 5 aa, Homo sapiens
IKKYV

SEQ ID NO: 738 - protein, 4 aa, Homo sapiens
SILN

SEQ ID NO: 739 - protein, 5 aa, Homo sapiens
LKKYL

SEQ ID NO: 740 - protein, 4 aa, Homo sapiens
KKYL

SEQ ID NO: 741 - protein, 4 aa, Homo sapiens
KKYL

SEQ ID NO: 742 - protein, 5 aa, Homo sapiens
VKKYL

SEQ ID NO: 743 - protein, 6 aa, Homo sapiens
LNSILN

SEQ ID NO: 744 - protein, 7 aa, Homo sapiens
YLNSILN

SEQ ID NO: 745 - protein, 5 aa, Homo sapiens
KKYLL

SEQ ID NO: 746 - protein, 5 aa, Homo sapiens
KKYLN

SEQ ID NO: 747 - protein, 6 aa, Homo sapiens
KKYLNS

SEQ ID NO: 748 - protein, 7 aa, Homo sapiens
KKYLNSI

SEQ ID NO: 749 - protein, 8 aa, Homo sapiens
KKYLNSIL

SEQ ID NO: 750 - protein, 6 aa, Homo sapiens
KKKYLD

SEQ ID NO: 751 - protein, 5 aa, Homo sapiens
KKYLC

SEQ ID NO: 752 - protein, 6 aa, Homo sapiens
CKKYLK

SEQ ID NO: 753 - protein, 4 aa, Homo sapiens
KKYA

SEQ ID NO: 754 - protein, 8 aa, Homo sapiens
WWTDTGLW

SEQ ID NO: 755 - protein, 8 aa, Homo sapiens
WWTDDGLW

SEQ ID NO: 756 - protein, 12 aa, Homo sapiens
WWDTRGLWVWTI

SEQ ID NO: 757 - protein, 12 aa, Homo sapiens
FWGNDGIWLESG

SEQ ID NO: 758 - protein, 12 aa, Homo sapiens
DWDQFGLWRGAA

SEQ ID NO: 759 - protein, 12 aa, Homo sapiens
RWDDNGLWVVVL

SEQ ID NO: 760 - protein, 12 aa, Homo sapiens
SGMWSHYGIWMG

SEQ ID NO: 761 - protein, 12 aa, Homo sapiens
GGRWDQAGLWVA

SEQ ID NO: 762 - protein, 12 aa, Homo sapiens
KLWSEQGIWMGE

SEQ ID NO: 763 - protein, 10 aa, Homo sapiens
CWSMHGLWLC

SEQ ID NO: 764 - protein, 12 aa, Homo sapiens
GCWDNTGIWVPC

SEQ ID NO: 765 - protein, 10 aa, Homo sapiens
DWDTRGLWVY

SEQ ID NO: 766 - protein, 10 aa, Homo sapiens
SLWDENGAWI

SEQ ID NO: 767 - protein, 10 aa, Homo sapiens
KWDDRGLWMH

SEQ ID NO: 768 - protein, 10 aa, Homo sapiens
QAWNERGLWT

SEQ ID NO: 769 - protein, 10 aa, Homo sapiens
QWDTRGLWVA

SEQ ID NO: 770 - protein, 9 aa, Homo sapiens
WNVHGIWQE

SEQ ID NO: 771 - protein, 10 aa, Homo sapiens
SWDTRGLWVE

SEQ ID NO: 772 - protein, 10 aa, Homo sapiens
DWDTRGLWVA

SEQ ID NO: 773 - protein, 10 aa, Homo sapiens
SWGRDGLWIE

SEQ ID NO: 774 - protein, 10 aa, Homo sapiens
EWTDNGLWAL

SEQ ID NO: 775 - protein, 10 aa, Homo sapiens
SWDEKGLWSA

SEQ ID NO: 776 - protein, 10 aa, Homo sapiens
SWDSSGLWMD

SEQ ID NO: 777 - protein, 6 aa, Homo sapiens
TFSDLW

SEQ ID NO: 778 - protein, 12 aa, Homo sapiens
QETFSDLWKLLP

SEQ ID NO: 779 - protein, 12 aa, Homo sapiens
QPTFSDLWKLLP

SEQ ID NO: 780 - protein, 12 aa, Homo sapiens
QETFSDYWKLLP

SEQ ID NO: 781 - protein, 12 aa, Homo sapiens
QPTFSDYWKLLP

SEQ ID NO: 782 - protein, 12 aa, Homo sapiens
MPRFMDYWEGLN

SEQ ID NO: 783 - protein, 12 aa, Homo sapiens
VQNFIDYWTQQF

SEQ ID NO: 784 - protein, 12 aa, Homo sapiens
TGPAFTHYWATF

SEQ ID NO: 785 - protein, 15 aa, Homo sapiens
IDRAPTFRDHWFALV

SEQ ID NO: 786 - protein, 15 aa, Homo sapiens
PRPALVFADYWETLY

SEQ ID NO: 787 - protein, 15 aa, Homo sapiens
PAFSRFWSDLSAGAH

SEQ ID NO: 788 - protein, 15 aa, Homo sapiens
PAFSRFWSKLSAGAH

SEQ ID NO: 789 - protein, 10 aa, Homo sapiens
  misc_feature 2, 4, 8, 9: Xaa at positions 2, 4, 8, and 9 is any amino acid
PXFXDYWXXL

SEQ ID NO: 790 - protein, 12 aa, Homo sapiens
QETFSDLWKLLP

SEQ ID NO: 791 - protein, 12 aa, Homo sapiens
QPTFSDLWKLLP

SEQ ID NO: 792 - protein, 12 aa, Homo sapiens
QETFSDYWKLLP

SEQ ID NO: 793 - protein, 12 aa, Homo sapiens
QPTFSDYWKLLP

SEQ ID NO: 794 - protein, 13 aa, Homo sapiens
SCVKWGKKEFCGS

SEQ ID NO: 795 - protein, 12 aa, Homo sapiens
SCWKYWGKECGS

SEQ ID NO: 796 - protein, 13 aa, Homo sapiens
SCYEWGKLRWCGS

SEQ ID NO: 797 - protein, 13 aa, Homo sapiens
SCLRWGKWSNCGS

SEQ ID NO: 798 - protein, 13 aa, Homo sapiens
SCWRWGKYQICGS

SEQ ID NO: 799 - protein, 13 aa, Homo sapiens
SCVSWGALKLCGS

SEQ ID NO: 800 - protein, 13 aa, Homo sapiens
SCIRWGQNTFCGS

SEQ ID NO: 801 - protein, 13 aa, Homo sapiens
SCWQWGNLKICGS

SEQ ID NO: 802 - protein, 13 aa, Homo sapiens
SCVRWGQLSICGS

SEQ ID NO: 803 - protein, 21 aa, Homo sapiens
LKKFNARRKLKGAILTTMLAK

SEQ ID NO: 804 - protein, 18 aa, Homo sapiens
RRWKKNFIAVSAANRFKK

SEQ ID NO: 805 - protein, 18 aa, Homo sapiens
RKWQKTGHAVRAIGRLSS

SEQ ID NO: 806 - protein, 14 aa, Homo sapiens
INLKALAALAKKIL

SEQ ID NO: 807 - protein, 18 aa, Homo sapiens
KIWSILAPLGTTLVKLVA

SEQ ID NO: 808 - protein, 14 aa, Homo sapiens
LKKLLKLLKKLLKL

SEQ ID NO: 809 - protein, 18 aa, Homo sapiens
LKWKKLLKLLKKLLKKLL

SEQ ID NO: 810 - protein, 17 aa, Homo sapiens
AEWPSLTEIKTLSHFSV

SEQ ID NO: 811 - protein, 17 aa, Homo sapiens
AEWPSPTRVISTTYFGS

SEQ ID NO: 812 - protein, 17 aa, Homo sapiens
AELAHWPPVKTVLRSFT

SEQ ID NO: 813 - protein, 17 aa, Homo sapiens
AEGSWLQLLNLMKQMNN

SEQ ID NO: 814 - protein, 10 aa, Homo sapiens
AEWPSLTEIK

SEQ ID NO: 815 - protein, 17 aa, Homo sapiens
SGSGVLKRPLPILPVTR

SEQ ID NO: 816 - protein, 17 aa, Homo sapiens
RWLSSRPLPPLPLPPRT

SEQ ID NO: 817 - protein, 20 aa, Homo sapiens
GSGSYDTLALPSLPLHPMSS

SEQ ID NO: 818 - protein, 20 aa, Homo sapiens
GSGSYDTRALPSLPLHPMSS

SEQ ID NO: 819 - protein, 20 aa, Homo sapiens
GSGSSGVTMYPKLPPHWSMA

SEQ ID NO: 820 - protein, 20 aa, Homo sapiens
GSGSSGVRMYPKLPPHWSMA

SEQ ID NO: 821 - protein, 20 aa, Homo sapiens
GSGSSSMRMVPTIPGSAKHG

SEQ ID NO: 822 - protein, 6 aa, Homo sapiens
RNRQKT

SEQ ID NO: 823 - protein, 4 aa, Homo sapiens
RNRQ

SEQ ID NO: 824 - protein, 5 aa, Homo sapiens
RNRQK

SEQ ID NO: 825 - protein, 5 aa, Homo sapiens
NRQKT

SEQ ID NO: 826 - protein, 4 aa, Homo sapiens
RQKT

SEQ ID NO: 827 - protein, 7 aa, Homo sapiens
RPLPPLP

SEQ ID NO: 828 - protein, 7 aa, Homo sapiens
RELPPLP

SEQ ID NO: 829 - protein, 7 aa, Homo sapiens
SPLPPLP

SEQ ID NO: 830 - protein, 7 aa, Homo sapiens
GPLPPLP

SEQ ID NO: 831 - protein, 7 aa, Homo sapiens
RPLPIPP

SEQ ID NO: 832 - protein, 7 aa, Homo sapiens
RPLPIPP

SEQ ID NO: 833 - protein, 7 aa, Homo sapiens
RRLPPTP

SEQ ID NO: 834 - protein, 7 aa, Homo sapiens
RRLPPTP

SEQ ID NO: 835 - protein, 7 aa, Homo sapiens
RQLPPTP

SEQ ID NO: 836 - protein, 7 aa, Homo sapiens
RPLPSRP

SEQ ID NO: 837 - protein, 7 aa, Homo sapiens
RPLPTRP

SEQ ID NO: 838 - protein, 7 aa, Homo sapiens
SRLPPLP

SEQ ID NO: 839 - protein, 7 aa, Homo sapiens
RALPSPP

SEQ ID NO: 840 - protein, 7 aa, Homo sapiens
RRLPRTP

SEQ ID NO: 841 - protein, 7 aa, Homo sapiens
RPVPPIT

SEQ ID NO: 842 - protein, 7 aa, Homo sapiens
ILAPPVP

SEQ ID NO: 843 - protein, 7 aa, Homo sapiens
RPLPMLP

SEQ ID NO: 844 - protein, 7 aa, Homo sapiens
RPLPILP

SEQ ID NO: 845 - protein, 7 aa, Homo sapiens
RPLPSLP

SEQ ID NO: 846 - protein, 7 aa, Homo sapiens
RPLPSLP

SEQ ID NO: 847 - protein, 7 aa, Homo sapiens
RPLPMIP

SEQ ID NO: 848 - protein, 7 aa, Homo sapiens
RPLPLIP

SEQ ID NO: 849 - protein, 7 aa, Homo sapiens
RPLPPTP

SEQ ID NO: 850 - protein, 7 aa, Homo sapiens
RSLPPLP

SEQ ID NO: 851 - protein, 7 aa, Homo sapiens
RPQPPPP

SEQ ID NO: 852 - protein, 7 aa, Homo sapiens
RQLPIPP

SEQ ID NO: 853 - protein, 12 aa, Homo sapiens
  misc_feature 1, 2, 3, 11: Xaa at positions 1, 2, 3, and 11 is any amino acid
XXXRPLPPLPXP

SEQ ID NO: 854 - protein, 12 aa, Homo sapiens
  misc_feature 1, 2, 3, 11, 12: Xaa at positions 1, 2, 3, 11, and 12 is any amino acid
XXXRPLPPIPXX

SEQ ID NO: 855 - protein, 12 aa, Homo sapiens
  misc_feature 1, 2, 3, 11, 12: Xaa at positions 1, 2, 3, 11 and 12 is any amino acid
XXXRPLPPLPXX

SEQ ID NO: 856 - protein, 12 aa, Homo sapiens
  misc_feature 2, 3, 11: Xaa at positions 2, 3, and 11 is any amino acid
RXXRPLPPLPXP

SEQ ID NO: 857 - protein, 12 aa, Homo sapiens
  misc_feature 2, 3: Xaa at positions 2 and 3 is any amino acid
RXXRPLPPLPPP

SEQ ID NO: 858 - protein, 12 aa, Homo sapiens
  misc_feature 11, 12: Xaa at positions 11 and 12 is any amino acid
PPPYPPPPIPXX

SEQ ID NO: 859 - protein, 12 aa, Homo sapiens
  misc_feature 11, 12: Xaa at positions 11 and 12 is any amino acid
PPPYPPPPVPXX

SEQ ID NO: 860 - protein, 10 aa, Homo sapiens
  misc_feature 2, 3, 8: Xaa at positions 2, 3, and 8 is any amino acid
LXXRPLPXTP

SEQ ID NO: 861 - protein, 10 aa, Homo sapiens
  misc_feature 1, 2, 3, 8: Xaa at positions 1, 2, 3 and 8 is any amino acid
XXXRPLPXLP

SEQ ID NO: 862 - protein, 10 aa, Homo sapiens
  misc_feature 3, 4, 5, 9: Xaa at positions 3, 4, 5 and 9 is any amino acid
PPXXXPPPXP

SEQ ID NO: 863 - protein, 11 aa, Homo sapiens
  misc_feature 1, 4, 9: Xaa at positions 1, 4, and 9 is any amino acid
XPPXPXKPXWL

SEQ ID NO: 864 - protein, 11 aa, Homo sapiens
  misc_feature 3, 4, 6, 8, 10: Xaa at positions 3, 4, 6, 8, and 10 is any amino acid
RPXXPXRXSXP

SEQ ID NO: 865 - protein, 11 aa, Homo sapiens
  misc_feature 8, 9: Xaa at positions 8 and 9 is any amino acid
PPVPPRPXXTL

SEQ ID NO: 866 - protein, 7 aa, Homo sapiens
  misc_feature 1, 3, 6: Xaa at positions 1, 3, and 6 is any amino acid
XPXLPXK

SEQ ID NO: 867 - protein, 10 aa, Homo sapiens
  misc_feature 1, 2, 4, 8: Xaa at positions 1, 2, 4, and 8 is any amino acid
XXDXPLPXLP

SEQ ID NO: 868 - protein, 12 aa, Homo sapiens
  misc_feature 1-2, 10, 12: Xaa at positions 1-2, 10, 12 is any amino acid
XXNFFWKTFXSX

SEQ ID NO: 869 - protein, 17 aa, Homo sapiens
DRMPCRNFFWKTFSSCK

SEQ ID NO: 870 - protein, 15 aa, Homo sapiens
MPCRNFFWKTFSSCK

SEQ ID NO: 871 - protein, 13 aa, Homo sapiens
CRNFFWKTFSSCK

SEQ ID NO: 872 - protein, 16 aa, Homo sapiens
DRMPCRNFFWKTFSSC

SEQ ID NO: 873 - protein, 14 aa, Homo sapiens
MPCRNFFWKTFSSC

SEQ ID NO: 874 - protein, 12 aa, Homo sapiens
CRNFFWKTFSSC

SEQ ID NO: 875 - protein, 16 aa, Homo sapiens
DRMPCKNFFWKTFSSC

SEQ ID NO: 876 - protein, 15 aa, Homo sapiens
MPCKNFFWKTFSSCK

SEQ ID NO: 877 - protein, 13 aa, Homo sapiens
CKNFFWKTFSSCK

SEQ ID NO: 878 - protein, 16 aa, Homo sapiens
DRMPCKNFFWKTFSSC

SEQ ID NO: 879 - protein, 14 aa, Homo sapiens
MPCKNFFWKTFSSC

SEQ ID NO: 880 - protein, 12 aa, Homo sapiens
CKNFFWKTFSSC

SEQ ID NO: 881 - protein, 17 aa, Homo sapiens
DRMPCRNFFWKTFTSCK

SEQ ID NO: 882 - protein, 15 aa, Homo sapiens
MPCRNFFWKTFTSCK

SEQ ID NO: 883 - protein, 13 aa, Homo sapiens
CRNFFWKTFTSCK

SEQ ID NO: 884 - protein, 16 aa, Homo sapiens
DRMPCRNFFWKTFTSC

SEQ ID NO: 885 - protein, 14 aa, Homo sapiens
MPCRNFFWKTFTSC

SEQ ID NO: 886 - protein, 12 aa, Homo sapiens
CRNFFWKTFTSC

SEQ ID NO: 887 - protein, 17 aa, Homo sapiens
DRMPCKNFFWKTFTSCK

SEQ ID NO: 888 - protein, 15 aa, Homo sapiens
MPCKNFFWKTFTSCK

SEQ ID NO: 889 - protein, 13 aa, Homo sapiens
CKNFFWKTFTSCK

SEQ ID NO: 890 - protein, 16 aa, Homo sapiens
DRMPCKNFFWKTFTSC

SEQ ID NO: 891 - protein, 14 aa, Homo sapiens
MPCKNFFWKTFTSC

SEQ ID NO: 892 - protein, 12 aa, Homo sapiens
CKNFFWKTFTSC

SEQ ID NO: 893 - protein, 17 aa, Homo sapiens
AEPMPHSLNFSQYLWYT

SEQ ID NO: 894 - protein, 17 aa, Homo sapiens
AEHTYSSLWDTYSPLAF

SEQ ID NO: 895 - protein, 17 aa, Homo sapiens
AELDLWMRHYPLSFSNR

SEQ ID NO: 896 - protein, 17 aa, Homo sapiens
AESSLWTRYAWPSMPSY

SEQ ID NO: 897 - protein, 17 aa, Homo sapiens
AEWHPGLSFGSYLWSKT

SEQ ID NO: 898 - protein, 17 aa, Homo sapiens
AEPALLNWSFFFNPGLH

SEQ ID NO: 899 - protein, 17 aa, Homo sapiens
AEWSFYNLHLPEPQTIF

SEQ ID NO: 900 - protein, 17 aa, Homo sapiens
AEWSFYNLHLPEPQTIF

SEQ ID NO: 901 - protein, 17 aa, Homo sapiens
AEPLDLWSLYSLPPLAM

SEQ ID NO: 902 - protein, 17 aa, Homo sapiens
AEPTLWQLYQFPLRLSG

SEQ ID NO: 903 - protein, 17 aa, Homo sapiens
AEISFSELMWLRSTPAF

SEQ ID NO: 904 - protein, 17 aa, Homo sapiens
AELSEADLWTTWFGMGS

SEQ ID NO: 905 - protein, 17 aa, Homo sapiens
AESSLWRIFSPSALMMS

SEQ ID NO: 906 - protein, 17 aa, Homo sapiens
AESLPTLTSILWGKESV

SEQ ID NO: 907 - protein, 17 aa, Homo sapiens
AETLFMDLWHDKHILLT

SEQ ID NO: 908 - protein, 17 aa, Homo sapiens
AEILNFPLWHEPLWSTE

SEQ ID NO: 909 - protein, 17 aa, Homo sapiens
AESQTGTLNTLFWNTLR

SEQ ID NO: 910 - protein, 17 aa, Homo sapiens
AEPVYQYELDSYLRSYY

SEQ ID NO: 911 - protein, 17 aa, Homo sapiens
AELDLSTFYDIQYLLRT

SEQ ID NO: 912 - protein, 17 aa, Homo sapiens
AEFFKLGPNGYVYLHSA

SEQ ID NO: 913 - protein, 11 aa, Homo sapiens
  misc_feature 4, 5, 6: Xaa at positions 4, 5, and 6 is any amino acid
FKLXXXGYVYL

SEQ ID NO: 914 - protein, 17 aa, Homo sapiens
AESTYHHLSLGYMYTLN

SEQ ID NO: 915 - protein, 11 aa, Homo sapiens
  misc_feature 3, 5, 6: Xaa at positions 3, 5, and 6 is any amino acid
YHXLXXGYMYT

SEQ ID NO: 916 - protein, 19 aa, Homo sapiens
VEPNCDIHVMWEWECFERL

SEQ ID NO: 917 - protein, 18 aa, Homo sapiens
GERWCFDGPLTWVCGEES

SEQ ID NO: 918 - protein, 20 aa, Homo sapiens
RGWVEICVADDNGMCVTEAQ

SEQ ID NO: 919 - protein, 19 aa, Homo sapiens
GWDECDVARMWEWECFAGV

SEQ ID NO: 920 - protein, 18 aa, Homo sapiens
GERWCFDGPRAWVCGWEI

SEQ ID NO: 921 - protein, 18 aa, Homo sapiens
EELWCFDGPRAWVCGYVK

SEQ ID NO: 922 - protein, 20 aa, Homo sapiens
RGWVEICAADDYGRCLTEAQ

SEQ ID NO: 923 - protein, 16 aa, Homo sapiens
RGWVEICESDVWGRCL

SEQ ID NO: 924 - protein, 16 aa, Homo sapiens
RGWVEICESDVWGRCL

SEQ ID NO: 925 - protein, 19 aa, Homo sapiens
GGNECDIARMWEWECFERL

SEQ ID NO: 926 - protein, 16 aa, Homo sapiens
RGWVEICAADDYGRCL

SEQ ID NO: 927 - protein, 10 aa, Homo sapiens
CTTHWGFTLC

SEQ ID NO: 928 - protein, 8 aa, Homo sapiens
  misc_feature 6: Xaa at position 6 is any amino acid
CLRSGXGC

SEQ ID NO: 929 - protein, 10 aa, Homo sapiens
  misc_feature 2, 3, 8, 9: Xaa at positions 2, 3, 8 and 9 is any amino acid
CXXHWGFXXC

SEQ ID NO: 930 - protein, 5 aa, Homo sapiens
  misc_feature 2, 4: Xaa at positions 2 and 4 is any amino acid
CXPXC

SEQ ID NO: 931 - protein, 10 aa, Homo sapiens
CRRHWGFEFC

SEQ ID NO: 932 - protein, 10 aa, Homo sapiens
STTHWGFTLS

SEQ ID NO: 933 - protein, 10 aa, Homo sapiens
CSLHWGFWWC

SEQ ID NO: 934 - protein, 15 aa, Homo sapiens
GFVCSGIFAVGVGRC

SEQ ID NO: 935 - protein, 15 aa, Homo sapiens
APGVRLGCAVLGRYC

SEQ ID NO: 936 - protein, 6 aa, Homo sapiens
LLGRMK

SEQ ID NO: 937 - protein, 27 aa, Homo sapiens
ICVVQDWGHHRCTAGHMANLTSHASAI

SEQ ID NO: 938 - protein, 13 aa, Homo sapiens
ICVVQDWGHHRCT

SEQ ID NO: 939 - protein, 11 aa, Homo sapiens
CVVQDWGHHAC

SEQ ID NO: 940 - protein, 27 aa, Homo sapiens
STGGFDDVYDWARGVSSALTTTLVATR

SEQ ID NO: 941 - protein, 27 aa, Homo sapiens
STGGFDDVYDWARRVSSALTTTLVATR

SEQ ID NO: 942 - protein, 30 aa, Homo sapiens
SRGVNFSEWLYDMSAAMKEASNVFPSRRSR

SEQ ID NO: 943 - protein, 30 aa, Homo sapiens
SSQNWDMEAGVEDLTAAMLGLLSTIHSSSR

SEQ ID NO: 944 - protein, 31 aa, Homo sapiens
SSPSLYTQFLVNYESAATRIQDLLIASRPSR

SEQ ID NO: 945 - protein, 31 aa, Homo sapiens
SSTGWVDLLGALQRAADATRTSIPPSLQNSR

SEQ ID NO: 946 - protein, 18 aa, Homo sapiens
DVYTKKELIECARRVSEK

SEQ ID NO: 947 - protein, 22 aa, Homo sapiens
EKGSYYPGSGIAQFHIDYNNVS

SEQ ID NO: 948 - protein, 22 aa, Homo sapiens
SGIAQFHIDYNNVSSAEGWHVN

SEQ ID NO: 949 - protein, 34 aa, Homo sapiens
LVTVEKGSYYPGSGIAQFHIDYNNVSSAEGWHVN

SEQ ID NO: 950 - protein, 14 aa, Homo sapiens
SGIAQFHIDYNNVS

SEQ ID NO: 951 - protein, 6 aa, Homo sapiens
LLGRMK

SEQ ID NO: 952 - protein, 8 aa, Homo sapiens
ALLGRMKG

SEQ ID NO: 953 - protein, 7 aa, Homo sapiens
LDPAFIR

SEQ ID NO: 954 - protein, 7 aa, Homo sapiens
  misc_feature 2, 3: Xaa at positions 2 and 3 is any amino acid
CXXRGDC

SEQ ID NO: 955 - protein, 7 aa, Homo sapiens
RPLPPLP

SEQ ID NO: 956 - protein, 6 aa, Homo sapiens
PPVPPR

SEQ ID NO: 957 - protein, 11 aa, Homo sapiens
  misc_feature 1, 3, 5, 7, 8, 10, 11: Xaa at positions 1, 3, 5, 7, 8, 10 and 11 is any amino acid
XFXDXWXXLXX

SEQ ID NO: 958 - protein, 20 aa, Homo sapiens
KACRRLFGPVDSEQLSRDCD

SEQ ID NO: 959 - protein, 20 aa, Homo sapiens
RERWNFDFVTETPLEGDFAW

SEQ ID NO: 960 - protein, 20 aa, Homo sapiens
KRRQTSMTDFYHSKRRLIFS

SEQ ID NO: 961 - protein, 20 aa, Homo sapiens
TSMTDFYHSKRRLIFSKRKP

SEQ ID NO: 962 - protein, 5 aa, Homo sapiens
RRLIF

SEQ ID NO: 963 - protein, 36 aa, Homo sapiens [sequence omitted]

SEQ ID NO: 964 - protein, 24 aa, Homo sapiens
KRRLIFSKRQIKIWFQNRRMKWKK

SEQ ID NO: 965 - protein, 25 aa, Homo sapiens
NQGRHFCGGALIHARFVMTAASCFQ

SEQ ID NO: 966 - protein, 27 aa, Homo sapiens
GTRCQVAGWGSQRSGGRLSRFPRFVNV

SEQ ID NO: 967 - protein, 15 aa, Homo sapiens
WHWRHRIPLQLAAGR

SEQ ID NO: 968 - protein, 6 aa, Homo sapiens
LKTPRV

SEQ ID NO: 969 - protein, 8 aa, Homo sapiens
NTLKTPRV

SEQ ID NO: 970 - protein, 11 aa, Homo sapiens
NTLKTPRVGGC

SEQ ID NO: 971 - protein, 6 aa, Homo sapiens
KDKATF

SEQ ID NO: 972 - protein, 10 aa, Homo sapiens
KDKATFGCHD

SEQ ID NO: 973 - protein, 12 aa, Homo sapiens
KDKATFGCHDGC

SEQ ID NO: 974 - protein, 6 aa, Homo sapiens
TLRVYK

SEQ ID NO: 975 - protein, 8 aa, Homo sapiens
ATLRVYKG

SEQ ID NO: 976 - protein, 10 aa, Homo sapiens
CATLRVYKGG

SEQ ID NO: 977 - protein, 14 aa, Homo sapiens
INLKALAALAKKIL

SEQ ID NO: 978 - protein, 12 aa, Homo sapiens
GWTLNSAGYLLG

SEQ ID NO: 979 - protein, 27 aa, Homo sapiens
GWTLNSAGYLLGKINLKALAALAKKIL

SEQ ID NO: 980 - protein, 125 aa, Homo sapiens
  MISC_FEATURE (1)..(125): Vh1 heavy chain variable region
QVQLLVQSGAEVKKPGASVKVSCKASGYTFTXWVRQAPGQGLEWMGXRVTMTRDTSTSTAYMELSSLRSEDTAVYYCARXWGQGTLVTVSSGSTKGPSVFPLAPSSKSTSGGTAALGCLVKDYFP

SEQ ID NO: 981 - protein, 124 aa, Homo sapiens
  MISC_FEATURE (1)..(124): Vh2 heavy chain variable region
QITLKESGPALVKPTQTLTLTCTFSGFSLSXWIRQPPGKALEWLAXRLTITKDTSKNQVVLTMTNMDPVDTATYYCARXWGQGTLVTVSSASPTSPKVFPLSLSSKSTSGGTAALGCLVKDYFP

SEQ ID NO: 982 - protein, 100 aa, Homo sapiens
  MISC_FEATURE (1)..(100): Vh3a heavy chain variable region
EVVQLVESGGGLVQPGGSLRLSCAASGFTFSXWVRQAPGKGLEWVSXRFTISRDNSKNTLYLQMNSLRAEDTAVYYCARXWGQGTLVTVSSGSTKAPSVF

SEQ ID NO: 983 - protein, 102 aa, Homo sapiens
  MISC_FEATURE (1)..(102): Vh3b heavy chain variable region
EVQLVESGGGLVKPGGSLRLSCAASGFTFSXWVRQAPGKGLEWVGXRFTISRDDSKNTLYLQMNSLKTEDTAVYYCTTXWGQGTLVTVSSASTKGPSVFPLA

SEQ ID NO: 984 - protein, 101 aa, Homo sapiens
  MISC_FEATURE (1)..(101): Vh3c heavy chain variable region
EVQLVESGGGLVQPGRSLRLSCTASGFTFGXWVRQAPGKGLEWVGXRFTISRDDSKSIAYLQMNSLKTEDTAVYYCTRNXWGQGTLVTVSSGSTKGPSVLP

SEQ ID NO: 985 - protein, 108 aa, Homo sapiens
  MISC_FEATURE (1)..(108): Vh4 heavy chain variable region
QVQLQESGPGLVKPSETLSLTCTVSGGSSISSSXWIRQPPGKGLEWIGXRVTISVDTSKNQFSLKLSSVTAADTAVYYCARXWGQGTLVTVSSAPTKAPDVFPIISGC

SEQ ID NO: 986 - protein, 132 aa, Homo sapiens
  MISC_FEATURE (1)..(132): Vh5 heavy chain variable region
EEVQLVQSGAEVKKPGESLKISCKGSGYSFTXWVRQMPGKGLEWMGXQVTISADKSISTAYLQWSSLKASDTAMYYCARXWGQGTLVTVSSASTKAPSVFPLVSCENSPSDTSSVAVGCLAQDFLPDSITFS

SEQ ID NO: 987 - protein, 125 aa, Homo sapiens
  MISC_FEATURE (1)..(125): Vh6 heavy chain variable region
QVQLQQSGPGLVKPSQTLSLTCAISGDSVSXWIRQSPSRGLEWLGXRITINPDTSKNQFSLQLNSVTPEDTAVYYCARXWGQGTLVTVSSGSASAPTLFPLVSCENSPSDTSSVAVGCLAQDFLP

SEQ ID NO: 988 - protein, 91 aa, Homo sapiens
  MISC_FEATURE (1)..(91): Vh7 heavy chain variable region
QVQLVQSGSELKKPGASVKVSCKASGYTFTXWVRQAPGQGLEWMGXRFVFSLDTSVSTAYLQISSLKAEDTAVYYCARXWGQGTLVTVSSS

SEQ ID NO: 989 - protein, 93 aa, Homo sapiens
  MISC_FEATURE (1)..(93): Kappa 1_4 light chain variable region
DIQMTQSPSSLSASVGDRRVTITCXWYQQKPGKAPKLLIYXGVPSRFSGSGSGTDFTLTISSLQPEDFATYYCXFGQGTKVEIKRTVAAPSVF

SEQ ID NO: 990 - protein, 92 aa, Homo sapiens
  MISC_FEATURE (1)..(92): Kappa2 light chain variable region
DIVMTQSPLSLPVTPGQPASISCXWYLQKPGQSPQLLIYXGVPDRFSGSGSGTDFTLKISRVEAEDVGVYYCXFGQGTKVEIKRTVAAPSVF

SEQ ID NO: 991 - protein, 91 aa, Homo sapiens
  MISC_FEATURE (1)..(91): Kappa3 light chain variable region
EIVLTQSPGTLSLSPGERATLSCXWYQQKPGQAPRLLIYXGIPDRFSGSGSGTDFTLTISRLEPEDFAVYYCXFGQGTKVEIKRTVAAPSV

SEQ ID NO: 992 - protein, 85 aa, Homo sapiens
  MISC_FEATURE (1)..(85): Kappa5 light chain variable region
ETTLTQSPAFMSATPGDKVNISCXWYQQKPGEAAIFIIQXGIPPRFSGSGYGTDFTLTINNIESEDAAYYFCXLRHFWPGDQAAG

SEQ ID NO: 993 - protein, 79 aa, Homo sapiens
  MISC_FEATURE (1)..(67): KappaNew1 light chain variable region
EIVMTQSPVNLSMSAGEXWYQQKPGQAPRLFIYXGISARFSGSGSGTDFTLTITSLQSEDFAVYYCXFGQGTKLDIKRT

SEQ ID NO: 994 - protein, 77 aa, Homo sapiens
  MISC_FEATURE (1)..(65): KappaNew2 light chain variable region
ELTQSPGTLSLSPGEXWYQHKPGQAPRLVIHXGISDRFSGSGSGTDFTLTITRLEPEDFALYYCXFGQGTKLDFKRT

SEQ ID NO: 995 - protein, 95 aa, Homo sapiens
  MISC_FEATURE (1)..(95): KappaNew3 light chain variable region
DIVMTQSPDSLAVSLGGRRATINCXWYQQKPGQPPKLLIYXGVPDRFSGSGSGTDFTLTISSLQAEDVAVYYCXFGGGTKVEIKRTVAAPSVFKF

SEQ ID NO: 996 - protein, 98 aa, Homo sapiens
  MISC_FEATURE (1)..(98): Lambda1a light chain variable region
QSVLTQPPSVSGAPGQRVTISCXWYQQLPGTAPKLLIYXGVPDRFSGSKSGTSASLAISGLQSEDEADYYCXFGGGTKLTVLGQPKAAPSVTLFPPSS

SEQ ID NO: 997 - protein, 99 aa, Homo sapiens
  MISC_FEATURE (1)..(99): Lambda1b light chain variable region
AQSVLTQPPSVSAAPGQKVTISCXWYQQLPGTAPKLLIYXGIPDRFSGSKSGTSATLGITGLQTGDEADYYCXFGGGTKLTVLGQPKAAPSVTLFPPSS

SEQ ID NO: 998 - protein, 99 aa, Homo sapiens
  MISC_FEATURE (1)..(72): Lambda2 light chain variable region
QSALTQPASVSGSPGQSITISCXWYQQHPGKAPKLMIYXGVSNRFSGSKSGNTASLTISGLQAEDEADYYCXFGGGTTKLTVLGQPKAAPSVTLFPPSS

SEQ ID NO: 999 - protein, 107 aa, Homo sapiens
  MISC_FEATURE (1)..(107): Lambda3a light chain variable region
SYELTQPPSVSVSPGQTARITCXWYQQKPGQAPVLVIYXGIPERFSGSSSGTTATLTISGVQAEDEADYYCXFGGGTKLTVLGQPKAAPSVTLFPPSSEELQANKAT

SEQ ID NO: 1000 - protein, 93 aa, Homo sapiens
  MISC_FEATURE (1)..(93): Lambda3b light chain variable region
SYVLTQPPSVSVAPGQTARITCXWYQQKPGQAPVLVVYDXGIPERFSGSNSGNTATLTISRVEAGDEADYYCXFGGGTKLTVLGQPKAAPTVT

SEQ ID NO: 1001 - protein, 98 aa, Homo sapiens
  MISC_FEATURE (1)..(98): Lambda3c light chain variable region
SYELTQPPSVSVSPGQTASITCXWYQQKPGQSPVLVIYXGIPERFSGSNSGNTATLTISGTQAMDEADYYCXFGGGTKLTVLGQPKAAPSRSLCPPPP

SEQ ID NO: 1002 - protein, 98 aa, Homo sapiens
  MISC_FEATURE (1)..(98): Lambda3e light chain variable region
SSELTQDPAVSVALGQTVRITCXWYQQKPGQAPVLVIYXGIPDRFSGSSSGNTASLTITGAQAEDEADYYCXFGGGTKLTVLGQPKAAPSVTLFPPSS

SEQ ID NO: 1003 - protein, 94 aa, Homo sapiens
  MISC_FEATURE (1)..(94): Lambda4a light chain variable region
QPVLTQSSSASASLGSSVKLTCXWHQQQPGKAPRYLMKXGVPDRFSGSSSGADRYLTISNLQSEDEADYYCXFGGGTKLTVLGQPKAAPSVTLF

SEQ ID NO: 1004 - protein, 95 aa, Homo sapiens
  MISC_FEATURE (1)..(95): Lambda4b light chain variable region
QLVLTQSPSASASLGASVKLTCXWHQQQPEKGPRYLMKXGIPDRFSGSSSGAERYLTISSLQSEDEADYYCXFGGIGGGTKLTVLGQPKAAPSVS

SEQ ID NO: 1005 - protein, 88 aa, Homo sapiens
  MISC_FEATURE (1)..(75): Lambda5 light chain variable region
QAVLTQPSSLSASPGASASLTCXWYQQKPGSPPQYLLRYXGVPSRFSGSKDASANAGILLISGLQSEDEADYYCXFGGGTKLTVLSQP

SEQ ID NO: 1006 - protein, 101 aa, Homo sapiens
  MISC_FEATURE (1)..(101): Lambda6 light chain variable region
NFMLTQPHSVSESPGKTVTISCXWYQQRPGSAPTTVIYXGVPDRFSGSIDSSSNSASLTISGLKTEDEADYYCXFGGGTKLTVLGQPKAAPSVTLFPPSSS

SEQ ID NO: 1007 - protein, 89 aa, Homo sapiens
  MISC_FEATURE (1)..(72): Lambda7 light chain variable region
QAVVTQEPSLTVSPGGTVTLTCXWFQQKPGQAPRALIYXWTPARFSGSLLGGKAALTLSGVQPEDEAEYYCXFGGGTKLTVLGQPKAAP

SEQ ID NO: 1008 - protein, 89 aa, Homo sapiens
  MISC_FEATURE (1)..(89): Lambda8 light chain variable region
QTVVTQEPSFSVSPGGTVTLTCXWYQQTPGQAPRTLIYXGVPDRFSGSILGNKAALTITGAQADDESDYYCXFGGGTKLTVLGQPKAAP

SEQ ID NO: 1009 - protein, 91 aa, Homo sapiens
  MISC_FEATURE (1)..(91): Lambda9 light chain variable region
QPVLTQPPSASASLGASVTLTCXWYQQRPGKGPRFVMRXGIPDRFSVLGSGLNRYLTIKNIQEEDESDYHCXFGGGTKLTVLGQPKAAPSV

SEQ ID NO: 1010 - protein, 87 aa, Homo sapiens
  MISC_FEATURE (1)..(87): Lambda10 light chain variable region
QAGLTQPPSVSKGLRQTATLTCXWLQQHQGHPPKLLSYXGISERFSASRSGNTASLTITGLQPEDEADYYCXFGGGTKLTVLGQPKA

SEQ ID NO: 1011 - protein, 354 aa, Homo sapiens
  MISC_FEATURE (1)..(354): IgA1 heavy chain constant region
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSZEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 1012 - protein, 340 aa, Homo sapiens
  MISC_FEATURE (1)..(340): IgA2 heavy chain constant region
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 1013 - protein, 384 aa, Homo sapiens
  MISC_FEATURE (1)..(384): IgD heavy chain constant region
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 1014 - protein, 497 aa, Homo sapiens
  MISC_FEATURE (1)..(497): IgE heavy chain constant region
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPVGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKDVCVEEAEGEAPWTWTGLCIFAALFLLSVSYSAALTLLMVQRFLSATRQGRPQTSLDYTNVLQPHA

SEQ ID NO: 1015 - protein, 339 aa, Homo sapiens
  MISC_FEATURE (1)..(339): IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESBNGQPENNYKTTPPVLDSDGSFFLYSKLTVDKSRWQQGNVFSCSVMHEALHNHYTQKSLSLSPGKTHTCPPCP

SEQ ID NO: 1016 - protein, 326 aa, Homo sapiens
  MISC_FEATURE (1)..(326): IgG2 heavy chain constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 1017 - protein, 377 aa, Homo sapiens
  MISC_FEATURE (1)..(377): IgG3 heavy chain constant region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 1018 - protein, 327 aa, Homo sapiens
  MISC_FEATURE (1)..(327): IgG4 heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1019 - protein, 476 aa, Homo sapiens
  MISC_FEATURE (1)..(476): IgM heavy chain constant region
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRSKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWQMQRGQPLSPEKYVTSAPMPEPQAPGRYFAHSILTVSEEEWNTGETYTCVVAHEALPNRVTERTVDKSTGKPTSADEEGFENLWATASTFIVLYNVSLVMSDTAGTCYVK

SEQ ID NO: 1020 - protein, 107 aa, Homo sapiens [sequence omitted]

SEQ ID NO: 1021 - protein, 107 aa, Homo sapiens
  MISC_FEATURE (1)..(107): Light chain lambda constant region (IgLambda)
GQPKAAPSVTLFPPSSEELQANKATLVCLISDFYPGAVTVAWKADSSPVKAGVETTTPSKQSNNKYAASSYLSLTPEQWKSHRKSYSCQVTHEGSTVEKTVAPTECS

(19) United States
(12) Patent Application Publication
(10) Pub. No.: US 2006/0127404 A1
(43) Pub. Date: Jun. 15, 2006

(54) Hinge core mimetibodies, compositions, methods and uses

(76) Inventors: ChiChi Huang, Berwyn, PA (US); George A. Heavner, Malvern, PA (US); David M. Knight, Berwyn, PA (US); John Ghrayeb, Downingtown, PA (US); Bernard J. Scallon, Wayne, PA (US); Thomas C. Nesspor, Collegeville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/953,613

(22) Filed: Sep. 29, 2004

Related U.S. Application Data
(60) Provisional application No. 60/507,231, filed on Sep. 30, 2003.

(51) Int. Cl. A61K 39/395; C07K 16/44

(52) U.S. Cl. 424/155.1; 424/178.1; 530/387.3; 530/388.8

(57) ABSTRACT

The present invention relates to at least one novel human hinge core mimetibody or specified portion or variant, including isolated nucleic acids that encode at least one hinge core mimetibody or specified portion or variant, hinge core mimetibody or specified portion or variants, vectors, host cells, transgenic animals or plants, and methods of making and using thereof, including therapeutic compositions, methods and devices.

This application claims priority to Provisional Application Ser. No. 60/507,231 filed Sep. 30, 2003, and is entirely incorporated herein by reference.

### BACKGROUND OF THE INVENTION

1. Field of the Invention

The present invention relates to hinge core mimetibodies, specified portions and variants specific for bologically active proteins, fragment or ligands, hinge core mimetibody encoding and complementary nucleic acids, host cells, and methods of making and using thereof, including therapeutic formulations, administration and devices.

2. Related Art

Recombinant proteins are an emerging class of therapeutic agents. Such recombinant therapeutics have engendered advances in protein formulation and chemical modification. Such modifications can potentially enhance the therapeutic utility of therapeutic proteins, such as by increaseing half lives (e.g., by blocking their exposure to proteolytic enzymes), enhancing biological activity, or reducing unwanted side effects. One such modification is the use of immunoglobulin fragments fused to receptor proteins, such as enteracept. Therapeutic proteins have also been constructed using the Fc domain to attempt to provide a longer half-life or to incorporate functions such as Fc receptor binding, protein A binding, and complement fixation.

Accordingly, there is a need to provide improved and/or modified versions of therapeutic proteins, which overcome one more of these and other problems known in the art.

### SUMMARY OF THE INVENTION

The present invention provides isolated human hinge core mimetibodies, including modified immunoglobulins, cleavage products and other specified portions and variants thereof, as well as hinge core mimetibody compositions, encoding or complementary nucleic acids, vectors, host cells, compositions, formulations, devices, transgenic animals, transgenic plants, and methods of making and using thereof, as described and/or enabled herein, in combination with what is known in the art.

The present invention also provides at least one hinge core mimetibody or specified portion or variant as described herein and/or as known in the art. The hinge core mimetibody can optionally comprise at least one CH3 region directly linked with at least one CH2 region directly linked with at least one portion of a truncated hinge region or fragment thereof (H) directly linked with at an optional linker sequence (L), directly linked to at least one therapeutic peptide (P), optionally further directly linked with at least a portion of at least one variable antibody sequence (V). In a preferred embodiment a pair of a IgG CH3-CH2-partial hinge (H) linker (L)-therapeutic peptide (P) with an optional N-terminal variable sequence, the pair optionally linked by association or covalent linkage, such as, but not limited to, at least one Cys-Cys disulfide bond or at least one CH4 or other immunglobulin sequence. In one embodiment, a hinge core mimetibody comprises formula (I):
((V(m)-P(n)-L(o)-H(p)-CH2(q)-CH3(r))(s),
where V is at least one portion of an N-terminus of an immunoglobulin variable region, P is at least one bioactive peptide, L is at least one linker polypeptide H is at least one portion of at least one immunoglobulin hinge region, CH2 is at least a portion of an immunoglobulin CH2 constant region, CH3 is at least a portion of an immunoglobulin CH3 constant region, m, n, o, p, q, r and s are independently an integer between 0, 1 or 2 and 10, mimicing different types of immunoglobulin molecules, e.g., but not limited to IgG1, IgG2, IgG3, IgG4, IgA, IgM, IgD, IgE, and the like, or any subclass thereof, or any combination thereof.

Thus, a hinge core mimetibody of the present invention mimics at least a portion of an antibody or immnuoglobulin structure or function with its inherent properties and functions, while providing a therapeutic peptide and its inherent or acquired in vitro, in vivo or in situ properties or activities. The various portions of the antibody and therapeutic peptide portions of at least one hinge core mimetibody of the present invention can vary as described herein in combination with what is known in the art.

At least one hinge core mimetibody or specified portion or variant of the invention mimics the binding of the P portion of the mimetibody to at least one ligand, or has at least one biological activity of, at least one protein, subunit, fragment, portion or any combination thereof.

The present invention also provides at least one isolated hinge core mimetibody or specified portion or variant as described herein and/or as known in the art, wherein the hinge core mimetibody or specified portion or variant has at least one activity, such as, but not limited to known biological activities of at least one bioactive peptide or polypeptide corresponding to the P portion of Formula I. A hinge core mimetibody can thus be screened for a corresponding activity according to known methods, such as at least one neutralizing activity towards a protein or fragment thereof.

In one aspect, the present invention provides at least one isolated hinge core mimetibody, comprising at least one P(n) region comprising at least a bilogically active portion of at least one of SEQ ID NOS:1-979, or optionally with one or more substitutions, deletions or insertions as described herein and/or as known in the art.

In another aspect, the present invention provides at least one isolated hinge core mimetibody, wherein the hinge core mimetibody specifically binds at least one epitope comprising at least 1-3, to the entire amino acid sequence of at least one ligand or binding region which ligand binds to at least a portion of at least one of SEQ ID NOS: 1-979, or optionally with one or more substitutions, deletions or insertions as described herein or as known in the art.

The at least one hinge core mimetibody can optionally further comprise at least one characteristic selected from (i) bind at least one protein with an affinity of at least 10⁻⁹ M, at least 10⁻¹⁰ M, at least 10⁻¹¹ M, or at least 10⁻¹² M; and/or (ii) substantially neutralize at least one activity of at least one protein or portion thereof.

The present invention provides, in one aspect, isolated nucleic acid molecules comprising, complementary, having significant identity or hybridizing to, a polynucleotide encoding specific mimetibodies or specified portions or variants thereof, comprising at least one specified sequence, domain, portion or variant thereof. The present invention further provides recombinant vectors comprising at least one of said isolated hinge core mimetibody nucleic acid molecules, host cells containing such nucleic acids and/or recombinant vectors, as well as methods of making and/or using such hinge core mimetibody nucleic acids, vectors and/or host cells.

Also provided is an isolated nucleic acid encoding at least one isolated hinge core mimetibody; an isolated nucleic acid vector comprising the isolated nucleic acid, and/or a prokaryotic or eukaryotic host cell comprising the isolated nucleic acid. The host cell can optionally be at least one selected from COS-1, COS-7, HEK293, BHK21, CHO, BSC-1, Hep G2, 653, SP2/0, 293, HeLa, myeloma, or lymphoma cells, or any derivative, immortalized or transformed cell thereof. Also provided is a method for producing at least one hinge core mimetibody, comprising translating the hinge core mimetibody encoding nucleic acid under conditions in vitro, in vivo or in situ, such that the hinge core mimetibody is expressed in detectable or recoverable amounts.

The present invention also provides at least one composition comprising (a) an isolated hinge core mimetibody or specified portion or variant encoding nucleic acid and/or hinge core mimetibody as described herein; and (b) a suitable carrier or diluent. The carrier or diluent can optionally be pharmaceutically acceptable, according to known methods. The composition can optionally further comprise at least one further compound, protein or composition.

Also provided is a composition comprising at least one isolated hinge core mimetibody and at least one pharmaceutically acceptable carrier or diluent. The composition can optionally further comprise an effective amount of at least one compound or protein selected from at least one of a detectable label or reporter, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an ophthalmic, otic or nasal drug, a topical drug, a nutritional drug, a TNF antagonist, an antirheumatic, a muscle relaxant, a narcotic, a non-steroid anti-inflammatory drug (NTHE), an analgesic, an anesthetic, a sedative, a local anethetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

The present invention further provides at least one anti-idiotype antibody to at least one hinge core mimetibody of the present invention. The anti-idiotype antibody includes any protein or peptide containing molecule that comprises at least a portion of an immunoglobulin molecule, such as but not limited to at least one complimetarity determing region (CDR) of a heavy or light chain or a ligand binding portion thereof, a heavy chain or light chain variable region, a heavy chain or light chain constant region, a framework region, or any portion thereof, that can be incorporated into a hinge core mimetibody of the present invention. A hinge core mimetibody of the invention can include or be derived from any mammal, such as but not limited to a human, a mouse, a rabbit, a rat, a rodent, a primate, and the like.

The present invention further provides an anti-idiotype antibody or fragment that specifically binds at least one hinge core mimetibody of the present invention.

The present invention provides, in one aspect, isolated nucleic acid molecules comprising, complementary, or hybridizing to, a polynucleotide encoding at least one hinge core mimetibody anti-idiotype antibody, comprising at least one specified sequence, domain, portion or variant thereof. The present invention further provides recombinant vectors comprising said hinge core mimetibody anti-idiotype antibody encoding nucleic acid molecules, host cells containing such nucleic acids and/or recombinant vectors, as well as methods of making and/or using such anti-idiotype antiobody nucleic acids, vectors and/or host cells.

The present invention also provides at least one method for expressing at least one hinge core mimetibody or specified portion or variant, or hinge core mimetibody anti-idiotype antibody, in a host cell, comprising culturing a host cell as described herein and/or as known in the art under conditions wherein at least one hinge core mimetibody or specified portion or variant, or anti-idiotype antibody is expressed in detectable and/or recoverable amounts.

The present invention further provides at least one hinge core mimetibody, specified portion or variant in a method or composition, when administered in a therapeutically effective amount, for modulation, for treating or reducing the symptoms of at least one of a bone and joint disorder, cardiovascular disoder, a dental or oral disorder, a dermatologic disorder, an ear, nose or throat disorder, an endocrine or metabolic disorder, a gastrointestinal disorder, a gynecologic disorder, a hepatic or biliary disorder, a an obstetric disorder, a hematologic disorder, an immunologic or allergic disorder, an infectious disease, a musculoskeletal disorder, a oncologic disorder, a neurologic disorder, a nutritrional disorder, an opthalmologic disorder, a pediatric disorder, a poisoning disorder, a psychiatric disorder, a renal disorder, a pulmonary disorder, or any other known disorder. (See., e.g., The Merck Manual, 17th ed., Merck Research Laboratories, Merck and Co., Whitehouse Station, N.J. (1999), entirely incoporated herein by reference), as needed in many different conditions, such as but not limited to, prior to, subsequent to, or during a related disease or treatment condition, as known in the art.

The present invention further provides at least one hinge core mimetibody, specified portion or variant in a method or composition, when administered in a therapeutically effective amount, for modulation, for treating or reducing the symptoms of, at least one immune, cardiovascular, infectious, malignant, and/or neurologic disease in a cell, tissue, organ, animal or patient and/or, as needed in many different conditions, such as but not limited to, prior to, subsequent to, or during a related disease or treatment condition, as known in the art and/or as described herein.

The present invention also provides at least one composition, device and/or method of delivery of a therapeutically or prophylactically effective amount of at least one hinge core mimetibody or specified portion or variant, according to the present invention.

The present invention also provides at least one composition comprising (a) an isolated hinge core mimetibody encoding nucleic acid and/or hinge core mimetibody as described herein; and (b) a suitable carrier or diluent. The carrier or diluent can optionally be pharmaceutically acceptable, according to known carriers or diluents. The composition can optionally further comprise at least one further compound, protein or composition

The present invention further provides at least one hinge core mimetibody method or composition, for administering a therapeutically effective amount to modulate or treat at least one protein related condition in a cell, tissue, organ, animal or patient and/or, prior to, subsequent to, or during a related condition, as known in the art and/or as described herein.

The present invention also provides at least one composition, device and/or method of delivery of a therapeutically or prophylactically effective amount of at least one hinge core mimetibody, according to the present invention.

The present invention further provides at least one hinge core mimetibody method or composition, for diagnosing at least one protein related condition in a cell, tissue, organ, animal or patient and/or, prior to, subsequent to, or during a related condition, as known in the art and/or as described herein.

The present invention also provides at least one composition, device and/or method of delivery for diagnosing of at least one hinge core mimetibody, according to the present invention.

Also provided is a method for diagnosing or treating a disease condition in a cell, tissue, organ or animal, comprising

(a) contacting or administering a composition comprising an effective amount of at least one isolated hinge core mimetibody of the invention with, or to, the cell, tissue, organ or animal. The method can optionally further comprise using an effective amount of 0.001-50 mg/kilogram of the cells, tissue, organ or animal. The method can optionally further comprise using the contacting or the administrating by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal. The method can optionally further comprise administering, prior, concurrently or after the (a) contacting or administering, at least one composition comprising an effective amount of at least one compound or protein selected from at least one of a detectable label or reporter, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an ophthalmic, otic or nasal drug, a topical drug, a nutritional drug, a TNF antagonist, an antirheumatic, a muscle relaxant, a narcotic, a non-steroid anti-inflammatory drug (NSAID), an analgesic, an anesthetic, a sedative, a local anethetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

Also provided is a medical device, comprising at least one isolated hinge core mimetibody of the invention, wherein the device is suitable to contacting or administerting the at least one hinge core mimetibody by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal.

Also provided is an article of manufacture for human pharmaceutical or diagnostic use, comprising packaging material and a container comprising a solution or a lyophilized form of at least one isolated hinge core mimetibody of the present invention. The article of manufacture can optionally comprise having the container as a component of a parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal delivery device or system.

Also provided is a method for producing at least one isolated hinge core mimetibody of the present invention, comprising providing a host cell or transgenic animal or transgenic plant or plant cell capable of expressing in recoverable amounts the hinge core mimetibody. Further provided in the present invention is at least one hinge core mimetibody produced by the above method.

The present invention further provides any invention described herein.

### DESCRIPTION OF THE FIGURES

FIGS. 1-41 show examples of heavy/light chain variable/constant region sequences, frameworks/subdomains and substitutions, portions of which can be used in Ig derived proteins of the present invention, as taught herein. Framework, CDR and hinge regions are labeled in boxes. Sequence residues are numbered for each amino acid postion. A list of amino acid substitutions or gaps (denoted by a “-”) observed at each position in the aligned sequences are shown below each sequence residue.

FIG. 1 depicts Vh1 heavy chain variable region sequences, frameworks and substitutions (SEQ ID NO:980).

FIG. 2 depicts Vh2 heavy chain variable region sequences, frameworks and substitutions (SEQ ID NO:981).

FIG. 3 depicts Vh3a heavy chain variable region sequences, frameworks and substitutions (SEQ ID NO:982).

FIG. 4 depicts Vh3b heavy chain variable region sequences, frameworks and substitutions (SEQ ID NO:983).

FIG. 5 depicts Vh3c heavy chain variable region sequences, frameworks and substitutions (SEQ ID NO:984).

FIG. 6 depicts Vh4 heavy chain variable region sequences, frameworks and substitutions (SEQ ID NO:985).

FIG. 7 depicts Vh5 heavy chain variable region sequences, frameworks and substitutions (SEQ ID NO:986).

FIG. 8 depicts Vh6 heavy chain variable region sequences, frameworks and substitutions (SEQ ID NO:987).

FIG. 9 depicts Vh7 heavy chain variable region sequences, frameworks and substitutions (SEQ ID NO:988).

FIG. 10 depicts κ1_{—}4 light chain variable region sequences, frameworks and substitutions (SEQ ID NO:989).

FIG. 11 depicts κ2 light chain variable region sequences, frameworks and substitutions (SEQ ID NO:990).

FIG. 12 depicts κ3 light chain variable region sequences, frameworks and substitutions (SEQ ID NO:991).

FIG. 13 depicts κ5 light chain variable region sequences, frameworks and substitutions (SEQ ID NO:992).

FIG. 14 depicts κNew1 light chain variable region sequences, frameworks and substitutions (SEQ ID NO:993).

FIG. 15 depicts κNew2 light chain variable region sequences, frameworks and substitutions (SEQ ID NO:994).

FIG. 16 depicts λ1a light chain variable region sequences, frameworks and substitutions (SEQ ID NO:995).

FIG. 17 depicts λ1b light chain variable region sequences, frameworks and substitutions (SEQ ID NO:996).

FIG. 18 depicts λ2 light chain variable region sequences, frameworks and substitutions (SEQ ID NO:997).

FIG. 19 depicts λ3a light chain variable region sequences, frameworks and substitutions (SEQ ID NO:998).

FIG. 20 depicts λ3b light chain variable region sequences, frameworks and substitutions (SEQ ID NO:999).

FIG. 21 depicts λ3c light chain variable region sequences, frameworks and substitutions (SEQ ID NO:1000).

FIG. 22 depicts λ3e light chain variable region sequences, frameworks and substitutions (SEQ ID NO:1001).

FIG. 23 depicts λ4a light chain variable region sequences, frameworks and substitutions (SEQ ID NO:1002).

FIG. 24 depicts λ4b light chain variable region sequences, frameworks and substitutions (SEQ ID NO:1003).

FIG. 25 depicts λ5 light chain variable region sequences, frameworks and substitutions (SEQ ID NO:1004).

FIG. 26 depicts λ6 light chain variable region sequences, frameworks and substitutions (SEQ ID NO:1005).

FIG. 27 depicts λ7 light chain variable region sequences, frameworks and substitutions (SEQ ID NO:1006).

FIG. 28 depicts λ8 light chain variable region sequences, frameworks and substitutions (SEQ ID NO:1007).

FIG. 29 depicts λ9 light chain variable region sequences, frameworks and substitutions (SEQ ID NO:1008).

FIG. 30 depicts λ10 light chain variable region sequences, frameworks and substitutions (SEQ ID NO:1009.

FIG. 31 depicts IgA1 heavy chain constant region sequences, subdomains and substitutions (SEQ ID NO:1010

FIG. 32 depicts IgA2 heavy chain constant region sequences, subdomains and substitutions (SEQ ID NO:1011

FIG. 33 depicts IgD heavy chain constant region sequences, subdomains and substitutions (SEQ ID NO:1012

FIG. 34 depicts IgE heavy chain constant region sequences, subdomains and substitutions (SEQ ID NO:1013

FIG. 35 depicts IgG1 heavy chain constant region sequences, subdomains and substitutions (SEQ ID NO:1014

FIG. 36 depicts IgG2 heavy chain constant region sequences, subdomains and substitutions (SEQ ID NO:1015

FIG. 37 depicts IgG3 heavy chain constant region sequences, subdomains and substitutions (SEQ ID NO:1016

FIG. 38 depicts IgG4 heavy chain constant region sequences, subdomains and substitutions (SEQ ID NO:1017

FIG. 39 depicts IgM heavy chain constant region sequences, subdomains and substitutions (SEQ ID NO:1018

FIG. 40 depicts Igκc light chain constant region sequences and substitutions (SEQ ID NO:1019

FIG. 41 depicts Igλc light chain constant region sequences and substitutions (SEQ ID NO:1020

### DESCRIPTION OF THE INVENTION

The present invention provides isolated, recombinant and/or synthetic mimetibodies or specified portions or variants, as well as compositions and encoding nucleic acid molecules comprising at least one polynucleotide encoding at least one hinge core mimetibody. Such mimetibodies or specified portions or variants of the present invention comprise specific hinge core mimetibody sequences, domains, fragments and specified variants thereof. The present invention also provides methods of making and using said nucleic acids and mimetibodies or specified portions or variants, including therapeutic compositions, methods and devices.

The present invention also provides at least one isolated hinge core mimetibody or specified portion or variant as described herein and/or as known in the art. The hinge core mimetibody can optionally comprise at least one CH3 region directly linked with at least one CH2 region directly linked with at least one hinge region or fragment thereof (H) directly linked with at least one optional linker sequence (L), directly linked to at least one therapeutic peptide (P), optionally further directly linked with at least a portion of at least one variable (V) antibody sequence.

In a preferred embodiment a hinge core mimetibody comprises formula (I):
((V(m)-P(n)-L(o)-H(p)-CH2(q)-CH3(r))(s),
where V is at least one portion of an N-terminus of an immunoglobulin variable region, P is at least one bioactive peptide, L is polypeptide that provides structural flexablity by allowing the mimietibody to have alternative orientations and binding properties, H is at least a portion of an immunoglobulin variable hinge region, CH2 is at least a portion of an immunoglobulin CH2 constant region, CH3 is at least a portion of an immunoglobulin CH3 constant region, and m, n, o, p, q, r, and s can be independently an integer between 0, 1 or 2 and 10, mimicing different types of immunoglobulin molecules, e.g., but not limited to IgG1, IgG2, IgG3, IgG4, IgA, IgM, IgD, IgE, and the like, or combination thereof. The monomer where m=1 can be linked to other monomers by association or covalent linkage, such as, but not limited to, a Cys-Cys disulfide bond or other immnuoglobulin sequence. Thus, a hinge core mimetibody of the present invention mimics an antibody structure with its inherent properties and functions, while providing a therapeutic peptide and its inherent or acquired in vitro, in vivo or in situ properties or activities. The various portions of the antibody and therapeutic peptide portions of at least one hinge core mimetibody of the present invention can vary as described herein in combinatoin with what is known in the art.

As used herein, a “hinge core mimetibody,” “hinge core mimetibody portion,” or “hinge core mimetibody fragment” and/or “hinge core mimetibody variant” and the like mimics, has or simulates at least one ligand binding or at least one biological activity of at least one protein, such as but not limited to at least one biologically active portion of at least one of SEQ ID NOS:1-979, in vitro, in situ and/or preferably in vivo. For example, a suitable hinge core mimetibody, specified portion or variant of the present invention can bind at least one protein ligand and includes at least one protein ligand, receptor, soluble receptor, and the like. A suitable hinge core mimetibody, specified portion, or variant can also modulate, increase, modify, activate, at least one protein receptor signaling or other measurable or detectable activity.

Mimetibodies useful in the methods and compositions of the present invention are characterized by suitable affinity binding to protein ligands or receptors and optionally and preferably having low toxicity. In particular, a hinge core mimetibody, where the individual components, such as the portion of variable region, constant region (without a CH1 portion) and framework, or any portion thereof (e.g., a portion of the J, D or V rgions of the variable heavy or light chain;, at least one portion of at least one hinge region, the constant heavy chain or light chain, and the like) individually and/or collectively optionally and preferably possess low immunogenicity, is useful in the present invention. The mimetibodies that can be used in the invention are optionally characterized by their ability to treat patients for extended periods with good to excellent alleviation of symptoms and low toxicity. Low immunogenicity and/or high affinity, as well as other undefined properties, may contribute to the therapeutic results achieved. “Low immunogenicity” is defined herein as raising significant HAMA, HACA or HAHA responses in less than about 75%, or preferably less than about 50, 45, 40, 35, 30, 35, 20, 15, 10, 9, 8, 7, 6, 5, 4, 3, 2, and/or 1% ofthe patients treated and/or raising low titres in the patient treated (less than about 300, preferably less than about 100 measured with a double antigen enzyme immunoassay) (see, e.g., Elliott et al., Lancet 344:1125-1127 (1994)).

Utility

The isolated nucleic acids of the present invention can be used for production of at least one hinge core mimetibody, fragment or specified variant thereof, which can be used to effect in an cell, tissue, organ or animal (including mammals and humans), to modulate, treat, alleviate, help prevent the incidence of, or reduce the symptoms of, at least one protein related condition, selected from, but not limited to, at least one of an immune disorder or disease, a cardiovascular disorder or disease, an infectious, malignant, and/or neurologic disorder or disease, an anemia; an immune/autoimmune; and/or an cancerous/infecteous, as well as other known or specified protein related conditions.

Such a method can comprise administering an effective amount of a composition or a pharmaceutical composition comprising at least one hinge core mimetibody or specified portion or variant to a cell, tissue, organ, animal or patient in need of such modulation, treatment, alleviation, prevention, or reduction in symptoms, effects or mechanisms. The effective amount can comprise an amount of about 0.0001 to 500 mg/kg per single or multiple administration, or to achieve a serum concentration of 0.0001-5000 μg/ml serum concentration per single or multiple adminstration, or any effective range or value therein, as done and determined using known methods, as described herein or known in the relevant arts.

Citations

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2003); Sambrook, et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2003); Colligan et al., Current Protocols in Protein Science, John Wiley & Sons, NY, N.Y., (1997-2003).

### MIMETIBODIES OF THE PRESENT INVENTION

The hinge core mimetibody can optionally comprise at least one CH3 region directly linked with at least one CH2 region directly linked with at least portion of at lesat one hinge region fragment (H), such as comprising at least one core hinge region, directly linked with an optional linker sequence (L), directly linked to at least one therapeutic peptide (P), optionally further directly linked with at least a portion of at least one variable antibody sequence (V). In a preferred embodiment of a pair of a CH3-CH2-H-L-V, the pair can be linked by association or covalent linkage. Thus, a hinge core mimetibody of the present invention mimics an antibody structure with its inherent properties and functions, while providing a therapeutic peptide and its inherent or acquired in vitro, in vivo or in situ properties or activities. The various portions of the antibody and therapeutic peptide portions of at least one hinge core mimetibody of the present invention can vary as described herein in combinatoin with what is known in the art.

Mimetibodies of the present invention thus provide at least one suitable property as compared to known proteins, such as, but not limited to, at least one of increased half-life, increased activity, more specific activity, increased avidity, increased or descrease off rate, a selected or more suitable subset of activities, less immungenicity, increased quality or duration of at least one desired therapeutic effect, less side effects, and the like.

Such fragments can be produced by enzymatic cleavage, synthetic or recombinant techniques, as known in the art and/or as described herein. For example, papain or pepsin cleavage can generate hinge core mimetibody Fab or F(ab′)₂ fragments, respectively. Other proteases with the requisite substrate specificity can also be used to generate Fab or F(ab′)₂ fragments or portions thereof. Mimetibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH1 domain and/or hinge region of the heavy chain. The various portions of mimetibodies can be joined together chemically by conventional techniques, or can be prepared as a contiguous protein using genetic engineering techniques. For example, a nucleic acid encoding the variable and constant regions of a human antibody chain can be expressed to produce a contiguous protein for use in mimetibodies of the present invention. See, e.g., Ladner et al., U.S. Pat. No. 4,946,778 and Bird, R. E. et al., Science, 242: 423-426 (1988), regarding single chain antibodies.

As used herein, the term “human mimetibody” refers to an antibody in which substantially every part of the protein (e.g., therapeutic peptide, framework, C_{L}, C_{H} domains (e.g., C_{H}2, C_{H}3), hinge, (V_{L}, V_{H})) is expected to be substantially non-immunogenic, with only minor sequence changes or variations. Such changes or variations optionally and preferably retain or reduce the immunogenicity in humans relative to non-modified human antibodies, or mimetibodies of the prsent invention. Thus, a human antibody and corresponding hinge core mimetibody of the present invention is distinct from a chimeric or humanized antibody. It is pointed out that a human antibody and hinge core mimetibody can be produced by a non-human animal or cell that is capable of expressing human immunoglobulins (e.g., heavy chain and/or light chain) genes, and for a hinge core mimetibody.

Human mimetibodies that are specific for at least one protein ligand or receptor thereof can be designed against an appropriate ligand, such as isolated and/or protein receptor or ligand, or a portion thereof (including synthetic molecules, such as synthetic peptides). Preparation of such mimetibodies are performed using known techniques to identify and characterize ligand binding regions or sequences of at least one protein or portion thereof.

In a preferred embodiment, at least one hinge core mimetibody or specified portion or variant of the present invention is produced by at least one cell line, mixed cell line, immortalized cell or clonal population of immortalized and/or cultured cells. Immortalized protein producing cells can be produced using suitable methods. Preferably, the at least one hinge core mimetibody or specified portion or variant is generated by providing nucleic acid or vectors comprising DNA derived or having a substantially similar sequence to, at least one human immunoglobulin locus that is functionally rearranged, or which can undergo functional rearrangement, and which further comprises a mimetibody structure as described herein, e.g., but not limited to Formula (I), wherein known portions of: C— and N-termiinal variable regions can be used for V, hinge regions for H, CH2 for CH2 and CH3 for CH3, as known in the art.

The term “functionally rearranged,” as used herein refers to a segment of nucleic acid from an immunoglobulin locus that has undergone V(D)J recombination, thereby producing an immunoglobulin gene that encodes an immunoglobulin chain (e.g., heavy chain, light chain), or any portion thereof. A functionally rearranged immunoglobulin gene can be directly or indirectly identified using suitable methods, such as, for example, nucleotide sequencing, hybridization (e.g., Southern blotting, Northern blotting) using probes that can anneal to coding joints between gene segments or enzymatic amplification of immunoglobulin genes (e.g., polymerase chain reaction) with primers that can anneal to coding joints between gene segments. Whether a cell produces an hinge core mimetibody or portion or variant comprising a particular variable region or a variable region comprising a particular sequence (e.g., at least one P sequence can also be determined using suitable methods.

Mimetibodies, specified portions and variants of the present invention can also be prepared using at least one hinge core mimetibody or specified portion or variant encoding nucleic acid to provide transgenic animals or mammals, such as goats, cows, horses, sheep, and the like, that produce such mimetibodies or specified portions or variants in their milk. Such animals can be provided using known methods as applied for antibody encoding sequences. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Mimetibodies, specified portions and variants of the present invention can additionally be prepared using at least one hinge core mimetibody or specified portion or variant encoding nucleic acid to provide transgenic plants and cultured plant cells (e.g., but not limited to tobacco and maize) that produce such mimetibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain mimetibodies (scFv's), including tobacco seeds and potato tubers. See, e.g., Conrad et al., Plant Mol. Biol. 38:101-109 (1998) and references cited therein. Thus, mimetibodies, specified portions and variants of the present invention can also be produced using transgenic plants, according to know methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. The above references are entirely incorporated herein by reference.

The mimetibodies of the invention can bind human protein ligands with a wide range of affinities (K_{D}). In a preferred embodiment, at least one human hinge core mimetibody of the present invention can optionally bind at least one protein ligand with high affinity. For example, at least one hinge core mimetibody of the present invention can bind at least one protein ligand with a K_{D} equal to or less than about 10⁻⁹ M or, more preferably, with a K_{D} equal to or less than about 0.1-9.9 (or any range or value therein) X 10⁻¹⁰ M, 10⁻¹¹, 10⁻¹², 10¹³ or any range or value therein.

The affinity or avidity of a hinge core mimetibody for at least one protein ligand can be determined experimentally using any suitable method, e.g., as used for determing antibody-antigen binding affinity or avidity. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular hinge core mimetibody-ligand interaction can vary if measured under different conditions (e.g., salt concentration, pH). Thus, measurements of affinity and other ligand-binding parameters (e.g., K_{D}, Kₐ, K_{d}) are preferably made with standardized solutions of hinge core mimetibody and ligand, and a standardized buffer, such as the buffer described herein.

Nucleic Acid Molecules

Using the information provided herein, such as the nucleotide sequences encoding at least 90-100% of the contiguous amino acids of at least one of SEQ ID NOS:1-979 as well as at least one portion of an antibody, wherein the above sequences are inserted as the P sequence of Formula (I) to provide a hinge core mimetibody of the present invention, further comprising specified fragments, variants or consensus sequences thereof, or a deposited vector comprising at least one of these sequences, a nucleic acid molecule of the present invention encoding at least one hinge core mimetibody or specified portion or variant can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combination thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the present invention can include nucleic acid molecules comprising an open reading frame (ORF), optionally with one or more introns, nucleic acid molecules comprising the coding sequence for a hinge core mimetibody or specified portion or variant; and nucleic acid molecules which comprise a nucleotide sequence substantially different from those described above but which, due to the degeneracy of the genetic code, still encode at least one hinge core mimetibody as described herein and/or as known in the art. Of course, the genetic code is well known in the art. Thus, it would be routine for one skilled in the art to generate such degenerate nucleic acid variants that code for specific hinge core mimetibody or specified portion or variants of the present invention. See, e.g., Ausubel, et al., supra, and such nucleic acid variants are included in the present invention.

As indicated herein, nucleic acid molecules of the present invention which comprise a nucleic acid encoding a hinge core mimetibody or specified portion or variant can include, but are not limited to, those encoding the amino acid sequence of a hinge core mimetibody fragment, by itself; the coding sequence for the entire hinge core mimetibody or a portion thereof; the coding sequence for a hinge core mimetibody, fragment or portion, as well as additional sequences, such as the coding sequence of at least one signal leader or fusion peptide, intron, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding a hinge core mimetibody or specified portion or variant can be fused to a marker sequence, such as a sequence encoding a peptide that facilitates purification of the fused hinge core mimetibody or specified portion or variant comprising a hinge core mimetibody fragment or portion.

Polynucleotides which Selectively Hybridize to a Polynucleotide as Described herein

The present invention provides isolated nucleic acids that hybridize under selective hybridization conditions to a polynucleotide disclosed herein, or others disclosed herein, including specified variants or portions thereof. Thus, the polynucleotides of this embodiment can be used for isolating, detecting, and/or quantifying nucleic acids comprising such polynucleotides.

Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 40-99% sequence identity and can be employed to identify orthologous or paralogous sequences.

Optionally, polynucleotides of this invention will encode at least a portion of a hinge core mimetibody or specified portion or variant encoded by the polynucleotides described herein. The polynucleotides of this invention embrace nucleic acid sequences that can be employed for selective hybridization to a polynucleotide encoding a hinge core mimetibody or specified portion or variant of the present invention. See, e.g., Ausubel, supra; Colligan, supra, each entirely incorporated herein by reference.

Construction of Nucleic Acids

The isolated nucleic acids of the present invention can be made using (a) recombinant methods, (b) synthetic techniques, (c) purification techniques, or combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention—excluding the coding sequence—is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. See, e.g., Ausubel, supra; or Sambrook, supra.

Recombinant Methods for Constructing Nucleic Acids

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under suitable stringency conditions, to the polynucleotides of the present invention are used to identify the desired sequence in a cDNA or genomic DNA library. The isolation of RNA, and construction of cDNA and genomic libraries, is well known to those of ordinary skill in the art. (See, e.g., Ausubel, supra; or Sambrook, supra).

Synthetic Methods for Constructing Nucleic Acids

The isolated nucleic acids of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

Recombinant Expression Cassettes

The present invention further provides recombinant expression cassettes comprising a nucleic acid of the present invention. A nucleic acid sequence of the present invention, for example a cDNA or a genomic sequence encoding a hinge core mimetibody or specified portion or variant of the present invention, can be used to construct a recombinant expression cassette that can be introduced into at least one desired host cell. A recombinant expression cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In some embodiments, isolated nucleic acids that serve as promoter, enhancer, or other elements can be introduced in the appropriate position (upstream, downstream or in intron) of a non-heterologous form of a polynucleotide of the present invention so as to up or down regulate expression of a polynucleotide of the present invention. For example, endogenous promoters can be altered in vivo or in vitro by mutation, deletion and/or substitution, as known in the art. A polynucleotide of the present invention can be expressed in either sense or anti-sense orientation as desired. It will be appreciated that control of gene expression in either sense or anti-sense orientation can have a direct impact on the observable characteristics. Another method of suppression is sense suppression. Introduction of nucleic acid configured in the sense orientation has been shown to be an effective means by which to block the transcription of target genes.

Vectors and Host Cells

The present invention also relates to vectors that include isolated nucleic acid molecules of the present invention, host cells that are genetically engineered with the recombinant vectors, and the production of at least one hinge core mimetibody or specified portion or variant by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced into a cell using suitable known methods, such as electroporation and the like, other known methods include the use of the vector as a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites optionally for at least one of transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one hinge core mimetibody or specified portion or variant of the present invention can be expressed in a modified form, such as a fusion protein, and can include not only secretion signals, but also additional heterologous functional regions. For instance, a region of additional amino acids, particularly charged amino acids, can be added to the N-terminus of a hinge core mimetibody or specified portion or variant to improve stability and persistence in the host cell, during purification, or during subsequent handling and storage. Also, peptide moieties can be added to a hinge core mimetibody or specified portion or variant of the present invention to facilitate purification. Such regions can be removed prior to final preparation of a hinge core mimetibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the present invention.

Illustrative of cell cultures useful for the production of the mimetibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, hepG2 cells, P3X63Ag8.653, SP2/0-Ag14, 293 cells, HeLa cells and the like, which are readily available from, for example, American Type Culture Collection, Manassas, Va. Preferred host cells include cells of lymphoid origin such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

Purification of an Hinge Core Mimetibody or Specified Portion or Variant thereof

A hinge core mimetibody or specified portion or variant can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2003), e.g., Chapters 1, 4, 6, 8, 9, 10, each entirely incorporated herein by reference.

Mimetibodies or specified portions or variants of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the hinge core mimetibody or specified portion or variant of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

Mimetibodies, Specified Fragments and/or Variants

The isolated mimetibodies of the present invention comprise a hinge core mimetibody or specified portion or variant encoded by any one of the polynucleotides of the present invention as discussed more fully herein, or any isolated or prepared hinge core mimetibody or specified portion or variant thereof.

Preferably, the hinge core mimetibody or ligand-binding portion or variant binds at least one protein ligand or receptor, and, thereby provides at least one biological activity of the corresponding protein or a fragment thereof. Different therapeutically or diagnostically significant proteins are well known in the art and suitable assays or biological activities of such proteins are also well known in the art. The following is a general discussion of the variety of proteins, peptides and biological molecules that may be used in the in accordance with the teachings herein. These descriptions do not serve to limit the scope of the invention, but rather illustrate the breadth of the invention.

Thus, an embodiment of the present invention may target one or more growth factors, or, conversely, derive the target-binding moiety from one or more growth factors. Briefly, growth factors are hormones or cytokine proteins that bind to receptors on the cell surface, with the primary result of activating cellular proliferation and/or differentiation. Many growth factors are quite versatile, stimulating cellular division in numerous different cell types; while others are specific to a particular cell-type. The following Table 1 presents several factors, but is not intended to be comprehensive or complete, yet introduces some of the more commonly known factors and their principal activities.

TABLE 1
Growth Factors

| Factor | Principal Source | Primary Activity | Comments |
|---|---|---|---|
| Platelet Derived | Platelets, endothelial | Promotes proliferation of | Dimer required for |
| Growth Factor | cells, placenta. | connective tissue, glial and | receptor binding. |
| (PDGF) | | smooth muscle cells. PDGF | Two different protein |
| | | receptor has intrinsic tyrosine | chains, A and B, |
| | | kinase activity. | form 3 distinct dimer |
| | | | forms. |
| Epidermal | Submaxillary gland, | promotes proliferation of | EGF receptor has |
| Growth Factor | Brunners gland. | mesenchymal, glial and | tyrosine kinase |
| (EGF) | | epithelial cells. | activity, activated in |
| | | | response to EGF |
| | | | binding. |
| Fibroblast Growth | Wide range of cells; | Promotes proliferation of | Four distinct |
| Factor (FGF) | protein is associated with | many cells including skeletal | receptors, all with |
| | the ECM; nineteen family | and nervous system; inhibits | tyrosine kinase |
| | members. Receptors | some stem cells; induces | activity. FGF |
| | widely distributed in | mesodermal differentiation. | implicated in mouse |
| | bone, implicated in | Non-proliferative effects | mammary tumors and |
| | several bone-related | include regulation of pituitary | Kaposi's sarcoma. |
| | diseases. | and ovarian cell function. | |
| NGF | | Promotes neurite outgrowth | Several related |
| | | and neural cell survival. | proteins first |
| | | | identified as proto- |
| | | | oncogenes; trkA |
| | | | (trackA), trkB, trkC. |
| Erythropoietin | Kidney. | Promotes proliferation and | Also considered a |
| (Epo) | | differentiation of erythrocytes. | ‘blood protein,’ and a |
| | | | colony stimulating |
| | | | factor. |
| Transforming | Common in transformed | Potent keratinocyte growth | Related to EGF. |
| Growth Factor a | cells, found in | factor. | |
| (TGF-a) | macrophages and | | |
| | keratinocytes. | | |
| Transforming | Tumor cells, activated | Anti-inflammatory (suppresses | Large family of |
| Growth Factor v | TH₁ cells (T-helper) and | cytokine production and class | proteins including |
| (TGF-b) | natural killer (NK) cells. | II MHC expression), | activin, inhibin and |
| | | proliferative effects on many | bone morpho-genetic |
| | | mesenchymal and epithelial | protein. Several |
| | | cell types, may inhibit | classes and |
| | | macrophage and lymphocyte | subclasses of cell- |
| | | proliferation. | surface receptors. |
| Insulin-Like | Primarily liver, produced | Promotes proliferation of | Related to IGF-II and |
| Growth Factor-I | in response to GH and | many cell types, autocrine and | proinsulin, also |
| (IGF-I) | then induces subsequent | paracrine activities in addition | called Somatomedin |
| | cellular activities, | to the initially observed | C. IGF-I receptor, |
| | particularly on bone | endocrine activities on bone. | like the insulin |
| | growth. | | receptor, has intrinsic |
| | | | tyrosine kinase |
| | | | activity. IGF-I can |
| | | | bind to the insulin |
| | | | receptor. |
| Insulin-Like | Expressed almost | Promotes proliferation of | IGF-II receptor is |
| Growth Factor-II | exclusively in embryonic | many cell types primarily of | identical to the |
| (IGF-II) | and neonatal tissues. | fetal origin. Related to IGF-I | mannose-6-phosphate |
| | | and proinsulin. | receptor that is |
| | | | responsible for the |
| | | | integration of |
| | | | lysosomal enzymes. |

Additional growth factors that may be produced in accordance with the present invention include Activin (Vale et al., 321 Nature 776 (1986); Ling et al., 321 Nature 779 (1986)), Inhibin (U.S. Pat. Nos. 4,737,578; 4,740,587), and Bone Morphongenic Proteins (BMPS) (U.S. Pat. No. 5,846,931; Wozney, Cellular & Molecular Biology of Bone 131-167 (1993)).

In addition to the growth factors discussed above, the present invention may target or use other cytokines. Secreted primarily from leukocytes, cytokines stimulate both the humoral and cellular immune responses, as well as the activation of phagocytic cells. Cytokines that are secreted from lymphocytes are termed lymphokines, whereas those secreted by monocytes or macrophages are termed monokines. A large family of cytokines are produced by various cells of the body. Many of the lymphokines are also known as interleukins (ILs), because they are not only secreted by leukocytes, but are also able to affect the cellular responses of leukocytes. More specifically, interleukins are growth factors targeted to cells of hematopoietic origin. The list of identified interleukins grows continuously. See, e.g., U.S. Pat. No. 6,174,995; U.S. Pat. No. 6,143,289; Sallusto et al., 18 Annu. Rev. Immunol. 593 (2000) Kunkel et al., 59 J. Leukocyto Biol. 81 (1996).

Additional growth factor/cytokines encompassed in the present invention include pituitary hormones such as human growth hormone (HGH), follicle stimulating hormones (FSH, FSHα, and FSHβ), Human Chorionic Gonadotrophins (HCG, HCGα, HCGβ), uFSH (urofollitropin), Gonatropin releasing hormone (GRH), Growth Hormone (GH), leuteinizing hormones (LH, LHα, LHβ), somatostatin, prolactin, thyrotropin (TSH, TSHα, TSHβ), thyrotropin releasing hormone (TRH), parathyroid hormones, estrogens, progesterones, testosterones, or structural or functional analog thereof. All of these proteins and peptides are known in the art.

The cytokine family also includes tumor necrosis factors, colony stimulating factors, and interferons. See, e.g., Cosman, 7 Blood Cell (1996); Gruss et al., 85 Blood 3378 (1995); Beutler et al., 7 Annu. Rev. Immunol. 625 (1989); Aggarwal et al., 260 J. Biol. Chem. 2345 (1985); Pennica et al., 312 Nature 724 (1984); R & D Systems, Cytokine Mini-Reviews, at http://www.rndsystems.com. Several cytokines are introduced, briefly, in Table 2 below.

TABLE 2
Cytokines

| Cytokine | Principal Source | Primary Activity |
|---|---|---|
| Interleukins | Primarily macrophages but also | Costimulation of APCs and T cells; |
| IL1-a and -b | neutrophils, endothelial cells, smooth | stimulates IL-2 receptor production and |
| | muscle cells, glial cells, astrocytes, B- | expression of interferon-γ; may induce |
| | and T-cells, fibroblasts, and | proliferation in non-lymphoid cells. |
| | keratinocytes | |
| IL-2 | CD4+ T-helper cells, activated TH₁ | Major interleukin responsible for clonal |
| | cells, NK cells | T-cell proliferation. IL-2 also exerts |
| | | effects on B-cells, macrophages, and |
| | | natural killer (NK) cells. IL-2 receptor is |
| | | not expressed on the surface of resting T- |
| | | cells, but expressed constitutively on NK |
| | | cells, that will secrete TNF-α, IFN-γ and |
| | | GM-CSF in response to IL-2, which in |
| | | turn activate macrophages. |
| IL-3 | Primarily T-cells | Also known as multi-CSF, as it stimulates |
| | | stem cells to produce all forms of |
| | | hematopoietic cells. |
| IL-4 | TH₂ and mast cells | B cell proliferation, eosinophil and mast |
| | | cell growth and function, IgE and class II |
| | | MHC expression on B cells, inhibition of |
| | | monokine production |
| IL-5 | TH₂ and mast cells | eosinophil growth and function |
| IL-6 | Macrophages, fibroblasts, endothelial | IL-6 acts in synergy with IL-1 and TNF-α |
| | cells and activated T-helper cells. | in many immune responses, including T- |
| | Does not induce cytokine expression. | cell activation; primary inducer of the |
| | | acute-phase response in liver; enhances |
| | | the differentiation of B-cells and their |
| | | consequent production of |
| | | immunoglobulin; enhances |
| | | Glucocorticoid synthesis. |
| IL-7 | thymic and marrow stromal cells | T and B lymphopoiesis |
| IL-8 | Monocytes, neutrophils, macrophages, | Chemoattractant (chemokine) for |
| | and NK cells | neutrophils, basophils and T-cells; |
| | | activates neutrophils to degranulate. |
| IL-9 | T cells | hematopoietic and thymopoietic effects |
| IL-10 | activated TH₂ cells, CD8⁺ T and B | inhibits cytokine production, promotes B |
| | cells, macrophages | cell proliferation and antibody |
| | | production, suppresses cellular immunity, |
| | | mast cell growth |
| IL-11 | stromal cells | synergisitc hematopoietic and |
| | | thrombopoietic effects |
| IL-12 | B cells, macrophages | proliferation of NK cells, INF-g |
| | | production, promotes cell-mediated |
| | | immune functions |
| IL-13 | TH₂ cells | IL-4-like activities |
| IL-18 | macrophages/Kupffer cells, | Interferon-gamma-inducing factor with |
| | keratinocytes, glucocorticoid-secreting | potent pro-inflammatory activity |
| | adrenal cortex cells, and osteoblasts | |
| IL-21 | Activated T cells | IL21 has a role in proliferation and |
| | | maturation of natural killer (NK) cell |
| | | populations from bone marrow, in the |
| | | proliferation of mature B-cell populations |
| | | co-stimulated with anti-CD40, and in the |
| | | proliferation of T cells co-stimulated with |
| | | anti-CD3. |
| IL-23 | Activated dendritic cells | A complex of p19 and the p40 subunit of |
| | | IL-12. IL-23 binds to IL-12R beta 1 but |
| | | not IL-12R beta 2; activates Stat4 in PHA |
| | | blast T cells; induces strong proliferation |
| | | of mouse memory T cells; stimulates IFN- |
| | | gamma production and proliferation in |
| | | PHA blast T cells, as well as in CD45RO |
| | | (memory) T cells. |
| TumorNecrosis | Primarily activated macrophages. | Once called cachectin; induces the |
| Factor | | expression of other autocrine growth |
| TNF-α | | factors, increases cellular responsiveness |
| | | to growth factors; induces signaling |
| | | pathways that lead to proliferation; |
| | | induces expression of a number of |
| | | nuclear proto-oncogenes as well as of |
| | | several interleukins. |
| (TNF-β) | T-lymphocytes, particularly cytotoxic | Also called lymphotoxin; kills a number |
| | T-lymphocytes (CTL cells); induced | of different cell types, induces terminal |
| | by IL-2 and antigen-T-Cell receptor | differentiation in others; inhibits |
| | interactions. | lipoprotein lipase present on the surface |
| | | of vascular endothelial cells. |
| Interferons | macrophages, neutrophils and some | Known as type I interferons; antiviral |
| INF-a and -b | somatic cells | effect; induction of class I MHC on all |
| | | somatic cells; activation of NK cells and |
| | | macrophages. |
| Interferon | Primarily CD8+ T-cells, activated TH₁ | Type II interferon; induces of class I |
| INF-γ | and NK cells | MHC on all somatic cells, induces class II |
| | | MHC on APCs and somatic cells, |
| | | activates macrophages, neutrophils, NK |
| | | cells, promotes cell-mediated immunity, |
| | | enhances ability of cells to present |
| | | antigens to T-cells; antiviral effects. |
| Monocyte | Peripheral blood | Attracts monocytes to sites of vascular |
| Chemoattractant | monocytes/macrophages | endothelial cell injury, implicated in |
| Protein-1 (MCP1) | | atherosclerosis. |
| Colony | | Stimulate the proliferation of specific |
| Stimulating | | pluripotent stem cells of the bone marrow |
| Factors (CSFs) | | in adults. |
| Granulocyte-CSF | | Specific for proliferative effects on cells |
| (G-CSF) | | of the granulocyte lineage; proliferative |
| | | effects on both classes of lymphoid cells. |
| Macrophage-CSF | | Specific for cells of the macrophage |
| (M-CSF) | | lineage. |
| Granulocyte- | | Proliferative effects on cells of both the |
| MacrophageCSF | | macrophage and granulocyte lineages. |
| (GM-CSF) | | |

Other cytokines of interest that may be produced by the invention described herein include adhesion molecules (R & D Systems, Adhesion Molecule I (1996), at http://www.rndsystems.com); angiogenin (U.S. Pat. No. 4,721,672; Moener et al., 226 Eur. J. Biochem. 483 (1994)); annexin V (Cookson et al., 20 Genomics 463 (1994); Grundmann et al., 85 Proc. Natl. Acad. Sci. USA 3708 (1988); U.S. Pat. No. 5,767,247); caspases (U.S. Pat. No. 6,214,858; Thornberry et al., 281 Science 1312 (1998)); chemokines (U.S. Pat. Nos. 6,174,995; 6,143,289; Sallusto et al., 18 Annu. Rev. Immunol. 593 (2000) Kunkel et al., 59 J. Leukocyte Biol. 81 (1996)); endothelin (U.S. Pat. Nos. 6,242,485; 5,294,569; 5,231,166); eotaxin (U.S. Pat. No. 6,271,347; Ponath et al., 97(3) J. Clin. Invest. 604-612 (1996)); Flt-3 (U.S. Pat. No. 6,190,655); heregulins (U.S. Pat. Nos. 6,284,535; 6,143,740; 6,136,558; 5,859,206; 5,840,525); Leptin (Leroy et al., 271(5) J. Biol. Chem. 2365 (1996); Maffei et al., 92 Proc. Natl. Acad. Sci. USA 6957 (1995); Zhang Y. et al. (1994) Nature 372: 425-432); Macrophage Stimulating Protein (MSP) (U.S. Pat. Nos. 6,248,560; 6,030,949; 5,315,000); Neurotrophic Factors (U.S. Pat. Nos. 6,005,081; 5,288,622); Pleiotrophin/Midkine (PTN/MK) (Pedraza et al., 117 J. Biochem. 845 (1995); Tamura et al., 3 Endocrine 21 (1995); U.S. Pat. No. 5,210,026; Kadomatsu et al., 151 Biochem. Biophys. Res. Commun. 1312 (1988)); STAT proteins (U.S. Pat. Nos. 6,030808; 6,030,780; Darnell et al., 277 Science 1630-1635 (1997)); Tumor Necrosis Factor Family (Cosman, 7 Blood Cell (1996); Gruss et al., 85 Blood 3378 (1995); Beutler et al., 7 Annu. Rev. Immunol. 625 (1989); Aggarwal et al., 260 J. Biol. Chem. 2345 (1985); Pennica et al., 312 Nature 724 (1984)).

Also of interest regarding cytokines are proteins or chemical moieties that interact with cytokines, such as Matrix Metalloproteinases (MMPs) (U.S. Pat. No. 6,307,089; Nagase, Matrix Metalloproteinases in Zinc Metalloproteinases in Health and Disease (1996)), and Nitric Oxide Synthases (NOS) (Fukuto, 34 Adv. Pharm 1 (1995); U.S. Pat. No. 5,268,465).

The present invention may also be used to affect blood proteins, a generic name for a vast group of proteins generally circulating in blood plasma, and important for regulating coagulation and clot dissolution. See, e.g., Haematologic Technologies, Inc., HTI CATALOG, at www.haemtech.com. Table 3 introduces, in a non-limiting fashion, some of the blood proteins contemplated by the present invention.

TABLE 3
Blood Proteins

| Protein | Principle Activity | Reference |
|---|---|---|
| Factor V | In coagulation, this glycoprotein pro- | Mann et al., 57 ANN. REV. BIOCHEM. |
| | cofactor, is converted to active cofactor, | 915 (1988); see also Nesheim et al., 254 |
| | factor Va, via the serine protease α- | J. BIOL. CHEM. 508 (1979); Tracy et al., |
| | thrombin, and less efficiently by its | 60 BLOOD 59 (1982); Nesheim et al., 80 |
| | serine protease cofactor Xa. The | METHODS ENZYMOL. 249 (1981); Jenny |
| | prothrombinase complex rapidly | et al., 84 PROC. NATL. ACAD. SCI. USA |
| | converts zymogen prothrombin to the | 4846 (1987). |
| | active serine protease, α-thrombin. | |
| | Down regulation of prothrombinase | |
| | complex occurs via inactivation of Va | |
| | by activated protein C. | |
| Factor VII | Single chain glycoprotein zymogen in | See generally, Broze et al., 80 |
| | its native form. Proteolytic activation | METHODS ENZYMOL. 228 (1981); Bajaj |
| | yields enzyme factor VIIa, which binds | et al., 256 J. BIOL. CHEM. 253 (1981); |
| | to integral membrane protein tissue | Williams et al., 264 J. BIOL. CHEM. |
| | factor, forming an enzyme complex that | 7536 (1989); Kisiel et al., 22 |
| | proteolytically converts factor X to Xa. | THROMBOSIS RES. 375 (1981); |
| | Also known as extrinsic factor Xase | Seligsohn et al., 64 J. CLIN. INVEST. |
| | complex. Conversion of VII to VIIa | 1056 (1979); Lawson et al., 268 J. BIOL. |
| | catalyzed by a number of proteases | CHEM. 767 (1993). |
| | including thrombin, factors IXa, Xa, | |
| | XIa, and XIIa. Rapid activation also | |
| | occurs when VII combines with tissue | |
| | factor in the presence of Ca, likely | |
| | initiated by a small amount of pre- | |
| | existing VIIa. Not readily inhibited by | |
| | antithrombin III/heparin alone, but is | |
| | inhibited when tissue factor added. | |
| Factor IX | Zymogen factor IX, a single chain | Thompson, 67 BLOOD, 565 (1986); |
| | vitamin K-dependent glycoprotein, | Hedner et al., HEMOSTASIS AND |
| | made in liver. Binds to negatively | THROMBOSIS 39-47 (R. W. Colman, J. Hirsh, |
| | charged phospholipid surfaces. | V. J. Marder, E. W. Salzman ed., |
| | Activated by factor XIα or the factor | 2^{nd} ed. J. P. Lippincott Co., Philadelphia) |
| | VIIa/tissue factor/phospholipid | 1987; Fujikawa et al., 45 METHODS IN |
| | complex. Cleavage at one site yields the | ENZYMOLOGY 74 (1974). |
| | intermediate IXα, subsequently | |
| | converted to fully active form IXaβ by | |
| | cleavage at another site. Factor IXaβ is | |
| | the catalytic component of the “intrinsic | |
| | factor Xase complex” (factor | |
| | VIIIa/IXa/Ca²⁺/phospholipid) that | |
| | proteolytically activates factor X to | |
| | factor Xa. | |
| Factor X | Vitamin K-dependent protein zymogen, | See Davie et al., 48 ADV. ENZYMOL 277 |
| | made in liver, circulates in plasma as a | (1979); Jackson, 49 ANN. REV. |
| | two chain molecule linked by a disulfide | BIOCHEM. 765 (1980); see also |
| | bond. Factor Xa (activated X) serves as | Fujikawa et al., 11 BIOCHEM. 4882 |
| | the enzyme component of | (1972); Discipio et al., 16 BIOCHEM. |
| | prothrombinase complex, responsible | 698 (1977); Discipio et al., 18 |
| | for rapid conversion of prothrombin to | BIOCHEM. 899 (1979); Jackson et al., 7 |
| | thrombin. | BIOCHEM. 4506 (1968); McMullen et |
| | | al., 22 BIOCHEM. 2875 (1983). |
| Factor XI | Liver-made glycoprotein homodimer | Thompson et al., 60 J. CLIN. INVEST. |
| | circulates, in a non-covalent complex | 1376 (1977); Kurachi et al., 16 |
| | with high molecular weight kininogen, | BIOCHEM. 5831 (1977); Bouma et al., |
| | as a zymogen, requiring proteolytic | 252 J. BIOL. CHEM. 6432 (1977); |
| | activation to acquire serine protease | Wuepper, 31 FED. PROC. 624 (1972); |
| | activity. Conversion of factor XI to | Saito et al., 50 BLOOD 377 (1977); |
| | factor XIa is catalyzed by factor XIIa. | Fujikawa et al., 25 BIOCHEM. 2417 |
| | XIa unique among the serine proteases, | (1986); Kurachi et al., 19 BIOCHEM. |
| | since it contains two active sites per | 1330 (1980); Scott et al., 69 J. CLIN. |
| | molecule. Works in the intrinsic | INVEST. 844 (1982). |
| | coagulation pathway by catalyzing | |
| | conversion of factor IX to factor IXa. | |
| | Complex form, factor XIa/HMWK, | |
| | activates factor XII to factor XIIa and | |
| | prekallikrein to kallikrein. Major | |
| | inhibitor of XIa is a₁-antitrypsin and to | |
| | lesser extent, antithrombin-III. Lack of | |
| | factor XI procoagulant activity causes | |
| | bleeding disorder: plasma | |
| | thromboplastin antecedent deficiency. | |
| Factor XII | Glycoprotein zymogen. Reciprocal | Schmaier et al., 18-38, and Davie, 242-267 |
| (Hageman | activation of XII to active serine | HEMOSTASIS & THROMBOSIS |
| Factor) | protease factor XIIa by kallikrein is | (Colman et al., eds., J. B. Lippincott Co., |
| | central to start of intrinsic coagulation | Philadelphia, 1987). |
| | pathway. Surface bound α-XIIa activates | |
| | factor XI to XIa. Secondary cleavage of | |
| | α-XIIa by kallikrein yields β-XIIa, and | |
| | catalyzes solution phase activation of | |
| | kallikrein, factor VII and the classical | |
| | complement cascade. | |
| Factor XIII | Zymogenic form of glutaminyl-peptide | See McDonaugh, 340-357 HEMOSTASIS |
| | γ-glutamyl transferase factor XIIIa | & THROMBOSIS (Colman et al., eds., |
| | (fibrinoligase, plasma transglutaminase, | J. B. Lippincott Co., Philadelphia, 1987); |
| | fibrin stabilizing factor). Made in the | Folk et al., 113 METHODS ENZYMOL. |
| | liver, found extracellularly in plasma | 364 (1985); Greenberg et al., 69 BLOOD |
| | and intracellularly in platelets, | 867 (1987). Other proteins known to be |
| | megakaryocytes, monocytes, placenta, | substrates for Factor XIIIa, that may be |
| | uterus, liver and prostrate tissues. | hemostatically important, include |
| | Circulates as a tetramer of 2 pairs of | fibronectin (Iwanaga et al., 312 ANN. |
| | nonidentical subunits (A₂B₂). Full | NY ACAD. SCI. 56 (1978)), a₂- |
| | expression of activity is achieved only | antiplasmin (Sakata et al., 65 J. CLIN. |
| | after the Ca²⁺- and fibrin(ogen)- | INVEST. 290 (1980)), collagen (Mosher |
| | dependent dissociation of B subunit | et al., 64 J. CLIN. INVEST. 781 (1979)), |
| | dimer from A₂′ dimer. Last of the | factor V (Francis et al., 261 J. BIOL. |
| | zymogens to become activated in the | CHEM. 9787 (1986)), von Willebrand |
| | coagulation cascade, the only enzyme in | Factor (Mosher et al., 64 J. CLIN. |
| | this system that is not a serine protease. | INVEST. 781 (1979)) and |
| | XIIIa stabilizes the fibrin clot by | thrombospondin (Bale et al., 260 J. BIOL. |
| | crosslinking the α and γ-chains of fibrin. | CHEM. 7502 (1985); Bohn, 20 |
| | Serves in cell proliferation in wound | MOL. CELL BIOCHEM. 67 (1978)). |
| | healing, tissue remodeling, | |
| | atherosclerosis, and tumor growth. | |
| Fibrinogen | Plasma fibrinogen, a large glycoprotein, | FURLAN, Fibrinogen, IN HUMAN |
| | disulfide linked dimer made of 3 pairs | PROTEIN DATA, (Haeberli, ed., VCH |
| | of non-identical chains (Aa, Bb and g), | Publishers, N.Y., 1995); Doolittle, in |
| | made in liver. Aa has N-terminal peptide | HAEMOSTASIS & THROMBOSIS, 491-513 |
| | (fibrinopeptide A (FPA), factor XIIIa | (3rd ed., Bloom et al., eds., Churchill |
| | crosslinking sites, and 2 | Livingstone, 1994); HANTGAN, et al., in |
| | phosphorylation sites. Bb has | HAEMOSTASIS & THROMBOSIS 269-89 |
| | fibrinopeptide B (FPB), 1 of 3 N-linked | (2d ed., Forbes et al., eds., Churchill |
| | carbohydrate moieties, and an N- | Livingstone, 1991). |
| | terminal pyroglutamic acid. The g chain | |
| | contains the other N-linked glycos. site, | |
| | and factor XIIIa cross-linking sites. Two | |
| | elongated subunits ((AaBbg)₂) align in | |
| | an antiparallel way forming a trinodular | |
| | arrangement of the 6 chains. Nodes | |
| | formed by disulfide rings between the 3 | |
| | parallel chains. Central node (n- | |
| | disulfide knot, E domain) formed by N- | |
| | termini of all 6 chains held together by | |
| | 11 disulfide bonds, contains the 2 IIa- | |
| | sensitive sites. Release of FPA by | |
| | cleavage generates Fbn I, exposing a | |
| | polymerization site on Aa chain. These | |
| | sites bind to regions on the D domain of | |
| | Fbn to form proto-fibrils. Subsequent IIa | |
| | cleavage of FPB from the Bb chain | |
| | exposes additional polymerization sites, | |
| | promoting lateral growth of Fbn | |
| | network. Each of the 2 domains between | |
| | the central node and the C-terminal | |
| | nodes (domains D and E) has parallel a- | |
| | helical regions of the Aa, Bb and g | |
| | chains having protease-(plasmin-) | |
| | sensitive sites. Another major plasmin | |
| | sensitive site is in hydrophilic | |
| | preturbance of a-chain from C-terminal | |
| | node. Controlled plasmin degradation | |
| | converts Fbg into fragments D and E. | |
| Fibronectin | High molecular weight, adhesive, | Skorstengaard et al., 161 Eur. J. |
| | glycoprotein found in plasma and | BIOCHEM. 441 (1986); Kornblihtt et al., |
| | extracellular matrix in slightly different | 4 EMBO J. 1755 (1985); Odermatt et |
| | forms. Two peptide chains | al., 82 PNAS 6571 (1985); Hynes, R. O., |
| | interconnected by 2 disulfide bonds, has | ANN. REV. CELL BIOL., 1, 67 (1985); |
| | 3 different types of repeating | Mosher 35 ANN. REV. MED. 561 |
| | homologous sequence units. Mediates | (1984); Rouslahti et al., 44 Cell 517 |
| | cell attachment by interacting with cell | (1986); Hynes 48 CELL 549 (1987); |
| | surface receptors and extracellular | Mosher 250 BIOL. CHEM. 6614 (1975). |
| | matrix components. Contains an Arg- | |
| | Gly-Asp-Ser (RGDS) cell attachment- | |
| | promoting sequence, recognized by | |
| | specific cell receptors, such as those on | |
| | platelets. Fibrin-fibronectin complexes | |
| | stabilized by factor XIIIa-catalyzed | |
| | covalent cross-linking of fibronectin to | |
| | the fibrin a chain. | |
| b₂-Glycoprotein I | Also called b₂I and Apolipoprotein H. | See, e.g., Lozier et al., 81 PNAS 2640-44 |
| | Highly glycosylated single chain protein | (1984); Kato & Enjyoi 30 BIOCHEM. |
| | made in liver. Five repeating mutually | 11687-94 (1997); Wurm, 16 INT'L J. |
| | homologous domains consisting of | BIOCHEM. 511-15 (1984); Bendixen et |
| | approximately 60 amino acids disulfide | al., 31 BIOCHEM. 3611-17 (1992); |
| | bonded to form Short Consensus | Steinkasserer et al., 277 BIOCHEM. J. |
| | Repeats (SCR) or Sushi domains. | 387-91 (1991); Nimpf et al., 884 |
| | Associated with lipoproteins, binds | BIOCHEM. BIOPHYS. ACTA 142-49 |
| | anionic surfaces like anionic vesicles, | (1986); Kroll et.al. 434 BIOCHEM. |
| | platelets, DNA, mitochondria, and | BIOPHYS. Acta 490-501 (1986); Polz et |
| | heparin. Binding can inhibit contact | al., 11 INT'L J. BIOCHEM. 265-73 |
| | activation pathway in blood coagulation. | (1976); McNeil et al., 87 PNAS 4120-24 |
| | Binding to activated platelets inhibits | (1990); Galli et al;. I LANCET 1544-47 |
| | platelet associated prothrombinase and | (1990); Matsuuna et al., II LANCET 177-78 |
| | adenylate cyclase activities. Complexes | (1990); Pengo et al., 73 THROMBOSIS |
| | between b₂I and cardiolipin have been | & HAEMOSTASIS 29-34 (1995). |
| | implicated in the anti-phospholipid | |
| | related immune disorders LAC and | |
| | SLE. | |
| Osteonectin | Acidic, noncollagenous glycoprotein | Villarreal et al., 28 BIOCHEM. 6483 |
| | (Mr = 29,000) originally isolated from | (1989); Tracy et al., 29 INT'L J. |
| | fetal and adult bovine bone matrix. May | BIOCHEM. 653 (1988); Romberg et al., |
| | regulate bone metabolism by binding | 25 BIOCHEM. 1176 (1986); Sage & |
| | hydroxyapatite to collagen. Identical to | Bornstein 266 J. BIOL. CHEM. 14831 |
| | human placental SPARC. An alpha | (1991); Kelm & Mann 4 J. BONE MIN. |
| | granule component of human platelets | RES. 5245 (1989); Kelm et al., 80 |
| | secreted during activation. A small | BLOOD 3112 (1992). |
| | portion of secreted osteonectin | |
| | expressed on the platelet cell surface in | |
| | an activation-dependent manner | |
| Plasminogen | Single chain glycoprotein zymogen with | See Robbins, 45 METHODS IN |
| | 24 disulfide bridges, no free sulfhydryls, | ENZYMOLOGY 257 (1976); COLLEN, |
| | and 5 regions of internal sequence | 243-258 BLOOD COAG. (Zwaal et al., |
| | homology, “kringles”, each five triple- | eds., New York, Elsevier, 1986); see |
| | looped, three disulfide bridged, and | also Castellino et al., 80 METHODS IN |
| | homologous to kringle domains in t-PA, | ENZYMOLOGY 365 (1981); Wohl et al., |
| | u-PA and prothrombin. Interaction of | 27 THROMB. RES. 523 (1982); Barlow |
| | plasminogen with fibrin and α2- | et al., 23 BIOCHEM. 2384 (1984); |
| | antiplasmin is mediated by lysine | SOTTRUP-JENSEN ET AL., 3 PROGRESS IN |
| | binding sites. Conversion of | CHEM. FIBRINOLYSIS & THROMBOLYSIS |
| | plasminogen to plasmin occurs by | 197-228 (Davidson et al., eds., Raven |
| | variety of mechanisms, including | Press, New York 1975). |
| | urinary type and tissue type | |
| | plasminogen activators, streptokinase, | |
| | staphylokinase, kallikrein, factors IXa | |
| | and XIIa, but all result in hydrolysis at | |
| | Arg560-Val561, yielding two chains | |
| | that remain covalently associated by a | |
| | disulfide bond. | |
| tissue | t-PA, a serine endopeptidase | See Plasminogen. |
| Plasminogen | synthesized by endothelial cells, is the | |
| Activator | major physiologic activator of | |
| | plasminogen in clots, catalyzing | |
| | conversion of plasminogen to plasmin | |
| | by hydrolising a specific arginine- | |
| | alanine bond. Requires fibrin for this | |
| | activity, unlike the kidney-produced | |
| | version, urokinase-PA. | |
| Plasmin | See Plasminogen. Plasmin, a serine | See Plasminogen. |
| | protease, cleaves fibrin, and activates | |
| | and/or degrades compounds of | |
| | coagulation, kinin generation, and | |
| | complement systems. Inhibited by a | |
| | number of plasma protease inhibitors in | |
| | vitro. Regulation of plasmin in vivo | |
| | occurs mainly through interaction with | |
| | a₂-antiplasmin, and to a lesser extent, a₂- | |
| | macroglobulin. | |
| Platelet Factor-4 | Low molecular weight, heparin-binding | Rucinski et al., 53 BLOOD 47 (1979); |
| | protein secreted from agonist-activated | Kaplan et al., 53 BLOOD 604 (1979); |
| | platelets as a homotetramer in complex | George 76 BLOOD 859 (1990); Busch et |
| | with a high molecular weight, | al., 19 THROMB. RES. 129 (1980); Rao |
| | proteoglycan, carrier protein. Lysine- | et al., 61 BLOOD 1208 (1983); Brindley, |
| | rich, COOH-terminal region interacts | et al., 72 J. CLIN. INVEST. 1218 (1983); |
| | with cell surface expressed heparin-like | Deuel et al., 74 PNAS 2256 (1981); |
| | glycosaminoglycans on endothelial | Osterman et al., 107 BIOCHEM. |
| | cells, PF-4 neutralizes anticoagulant | BIOPHYS. RES. COMMUN. 130 (1982); |
| | activity of heparin exerts procoagulant | Capitanio et al., 839 BIOCHEM. |
| | effect, and stimulates release of | BIOPHYS. ACTA 161 (1985). |
| | histamine from basophils. Chemotactic | |
| | activity toward neutrophils and | |
| | monocytes. Binding sites on the platelet | |
| | surface have been identified and may be | |
| | important for platelet aggregation. | |
| Protein C | Vitamin K-dependent zymogen, protein | See Esmon, 10 PROGRESS IN THROMB. |
| | C, made in liver as a single chain | & HEMOSTS. 25 (1984); Stenflo, 10 |
| | polypeptide then converted to a | SEMIN. IN THROMB. & HEMOSTAS. 109 |
| | disulfide linked heterodimer. Cleaving | (1984); Griffen et al., 60 BLOOD 261 |
| | the heavy chain of human protein C | (1982); Kisiel et al., 80 METHODS |
| | converts the zymogen into the serine | ENZYMOL. 320 (1981); Discipio et al., |
| | protease, activated protein C. Cleavage | 18 BIOCHEM. 899 (1979). |
| | catalyzed by a complex of α-thrombin | |
| | and thrombomodulin. Unlike other | |
| | vitamin K dependent coagulation | |
| | factors, activated protein C is an | |
| | anticoagulant that catalyzes the | |
| | proteolytic inactivation of factors Va | |
| | and VIIIa, and contributes to the | |
| | fibrinolytic response by complex | |
| | formation with plasminogen activator | |
| | inhibitors. | |
| Protein S | Single chain vitamin K-dependent | Walker 10 SEMIN. THROMB. |
| | protein functions in coagulation and | HEMOSTAS. 131 (1984); Dahlback et al., |
| | complement cascades. Does not possess | 10 SEMIN. THROMB. HEMOSTAS., 139 |
| | the catalytic triad. Complexes to C4b | (1984); Walker 261 J. BIOL. CHEM. |
| | binding protein (C4BP) and to | 10941 (1986). |
| | negatively charged phospholipids, | |
| | concentrating C4BP at cell surfaces | |
| | following injury. Unbound S serves as | |
| | anticoagulant cofactor protein with | |
| | activated Protein C. A single cleavage | |
| | by thrombin abolishes protein S cofactor | |
| | activity by removing gla domain. | |
| Protein Z | Vitamin K-dependent, single-chain | Sejima et al., 171 BIOCHEM. |
| | protein made in the liver. Direct | BIOPHYSICS RES. COMM. 661 (1990); |
| | requirement for the binding of thrombin | Hogg et al., 266 J. BIOL. CHEM. 10953 |
| | to endothelial phospholipids. Domain | (1991); Hogg et al., 17 BIOCHEM. |
| | structure similar to that of other vitamin | BIOPHYSICS RES. COMM. 801 (1991); |
| | K-dependant zymogens like factors VII, | Han et al., 38 BIOCHEM. 11073 (1999); |
| | IX, X, and protein C. N-terminal region | Kemkes-Matthes et al., 79 THROMB. |
| | contains carboxyglutamic acid domain | RES. 49 (1995). |
| | enabling phospholipid membrane | |
| | binding. C-terminal region lacks | |
| | “typical” serine protease activation site. | |
| | Cofactor for inhibition of coagulation | |
| | factor Xa by serpin called protein Z- | |
| | dependant protease inhibitor. Patients | |
| | diagnosed with protein Z deficiency | |
| | have abnormal bleeding diathesis during | |
| | and after surgical events. | |
| Prothrombin | Vitamin K-dependent, single-chain | Mann et al., 45 METHODS IN |
| | protein made in the liver. Binds to | ENZYMOLOGY 156 (1976); Magnusson |
| | negatively charged phospholipid | et al., PROTEASES IN BIOLOGICAL |
| | membranes. Contains two “kringle” | CONTROL 123-149 (Reich et al., eds. |
| | structures. Mature protein circulates in | Cold Spring Harbor Labs., New York |
| | plasma as a zymogen and, during | 1975); Discipio et al., 18 BIOCHEM. 899 |
| | coagulation, is proteolytically activated | (1979). |
| | to the potent serine protease α-thrombin. | |
| α-Thrombin | See Prothrombin. During coagulation, | 45 METHODS ENZYMOL. 156 (1976). |
| | thrombin cleaves fibrinogen to form | |
| | fibrin, the terminal proteolytic step in | |
| | coagulation, forming the fibrin clot. | |
| | Thrombin also responsible for feedback | |
| | activation of procofactors V and VIII. | |
| | Activates factor XIII and platelets, | |
| | functions as vasoconstrictor protein. | |
| | Procoagulant activity arrested by | |
| | heparin cofactor II or the antithrombin | |
| | III/heparin complex, or complex | |
| | formation with thrombomodulin. | |
| | Formation of thrombin/thrombomodulin | |
| | complex results in inability of thrombin | |
| | to cleave fibrinogen and activate factors | |
| | V and VIII, but increases the efficiency | |
| | of thrombin for activation of the | |
| | anticoagulant, protein C. | |
| b-Thrombo- | Low molecular weight, heparin-binding, | See, e.g., George 76 BLOOD 859 (1990); |
| globulin | platelet-derived tetramer protein, | Holt & Niewiarowski 632 BIOCHIM. |
| | consisting of four identical peptide | BIOPHYS. ACTA 284 (1980); |
| | chains. Lower affinity for heparin than | Niewiarowski et al., 55 BLOOD 453 |
| | PF-4. Chemotactic activity for human | (1980); Varma et al., 701 BIOCHIM. |
| | fibroblasts, other functions unknown. | BIOPHYS. ACTA 7 (1982); Senior et al., |
| | | 96 J. CELL. BIOL. 382 (1983). |
| Thrombopoietin | Human TPO (Thrombopoietin, Mpl- | Horikawa et al., 90(10) BLOOD 4031-38 |
| | ligand, MGDF) stimulates the | (1997); de Sauvage et al., 369 NATURE |
| | proliferation and maturation of | 533-58 (1995). |
| | megakaryocytes and promotes increased | |
| | circulating levels of platelets in vivo. | |
| | Binds to c-Mpl receptor. | |
| Thrombo-spondin | High-molecular weight, heparin-binding | Dawes et al., 29 THROMB. RES. 569 |
| | glycoprotein constituent of platelets, | (1983); Switalska et al., 106 J. LAB. |
| | consisting of three, identical, disulfide- | CLIN. MED. 690 (1985); Lawler et al. |
| | linked polypeptide chains. Binds to | 260 J. BIOL. CHEM. 3762 (1985); Wolff |
| | surface of resting and activated | et al., 261 J. BIOL. CHEM. 6840 (1986); |
| | platelets, may effect platelet adherence | Asch et al., 79 J. CLIN. CHEM. 1054 |
| | and aggregation. An integral component | (1987); Jaffe et al., 295 NATURE 246 |
| | of basement membrane in different | (1982); Wright et al., 33 J. HISTOCHEM. |
| | tissues. Interacts with a variety of | CYTOCHEM. 295 (1985); Dixit et al., |
| | extracellular macromolecules including | 259 J. BIOL. CHEM. 10100 (1984); |
| | heparin, collagen, fibrinogen and | Mumby et al., 98 J. CELL. BIOL. 646 |
| | fibronectin, plasminogen, plasminogen | (1984); Lahav et al, 145 EUR. J. |
| | activator, and osteonectin. May | BIOCHEM. 151 (1984); Silverstein et al, |
| | modulate cell-matrix interactions. | 260 J. BIOL. CHEM. 10346 (1985); |
| | | Clezardin et al. 175 EUR. J. BIOCHEM. |
| | | 275 (1988); Sage & Bornstein (1991). |
| Von Willebrand | Multimeric plasma glycoprotein made | Hoyer 58 BLOOD 1 (1981); Ruggeri & |
| Factor | of identical subunits held together by | Zimmerman 65 J. CLIN. INVEST. 1318 |
| | disulfide bonds. During normal | (1980); Hoyer & Shainoff 55 BLOOD |
| | hemostasis, larger multimers of vWF | 1056 (1980); Meyer et al., 95 J. LAB. |
| | cause platelet plug formation by | CLIN. INVEST. 590 (1980); Santoro 21 |
| | forming a bridge between platelet | THROMB. RES. 689 (1981); Santoro, & |
| | glycoprotein IB and exposed collagen in | Cowan 2 COLLAGEN RELAT. RES. 31 |
| | the subendothelium. Also binds and | (1982); Morton et al., 32 THROMB. RES. |
| | transports factor VIII (antihemophilic | 545 (1983); Tuddenham et al., 52 BRIT. |
| | factor) in plasma. | J. HAEMATOL. 259 (1982). |

Additional blood proteins contemplated herein include the following human serum proteins, which may also be placed in another category of protein (such as hormone or antigen): Actin, Actinin, Amyloid Serum P, Apolipoprotein E, B2-Microglobulin, C-Reactive Protein (CRP), Cholesterylester transfer protein (CETP), Complement C3B, Ceruplasmin, Creatine Kinase, Cystatin, Cytokeratin 8, Cytokeratin 14, Cytokeratin 18, Cytokeratin 19, Cytokeratin 20, Desmin, Desmocollin 3, FAS (CD95), Fatty Acid Binding Protein, Ferritin, Filamin, Glial Filament Acidic Protein, Glycogen Phosphorylase Isoenzyme BB (GPBB), Haptoglobulin, Human Myoglobin, Myelin Basic Protein, Neurofilament, Placental Lactogen, Human SHBG, Human Thyroid Peroxidase, Receptor Associated Protein, Human Cardiac Troponin C, Human Cardiac Troponin I, Human Cardiac Troponin T, Human Skeletal Troponin I, Human Skeletal Troponin T, Vimentin, Vinculin, Transferrin Receptor, Prealbumin, Albumin, Alpha-1-Acid Glycoprotein, Alpha-1-Antichymotrypsin, Alpha-1-Antitrypsin, Alpha-Fetoprotein, Alpha-1-Microglobulin, Beta-2-microglobulin, C-Reactive Protein, Haptoglobulin, Myoglobulin, Prealbumin, PSA, Prostatic Acid Phosphatase, Retinol Binding Protein, Thyroglobulin, Thyroid Microsomal Antigen, Thyroxine Binding Globulin, Transferrin, Troponin I, Troponin T, Prostatic Acid Phosphatase, Retinol Binding Globulin (RBP). All of these proteins, and sources thereof, are known in the art. Many of these proteins are available commercially from, for example, Research Diagnostics, Inc. (Flanders, N.J.).

The target in the present invention may also incorporate or target neurotransmitters, or functional portions thereof. Neurotransmitters are chemicals made by neurons and used by them to transmit signals to the other neurons or non-neuronal cells (e.g., skeletal muscle; myocardium, pineal glandular cells) that they innervate. Neurotransmitters produce their effects by being released into synapses when their neuron of origin fires (i.e., becomes depolarized) and then attaching to receptors in the membrane of the post-synaptic cells. This causes changes in the fluxes of particular ions across that membrane, making cells more likely to become depolarized, if the neurotransmitter happens to be excitatory, or less likely if it is inhibitory. Neurotransmitters can also produce their effects by modulating the production of other signal-transducing molecules (“second messengers”) in the post-synaptic cells. See generally COOPER, BLOOM & ROTH, THE BIOCHEMICAL BASIS OF NEUROPHARMACOLOGY (7th Ed. Oxford Univ. Press, NYC, 1996); http://web.indstate.edu/theme/mwking/nerves. Neurotransmitters contemplated in the present invention include, but are not limited to, Acetylcholine, Serotonin, γ-aminobutyrate (GABA), Glutamate, Aspartate, Glycine, Histamine, Epinephrine, Norepinephrine, Dopamine, Adenosine, ATP, Nitric oxide, and any of the peptide neurotransmitters such as those derived from pre-opiomelanocortin (POMC), as well as antagonists and agonists of any of the foregoing.

Numerous other proteins or peptides may serve as either targets, or as a source of target-binding moieties as described herein. Table 4 presents a non-limiting list and description of some pharmacologically active peptides that may serve as, or serve as a source of a functional derivative of, the target of the present invention.

TABLE 4
Pharmacologically active peptides

| Binding partner/ | | |
|---|---|---|
| Protein of interest | | |
| (form of peptide) | Pharmacological activity | Reference |
| c-Mpl | TPO-mimetic | Cwirla et al., 276 SCIENCE 1696-9 (1997); |
| (linear) | | U.S. Pat. No. 5,869,451, issued Feb. |
| | | 9, 1999; U.S. Pat. No. 5,932,946, issued |
| | | Aug. 3, 1999. |
| c-Mpl | TPO-mimetic | Cwirla et al., 276 SCIENCE 1696-9 (1997). |
| (C-terminally cross- | | |
| linked dimer) | | |
| (disulfide-linked | stimulation of | Paukovits et al., 364 HOPPE-SEYLERS Z. |
| dimer) | hematopoesis | PHYSIOL. CHEM. 30311 (1984); |
| | (“G-CSF-mimetic”) | Laerurngal., 16 EXP. HEMAT. 274-80 |
| | | (1988). |
| (alkylene-linked dimer) | G-CSF-mimetic | Batnagar et al., 39 J. MED. CHEM. 38149 |
| | | (1996); Cuthbertson et al., 40 J. MED. |
| | | CHEM. 2876-82 (1997); King et al., 19 |
| | | EXP. HEMATOL. 481 (1991); King et al., |
| | | 86(Suppl. 1) BLOOD 309 (1995). |
| IL-1 receptor | inflammatory and | U.S. Pat. No. 5,608,035; U.S. Pat. No. |
| (linear) | autoimmune diseases (“IL-1 | 5,786,331; U.S. Pat. No. 5,880,096; |
| | antagonist” or “IL-1 ra- | Yanofsky et al., 93 PNAS 7381-6 (1996); |
| | mimetic”) | Akeson et al., 271 J. BIOL. CHEM. 30517-23 |
| | | (1996); Wiekzorek et al., 49 POL. J. |
| | | PHARMACOL. 107-17 (1997); Yanofsky, |
| | | 93 PNAS 7381-7386 (1996). |
| Facteur thyrnique | stimulation of lymphocytes | Inagaki-Ohara et al., 171 CELLULAR |
| (linear) | (FTS-mimetic) | IMMUNOL. 30-40 (1996); Yoshida, 6 J. |
| | | IMMUNOPHARMACOL 141-6 (1984). |
| CTLA4 MAb | CTLA4-mimetic | Fukumoto et al., 16 NATURE BIOTECH. |
| (intrapeptide di-sulfide | | 267-70 (1998). |
| bonded) | | |
| TNF-a receptor | TNF-a antagonist | Takasaki et al., 15 NATURE BIOTECH. |
| (exo-cyclic) | | 1266-70 (1997); WO 98/53842, published |
| | | Dec. 3, 1998. |
| TNF-a receptor | TNF-a antagonist | Chirinos-Rojas, J. IMM., 5621-26. |
| (linear) | | |
| C3b | inhibition of complement | Sahu et al., 157 IMMUNOL. 884-91 (1996); |
| (intrapeptide di-sulfide | activation; autoimmune | Morikis et al., 7 PROTEIN SCI. 619-27 |
| bonded) | diseases (C3b antagonist) | (1998). |
| vinculin | cell adhesion processes, cell | Adey et al., 324 BIOCHEM. J. 523-8 |
| (linear) | growth, differentiation | (1997). |
| | wound healing, tumor | |
| | metastasis (“vinculin | |
| | binding”) | |
| C4 binding protein (C413P) | anti-thrombotic | Linse et al. 272 BIOL. CHEM. 14658-65 |
| (linear) | | (1997). |
| urokinase receptor | processes associated with | Goodson et al., 91 PNAS 7129-33 (1994); |
| (linear) | urokinase interaction with its | International patent application WO |
| | receptor (e.g. angiogenesis, | 97/35969, published Oct. 2, 1997. |
| | tumor cell invasion and | |
| | metastasis; (URK | |
| | antagonist) | |
| Mdm2, Hdm2 | Inhibition of inactivation of | Picksley et al., 9 ONCOGENE 2523-9 |
| (linear) | p53 mediated by Mdm2 or | (1994); Bottger et al. 269 J. MOL. BIOL. |
| | hdm2; anti-tumor | 744-56 (1997); Bottger et al., 13 |
| | (“Mdm/hdm antagonist”) | ONCOGENE 13: 2141-7 (1996). |
| p21^{WAF1} | anti-tumor by mimicking the | Ball et al., 7 CURR. BIOL. 71-80 (1997). |
| (linear) | activity of p21^{WAF1} | |
| farnesyl transferase | anti-cancer by preventing | Gibbs et al., 77 CELL 175-178 (1994). |
| (linear) | activation of ras oncogene | |
| Ras effector domain | anti-cancer by inhibiting | Moodie et at., 10 TRENDS GENEL 44-48 |
| (linear) | biological function of the ras | (1994); Rodriguez et al., 370 NATURE |
| | oncogene | 527-532 (1994). |
| SH2/SH3 domains | anti-cancer by inhibiting | Pawson et al, 3 CURR. BIOL. 434-432 |
| (linear) | tumor growth with activated | (1993); Yu et al., 76 CELL 933-945 |
| | tyrosine kinases | (1994). |
| p16^{INK4} | anti-cancer by mimicking | Fahraeus et al., 6 CURR. BIOL. 84-91 |
| (linear) | activity of p16; e.g., | (1996). |
| | inhibiting cyclin D-Cdk | |
| | complex (“p, 16-mimetic”) | |
| Src, Lyn | inhibition of Mast cell | Stauffer et al., 36 BIOCHEM. 9388-94 |
| (linear) | activation, IgE-related | (1997). |
| | conditions, type I | |
| | hypersensitivity (“Mast cell | |
| | antagonist”). | |
| Mast cell protease | treatment of inflammatory | International patent application WO |
| (linear) | disorders mediated by | 98/33812, published Aug. 6, 1998. |
| | release of tryptase-6 (“Mast | |
| | cell protease inhibitors”) | |
| SH3 domains | treatment of SH3-mediated | Rickles et al., 13 EMBO J. |
| (linear) | disease states (“SH3 | 5598-5604 (1994); Sparks et al., |
| | antagonist”) | 269 J. BIOL. CHEM. 238536 |
| | | (1994); Sparks et al., 93 PNAS |
| | | 1540-44 (1996). |
| HBV core antigen (HBcAg) | treatment of HBV viral | Dyson & Muray, PNAS 2194-98 |
| (linear) | antigen (HBcAg) infections | (1995). |
| | (“anti-HBV”) | |
| selectins | neutrophil adhesion | Martens et al., 270 J. BIOL. |
| (linear) | inflammatory diseases | CHEM. 21129-36 (1995); |
| | (“selectin antagonist”) | European Pat. App. EP 0 714 |
| | | 912, published Jun. 5, 1996. |
| calmodulin | calmodulin | Pierce et al., 1 MOLEC. |
| (linear, cyclized) | antagonist | DIVEMILY 25965 (1995); |
| | | Dedman et al., 267 J. BIOL. |
| | | CHEM. 23025-30 (1993); Adey |
| | | & Kay, 169 GENE 133-34 |
| | | (1996). |
| integrins | tumor-homing; treatment for | International patent applications WO |
| (linear, cyclized) | conditions related to | 95/14714, published Jun. 1, 1995; WO |
| | integrin-mediated cellular | 97/08203, published Mar. 6, 1997; WO |
| | events, including platelet | 98/10795, published Mar. 19, 1998; WO |
| | aggregation, thrombosis, | 99/24462, published May 20, 1999; Kraft |
| | wound healing, osteoporosis, | et al., 274 J. BIOL. CHEM. 1979-85 |
| | tissue repair, angiogenesis | (1999). |
| | (e.g., for treatment of | |
| | cancer) and tumor invasion | |
| | (“integrin-binding”) | |
| fibronectin and extracellular | treatment of inflammatory | International patent application WO |
| matrix components of T-cells | and autoimmune conditions | 98/09985, published Mar. 12, 1998. |
| and macrophages | | |
| (cyclic, linear) | | |
| somatostatin and cortistatin | treatment or prevention of | European patent application EP 0 911 |
| (linear) | hormone-producing tumors, | 393, published Apr. 28, 1999. |
| | acromegaly, giantism, | |
| | dementia, gastric ulcer, | |
| | tumor growth, inhibition of | |
| | hormone secretion, | |
| | modulation of sleep or | |
| | neural activity | |
| bacterial lipopoly-saccharide | antibiotic; septic shock; | U.S. Pat. No. 5,877,151, issued Mar. 2, |
| (linear) | disorders modulatable by | 1999. |
| | CAP37 | |
| parclaxin, mellitin | antipathogenic | International patent application WO |
| (linear or cyclic) | | 97/31019, published 28 Aug. 1997. |
| VIP | impotence, neuro- | International patent application WO |
| (linear, cyclic) | degenerative disorders | 97/40070, published Oct. 30, 1997. |
| CTLs | cancer | European patent application EP 0 770 |
| (linear) | | 624, published May 2, 1997. |
| THF-gamma2 | | Burnstein, 27 BIOCHEM. 4066-71 (1988). |
| (linear) | | |
| Amylin | | Cooper, 84 PNAS 8628-32 (1987). |
| (linear) | | |
| Adreno-medullin | | Kitamura, 192 BBRC 553-60 (1993). |
| (linear) | | |
| VEGF | anti-angiogenic; cancer, | Fairbrother, 37 BIOCHEM. 17754-64 |
| (cyclic, linear) | rheumatoid arthritis, diabetic | (1998). |
| | retinopathy, psoriasis | |
| | (“VEGF antagonist'”) | |
| MMP | inflammation and | Koivunen, 17 NATURE BIOTECH. 768-74 |
| (cyclic) | autoimmune disorders; | (1999). |
| | tumor growth (“MMP | |
| | inhibitor”) | |
| HGH fragment | | U.S. Pat. No. 5,869,452, issued |
| (linear) | | Feb. 9, 1999. |
| Echistatin | inhibition of platelet | Gan, 263 J. BIOL. 19827-32 (1988). |
| | aggregation | |
| SLE autoantibody | SLE | International patent application WO |
| (linear) | | 96/30057, published Oct. 3, 1996. |
| GD1 alpha | suppression of tumor | Ishikawa et al., 1 FEBS LETT. 20-4 |
| | metastasis | (1998). |
| anti-phospholipid β-2 | endothelial cell activation, | Blank Mal., 96 PNAS 5164-8 (1999). |
| glycoprotein-1 (β2GPI) | anti-phospholipid syndrome | |
| antibodies | (APS), thromboembolic | |
| | phenomena, | |
| | thrombocytopenia, and | |
| | recurrent fetal loss | |
| T-Cell Receptor β chain | diabetes | International patent application WO |
| (linear) | | 96/101214, published Apr. 18, 1996. |
| Binding partner/ | | |
| Protein of interest | | |
| (form of peptide) | Pharmacological activity | Reference |
| EPO receptor | EPO mimetic | Wrighton et al. (1996), Science 273: |
| (intrapeptide | | 458-63; U.S. Pat. No. 5,773,569, issued |
| disulfide-bonded) | | Jun. 30, 1998 to Wrighton et al. |
| EPO receptor | EPO mimetic | Livnah et al. (1996), Science 273: 464-71; |
| (C-terminally cross- | | Wrighton et al. (1997), Nature |
| linked dimer) | | Biotechnology 15: 1261-5; int'l patent |
| | | application WO 96/40772, published |
| | | Dec. 19, 1996 |
| EPO receptor | EPO mimetic | Naranda et al., 96 PNAS 7569-74 (1999) |
| (linear) | | |
| c-Mpl | TPO-mimetic | Cwirla et al.(1997) Science 276: 1696-9; |
| (linear) | | U.S. Pat. No. 5,869,451, issued Feb. |
| | | 9, 1999; U.S. Pat. No. 5,932,946, issued |
| | | Aug. 3, 1999 |
| c-Mpl | TPO-mimetic | Cwirla et al. (1997) Science 276: 1696-9 |
| (C-terminally cross- | | |
| linked dimer) | | |
| (disulfide-linked | stimulation of | Paukovits et al. (1984), Hoppe-Seylers Z. |
| dimer) | hematopoesis | Physiol. Chem. 365: 30311; Laerurn gal. |
| | (“G-CSF-mimetic”) | (1988), Exp. Hemat. 16: 274-80 |
| (alkylene-linked dimer) | G-CSF-mimetic | Batnagar 91-al. (1996), linked dimer J. |
| | | Med. Chem. 39: 38149; Cuthbertson et al. |
| | | (1997), J. Med. Chem. 40: 2876-82; King |
| | | et al. (1991), Exp. Hematol. 19: 481; King |
| | | et al. (1995), Blood 86 (Suppl. 1): 309 |
| IL-1 receptor | inflammatory and | U.S. Pat. No. 5,608,035; U.S. Pat. No. |
| (linear) | autoimmune diseases (“IL-1 | 5,786,331; U.S-Pat. No. 5,880,096; |
| | antagonist” or “IL-1 ra- | Yanofsky 91-al. (1996) PNAS 93: 7381-6; |
| | mimetic”) | Akeson et al. (1996), J. Biol. Chem. 271: |
| | | 30517-23; Wiekzorek et al. (1997), Pol. J. |
| | | Pharmacol. 49: 107-17; Yanofsky (1996), |
| | | PNAs, 93: 7381-7386. |
| Facteur thyrnique | stimulation of lymphocytes | Inagaki-Ohara et al. (1996), Cellular |
| (linear) | (FTS-mimetic) | Immunol. 171: 30-40; Yoshida (1984), J. |
| | | Immunopharmacol, 6: 141-6. |
| CTLA4 MAb | CTLA4-mimetic | Fukumoto et al. (1998), Nature Biotech. |
| (intrapeptide di-sulfide | | 16: 267-70 |
| bonded) | | |
| TNF-a receptor | TNF-a antagonist | Takasaki et al. (1997), Nature Biotech. |
| (exo-cyclic) | | 15: 1266-70; WO 98/53842, published |
| | | Dec. 3, 1998. |
| TNF-a receptor | TNF-a antagonist | Chirinos-Rojas J. Imm., 5621-26. |
| (linear) | | |
| C3b | inhibition of complement | Sahu et al. (1996), Immunol. 157: 884-91; |
| (intrapeptide di-sulfide | activation; autoimmune | Morikis et al. (1998), Protein Sci. 7: 619-27. |
| bonded) | diseases (C3b antagonist) | |
| vinculin | cell adhesion processes, cell | Adey et al. (1997), Biochem. J. 324: 523-8 |
| (linear) | growth, differentiation | |
| | wound healing, tumor | |
| | metastasis (“vinculin | |
| | binding”) | |
| C4 binding protein (C413P) | anti-thrombotic | Linse et al. 272 Biol. Chem. 14658-65 |
| (linear) | | (1997) |
| urokinase receptor | processes associated with | Goodson et al. (1994), 91 PNAS 7129-33; |
| (linear) | urokinase interaction with its | International application WO 97/35969, |
| | receptor (e.g. angiogenesis, | published Oct. 2, 1997 |
| | tumor cell invasion and | |
| | metastasis; (URK | |
| | antagonist) | |
| Mdm2, Hdm2 | Inhibition of inactivation of | Picksley et al. (1994), Oncogene 9: 2523-9; |
| (linear) | p53 mediated by Mdm2 or | Bottger et al. (1997) J. Mol. Biol. 269: |
| | hdm2; anti-tumor | 744-56; Bottger et al. (1996), Oncogene |
| | (“Mdm/hdm antagonist”) | 13: 2141-7 |
| p21^{WAF1} | anti-tumor by mimicking the | Ball et al.(1997), Curr. Biol. 7: 71-80. |
| (linear) | activity of p21^{WAF1} | |
| farnesyl transferase | anti-cancer by preventing | Gibbs et al. (1994), Cell 77: 175-178 |
| (linear) | activation of ras oncogene | |
| Ras effector domain | anti-cancer by inhibiting | Moodie et at. (1994), Trends Genel 10: 44-48 |
| (linear) | biological function of the ras | Rodriguez et al. (1994), Nature |
| | oncogene | 370: 527-532. |
| SH2/SH3 domains | anti-cancer by inhibiting | Pawson et al (1993), Curr. Biol. 3: 434-432, |
| (linear) | tumor growth with activated | Yu et al. (1994), Cell 76: 933-945. |
| | tyrosine kinases | |
| p16^{INK4} | anti-cancer by mimicking | Fahraeus et al. (1996), Curr. Biol. 6: 84-91 |
| (linear) | activity of p16; e.g., | |
| | inhibiting cyclin D-Cdk | |
| | complex (“p, 16-mimetic”) | |
| Src, Lyn | inhibition of Mast cell | Stauffer et al. (1997), Biochem. 36: 9388-94. |
| (linear) | activation, IgE-related | |
| | conditions, type I | |
| | hypersensitivity (“Mast cell | |
| | antagonist”). | |
| Mast cell protease | treatment of inflammatory | International application WO 98/33812, |
| (linear) | disorders mediated by | published Aug. 6, 1998 |
| | release of tryptase-6 (“Mast | |
| | cell protease inhibitors”) | |
| SH3 domains | treatment of SH3-mediated | Rickles et al. (1994), EMBO J. |
| (linear) | disease states (“SH3 | 13: 5598-5604; Sparks aLal. |
| | antagonist”) | (1994), J. Biol. Chem. 269: |
| | | 238536; Sparks et al. (1996), |
| | | PNAS 93: 1540-44. |
| HBV core antigen (HBcAg) | treatment of HBV viral | Dyson & Muray (1995), Proc. |
| (linear) | antigen (HBcAg) infections | NatI. Acad. Sci. 92: 2194-98. |
| | (“anti-HBV”) | |
| selectins | neutrophil adhesion | Martens et al. (1995), J. Biol. |
| (linear) | inflammatory diseases | Chem. 270: 21129-36; European |
| | (“selectin antagonist”) | pat. app.EP 0 714 912, published |
| | | Jun. 5, 1996 |
| calmodulin | calmodulin | Pierce et al. (1995), Molec. |
| (linear, cyclized) | antagonist | Divemily 1: 25965; Dedman et |
| | | al. (1993), J. Biol. Chem. 268: |
| | | 23025-30; Adey & Kay (1996), |
| | | Gene 169: 133-34. |
| integrins | tumor-homing; treatment for | International applications WO 95/14714, |
| (linear, cyclized) | conditions related to | published Jun. 1, 1995; WO 97/08203, |
| | integrin-mediated cellular | published Mar. 6, 1997; WO 98/10795, |
| | events, including platelet | published Mar. 19, 1998; WO 99/24462, |
| | aggregation, thrombosis, | published May 20, 1999; Kraft et al. |
| | wound healing, osteoporosis, | (1999), J. Biol. Chem. 274: 1979-85. |
| | tissue repair, angiogenesis | |
| | (e.g., for treatment of | |
| | cancer) and tumor invasion | |
| | (“integrin-binding”) | |
| fibronectin and extracellular | treatment of inflammatory | WO 98/09985, published Mar. 12, 1998. |
| matrix components of T-cells | and autoimmune conditions | |
| and macrophages | | |
| (cyclic, linear) | | |
| somatostatin and cortistatin | treatment or prevention of | European patent application 0 911 393, |
| (linear) | hormone-producing tumors, | published Apr. 28, 1999. |
| | acromegaly, giantism, | |
| | dementia, gastric ulcer, | |
| | tumor growth, inhibition of | |
| | hormone secretion, | |
| | modulation of sleep or | |
| | neural activity | |
| bacterial lipopoly-saccharide | antibiotic; septic shock; | U.S. Pat. No. 5,877,151, issued Mar. |
| (linear) | disorders modulatable by | 2, 1999. |
| | CAP37 | |
| parclaxin, mellitin | antipathogenic | WO 97/31019, published 28 Aug. |
| (linear or cyclic) | | 1997. |
| VIP | impotence, neuro- | WO 97/40070, published Oct. 30, |
| (linear, cyclic) | degenerative disorders | 1997. |
| CTLs | cancer | EP 0 770 624, published May 2, 1997. |
| (linear) | | |
| THF-gamma2 | | Burnstein (1988), Biochem., 27: 4066-71 |
| (linear) | | |
| Amylin | | Cooper (1987), PNAS 84: 8628-32. |
| (linear) | | |
| Adreno-medullin | | Kitamura (1993), BBRC, 192: 553-60 |
| (linear) | | |
| VEGF | anti-angiogenic; cancer, | Fairbrother (1998), Biochem., 37: 17754-64. |
| (cyclic, linear) | rheumatoid arthritis, diabetic | |
| | retinopathy, psoriasis | |
| | (“VEGF antagonist”) | |
| MMP | inflammation and | Koivunen 17 Nature Biotech., 768-74 |
| (cyclic) | autoimmune disorders; | (1999). |
| | tumor growth (“MMP | |
| | inhibitor”) | |
| HGH fragment | | U.S. Pat. No. 5,869,452. |
| (linear) | | |
| Echistatin | inhibition of platelet | Gan (1988), J. Biol. 263: 19827-32. |
| | aggregation | |
| SLE autoantibody | SLE | WO 96/30057, published Oct. 3, 1996. |
| (linear) | | |
| GD1 alpha | suppression of tumor | Ishikawa et al., 1 FEBS Lett. 20-4 (1998). |
| | metastasis | |
| anti-phospholipid β-2 | endothelial cell activation, | Blank Mal. (1999), PNAS 96: 5164-8. |
| glycoprotein-1 (β2GPI) | anti-phospholipid syndrome | |
| antibodies | (APS), thromboembolic | |
| | phenomena, | |
| | thrombocytopenia, and | |
| | recurrent fetal loss | |
| T-Cell Receptor β chain | diabetes | WO 96/101214, published Apr. 18, 1996. |
| (linear) | | |

Peptides

Any number of peptides may be used in conjunction with the present invention. Of particular interest are peptides that mimic the activity of EPO, TPO, growth hormone, G-CSF, GM-CSF, IL-1ra, leptin. CTLA4, TRAIL, TGF-α, and TGF-β. Peptide antagonists are also of interest, particularly those antagonistic to the activity of TNF, leptin, any of the interleukins (IL-1-IL-23, etc.), and proteins involved in complement activation (e.g., C3b). Targeting peptides are also of interest, including tumor-homing peptides, membrane-transporting peptides, and the like. All of these classes of peptides may be discovered by methods described in the references cited in this specification and other references.

A particularly preferred group of peptides are those that bind to cytokine receptors. Cytokines have recently been classified according to their receptor code. See Inglot (1997), Archivum Immunologiae e Therapiae Experimentalis 45: 353-7, which is hereby incorporated entirely by reference.

Non-limiting examples of suitable peptides for this invention appear in Tables 5 through 21 below. These peptides may be prepared by methods disclosed and/or known in the art. Single letter amino acid abbreviations are used in most cases. The X in these sequences (and throughout this specification, unless specified otherwise in a particular instance) means that any of the 20 naturally occurring amino acid residues or know derivatives thereof may be present, or any know modified amino acid thereof. Any of these peptides may be linked in tandem (i.e., sequentially), with or without linkers, and a few tandem linked examples are provided in the table. Linkers are listed as “Δ” and may be any of the linkers described herein. Tandem repeats and linkers are shown separated by dashes for clarity. Any peptide containing a cysteinyl residue may optionally be cross-linked with another Cys-containing peptide, either or both of which may be linked to a vehicle. A few crosslinked examples are provided in the table. Any peptide having more than one Cys residue may form an intrapeptide disulfide bond, as well; see, for example, EPO-mimetic peptides in Table 5. A few examples of intrapeptide disulfide-bonded peptides are specified in the table. Any of these peptides may be derivatized as described herein, and a few derivatized examples are provided in the table. For derivatives in which the carboxyl terminus may be capped with an amino group, the capping amino group is shown as —NH₂. For derivatives in which amino acid residues are substituted by moieties other than amino acid residues, the substitutions are denoted by a δ, which signifies any of the moieties known in the art, e.g., as described in Bhatnagar et al. (1996), J. Med. Chem. 39: 3814-9 and Cuthbertson et al. (1997), J. Med. Chem. 40:2876-82, which are entirely incorporated by reference. The J substituent and the Z substituents (Z₅, Z₆, . . . Z₄₀) are as defined in U.S. Pat. Nos. 5,608,035, 5,786,331, and 5,880,096, which are entirely incorporated herein by reference. For the EPO-mimetic sequences (Table 5), the substituents X₂ through X₁₁ and the integer “n” are as defined in WO 96/40772, which is entirely incorporated by reference. The substituents “Ψ” “Θ,” “+” are as defined in Sparks et al. (1996), Proc. Natl. Acad. Sci. 93: 1540-4, which is entirely incorporated by reference. X4, X5, X6, and X7 are as defined in U.S. Pat. No. 5,773,569, which is hereby entirely incorporated by reference, except that: for integrin-binding peptides, X1, X2, X3, X4, X5, X6, X7, and X8 (Table 10), are as defined in PCT applications WO 95/14714, published Jun. 1, 1995 and WO 97/08203, published Mar. 6, 1997, which are also entirely incorporated by reference; and for VIP-mimetic peptides (Table 13), X₁, X₁′, X₁″, X₂, X₃, X₄, X₅, X₆, and Z; and the integers m and n are as defined in WO 97/40070, published Oct. 30, 1997, which is also entirely incorporated herein by reference. Xaa and Yaa below are as defined in WO 98/09985, published Mar. 12, 1998, which is entirely incorporated herein by reference. AA₁, AA₂, AB₁, AB₂, and AC are as defined in International application WO 98/53842, published Dec. 3, 1998, which is entirely incorporated by reference. X¹, X², X³, and X⁴ in Table 18 only are as, defined in European application EP 0 911 393, published Apr. 28, 1999, entirely incorporated herein by reference. Residues appearing in boldface are D-amino acids, but can be optionally L-amino acids. All peptides are linked through peptide bonds unless otherwise noted. Abbreviations are listed at the end of this specification. In the “SEQ ID NO.” column, “NR” means that no sequence listing is required for the given sequence.

TABLE 5

EPO-mimetic peptide sequences

| | |
|---|---|
| Sequence/structure: | SEQ ID NO: |

| | |
|---|---|
| YXCXXGPXTWXCXP | 1 |
| YXCXXGPXTWXCXP-YXCXXGPXTWXCXP | 2 |
| YXCXXGPXTWXCXP-A-YXCXXGPXTWXCXP | 3 |
| YXCXXGPXTWXCXP-Δ-ε-amine) | 4 |
| \ | |
| K | |
| / | |
| YXCXXGPXTWXCXP-Δ-(α-amine) | 4 |
| GGTYSCHFGPLTWVCKPQGG | 5 |
| GGDYHCRMGPLTWVCKPLGG | 6 |
| GGVYACRMGPITWVCSPLGG | 7 |
| VGNYMCHFGPITWVCRPGGG | 8 |
| GGLYLCRFGPVTWDCGYKGG | 9 |
| GGTYSCHFGPLTWVCKPQGG- | 10 |
| GGTYSCHFGPLTWVCKPQGG-Δ- | 11 |
| GGTYSCHFGPLTWVCKPQGG | |
| GGTYSCHFGPLTWVCKPQGGSSK | 12 |
| GGTYSCHFGPLTWVCKPQGGSSK | 13 |
| GGTYSCHFGPLTWVCKPQGGSSK | 14 |
| GGTYSCHFGPLTWVCKPQGGSSK-Δ- | |
| GGTYSCHFGPLTWVCKPQGGSSK | |
| GGTYSCHFGPLTWVCKPQGGSS-Δ-ε-amine) | |
| \ | |
| K | |
| / | |
| GGTYSCHFGPLTWVCKPQGGSS-Δ-(α-amine) | 15 |
| GGTYSCHFGPLTWVCKPQGGSSK(-Δ-biotin) | 16 |
| CX₄X₅GPX₆TWX₇C | 17 |
| GGTYSCHGPLTWVCKPQGG | 18 |
| VGNYMAHMGPITWVCRPGG | 19 |
| GGPHHVYACRMGPLTWIC | 20 |
| GGTYSCHFGPLTWVCKPQ | 21 |
| GGLYACHMGPMTWVCQPLRG | 22 |
| TIAQYICYMGPETWECRPSPKA | 23 |
| YSCHFGPLTWVCK | 24 |
| YCHFGPLTWVC | 25 |
| X₃X₄X₅GPX₆TWX₇X₈ | 26 |
| YX₂X₃X₄X₅GPX₆TWX₇X₈ | 27 |
| X₁YX₂X₃X₄X₅GPX₆X₇X₈X₉X₁₀X₁₁ | 28 |
| X₁YX₂CX₄X₅GPX₆TWX₇CX₉X₁₀X₁₁ | 29 |
| GGLYLCRFGPVTWDCGYKGG | 30 |
| GGTYSCHFGPLTWVCKPQGG | 31 |
| GGDYHCRMGPLTWVCKPLGG | 32 |
| VGNYMCHFGPITWVCRPGGG | 33 |
| GGVYACRMGPITWVCSPLGG | 34 |
| VGNYMAHMGPITWVCRPGG | 35 |
| GGTYSCHFGPLTWVCKPQ | 36 |
| GGLYACHMGPMTWVCQPLRG | 37 |
| TIAQYICYMGPETWECRPSPKA | 38 |
| YSCHFGPLTWVCK | 39 |
| YCHFGPLTWVC | 40 |
| SCHFGPLTWVCK | 41 |
| (AX₂)ₙX₃X₄X₅GPX₆TWX₇X₈ | 42 |

TABLE 6

IL-1 antagonist peptide sequences

| | |
|---|---|
| SEQUENCE/STRUCTURE | SEQ ID NO: |

| | |
|---|---|
| Z₁₁Z₇Z₈ZQZ₅YZ₆Z₉Z₁₀ | 43 |
| XXQZ₅YZ₆XX | 44 |
| Z₇XQZ₅YZ₆XX | 45 |
| Z₇Z₈QZ₅YZ₆Z₉Z₁₀ | 46 |
| Z₁₁Z₇Z₈QZ₅YZ₆Z₉Z₁₀ | 47 |
| Z₁₂Z₁₃Z₁₄Z₁₅Z₁₆Z₁₇Z₁₈Z₁₉Z₂₀Z₂₁Z₂₂Z₁₁Z₇Z₈QZ₅YZ₆Z₉Z₁₀L | 48 |
| Z₂₃NZ₂₄Z₃₉Z₂₅Z₂₆Z₂₇Z₂₈Z₂₉Z₃₀Z₄₀ | 49 |
| TANVSSFEWTPYYWQPYALPL | 50 |
| SWTDYGYWQPYALPISGL | 51 |
| ETPFTWEESNAYYWQPYALPL | 52 |
| ENTYSPNWADSMYWQPYALPL | 53 |
| SVGEDHNFWTSEYWQPYALPL | 54 |
| DGYDRWRQSGERYWQPYALPL | 55 |
| FEWTPGYWQPY | 56 |
| FEWTPGYWQHY | 57 |
| FEWTPGWYQJY | 58 |
| AcFEWTPGWYQJY | 59 |
| FEVffPGWpYQJY | 60 |
| FAWTPGYWQJY | 61 |
| FEWAPGYWQJY | 62 |
| FEWVPGYWQJY | 63 |
| FEWTPGYWQJY | 64 |
| AcFEWTPGYWQJY | 65 |
| FEWTPaWYQJY | 66 |
| FEWTPSarWYQJY | 67 |
| FEWTPGYYQPY | 68 |
| FEWTPGWWQPY | 69 |
| FEWTPNYWQPY | 70 |
| FEVffPvYWQJY | 71 |
| FEWTPecGYWQJY | 72 |
| FEWTPAibYWQJY | 73 |
| FEVffSarGYWQJY | 74 |
| FEWTPGYWQPY | 75 |
| FEWTPGYWQHY | 76 |
| FEWTPGWYQJY | 77 |
| AcFEWTPGWYQJY | 78 |
| FEWTPGW-pY-QJY | 79 |
| FAWTPGYWQJY | 80 |
| FEWAPGYWQJY | 81 |
| FEWVPGYWQJY | 82 |
| FEWTPGYWQJY | 83 |
| AcFEWTPGYWQJY | 84 |
| FEWTPAWYQJY | 85 |
| FEWTPSarWYQJY | 86 |
| FEWTPGYYQPY | 87 |
| FEWTPGWWQPY | 88 |
| FEWTPNYWQPY | 89 |
| FEWTPVYWQJY | 90 |
| FEWTPecGYWQJY | 91 |
| FEWTPAibYWQJY | 92 |
| FEWTSarGYWQJY | 93 |
| FEWTPGYWQPYALPL | 94 |
| NapEWTPGYYQJY | 95 |
| YEWTPGYYQJY | 96 |
| FEWVPGYYQJY | 97 |
| FEWTPSYYQJY | 99 |
| FEWTPNYYQJY | 99 |
| TKPR | 100 |
| RKSSK | 101 |
| RKQDK | 102 |
| NRKQDK | 103 |
| RKQDKR | 104 |
| ENRKQDKRF | 105 |
| VTKFYF | 106 |
| VTKFY | 107 |
| VTDFY | 108 |
| SHLYWQPYSVQ | 109 |
| TLVYWQPYSLQT | 110 |
| RGDYWQPYSVQS | 111 |
| VHVYWQPYSVQT | 112 |
| RLVYWQPYSVQT | 113 |
| SRVWFQPYSLQS | 114 |
| NMVYWQPYSIQT | 115 |
| SVVFWQPYSVQT | 116 |
| TFVYWQPYALPL | 117 |
| TLVYWQPYSIQR | 118 |
| RLVYWQPYSVQR | 119 |
| SPVFWQPYSIQI | 120 |
| WIEWWQPYSVQS | 121 |
| SLIYWQPYSLQM | 122 |
| TRLYWQPYSVQR | 123 |
| RCDYWQPYSVQT | 124 |
| MRVFWQPYSVQN | 125 |
| KIVYWQPYSVQT | 126 |
| RHLYWQPYSVQR | 127 |
| ALVWWQPYSEQI | 128 |
| SRVWFQPYSLQS | 129 |
| WEQPYALPLE | 130 |
| QLVWWQPYSVQR | 131 |
| DLRYWQPYSVQV | 132 |
| ELVWWQPYSLQL | 133 |
| DLVWWQPYSVQW | 134 |
| NGNYWQPYSFQV | 135 |
| ELVYWQPYSIQR | 136 |
| ELMY)AIQPYSVQE | 137 |
| NLLYWQPYSMQD | 138 |
| GYEWYQPYSVQR | 139 |
| SRVWYQPYSVQR | 140 |
| LSEQYQPYSVQR | 141 |
| GGGWWQPYSVQR | 142 |
| VGRWYQPYSVQR | 143 |
| VHVYWQPYSVQR | 144 |
| QARWYQPYSVQR | 145 |
| VHVYWQPYSVQT | 146 |
| RSVYWQPYSVQR | 147 |
| TRVWFQPYSVQR | 148 |
| GRIWFQPYSVQR | 149 |
| GRVWFQPYSVQR | 150 |
| ARTWYQPYSVQR | 151 |
| ARVWWQPYSVQM | 152 |
| RLMFYQPYSVQR | 153 |
| ESMWYQPYSVQR | 154 |
| HFGWWQPYSVHM | 155 |
| ARFWWQPYSVQR | 156 |
| RLVYWQPYAPIY | 157 |
| RLVYWQPYSYQT | 158 |
| RLVYWQPYSLPI | 159 |
| RLVYWQPYSVQA | 160 |
| SRVWYQPYAKGL | 161 |
| SRVWYQPYAQGL | 162 |
| SRVWYQPYAMPL | 163 |
| SRVWYQPYSVQA | 164 |
| SRVWYQPYSLGL | 165 |
| SRVWYQPYAREL | 166 |
| SRVWYQPYSRQP | 167 |
| SRVWYQPYFVQP | 168 |
| EYEWYQPYALPL | 169 |
| IPEYWQPYALPL | 170 |
| SRIWWQPYALPL | 171 |
| DPLFWQPYALPL | 172 |
| SRQWVQPYALPL | 173 |
| IRSWWQPYALPL | 174 |
| RGYWQPYALPL | 175 |
| RLLWVQPYALPL | 176 |
| EYRWFQPYALPL | 177 |
| DAYWVQPYALPL | 178 |
| WSGYFQPYALPL | 179 |
| NIEFWQPYALPL | 180 |
| TRDWVQPYALPL | 181 |
| DSSWYQPYALPL | 182 |
| IGNWYQPYALPL | 183 |
| NLRWDQPYALPL | 184 |
| LPEFWQPYALPL | 185 |
| DSYWWQPYALPL | 186 |
| RSQYYQPYALPL | 187 |
| ARFWLQPYALPL | 188 |
| NSYFWQPYALPL | 189 |
| RFMYWQPYSVQR | 190 |
| AHLFWQPYSVQR | 191 |
| WWQPYALPL | 192 |
| YYQPYALPL | 193 |
| YFQPYALGL | 194 |
| YWYQPYALPL | 195 |
| RWWQPYATPL | 196 |
| GWYQPYALGF | 197 |
| YWYQPYALGL | 198 |
| IWYQPYAMPL | 199 |
| SNMQPYQRLS | 200 |
| TFVYWQPYAVGLPAAETACN | 201 |
| TFVYWQPYSVQMTITGKVTM | 202 |
| TFVYWQPYSSHXXVPXGFPL | 203 |
| TFVYWQPYYGNPQWAIHVRH | 204 |
| TFVYWQPYVLLELPEGAVRA | 205 |
| TFVYWQPYVDYVWPIPIAQV | 206 |
| GWYQPYVDGWR | 207 |
| RWEQPYVKDGWS | 208 |
| EWYQPYALGWAR | 209 |
| GWWQPYARGL | 210 |
| LFEQPYAKALGL | 211 |
| GWEQPYARGLAG | 212 |
| AWVQPYATPLDE | 213 |
| MWYQPYSSQPAE | 214 |
| GWTQPYSQQGEV | 215 |
| DWFQPYSIQSDE | 216 |
| PWIQPYARGFG | 217 |
| RPLYWQPYSVQV | 218 |
| TLIYWQPYSVQI | 219 |
| RFDYWQPYSDQT | 220 |
| WHQFVQPYALPL | 221 |
| EWDSVYWQPYSVQTLLR | 223 |
| WEQNVYWQPYSVQSFAD | 224 |
| SDVVYWQPYSVQSLEM | 225 |
| YYDGVYWQPYSVQVMPA | 226 |
| SDIWYQPYALPL | 227 |
| QRIWWQPYALPL | 228 |
| SRIWWQPYALPL | 229 |
| RSLYWQPYALPL | 230 |
| TIIWEQPYALPL | 231 |
| WETWYQPYALPL | 232 |
| SYDWEQPYALPL | 233 |
| SRIWCQPYALPL | 234 |
| EIMFWQPYALPL | 235 |
| DYVWQQPYALPL | 236 |
| MDLLVQWYQPYALPL | 237 |
| GSKVILWYQPYALPL | 238 |
| RQGANIWYQPYALPL | 239 |
| GGGDEPWYQPYALPL | 240 |
| SQLERTWYQPYALPL | 241 |
| ETWVREWYQPYALPL | 242 |
| KKGSTQWYQPYALPL | 243 |
| LQARMNWYQPYALPL | 244 |
| EPRSQKWYQPYALPL | 245 |
| VKQKWRWYQPYALPL | 246 |
| LRRHDVWYQPYALPL | 247 |
| RSTASIWYQPYALPL | 248 |
| ESKEDQWYQPYALPL | 249 |
| EGLTMKWYQPYALPL | 250 |
| EGSREGWYQPYALPL | 251 |
| VIEWWQPYALPL | 252 |
| VWYWEQPYALPL | 253 |
| ASEWWQPYALPL | 254 |
| FYEWWQPYALPL | 255 |
| EGWWVQPYALPL | 256 |
| WGEWLQPYALPL | 257 |
| DYVWEQPYALPL | 258 |
| AHTWWQPYALPL | 259 |
| FIEWFQPYALPL | 260 |
| WLAWEQPYALPL | 261 |
| VMEWWQPYALPL | 262 |
| ERMWQPYALPL | 263 |
| NXXWXXPYALPL | 264 |
| WGNWYQPYALPL | 265 |
| TLYWEQPYALPL | 266 |
| VWRWEQPYALPL | 267 |
| LLWTQPYALPL | 268 |
| SRIWXX PYALPL | 269 |
| SDIWYQPYALPL | 270 |
| WGYYXX PYALPL | 271 |
| TSGWYQPYALPL | 272 |
| VHPYXXPYALPL | 273 |
| EHSYFQPYALPL | 274 |
| XXIWYQPYALPL | 275 |
| AQLHSQPYALPL | 276 |
| WANWFQPYALPL | 277 |
| SRLYSQPYALPL | 278 |
| GVTFSQPYALPL | 279 |
| SIVWSQPYALPL | 280 |
| SRDLVQPYALPL | 281 |
| HWGHVYWQPYSVQDDLG | 282 |
| SWHSVYWQPYSVQSVPE | 283 |
| WRDSVYWQPYSVQPESA | 284 |
| TWDAVYWQPYSVQKWLD | 285 |
| TPPWVYWQPYSVQSLDP | 286 |
| YWSSVYWQPYSVQSVHS | 287 |
| YWYQPYALGL | 288 |
| YWYQPYALPL | 289 |
| EWIQPYATGL | 290 |
| NWEQPYAKPL | 291 |
| AFYQPYALPL | 292 |
| FLYQPYALPL | 293 |
| VCKQPYLEWC | 294 |
| ETPFTWEESNAYYWQPYALPL | 295 |
| QGWLTWQDSVDMYWQPYALPL | 296 |
| FSEAGYTWPENTYWQPYALPL | 297 |
| TESPGGLDWAKIYWQPYALPL | 298 |
| DGYDRWRQSGERYWQPYALPL | 299 |
| TANVSSFEWTPGYWQPYALPL | 300 |
| SVGEDHNFWTSE YWQPYALPL | 301 |
| MNDQTSEVSTFPYWQPYALPL | 302 |
| SWSEAFEQPRNLYWQPYALPL | 303 |
| QYAEPSALNDWGYWQPYALPL | 304 |
| NGDWATADWSNYYWQPYALPL | 305 |
| THDEHIYWQPYALPL | 306 |
| MLEKTYTTWTPG YWQPYALPL | 307 |
| WSDPLTRDADLYWQPYALPL | 308 |
| SDAFTTQDSQAMYWQPYALPL | 309 |
| GDDAAWRTDSLTYWQPYALPL | 310 |
| AIIRQLYRWSEMYWQPYALPL | 311 |
| ENTYSPNWADSMYWQPYALPL | 312 |
| MNDQTSEVSTFPYWQPYALPL | 313 |
| SVGEDHNFWTSEYWQPYALPL | 314 |
| QTPFTWEESNAYYWQPYALPL | 315 |
| ENPFTWQESNAYYWQPYALPL | 316 |
| VTPFTWEDSNVF YWQPYALPL | 317 |
| QIPFTWEQSNAYYWQPYALPL | 318 |
| QAPLTWQESAAYYWQPYALPL | 319 |
| EPTFTWEESKAT YWQPYALPL | 320 |
| TTTLTWEESNAYYWQPYALPL | 321 |
| ESPLTWEESSALYWQPYALPL | 322 |
| ETPLTWEESNAYYWQPYALPL | 323 |
| EATFTWAESNAYYWQPYALPL | 324 |
| EALFTWKESTAYYWQPYALPL | 325 |
| STP-TWEESNAYYWQPYALPL | 326 |
| ETPFTWEESNAYYWQPYALPL | 327 |
| KAPFTWEESQAYYWQPYALPL | 328 |
| STSFTWEESNAYYWQPYALPL | 329 |
| DSTFTWEESNAYYWQPYALPL | 330 |
| YIPFTWEESNAYYWQPYALPL | 331 |
| QTAFTWEESNAYYWQPYALPL | 332 |
| ETLFTWEESNAT YWQPYALPL | 333 |
| VSSFTWEESNAYYWQPYALPL | 334 |
| QPYALPL | 335 |
| Py-1-NapPYQJYALPL | 336 |
| TANVSSFEWTPG YWQPYALPL | 337 |
| FEWTPGYWQPYALPL | 338 |
| FEWTPGYWQJYALPL | 339 |
| FEWTPGYYQJYALPL | 340 |
| ETPFTWEESNAYYWQPYALPL | 341 |
| FTWEESNAYYWQJYALPL | 342 |
| ADVLYWQPYAPVTLWV | 343 |
| GDVAEYWQPYALPLTSL | 344 |
| SWTDYGYWQPYALPISGL | 345 |
| FEWTPGYWQPYALPL | 346 |
| FEWTPGYWQJYALPL | 347 |
| FEWTPGWYQPYALPL | 348 |
| FEWTPGWYQJYALPL | 349 |
| FEWTPGYYQPYALPL | 350 |
| FEWTPGYYQJYALPL | 351 |
| TANVSSFEWTPGYWQPYALPL | 352 |
| SWTDYGYWQPYALPISGL | 353 |
| ETPFTWEESNAWAIQPYALPL | 354 |
| ENTYSPNWADSMYWQPYALPL | 355 |
| SVGEDHNFWTSEYWQPYALPL | 356 |
| DGYDRWRQSGERYWQPYALPL | 357 |
| FEWTPGYWQPYALPL | 358 |
| FEWTPGYWQPY | 359 |
| FEWTPGYWQJY | 360 |
| EWTPGYWQPY | 361 |
| FEWTPGWYQJY | 362 |
| AEWTPGYWQJY | 363 |
| FAWTPGYWQJY | 364 |
| FEATPGYWQJY | 365 |
| FEWAPGYWQJY | 366 |
| FEWTAGYWQJY | 367 |
| FEWTPAYWQJY | 368 |
| FEWTPGAWQJY | 369 |
| FEWTPGYAQJY | 370 |
| FEWTPGYWQJA | 371 |
| FEWTGGYWQJY | 372 |
| FEWTPGYWQJY | 373 |
| FEWTJGYWQJY | 374 |
| FEVff PecGYWQJY | 375 |
| FEWTPAibYWQJY | 376 |
| FEWTPSarWYQJY | 377 |
| FEWTSarGYWQJY | 378 |
| FEWTPNYWQJY | 379 |
| FEWTPVYWQJY | 380 |
| FEWTVPYWQJY | 381 |
| AcFEWTPGVVYQJY | 382 |
| AcFEVffPGYWQJY | 383 |
| INap-EVff PGYYQJY | 384 |
| YEWTPGYYQJY | 385 |
| FEWVPGYYQJY | 386 |
| FEVff PGYYQJY | 387 |
| FEVff PsYYQJY | 388 |
| FEWTPnYYQJY | 389 |
| SHLY-Nap-QPYSVQM | 390 |
| TLVY-Nap-LDPYSLQT | 391 |
| RGDY-Nap-QPYSVQS | 392 |
| NMVY-Nap-QPYSIQT | 393 |
| VYWQPYSVQ | 394 |
| VY-Nap-QPYSVQ | 395 |
| TFVYWQJYALPL | 396 |
| FEWTPGYYQJ-Bpa | 397 |
| XaaFEWTPGYYQJ-Bpa | 398 |
| FEWTPGY-Bpa-QJY | 399 |
| AeFEWTPGY-Bpa-QJY | 400 |
| FEWTPG-Bpa-YQJY | 401 |
| AcFEWTPG-Bpa-YQJY | 402 |
| AcFE-Bpa-TPGYYQJY | 403 |
| AcFE-Bpa-TPGYYQJY | 404 |
| Bpa-EWTPGYYQJY | 405 |
| AcBpa-EWTPGYYQJY | 406 |
| VYWQPYSVQ | 407 |
| RLVYWQPYSVQR | 408 |
| RLVY-Nap-QPYSVQR | 409 |
| RLDYWQPYSVQR | 410 |
| RLVWFQPYSVQR | 411 |
| RLVYWQPYSIQR | 412 |
| DNSSWYDSFLL | 413 |
| DNTAWYESFLA | 414 |
| DNTAWYENFLL | 415 |
| PAREDNTAWYDSFLIWC | 416 |
| TSEYDNTTWYEKFLASQ | 417 |
| SQIPDNTAWYQSFLLHG | 418 |
| SPFIDNTAWYENFLLTY | 419 |
| EQIYDNTAWYDHFLLSY | 420 |
| TPFIDNTAWYENFLLTY | 421 |
| TYTYDNTAWYERFLMSY | 422 |
| TMTQDNTAWYENFLLSY | 423 |
| TIDNTAWYANLVQTYPQ | 424 |
| TIDNTAWYERFLAQYPD | 425 |
| HIDNTAWYENFLLTYTP | 426 |
| SQDNTAWYENFLLSYKA | 427 |
| QIDNTAWYERFLLQYNA | 428 |
| NQDNTAWYESFLLQYNT | 429 |
| TIDNTAWYENFLLNHNL | 430 |
| HYDNTAWYERFLQQGWH | 431 |
| ETPFTWEESNAYYWQPYALPL | 432 |
| YIPFTWEESNAYYWQPYALPL | 433 |
| DGYDRWRQSGERYWQPYALPL | 434 |
| pY-INap-pY-QJYALPL | 435 |
| TANVSSFEWTPGYWQPYALPL | 436 |
| FEWTPGYWQJYALPL | 437 |
| FEWTPGYWQPYALPLSD | 438 |
| FEWTPGYYQJYALPL | 439 |
| FEWTPGYWQJY | 440 |
| AcFEVVTPGYWQJY | 441 |
| AcFEWTPGWYQJY | 442 |
| AcFEWTPGYYQJY | 443 |
| AcFEWTPaYWQJY | 444 |
| AcFEWTPaWYQJY | 445 |
| AcFEWTPaYYQJY | 446 |
| FEWTPGYYQJYALPL | 447 |
| FEWTPGYWQJYALPL | 448 |
| FEWTPGWYQJYALPL | 449 |
| TANVSSFEWTPGYWQPYALPL | 450 |
| AcFEWTPGYWQJY | 451 |
| AcFEWTPGWYQJY | 452 |
| AcFEWTPGYYQJY | 453 |
| AcFEWTPAYWQJY | 454 |
| AcFEWTPAWYQJY | 455 |
| AcFEWTPAYYQJY | 456 |

TABLE 7

TPO-mimetic peptide sequences

| | |
|---|---|
| Sequence/structure | SEQ-ID-NO: |

| | |
|---|---|
| EGPTLRQWLAARA | 457 |
| IEGPTLRQWLAAKA | 458 |
| IEGPTLREWLAARA | 459 |
| IEGPTLRQWLAARA-A- | 460 |
| IEGPTLRQWLAARA | |
| IEGPTLRQWLAAKA-A- | 461 |
| IEGPTLRQWLAAKA | |
| IEGPTLRQCLAARA-A- | 462 |
| IEGPTLRQCLAARA | |
| IEGPTLRQWLAARA-A-K(BrAc)-A- | 463 |
| IEGPTLRQWLAARA | |
| IEGPTLRQWLAARA-A-K(PEG)-A- | 464 |
| IEGPTLRQWLAARA | |
| IEGPTLRQCLAARA-A- | 465 |
| IEGPTLRQWLAARA | |
| IEGPTLRQCLAARA-Δ- | 466 |
| IEGPTLRQCLAARA | |
| IEGPTLRQWLAARA-A- | 467 |
| IEGPTLRQULA/AtIA | |
| VRDQIXXXL | 468 |
| TLREWL | 469 |
| GRVRDQVAGW | 470 |
| GRVKDQIAQL | 471 |
| GVRDQVSWAL | 472 |
| ESVREQVMKY | 473 |
| SVRSQISASL | 474 |
| GVRETVYRHM | 475 |
| GVREVIVMHML | 476 |
| GRVRDQIWAAL | 477 |
| AGVRDQILIWL | 478 |
| GRVRDQIMLSL | 479 |
| GRVRDQI(X)₃L | 480 |
| CTLRQWLQGC | 481 |
| CTLQEFLEGC | 482 |
| CTRTEWLHGC | 483 |
| CTLREWLHGGFC | 484 |
| CTLREWVFAGLC | 485 |
| CTLRQWLILLGMC | 486 |
| CTLAEFLASGVEQC | 487 |
| CSLQEFLSHGGYVC | 488 |
| CTLREFLDPTTAVC | 489 |
| CTLKEWLVSHEVWC | 490 |
| CTLREWL(X)₂₋₆C | 491-495 |
| REGPTLRQWM | 496 |
| EGPTLRQWLA | 497 |
| ERGPFWAKAC | 498 |
| REGPRCVMWM | 499 |
| CGTEGPTLSTWLDC | 500 |
| CEQDGPTLLEWLKC | 501 |
| CELVGPSLMSWLTC | 502 |
| CLTGPFVTQWLYEC | 503 |
| CRAGPTLLEWLTLC | 504 |
| CADGPTLREWISFC | 505 |
| C(X)₁₋₂EGPTLREWL(X)₁₋₂C | 506-510 |
| GGCTLREWLHGGFCGG | 511 |
| GGCADGPTLREWISFCGG | 512 |
| GNADGPTLRQWLEGRRPKN | 513 |
| LAIEGPTLRQWLHGNGRDT | 514 |
| HGRVGPTLREWKTQVATKK | 515 |
| TIKGPTLRQWLKSREHTS | 516 |
| ISDGPTLKEWLSVTRGAS | 517 |
| SIEGPTLREWLTSRTPHS | 518 |

TABLE 8

G-CSF-mimetic peptide sequences

| | |
|---|---|
| Sequence/structure | SEQ ID NO: |

| | |
|---|---|
| EEDCK | 519 |
| EEDαK | 520 |
| pGluEDαK | 521 |
| PicSDαK | 522 |
| EEDCK-Δ-EEDCK | 523 |
| EEDXK-Δ-EEDXK | 524 |

TABLE 9

TNF-antagonist peptide sequences

| | |
|---|---|
| Sequence/structure | SEQ ID NO: |

| | |
|---|---|
| YCFTASENHCY | 525 |
| YCFTNSENHCY | 526 |
| YCFTRSENHCY | 527 |
| FCASENHCY | 528 |
| YCASENHCY | 529 |
| FCNSENHCY | 530 |
| FCNSENRCY | 531 |
| FCNSVENRCY | 532 |
| YCSQSVSNDCF | 533 |
| FCVSNDRCY | 534 |
| YCRKELGQVCY | 535 |
| YCKEPGQCY | 536 |
| YCRKEMGCY | 537 |
| FCRKEMGCY | 538 |
| YCWSQNLCY | 539 |
| YCELSQYLCY | 540 |
| YCWSQNYCY | 541 |
| YCWSQYLCY | 542 |
| DFLPHYKNTSLGHRP | 543 |

TABLE 10

Integrin-binding peptide sequences

| | |
|---|---|
| Sequence/structure | SEQ ID NO: |

| | |
|---|---|
| RX₁ETX2WX₃ | 544 |
| RX₁ETX₂WX₃ | 545 |
| RGDGX | 546 |
| CRGDGXC | 547 |
| CX₁X₂RLDX₃X₄C | 548 |
| CARRLDAPC | 549 |
| CPSRLDSPC | 550 |
| X₁X₂X₃RGDX₄X₅X₆ | 551 |
| CX₂CRGDCX₅C | 552 |
| CDCRGDCFC | 553 |
| CDCRGDCLC | 554 |
| CLCRGDCIC | 555 |
| X₁X₂DDX₄X₅X₇X₈ | 556 |
| X₁X₂X₃DDX₄X₅X₆X₇X₈ | 557 |
| CWDDGWL | 558 |
| CWDDLWWLC | 559 |
| CWDDGLMC | 560 |
| CWDDGWMC | 561 |
| CSWDDGWLC | 562 |
| CPDDLWWLC | 563 |
| NGR | NR |
| GSL | NR |
| RGD | NR |
| CGRECPRLCQSSC | 564 |
| CNGRCVSGCAGRC | 565 |
| CLSGSLSC | 566 |
| RGD | NR |
| NGR | NR |
| GSL | NR |
| NGRAHA | 567 |
| CNGRC | 568 |
| CDCRGDCFC | 569 |
| CGSLVRC | 570 |
| DLXXL | 571 |
| RTDLDSLRTYTL | 572 |
| RTDLDSLRTY | 573 |
| RTDLDSLRT | 574 |
| RTDLDSLR | 575 |
| GDLDLLKLRLTL | 576 |
| GDLHSLRQLLSR | 577 |
| RDDLHMLRLQLW | 578 |
| SSDLHALKKRYG | 579 |
| RGDLKQLSELTW | 580 |
| RGDLAALSAPPV | 581 |

TABLE 11

Selectin antagonist peptide sequences

| | |
|---|---|
| Sequence/structure | SEQ ID NO: |

| | |
|---|---|
| DITWDQLWDLMK | 582 |
| DITWDELWKIMN | 583 |
| DYTWFELWDMMQ | 584 |
| QITWAQLWNMMK | 585 |
| DMTWHDLWTLMS | 586 |
| DYSWHDLWEMMS | 587 |
| EITWDQLWEVMN | 588 |
| HVSWEQLWDIMN | 589 |
| HITWDQLWRIMT | 590 |
| RNMSWLELWEHMK | 591 |
| AEWTWDQLWHVMNPAESQ | 592 |
| HRAEWLALWEQMSP | 593 |
| KKEDWLALWRIMSV | 594 |
| ITWDQLWDLMK | 595 |
| DITWDQLWDLMK | 596 |
| DITWDQLWDLMK | 597 |
| DITWDQLWDLMK | 598 |
| CQNRYTDLVAIQNKNE | 599 |
| AENWADNEPNNKRNNED | 600 |
| RKNNKTWTWVGTKKALTNE | 601 |
| KKALTNEAENWAD | 602 |
| CQXRYTDLVAIQNKXE | 603 |
| RKXNXXWTWVGTXKXLTEE | 604 |
| AENWADGEPNNKXNXED | 605 |
| CXXXYTXLVAIQNKXE | 606 |
| RKXXXXWXWVGTXKXLTXE | 607 |
| AXNWXXXEPNNXXXED | 608 |
| XKXKTXEAXNWXX | 609 |

TABLE 12

Antipathogenic peptide sequences

| | |
|---|---|
| Sequence/structure | SEQ ID NO: |

| | |
|---|---|
| GFFALIPKIISSPLFKTLLSAVGSALSSSGGQQ | 610 |
| GFFALIPKIISSPLFKTLLSAVGSALSSSGGQE | 611 |
| GFFALIPKIISSPLFKTLLSAV | 612 |
| GFFALIPKIISSPLFKTLLSAV | 613 |
| KGFFALIPKIISSPLFKTLLSAV | 614 |
| KKGFFALIPKIISSPLFKTLLSAV | 615 |
| KKGFFALIPKIISSPLFKTLLSAV | 616 |
| GFFALIPKIIS | 617 |
| GIGAVLKVLTTGLPALISWIKRKRQQ | 618 |
| GIGAVLKVLTTGLPALISWIKRKRQQ | 619 |
| GIGAVLKVLTTGLPALISWIKRKRQQ | 620 |
| GIGAVLKVLTTGLPALISWIKR | 621 |
| AVLKVLTTGLPALISWIKR | 622 |
| KLLLLLKLLLLK | 623 |
| KLLLKLLLKLLK | 624 |
| KLLLKLKLKLLK | 625 |
| KKLLKLKLKLKK | 626 |
| KLLLKLLLKLLK | 627 |
| KLLLKLKLKLLK | 628 |
| KLLLLK | 629 |
| KLLLKLLK | 630 |
| KLLLKLKLKLLK | 631 |
| KLLLKLKLKLLK | 632 |
| KLLLKLKLKLLK | 633 |
| KAAAKAAAKAAK | 634 |
| KVVVKVVVKVVK | 635 |
| KVVVKVKVKVVK | 636 |
| KVVVKVKVKVK | 637 |
| KVVVKVKVKVVK | 638 |
| KLILKL | 639 |
| KVLHLL | 640 |
| LKLRLL | 641 |
| KPLHLL | 642 |
| KLILKLVR | 643 |
| KVFHLLHL | 644 |
| HKFRILKL | 645 |
| KPFHILHL | 646 |
| KIiIKIKIKIIK | 647 |
| KIIIKIKIKIIK | 648 |
| KIIIKIKIKIIK | 649 |
| KIPIKIKIKIPK | 650 |
| KIPIKIKIKIVK | 651 |
| RIIIRIRIRIIR | 652 |
| RIIiRIRIRIIR | 653 |
| RI[IRIRLRIIR | 654 |
| RIVIRIRIRLIR | 655 |
| RIIVRIRLRIIR | 656 |
| RIGIRLRVRIIR | 657 |
| KIVIRIRIRLIR | 658 |
| RIAVKWRLRFIK | 659 |
| KIGWKLRVRIIR | 660 |
| KKIGWLIIRVRR | 661 |
| RIVIRIRIRLIRIR | 662 |
| RIIVRIRLRIIRVR | 663 |
| RIGIRLRVRIIRRV | 664 |
| KIVIRIRARLIRIRIR | 665 |
| RIIVKIRLRIIKKIRL | 666 |
| KIGIKARVRIIRVKII | 667 |
| RIIVHIRLRIIHHIRL | 668 |
| HIGIKAHVRIIRVHII | 669 |
| RIYVKIHLRYIKKIRL | 670 |
| KIGHKARVHIIRYKII | 671 |
| RIYVKPHPRYIKKIRL | 672 |
| KPGHKARPHIIRYKII | 673 |
| KIVIRLIRIRLIRIRIRKIV | 674 |
| RIIVKIRLRIIKKIRLIKK | 675 |
| KIGWKLRVRIIRVKIGRLR | 676 |
| KLVIRIRIRLIRIRIRKIVKVKRIR | 677 |
| RFAVKIRLRIIKKIRLIKKIRKRVIK | 678 |
| KAGWKLRVRIIRVKIGRLRKIGWKKRVRIK | 679 |
| RIYVKPHPRYIKKIRL | 680 |
| KPGHKARPHIIRYKII | 681 |
| KIVIRIRIRLIRIRIRKIV | 682 |
| RIIVKIRLRIIKKIRLIKK | 683 |
| RIYVSKISIYIKKIRL | 684 |
| KIVIFTRIRLTSIRIRSIV | 685 |
| KPIHKARPTIIRYKMI | 686 |
| cyclicCKGFFALIPKIISSPLFKTLLSAVC | 687 |
| CKKGFFALIPKIISSPLFKTLLSAVC | 688 |
| CKKKGFFALIPKIISSPLFKTLLSAVC | 689 |
| CyclicCRIVIRIRIRLIRIRC | 690 |
| CyclicCKPGHKARPHIIRYKIIC | 691 |
| CyclicCRFAVKIRLRIIKKIRLIKKIRKRVIKC | 692 |
| KLLLKLLL KLLKC | 693 |
| KLLLKLLLKLLK | 694 |
| KLLLKLKLKLLKC | 695 |
| KLLLKLLLKLLK | 696 |

TABLE 13

VIP-mimetic peptide sequences

| | |
|---|---|
| Sequence/structure | SEQ ID NO: |

| | |
|---|---|
| HSDAVFYDNYTR LRKQMAVKKYLN SILN | 697 |
| Me HSDAVFYDNYTR LRKQMAVKKYLN SILN | 698 |
| X₁ X₁‘X₁” X₂ | 699 |
| X₃SX₄LN | 700 |
| KKYL | 701 |
| NSILN | 702 |
| KKYL | 703 |
| KKYA | 704 |
| AVKKYL | 705 |
| NSILN | 706 |
| KKYV | 707 |
| SILauN | 708 |
| KKYLNIe | 709 |
| NSYLN | 710 |
| NSIYN | 711 |
| KKYLPPNSILN | 712 |
| LauKKYL | 713 |
| CapKKYL | 714 |
| KYL | NR |
| KKYNIe | 715 |
| VKKYL | 716 |
| LNSILN | 717 |
| YLNSILN | 718 |
| KKYLN | 719 |
| KKYLNS | 720 |
| KKYLNSI | 721 |
| KKYLNSIL | 722 |
| KKYL | 723 |
| KKYDA | 724 |
| AVKKYL | 725 |
| NSILN | 726 |
| KKYV | 727 |
| SILauN | 728 |
| NSYLN | 729 |
| NSIYN | 730 |
| KKYLNIe | 731 |
| KKYLPPNSILN | 732 |
| KKYL | 733 |
| KKYDA | 734 |
| AVKKYL | 735 |
| NSILN | 736 |
| IKKYV | 737 |
| SILauN | 738 |
| LauKKYL | 739 |
| CapKKYL | 740 |
| KYL | NR |
| KYL | NR |
| KKYNIe | 741 |
| VKKYL | 742 |
| LNSILN | 743 |
| YLNSILN | 744 |
| KKYLNIe | 745 |
| KKYLN | 746 |
| KKYLNS | 747 |
| KKYLNSI | 748 |
| KKYLNSIL | 749 |
| KKKYLD | 750 |
| cyclicCKKYLC | 751 |
| CKKYLK | 752 |
| KKYA | 753 |
| WWTDTGLW | 754 |
| WWTDDGLW | 755 |
| WWDTRGLWVWTI | 756 |
| FWGNDGIWLESG | 757 |
| DWDQFGLWRGAA | 758 |
| RWDDNGLWVVVL | 759 |
| SGMWSHYGIWMG | 760 |
| GGRWDQAGLWVA | 761 |
| KLWSEQGIWMGE | 762 |
| CWSMHGLWLC | 763 |
| GCWDNTGIWVPC | 764 |
| DWDTRGLWVY | 765 |
| SLWDENGAWI | 766 |
| KWDDRGLWMH | 767 |
| QAWNERGLWT | 768 |
| QWDTRGLWVA | 769 |
| WNVHGIWQE | 770 |
| SWDTRGLWVE | 771 |
| DWDTRGLWVA | 772 |
| SWGRDGLWIE | 773 |
| EWTDNGLWAL | 774 |
| SWDEKGLWSA | 775 |
| SWDSSGLWMD | 776 |

TABLE 14

Mdm/hdm antagonist peptide sequences

| | |
|---|---|
| Sequence/structure | SEQ ID NO: |

| | |
|---|---|
| TFSDLW | 777 |
| QETFSDLWKLLP | 778 |
| QPTFSDLWKLLP | 779 |
| QETFSDYWKLLP | 780 |
| QPTFSDYWKLLP | 781 |
| MPRFMDYWEGLN | 782 |
| VQNFIDYWTQQF | 783 |
| TGPAFTHYWATF | 784 |
| IDRAPTFRDHWFALV | 785 |
| PRPALVFADYWETLY | 786 |
| PAFSRFWSDLSAGAH | 787 |
| PAFSRFWSKLSAGAH | 788 |
| PXFXDYWXXL | 789 |
| QETFSDLWKLLP | 790 |
| QPTFSDLWKLLP | 791 |
| QETFSDYWKLLP | 792 |
| QPTFSDYWKLLP | 793 |

TABLE 15

Calmodulin antagonist peptide sequences

| | |
|---|---|
| Sequence/structure | SEQ ID NO: |

| | |
|---|---|
| SCVKWGKKEFCGS | 794 |
| SCWKYWGKECGS | 795 |
| SCYEWGKLRWCGS | 796 |
| SCLRWGKWSNCGS | 797 |
| SCWRWGKYQICGS | 798 |
| SCVSWGALKLCGS | 799 |
| SCIRWGQNTFCGS | 800 |
| SCWQWGNLKICGS | 801 |
| SCVRWGQLSICGS | 802 |
| LKKFNARRKLKGAILTTMLAK | 803 |
| RRWKKNFIAVSAANRFKK | 804 |
| RKWQKTGHAVRAIGRLSS | 805 |
| INLKALAALAKKIL | 806 |
| KIWSILAPLGTTLVKLVA | 807 |
| LKKLLKLLKKLLKL | 808 |
| LKWKKLLKLLKKLLKKLL | 809 |
| AEWPSLTEIKTLSHFSV | 810 |
| AEWPSPTRVISTTYFGS | 811 |
| AELAHWPPVKTVLRSFT | 812 |
| AEGSWLQLLNLMKQMNN | 813 |
| AEWPSLTEIK | 814 |

TABLE 16

Mast cell antagonists/Mast cell protease
inhibitor peptide sequences

| | |
|---|---|
| Sequence/structure | SEQ ID NO: |

| | |
|---|---|
| SGSGVLKRPLPILPVTR | 815 |
| RWLSSRPLPPLPLPPRT | 816 |
| GSGSYDTLALPSLPLHPMSS | 817 |
| GSGSYDTRALPSLPLHPMSS | 818 |
| GSGSSGVTMYPKLPPHWSMA | 819 |
| GSGSSGVRMYPKLPPHWSMA | 820 |
| GSGSSSMRMVPTIPGSAKHG | 821 |
| RNR | NR |
| QT | NR |
| RQK | NR |
| NRQ | NR |
| RQK | NR |
| RNRQKT | 822 |
| RNRQ | 823 |
| RNRQK | 824 |
| NRQKT | 825 |
| RQKT | 826 |

TABLE 17

SH3 antagonist peptide sequences

| | |
|---|---|
| Sequence/structure | SEQ ID NO: |

| | |
|---|---|
| RPLPPLP | 827 |
| RELPPLP | 828 |
| SPLPPLP | 829 |
| GPLPPLP | 830 |
| RPLPIPP | 831 |
| RPLPIPP | 832 |
| RRLPPTP | 834 |
| RQLPPTP | 835 |
| RPLPSRP | 836 |
| RPLPTRP | 837 |
| SRLPPLP | 838 |
| RALPSPP | 839 |
| RRLPRTP | 840 |
| RPVPPIT | 841 |
| ILAPPVP | 842 |
| RPLPMLP | 843 |
| RPLPILP | 844 |
| RPLPSLP | 845 |
| RPLPSLP | 846 |
| RPLPMIP | 847 |
| RPLPLIP | 848 |
| RPLPPTP | 849 |
| RSLPPLP | 850 |
| RPQPPPP | 851 |
| RQLPIPP | 852 |
| XXXRPLPPLPXP | 853 |
| XXXRPLPPIPXX | 854 |
| XXXRPLPPLPXX | 855 |
| RXXRPLPPLPXP | 856 |
| RXXRPLPPLPPP | 857 |
| PPPYPPPPIPXX | 858 |
| PPPYPPPPVPXX | 859 |
| LXXRPLPXTP | 860 |
| ΨXXRPLPXLP | 861 |
| PPXΘXPPPΨP | 862 |
| +PPΨPXKPXWL | 863 |
| RPXΨPΨR+SXP | 864 |
| PPVPPRPXXTL | 865 |
| ΨPΨLPΨK | 866 |
| +ΘDXPLPXLP | 867 |

TABLE 18

Somatostatin or cortistatin mimetic peptide
sequences

| | SEQ |
|---|---|
| | ID |
| Sequence/structure | NO: |
| X¹X²-Asn-Phe-Phe-Trp-Lys-Thr-Phe-X³-Ser-X⁴ | 868 |
| Asp Arg Met Pro Cys Arg Asn Phe Phe Trp Lys Thr | 869 |
| Phe Ser Ser Cys Lys | |
| Met Pro Cys Arg Asn Phe Phe Trp Lys Thr Phe Ser | 870 |
| Ser Cys Lys | |
| Cys Arg Asn Phe Phe Trp Lys Thr Phe Ser Ser Cys | 871 |
| Lys | |
| Asp Arg Met Pro_Cys Arg Asn Phe Phe Trp Lys Thr | 872 |
| Phe Ser Ser Cys | |
| Met Pro Cys Arg Asn Phe Phe Trp Lys Thr Phe Ser | 873 |
| Ser Cys | |
| Cys Arg Asn Phe Phe Trp Lys Thr Phe Ser Ser Cys | 874 |
| Asp Arg Met Pro Cys Lys Asn Phe Phe Trp Lys Thr | 875 |
| Phe Ser Ser Cys | |
| Met Pro Cys Lys Asn Phe Phe Trp Lys Thr Phe Ser | 876 |
| Ser Cys Lys | |
| Cys Lys Asn Phe Phe Trp Lys Thr Phe Ser Ser Cys | 877 |
| Lys | |
| Asp Arg Met Pro Cys Lys Asn Phe Phe Trp Lys Thr | 878 |
| Phe Ser Ser Cys | |
| Met Pro Cys Lys Asn Phe Phe Trp Lys Thr Phe Ser | 879 |
| Ser Cys | |
| Cys Lys Asn Phe Phe Trp Lys Thr Phe Ser Ser Cys | 880 |
| Asp Arg Met Pro Cys Arg Asn Phe Phe Trp Lys Thr | 881 |
| Phe Thr Ser Cys Lys | |
| Met Pro Cys Arg Asn Phe Phe Trp Lys Thr Phe Thr | 882 |
| Ser Cys Lys | |
| Cys Arg Asn Phe Phe Trp Lys Thr Phe Thr Ser Cys | 883 |
| Lys | |
| Asp Arg Met Pro Cys Arg Asn Phe Phe Trp Lys Thr | 884 |
| Phe Thr Ser Cys | |
| Met Pro Cys Arg Asn Phe Phe Trp Lys Thr Phe Thr | 885 |
| Ser Cys | |
| Cys Arg Asn Phe Phe Trp Lys Thr Phe Thr Ser Cys | 886 |
| Asp Arg Met Pro Cys Lys Asn Phe Phe Trp Lys Thr | 887 |
| Phe Thr Ser Cys Lys | |
| Met Pro Cys Lys Asn Phe Phe Trp Lys Thr Phe Thr | 889 |
| Ser Cys Lys | |
| Cys Lys Asn Phe Phe Trp Lys Thr Phe Thr Ser Cys | 890 |
| Lys | |
| Asp Arg Met Pro Cys Lys Asn Phe Phe Trp Lys Thr | 891 |
| Phe Thr Ser Cys | |
| Met Pro Cys Lys Asn Phe Phe Trp Lys Thr Phe Thr | 892 |
| Ser Cys | |
| Cys Lys Asn Phe Phe Trp Lys Thr Phe Thr Ser Cys | 893 |

TABLE 19

UKR antagonist peptide sequences

| | |
|---|---|
| Sequence/structure | SEQ ID NO: |

| | |
|---|---|
| AEPMPHSLNFSQYLWYT | 894 |
| AEHTYSSLWDTYSPLAF | 895 |
| AELDLWMRHYPLSFSNR | 896 |
| AESSLWTRYAWPSMPSY | 897 |
| AEWHPGLSFGSYLWSKT | 898 |
| AEPALLNWSFFFNPGLH | 899 |
| AEWSFYNLHLPEPQTIF | 900 |
| AEPLDLWSLYSLPPLAM | 901 |
| AEPTLWQLYQFPLRLSG | 902 |
| AEISFSELMWLRSTPAF | 903 |
| AELSEADLWTTWFGMGS | 904 |
| AESSLWRIFSPSALMMS | 905 |
| AESLPTLTSILWGKESV | 906 |
| AETLFMDLWHDKHILLT | 907 |
| AEILNFPLWHEPLWSTE | 908 |
| AESQTGTLNTLFWNTLR | 909 |
| AEPVYQYELDSYLRSYY | 910 |
| AELDLSTFYDIQYLLRT | 911 |
| AEFFKLGPNGYVYLHSA | 912 |
| FKLXXXGYVYL | 913 |
| AESTYHHLSLGYMYTLN | 914 |
| YHXLXXGYMYT | 915 |

TABLE 20

Macrophage and/or T-cell inhibiting peptide
sequences

| | |
|---|---|
| Sequence/structure | SEQ ID NO: |

| | |
|---|---|
| Xaa-Yaa-Arg | NR |
| Arg-Yaa-Xaa | NR |
| Xaa-Arg-Yaa | NR |
| Yaa-Arg-Xaa | NR |
| Ala-Arg | NR |
| Arg—Arg | NR |
| Asn-Arg | NR |
| Asp-Arg | NR |
| Cys-Arg | NR |
| Gln-Arg | NR |
| Glu-Arg | NR |
| Gly-Arg | NR |
| His-Arg | NR |
| Ile-Arg | NR |
| Leu-Arg | NR |
| Lys-Arg | NR |
| Met-Arg | NR |
| Phe-Arg | NR |
| Ser-Arg | NR |
| Thr-Arg | NR |
| Trp-Arg | NR |
| Tyr-Arg | NR |
| Val-Arg | NR |
| Ala-Glu-Arg | NR |
| Arg-Glu-Arg | NR |
| Asn-Glu-Arg | NR |
| Asp-Glu-Arg | NR |
| Cys-Glu-Arg | NR |
| Gln-Glu-Arg | NR |
| Glu-Glu-Arg | NR |
| Gly-Glu-Arg | NR |
| His-Glu-Arg | NR |
| Ile-Glu-Arg | NR |
| Leu-Glu-Arg | NR |
| Lys-Glu-Arg | NR |
| Met-Glu-Arg | NR |
| Phe-Glu-Arg | NR |
| Pro-Glu-Arg | NR |
| Ser-Glu-Arg | NR |
| Thr-Glu-Arg | NR |
| Trp-Glu-Arg | NR |
| Tyr-Glu-Arg | NR |
| Val-Glu-Arg | NR |
| Arg-Ala | NR |
| Arg-Asp | NR |
| Arg-Cys | NR |
| Arg-Gln | NR |
| Arg-Glu | NR |
| Arg-Gly | NR |
| Arg-His | NR |
| Arg-Ile | NR |
| Arg-Leu | NR |
| Arg-Lys | NR |
| Arg-Met | NR |
| Arg-Phe | NR |
| Arg-Pro | NR |
| Arg-Ser | NR |
| Arg-Thr | NR |
| Arg-Trp | NR |
| Arg-Tyr | NR |
| Arg-Val | NR |
| Arg-Glu-Ala | NR |
| Arg-Glu-Asn | NR |
| Arg-Glu-Asp | NR |
| Arg-Glu-Cys | NR |
| Arg-Glu-Gln | NR |
| Arg-Glu-Glu | NR |
| Arg-Glu-Gly | NR |
| Arg-Glu-His | NR |
| Arg-Glu-Ile | NR |
| Arg-Glu-Leu | NR |
| Arg-Glu-Lys | NR |
| Arg-Glu-Met | NR |
| Arg-Glu-Phe | NR |
| Arg-Glu-Pro | NR |
| Arg-Glu-Ser | NR |
| Arg-Glu-Thr | NR |
| Arg-Glu-Trp | NR |
| Arg-Glu-Tyr | NR |
| Arg-Glu-Val | NR |
| Ala-Arg-Glu | NR |
| Arg-Arg-Glu | NR |
| Asn-Arg-Glu | NR |
| Asp-Arg-Glu | NR |
| Cys-Arg-Glu | NR |
| Gln-Arg-Glu | NR |
| Glu-Arg-Glu | NR |
| Gly-Arg-Glu | NR |
| His-Arg-Glu | NR |
| Ile-Arg-Glu | NR |
| Leu-Arg-Glu | NR |
| Lys-Arg-Glu | NR |
| Met-Arg-Glu | NR |
| Phe-Arg-Glu | NR |
| Pro-Arg-Glu | NR |
| Ser-Arg-Glu | NR |
| Thr-Arg-Glu | NR |
| Trp-Arg-Glu | NR |
| Tyr-Arg-Glu | NR |
| Val-Arg-Glu | NR |
| Glu-Arg-Ala | NR |
| Glu-Arg-Arg | NR |
| Glu-Arg-Asn | NR |
| Glu-Arg-Asp | NR |
| Glu-Arg-Cys | NR |
| Glu-Arg-Gln | NR |
| Glu-Arg-Gly | NR |
| Glu-Arg-His | NR |
| Glu-Arg-Ile | NR |
| Glu-Arg-Leu | NR |
| Glu-Arg-Lys | NR |
| Glu-Arg-Met | NR |
| Glu-Arg-Phe | NR |
| Glu-Arg-Pro | NR |
| Glu-Arg-Ser | NR |
| Glu-Arg-Thr | NR |
| Glu-Arg-Trp | NR |
| Glu-Arg-Tyr | NR |
| Glu-Arg-Val | NR |

TABLE 21

Additional Exemplary Pharmacologically Active
Peptides

| | SEQ | |
|---|---|---|
| | ID | |
| Sequence/Structure | NO: | Activity |
| VEPNCDIHVMWEWECFERL | 916 | VEGF- |
| | | antagonist |
| GERWCFDGPLTWVCGEES | 917 | VEGF- |
| | | antagonist |
| RGWVEICVADDNGMCVTEAQ | 918 | VEGF- |
| | | antagonist |
| GWDECDVARMWEWECFAGV | 919 | VEGF- |
| | | antagonist |
| GERWCFDGPRAWVCGWEI | 920 | VEGF- |
| | | antagonist |
| EELWCFDGPRAWVCGYVK | 921 | VEGF- |
| | | antagonist |
| RGWVEICAADDYGRCLTEAQ | 922 | VEGF- |
| | | antagonist |
| RGWVEICESDVWGRCL | 923 | VEGF- |
| | | antagonist |
| RGWVEICESDVWGRCL | 924 | VEGF- |
| | | antagonist |
| GGNECDIARMWEWECFERL | 925 | VEGF- |
| | | antagonist |
| RGWVEICAADDYGRCL | 926 | VEGF- |
| | | antagonist |
| CTTHWGFTLC | 927 | MMP |
| | | inhibitor |
| CLRSGXGC | 928 | MMP |
| | | inhibitor |
| CXXHWGFXXC | 929 | MMP |
| | | inhibitor |
| CXPXC | 930 | MMP |
| | | inhibitor |
| CRRHWGFEFC | 931 | MMP |
| | | inhibitor |
| STTHWGFTLS | 932 | MMP |
| | | inhibitor |
| CSLHWGFWWC | 933 | CTLA4- |
| | | mimetic |
| GFVCSGIFAVGVGRC | 934 | CTLA4- |
| | | mimetic |
| APGVRLGCAVLGRYC | 935 | CTLA4- |
| | | mimetic |
| LLGRMK | 936 | Antiviral |
| | | (HBV) |
| ICVVQDWGHHRCTAGHMANLTSHASAI | 937 | C3b |
| | | antagonist |
| ICVVQDWGHHRCT | 938 | C3b |
| | | antagonist |
| CVVQDWGHHAC | 939 | C3b |
| | | antagonist |
| STGGFDDVYDWARGVSSALTTTLVATR | 940 | Vinculin- |
| | | binding |
| STGGFDDVYDWARRVSSALTTTLVATR | 941 | Vinculin- |
| | | binding |
| SRGVNFSEWLYDMSAAMKEASNVFPSRRSR | 942 | Vinculin- |
| | | binding |
| SSQNWDMEAGVEDLTAAMLGLLSTIHSSSR | 943 | Vinculin- |
| | | binding |
| SSPSLYTQFLVNYESAATRIQDLLIASRPSR | 944 | Vinculin- |
| | | binding |
| SUGMIDILLGAILQRAADATRTSIPIPSLQNSIR | 945 | Vinculin- |
| | | binding |
| DVYTKKELIECARRVSEK | 946 | Vinculin- |
| | | binding |
| EKGSYYPGSGIAQFHIDYNNVS | 947 | C4BP- |
| | | binding |
| SGIAQFHIDYNNVSSAEGWHVN | 948 | C41BP- |
| | | binding |
| LVTVEKGSYYPGSGIAQFHIDYNNVSSAEGWHVN | 949 | 4BP- |
| | | binding |
| SGIAQFHIDYNNVS | 950 | C4BP- |
| | | binding |
| LLGRMK | 951 | anti-HBV |
| ALLGRMKG | 952 | anti-HBV |
| LDPAFIR | 953 | anti-HBV |
| CXXRGDC | 954 | Inhibition |
| | | of platelet |
| | | aggrepation |
| RPLPPLP | 955 | Src |
| | | antagonist |
| PPVPPR | 956 | Src |
| | | antagonist |
| XFXDXWXXLXX | 957 | Anti-cancer |
| KACRRLFGPVDSEQLSRDCD | 958 | p16-mimetic |
| RERWNFDFVTETPLEGDFAW | 959 | p16-mimetic |
| KRRQTSMTDFYHSKRRLIFS | 960 | p16-mimetic |
| TSMTDFYHSKRRLIFSKRKP | 961 | p16-mimetic |
| RRLIF | 962 | p16-mimetic |
| KRRQTSATDFYHSKRRLIFSRQIKIWFQNRRMKWKK | 963 | p16-mimetic |
| KRRLIFSKRQIKIWFQNRRMKWKK | 964 | p16-mimetic |
| Asn Gin Gly Arg His Phe Cys Gly Gly | 965 | CAP37 |
| Ala Leu Ile His Ala Arq Phe Val Met | | mimetic/LPs |
| Thr Ala Ala Ser Cys Phe Gln | | bindin |
| Arg His Phe Cys Gly Gly Ala Leu Ile | 499 | CAP37 |
| His Ala Arg Phe Val Met Thr Ala Ala | | mimetic/LPS |
| Ser Cys | | binding |
| Gly Thr Arg Cys Gin Val Ala Gly Trp | 966 | CAP37 |
| Gly Ser Gln Arg Ser Gly Gly Arg Leu | | mimetic/LPS |
| Ser Arg Phe Pro Arg Phe Val Asn Val | | binding |
| WHWRHRIPLQLAAGR | 967 | carbohy- |
| | | drate (GID1 |
| | | alpha) |
| | | mimetic |
| LKTPRV | 968 | I32GPI Ab |
| | | binding |
| NTLKTPRV | 969 | I32GPI Ab |
| | | binding |
| NTLKTPRVGGC | 970 | 02GPI Ab |
| | | binding |
| KDKATF | 971 | 02GPI Ab |
| | | binding |
| KDKATFGCHD | 972 | P2GPI Ab |
| | | binding |
| KDKATFGCHDGC | 973 | 02GPI Ab |
| | | bindinq |
| TLRVYK | 974 | 02GPI Ab |
| | | binding |
| ATLRVYKG | 975 | 02GPI Ab |
| | | binding |
| CATLRVYKGG | 976 | I32GPI Ab |
| | | binding |
| INLKALAALAKKIL | 977 | Membrane |
| | | trans- |
| | | porting |
| GWT | NR | Membrane |
| | | trans- |
| | | porting |
| GWTLNSAGYLLG | 978 | Membrane |
| | | trans- |
| | | porting |
| GWTLNSAGYLLGKINLKALAALAKKIL | 979 | Membrane |
| | | trans- |
| | | porting |

The present invention is also particularly useful with peptides having activity in treatment of: a VEGF related condition, e.g., but not limited to, cancer, wherein the peptide is a VEGF-mimetic or a VEGF receptor antagonist, a HER2 agonist or antagonist, a CD20 antagonist and the like; asthma, wherein the protein of interest is a CKR3 antagonist, an IL-5 receptor antagonist, and the like; thrombosis, wherein the protein of interest is a GPIIb antagonist, a GPIIIa antagonist, and the like; autoimmune diseases and other conditions involving immune modulation, wherein the protein of interest is an IL-2 receptor antagonist, a CD40 agonist or antagonist, a CD40L agonist or antagonist, a thymopoietin mimetic and the like.

For example, EPO biological activities are well known in the art. See, e.g., Anagnostou A et al Erythropoietin has a mitogenic and positive chemotactic effect on endothelial cells. Proceedings of the National Academy of Science (USA) 87: 5978-82 (1990); Fandrey J and Jelkman W E Interleukin 1 and tumor necrosis factor-alpha inhibit erythropoietin production in vitro. Annals of the New York Academy of Science 628: 250-5 (1991); Geissler K et al Recombinant human erythropoietin: A multipotential hemopoietic growth factor in vivo and in vitro. Contrib. Nephrol. 87: 1-10 (1990); Gregory C J Erythropoietin sensitivity as a differentiation marker in the hemopoietic system. Studies of three erythropoietic colony responses in culture. Journal of Cellular Physiology 89: 289-301 (1976); Jelkman W et al Monokines inhibiting erythropoietin production in human hepatoma cultures and in isolated perfused rat kidneys. Life Sci. 50: 301-8 (1992); Kimata H et al Human recombinant erythropoietin directly stimulates B cell immunoglobulin production and proliferation in serum-free medium. Clinical and Experimental Immunology 85: 151-6 (1991); Kimata H et al Erythropoietin enhances immunoglobulin production and proliferation by human plasma cells in a serum-free medium. Clin. Immunology Immunopathol. 59: 495-501 (1991); Kimata H et al Effect of recombinant human erythropoietin on human IgE production in vitro Clinical and Experimental Immunology 83: 483-7 (1991); Koury M J and Bondurant M C Erythropoietin retards DNA breakdown and prevents programmed cell death in erythroid progenitor cells. Science 248: 378-81 (1990); Lim V S et al Effect of recombinant human erythropoietin on renal function in humans. Kidney International 37: 131-6 (1990); Mitjavila M T et al Autocrine stimulation by erythropoietin and autonomous growth of human erythroid leukemic cells in vitro. Journal of Clinical Investigation 88: 789-97 (1991); Andre M et al Performance of an immunoradiometric assay of erythropoietin and results for specimens from anemic and polycythemic patients. Clinical Chemistry 38: 758-63 (1992); Hankins W D et al Erythropoietin-dependent and erythropoietin-producing cell lines. Implications for research and for leukemia therapy. Annals of the New York Academy of Science 554: 21-8 (1989); Kendall R G T et al Storage and preparation of samples for erythropoietin radioimmunoassay. Clin. Lab. Haematology 13: 189-96 (1991); Krumvieh D et al Comparison of relevant biological assays for the determination of biological active erythropoietin. Dev. Biol. Stand. 69: 15-22 (1988); Ma D D et al Assessment of an EIA for measuring human serum erythropoietin as compared with RIA and an in-vitro bioassay. British Journal of Haematology 80: 431-6 (1992); Noe G et al A sensitive sandwich ELISA for measuring erythropoietin in human serum British Journal of Haematology 80: 285-92 (1992); Pauly J U et al Highly specific and highly sensitive enzyme immunoassays for antibodies to human interleukin 3 (IL3) and human erythropoietin (EPO) in serum. Behring Institut Mitteilungen 90: 112-25 (1991); Sakata S and Enoki Y Improved microbioassay for plasma erythropoietin based on CFU-E colony formation. Ann. Hematology 64: 224-30 (1992); Sanengen T et al Immunoreactive erythropoietin and erythropoiesis stimulating factor(s) in plasma from hypertransfused neonatal and adult mice. Studies with a radioimmunoassay and a cell culture assay for erythropoietin. Acta Physiol. Scand. 135: 11-6 (1989); Widness J A et al A sensitive and specific erythropoietin immunoprecipitation assay: application to pharmacokinetic studies. Journal of Lab. Clin. Med. 119: 285-94 (1992); for further information see also individual cell lines used in individual bioassays. Each of the above references are entirely incorporated herein by reference. EPO can be assayed by employing cell lines such as HCD57, NFS-60, TF-1 and UT-7, which respond to the factor. EPO activity can be assessed also in a Colony formation assay by determining the number of CFU-E from bone marrow cells. An alternative and entirely different detection method is RT-PCR quantitation of cytokines.

A hinge core mimetibody, or specified portion or variant thereof, that partially or preferably substantially provides at least one biological activity of at least one protein or fragment, can bind the protein or fragment ligand and thereby provide at least one activity that is otherwise mediated through the binding of protein to at least one protein ligand or receptor or through other protein-dependent or mediated mechanisms. As used herein, the term “hinge core mimetibody activity” refers to a hinge core mimetibody that can modulate or cause at least one protein-dependent activity by about 20-10,000%, preferably by at least about 60, 70, 80, 90, 91, 92, 93, 94, 95, 96, 97, 98, 99, 100, 110, 120, 130, 140, 150, 160, 170, 180, 190, 200, 250, 300, 350, 400, 450, 500, 550, 600, 700, 800, 900, 1000, 2000, 3000, 4000, 5000, 6000, 7000, 8000, 9000% or more depending on the assay.

The capacity of a hinge core mimetibody or specified portion or variant to provide at least one protein-dependent activity is preferably assessed by at least one suitable protein biological assay, as described herein and/or as known in the art. A human hinge core mimetibody or specified portion or variant of the invention can be similar to any class (IgG, IgA, IgM, etc.) or isotype and can comprise at least a portion of a kappa or lambda light chain. In one embodiment, the human hinge core mimetibody or specified portion or variant comprises an IgG heavy chain or defined fragment, for example, at least one of isotypes, IgG1, IgG2, IgG3 or IgG4. In another embodiment, the human protein human hinge core mimetibody or specified portion or variant thereof comprises an IgG1 heavy chain and an IgG1 light chain.

At least one hinge core mimetibody or specified portion or variant of the invention binds at least one specified ligand specific to at least one protein, subunit, fragment, portion or any combination thereof. The at least one therapeutic peptide portion (P) of at least one mimetibody of the invention can optionally bind at least one specified ligand epitope of the ligand. The binding epitope can comprise any combination of at least one amino acid sequence of at least 1-3 amino acids to the entire specified portion of contiguous amino acids of the sequences selected from the group consisting of a protein ligand, such as a receptor or portion thereof.

The hinge core mimetibody can comprise at least one N terminal heavy or light chain variable region having a defined amino acid sequence. Mimetibodies that bind to human protein ligands or receptors and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. The hinge core mimetibody, specified portion or variant can be expressed using the encoding nucleic acid or portion thereof in a suitable host cell.

The invention also relates to mimetibodies, ligand-binding fragments, immunoglobulin chains comprising amino acids in a sequence that is substantially the same as an amino acid sequence described herein. Preferably, such mimetibodies or ligand-binding fragments and mimetibodies comprising such chains can bind human protein ligands with high affinity (e.g., K_{D} less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Amino Acid Codes

The amino acids that make up mimetibodies or specified portions or variants of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994), as presented in the following Table 22:

TABLE 22

| SINGLE | THREE | | |
|---|---|---|---|
| LETTER | LETTER | | THREE NUCLEOTIDE |
| CODE | CODE | NAME | CODON(S) |
| A | Ala | Alanine | GCA, GCC, GCG, GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, CUC, |
| | | | CUG, CUU |
| M | Met | Methionine | AUG |
| N | Asn | Asparagine | AAC, AAU |
| P | Pro | Proline | CCA, CCC, CCG, CCU |
| Q | Gln | Glutamine | CAA, CAG |
| R | Arg | Arginine | AGA, AGG, CGA, CGC, |
| | | | CGG, CGU |
| S | Ser | Serine | AGC, AGU, UCA, UCC, |
| | | | UCG, UCU |
| T | Thr | Threonine | ACA, ACC, ACG, ACU |
| V | Val | Valine | GUA, GUC, GUG, GUU |
| W | Trp | Tryptophan | UGG |
| Y | Tyr | Tyrosine | UAC, UAU |

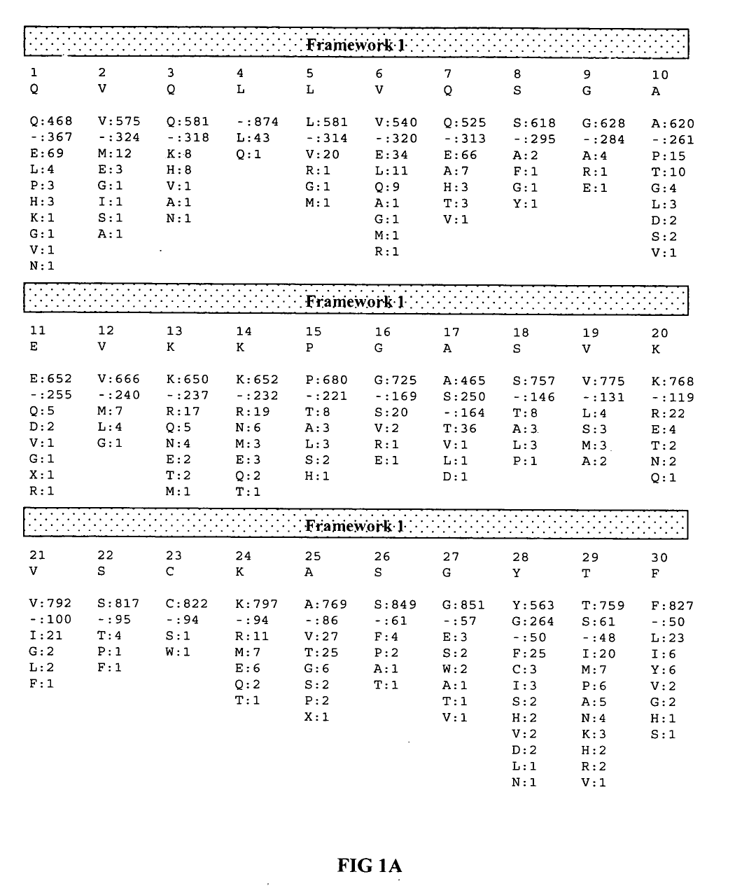

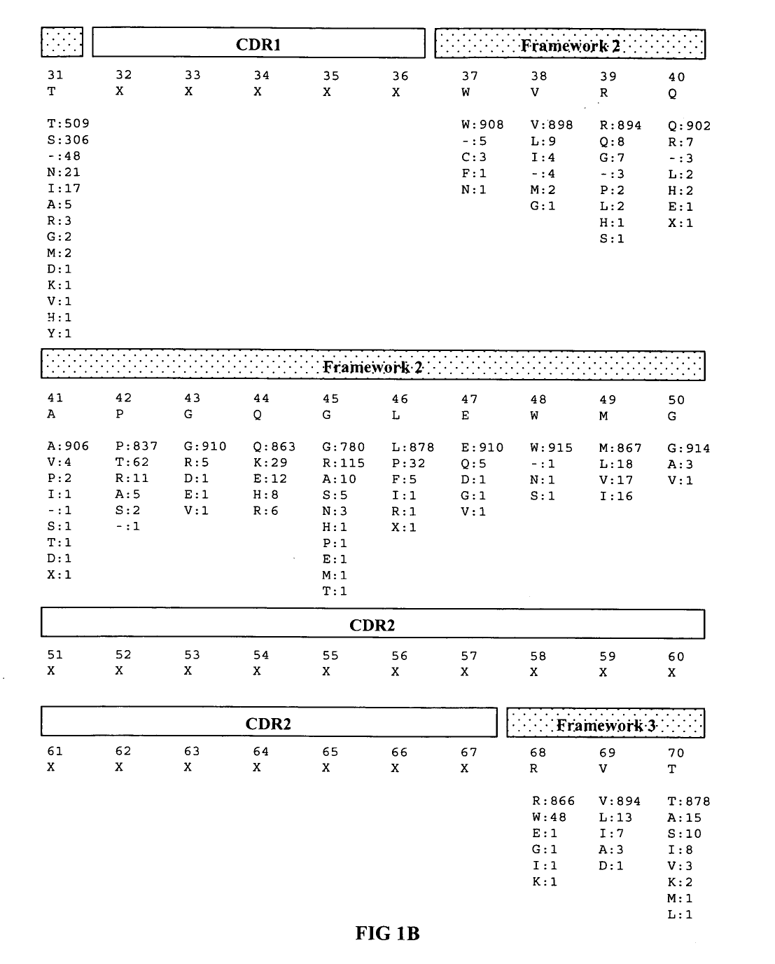

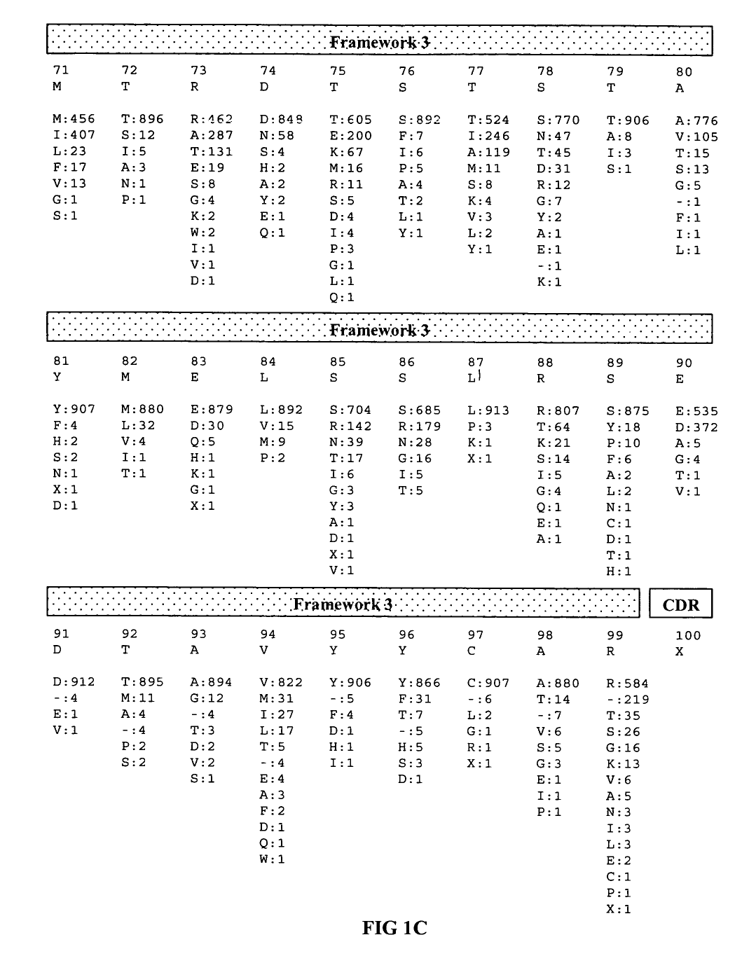

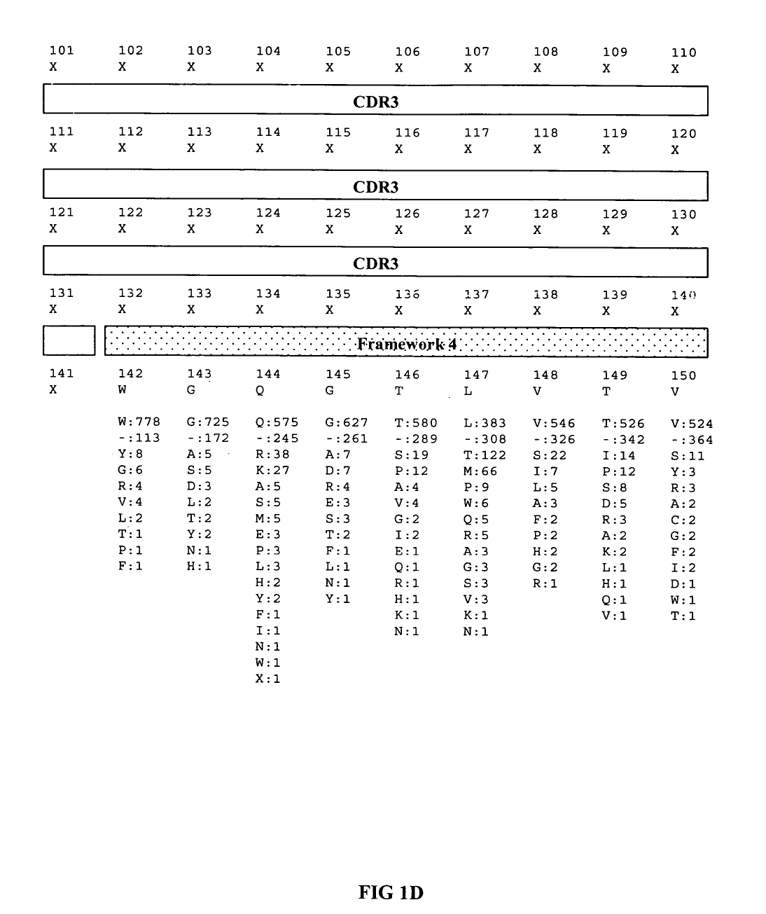

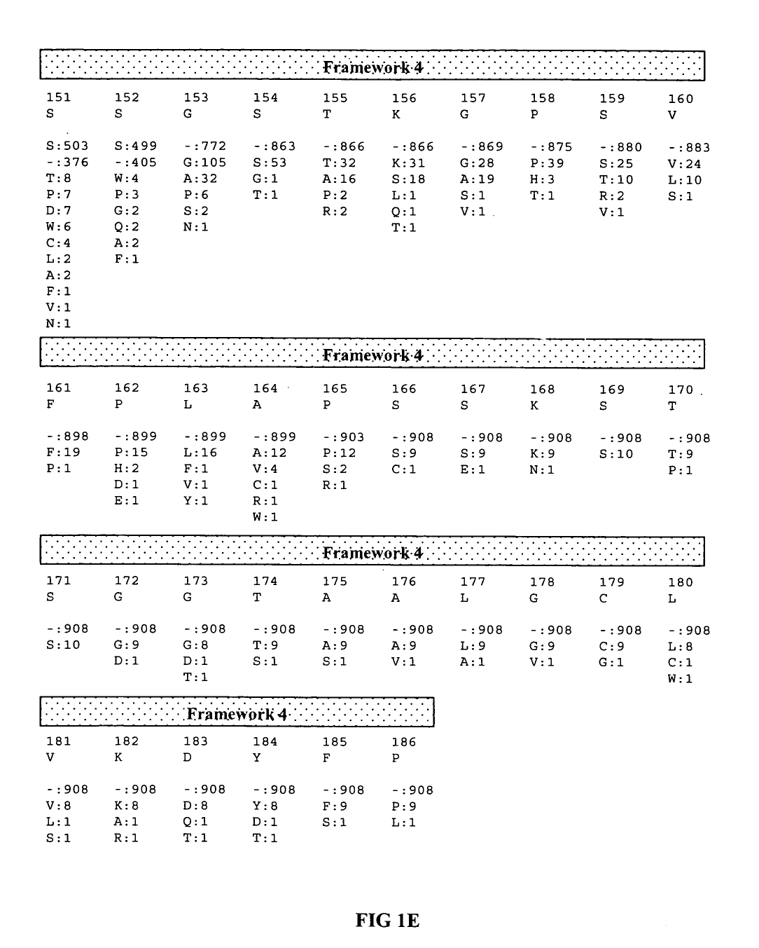

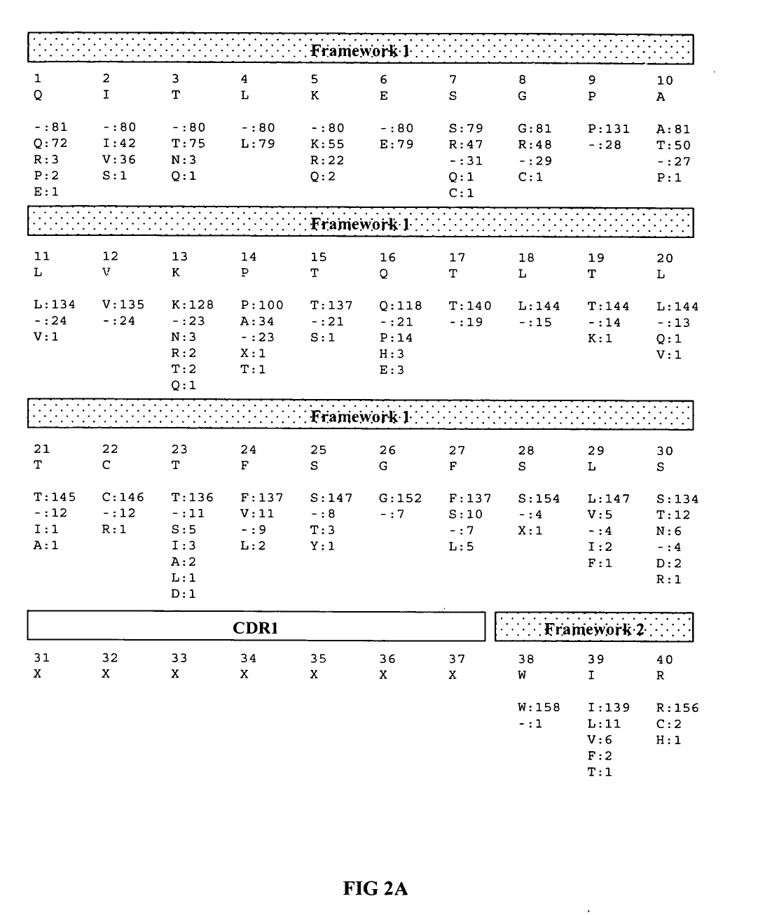

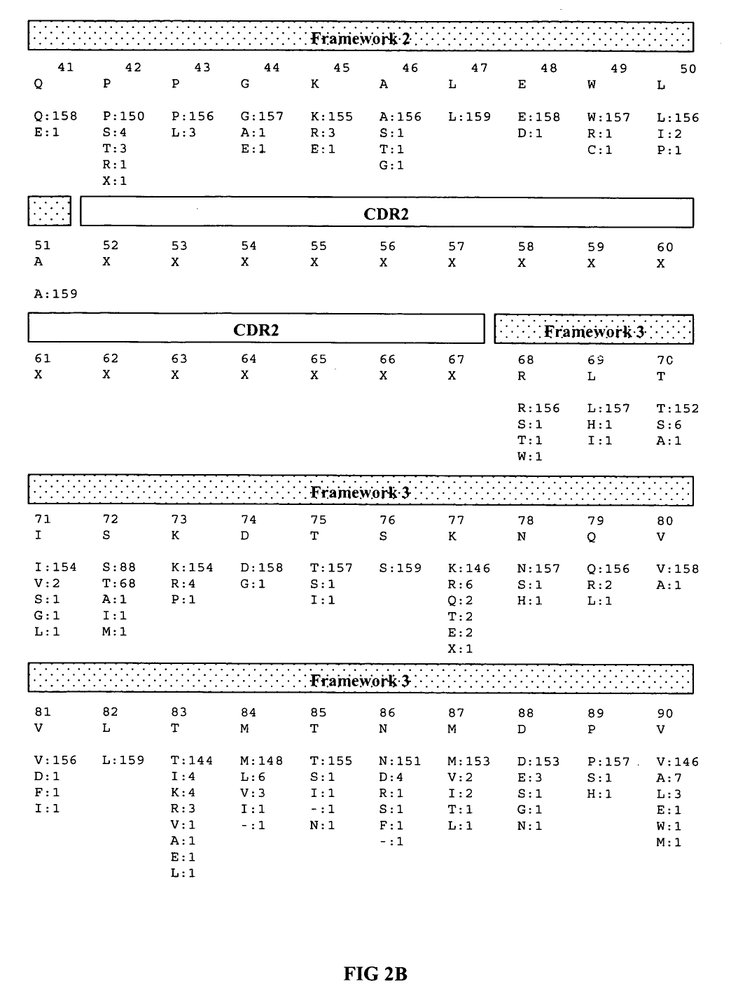

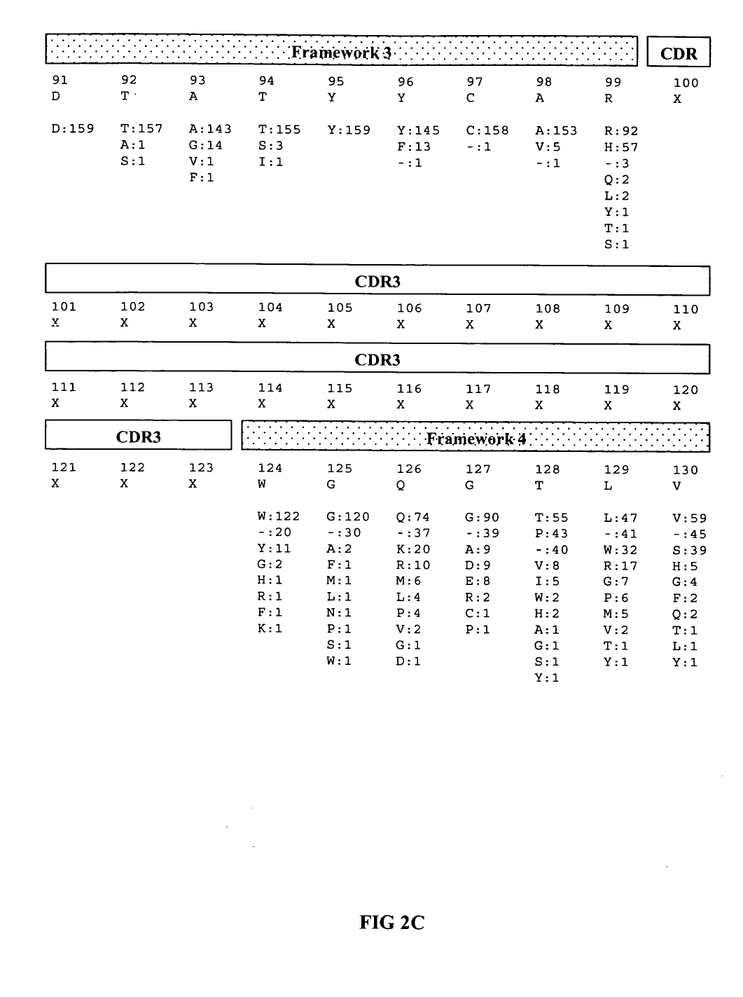

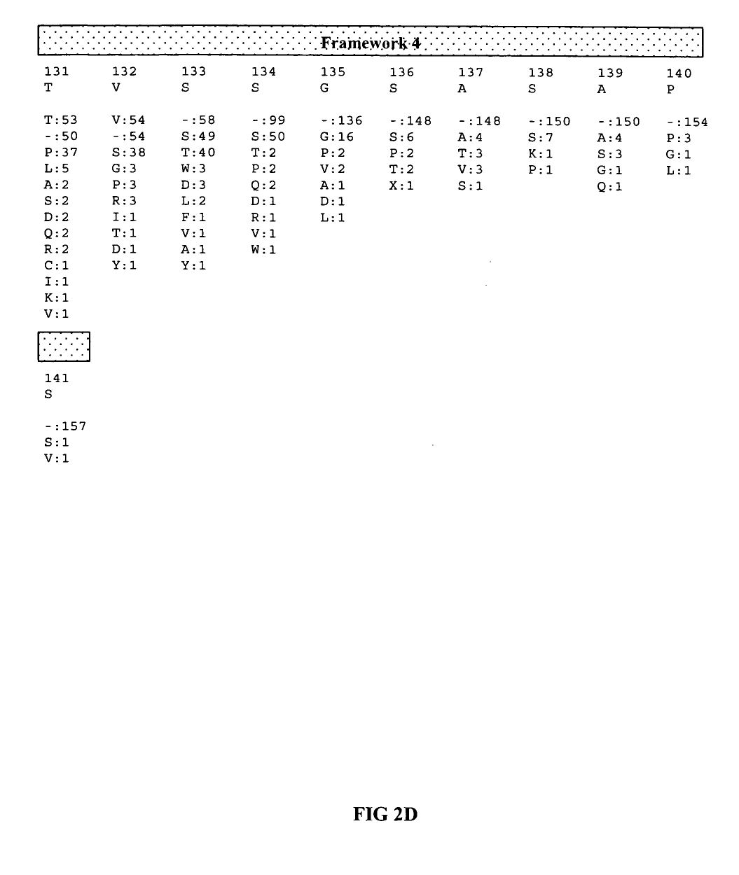

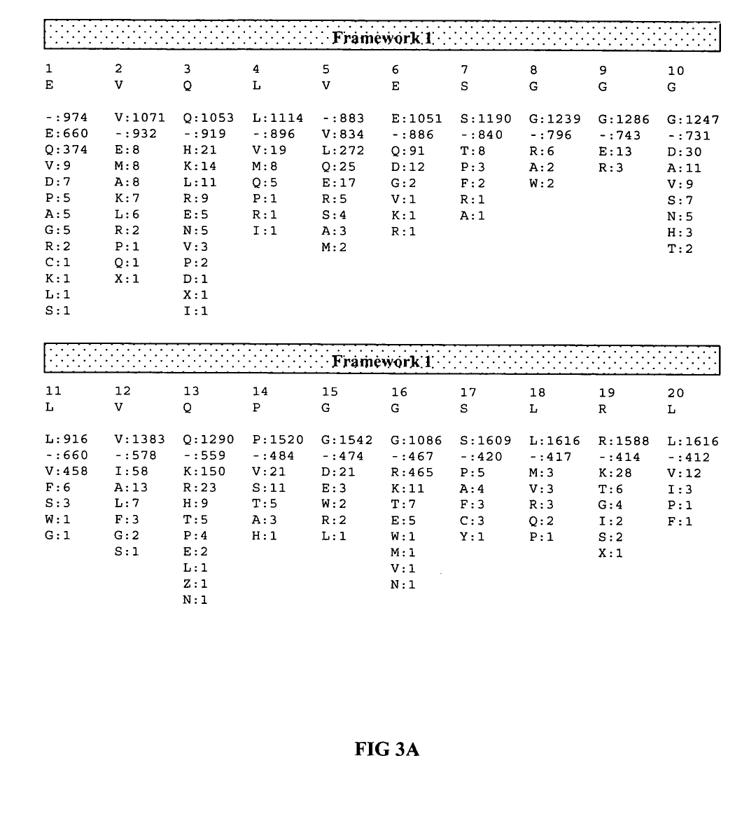

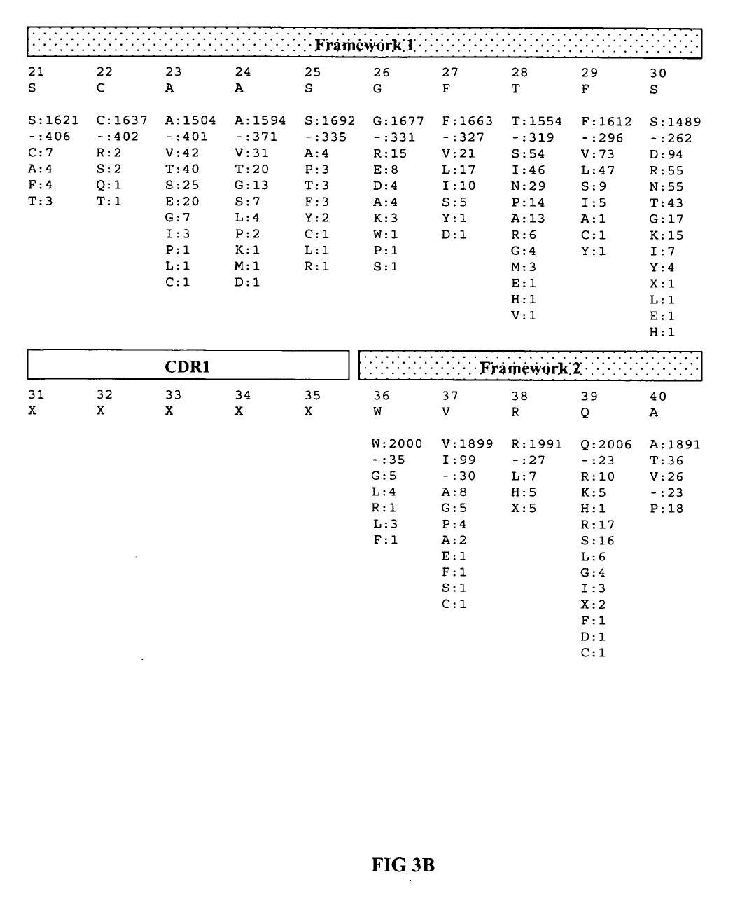

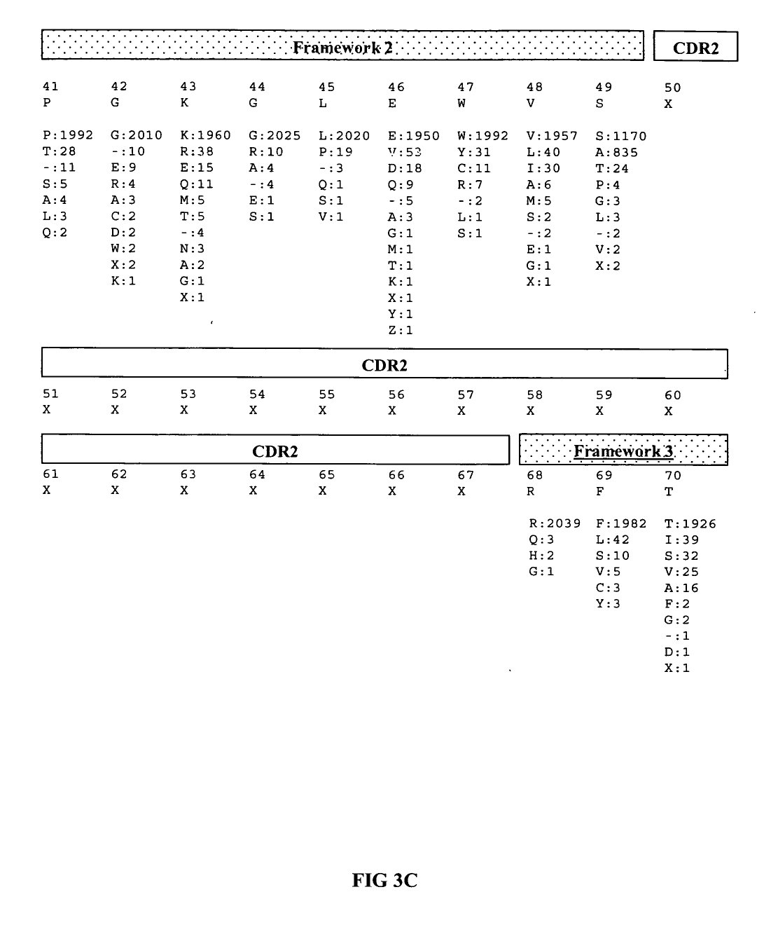

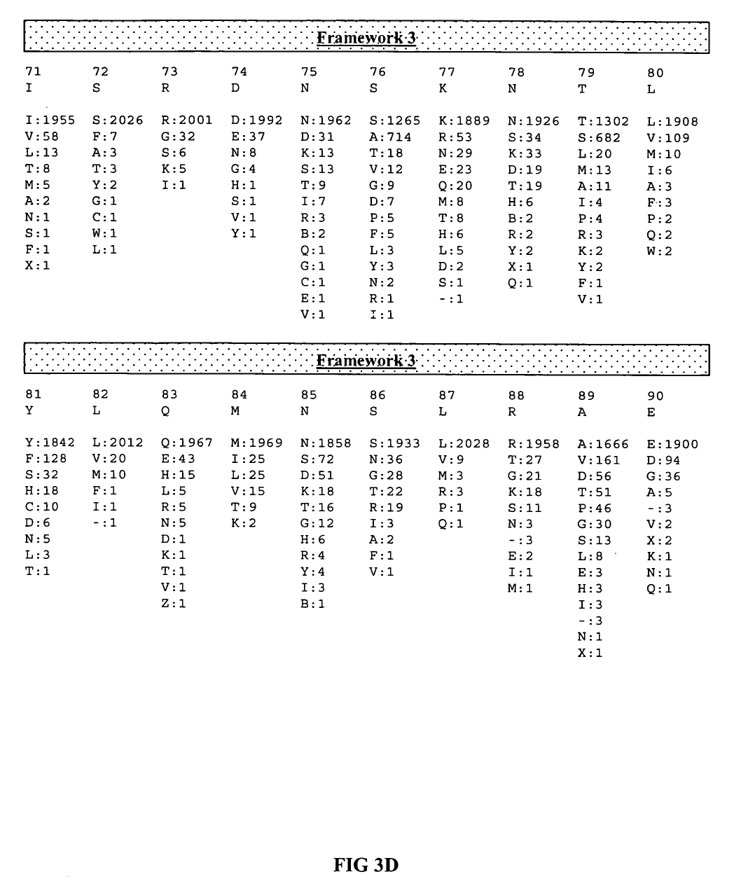

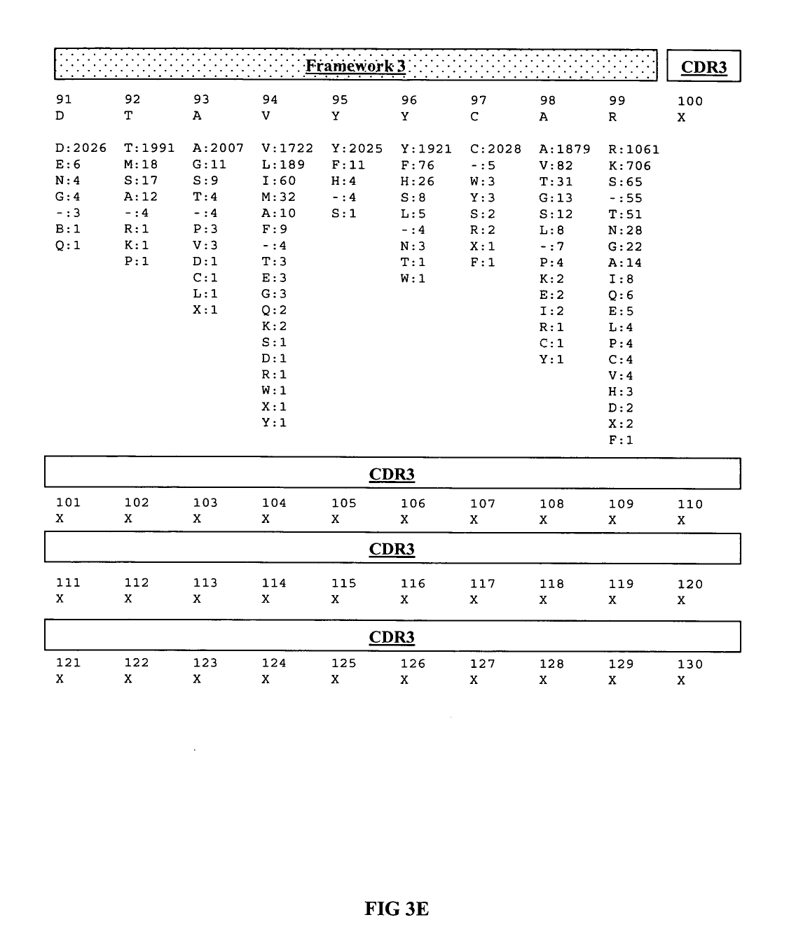

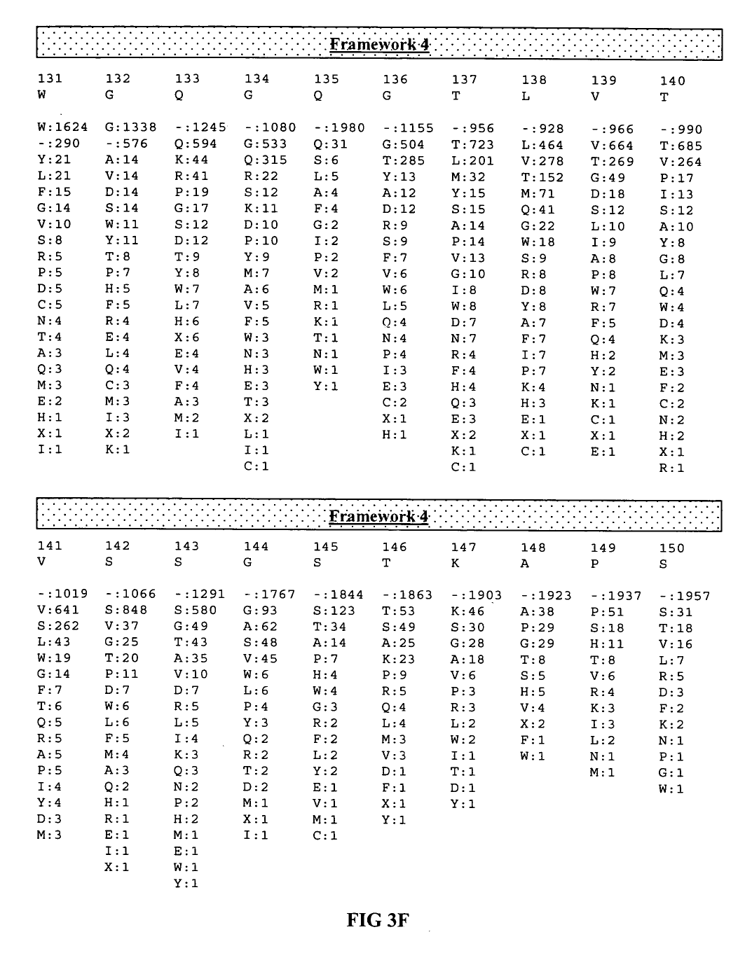

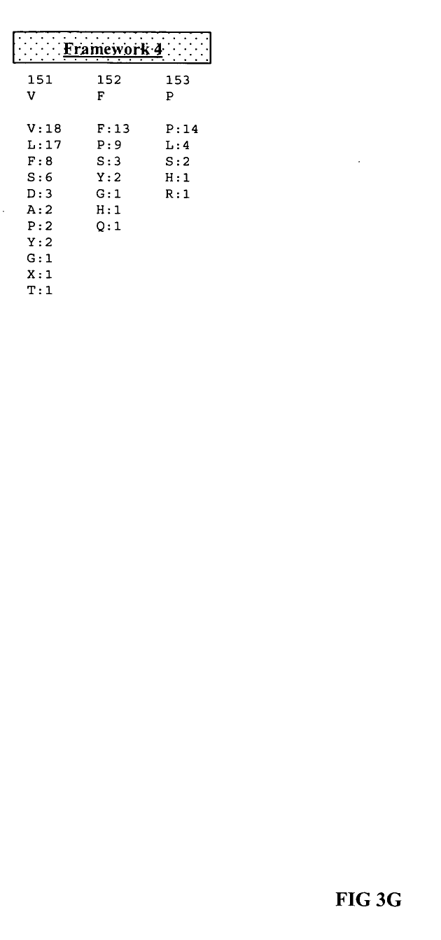

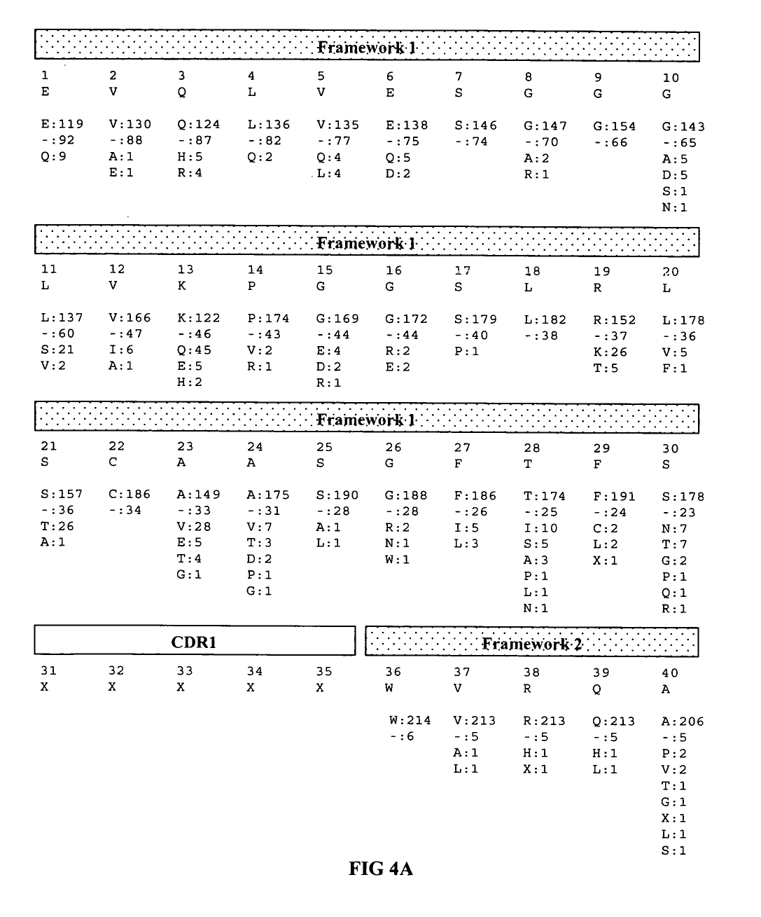

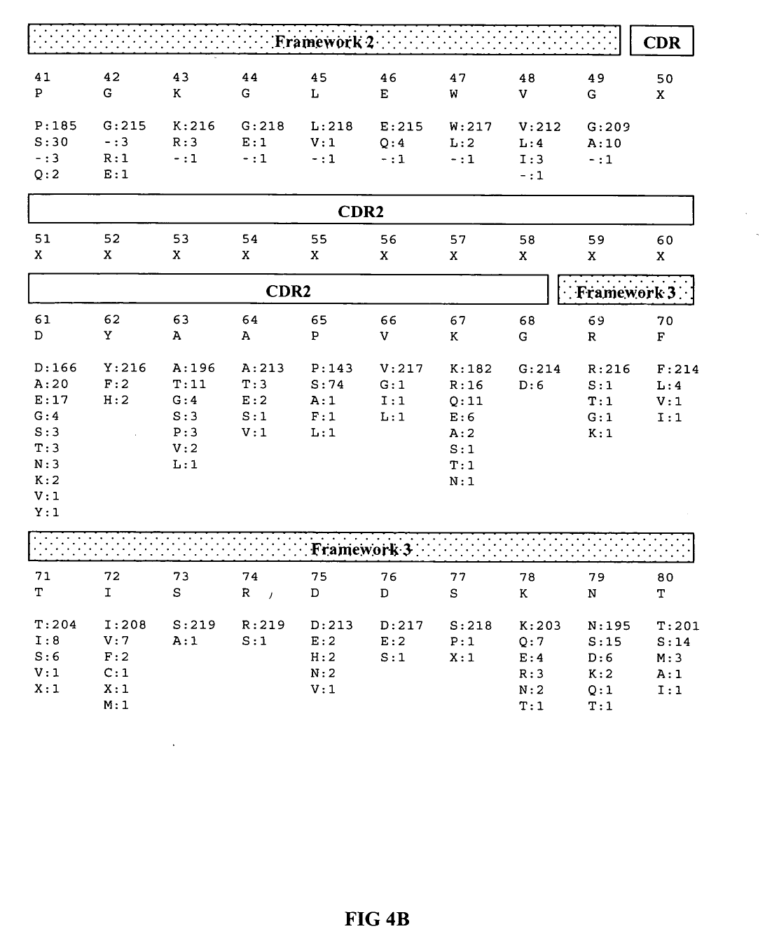

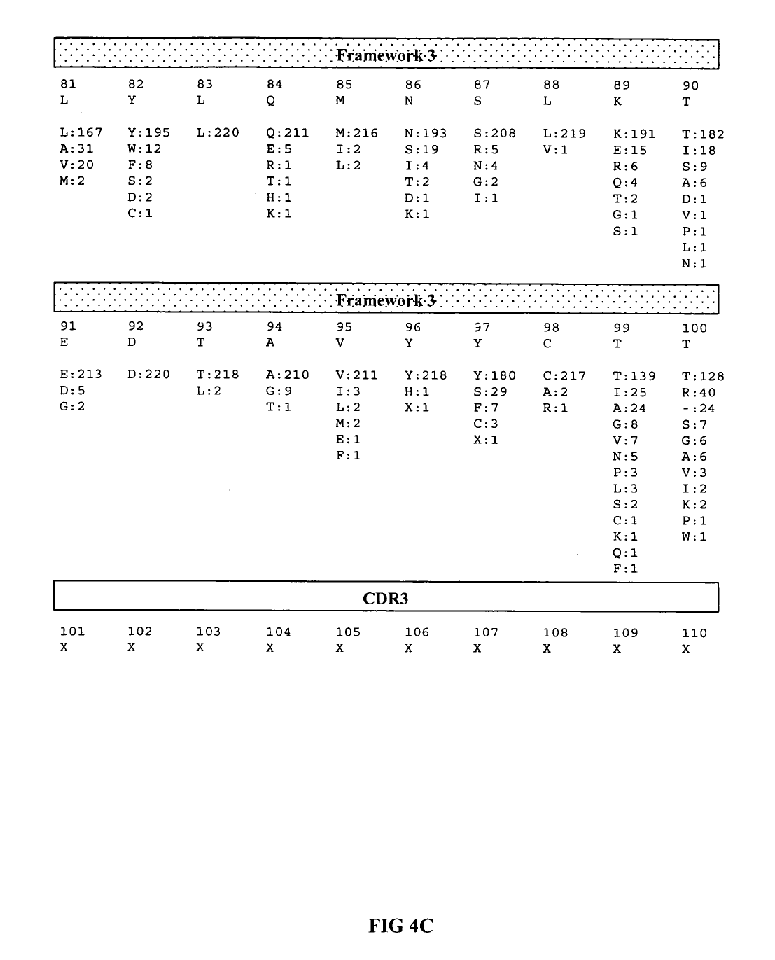

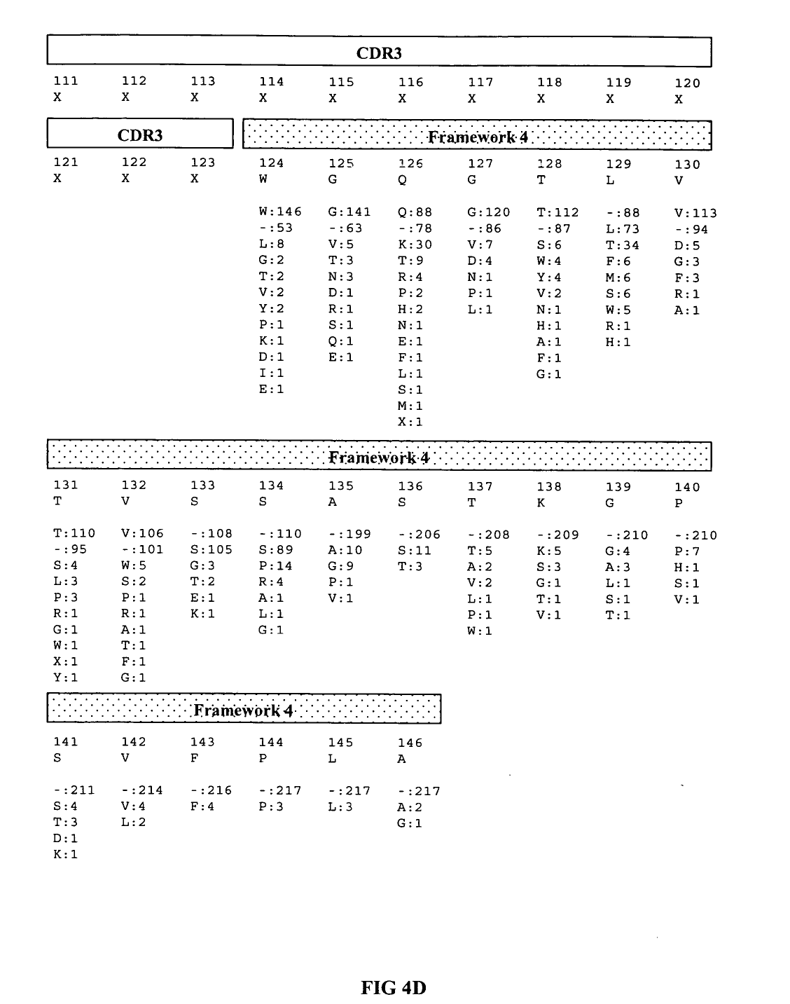

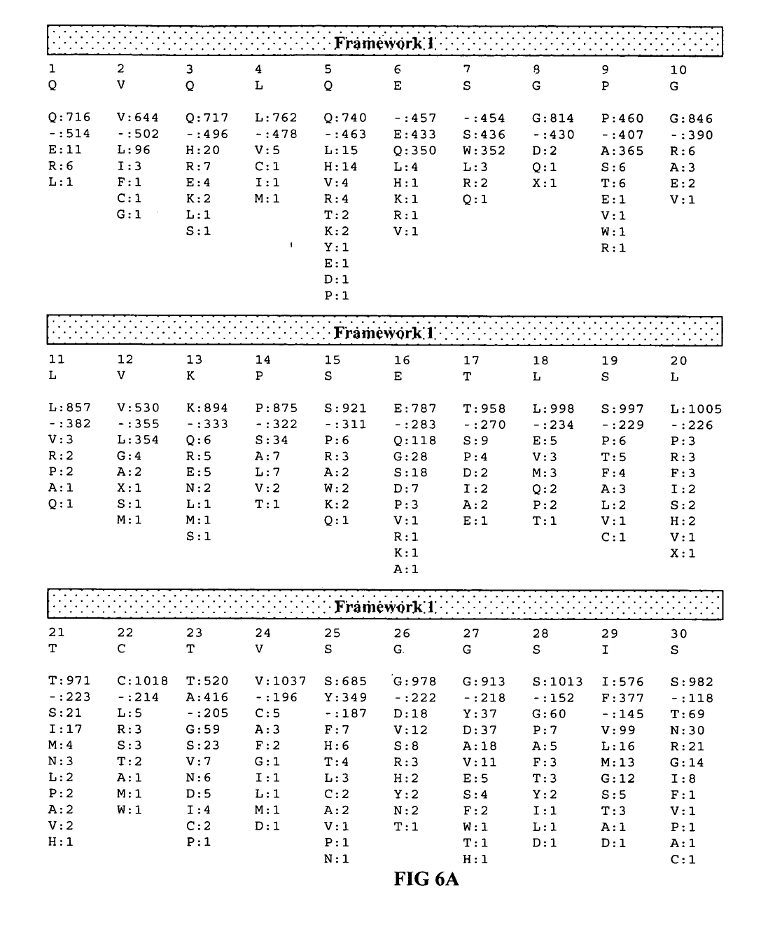

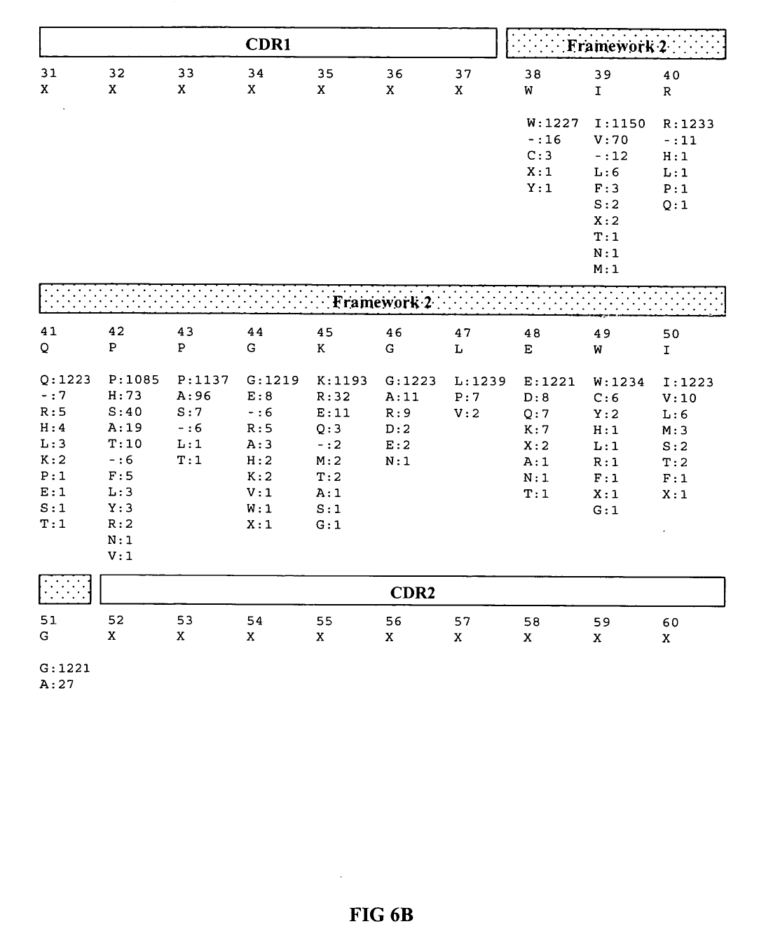

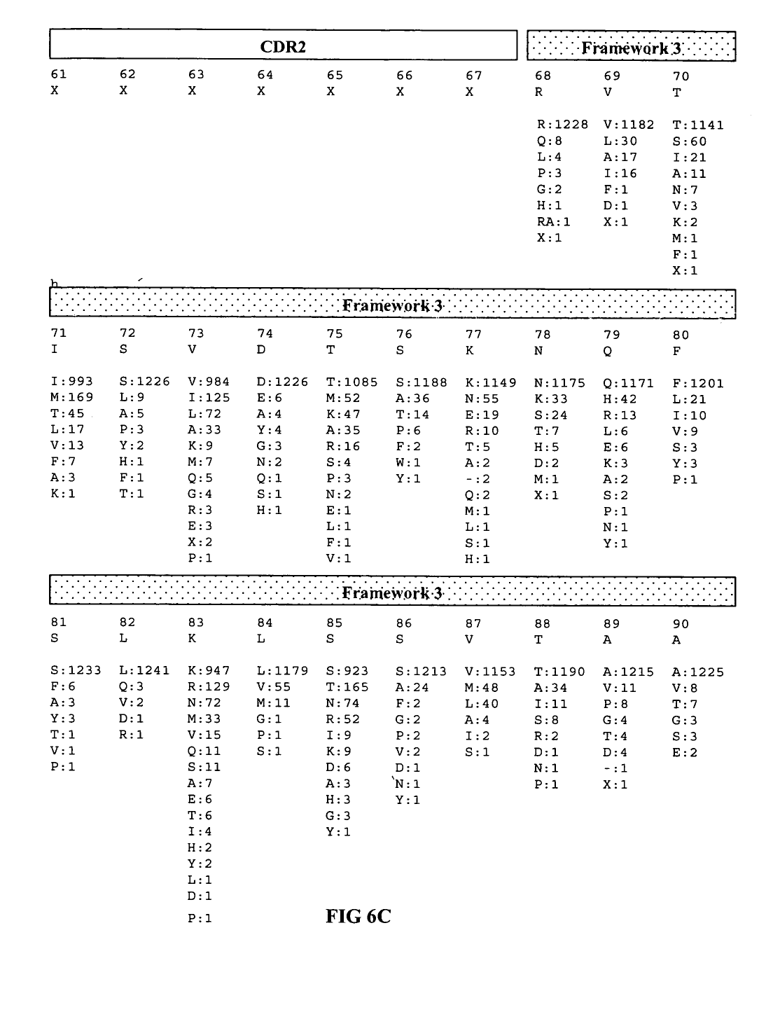

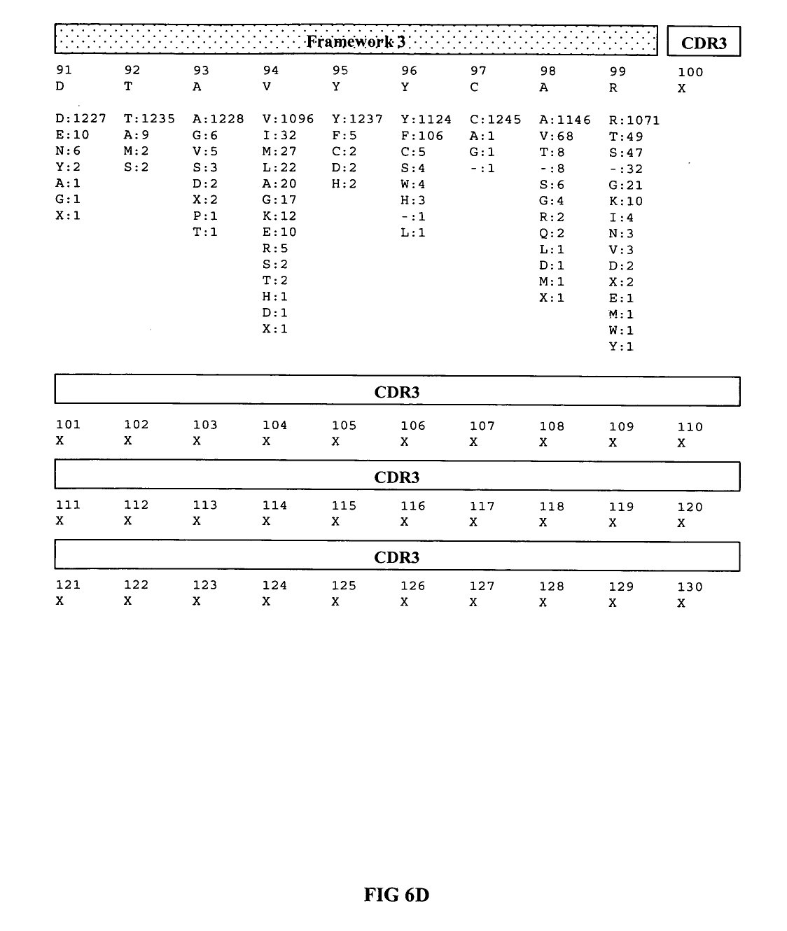

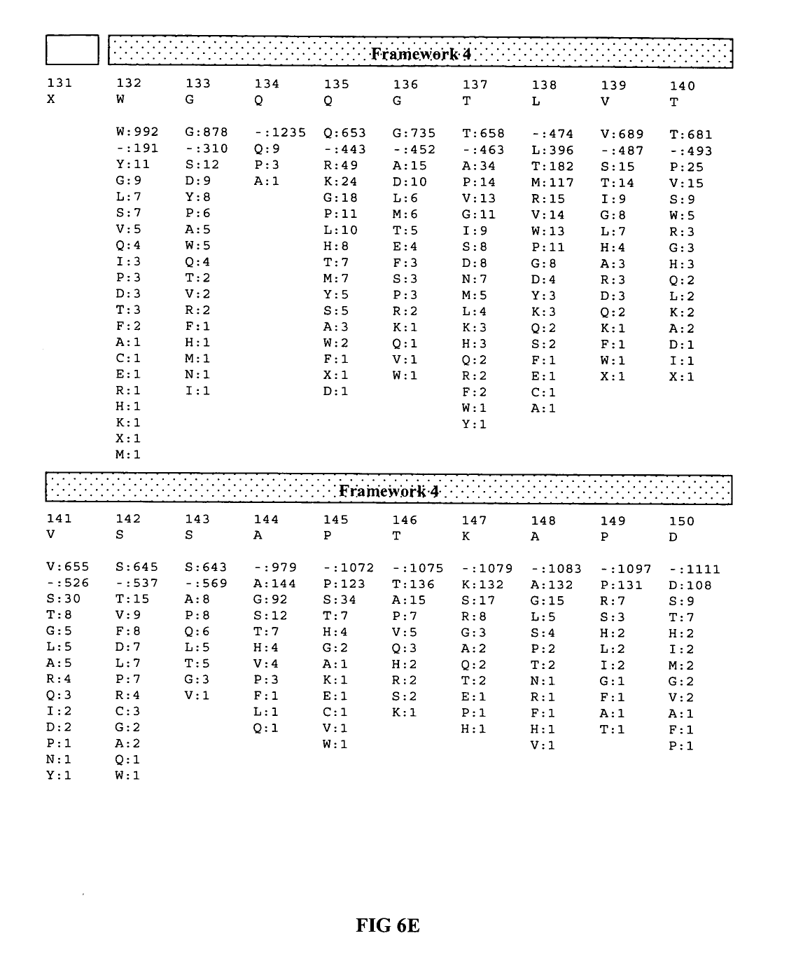

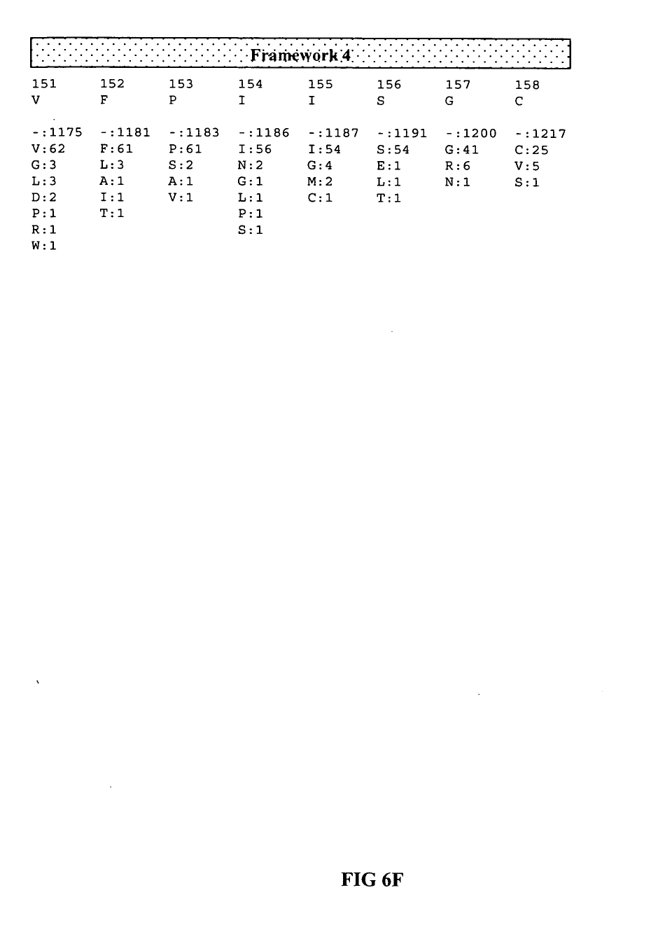

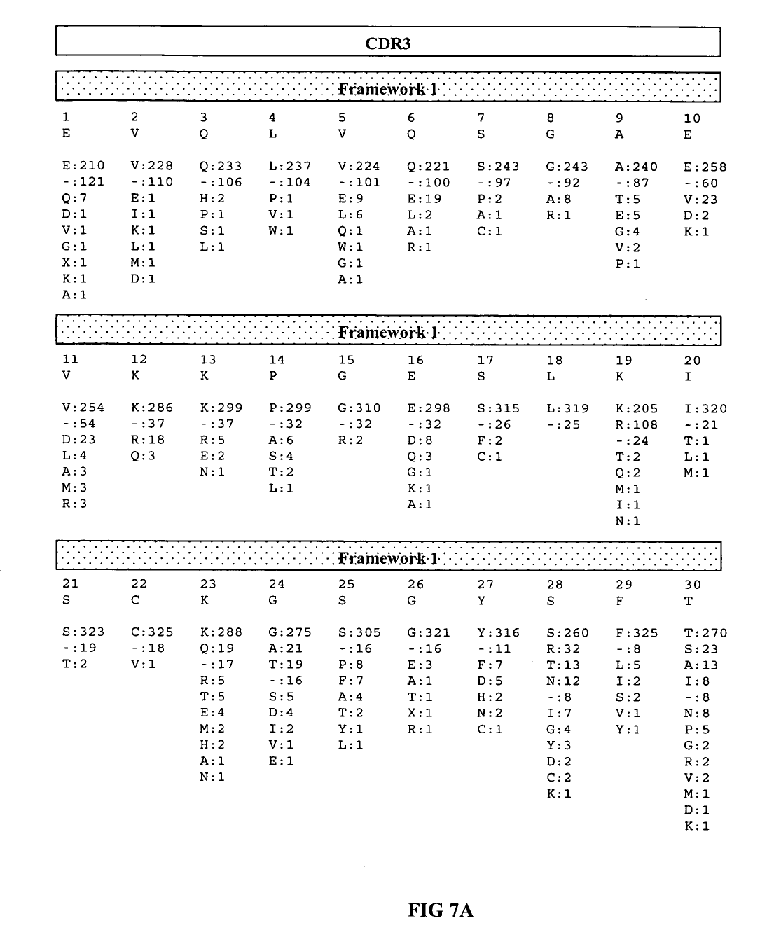

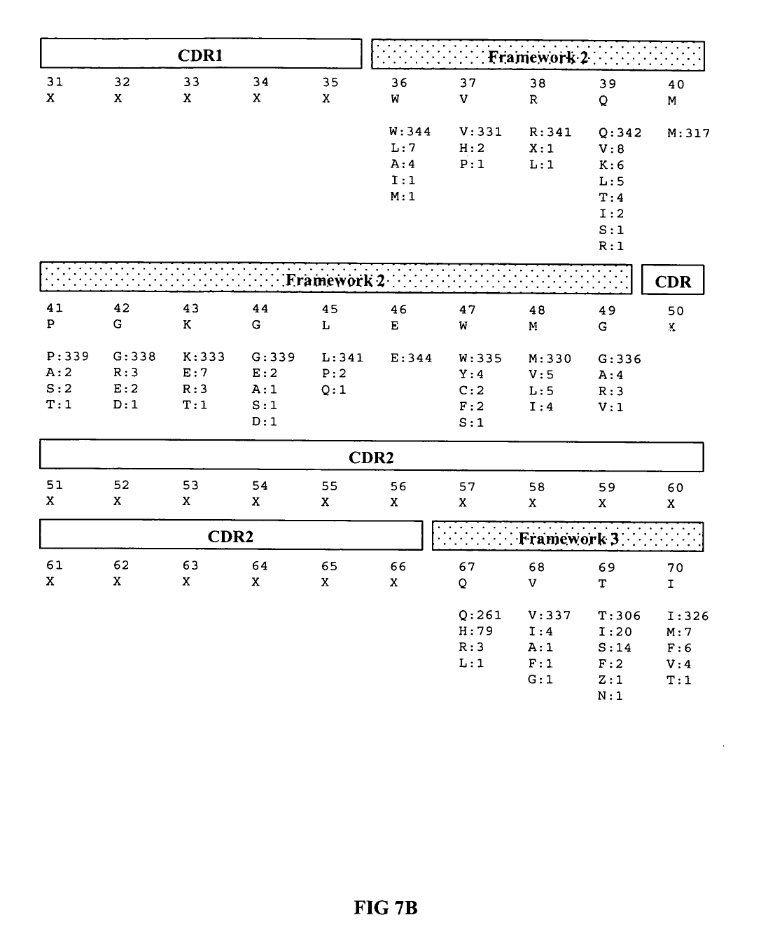

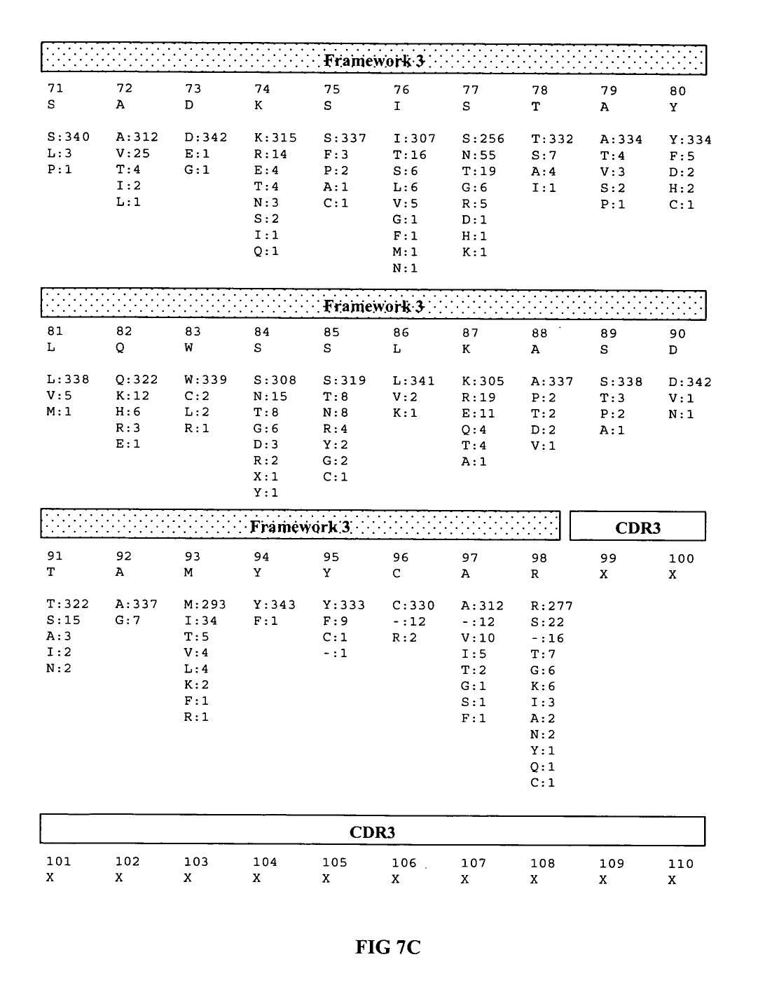

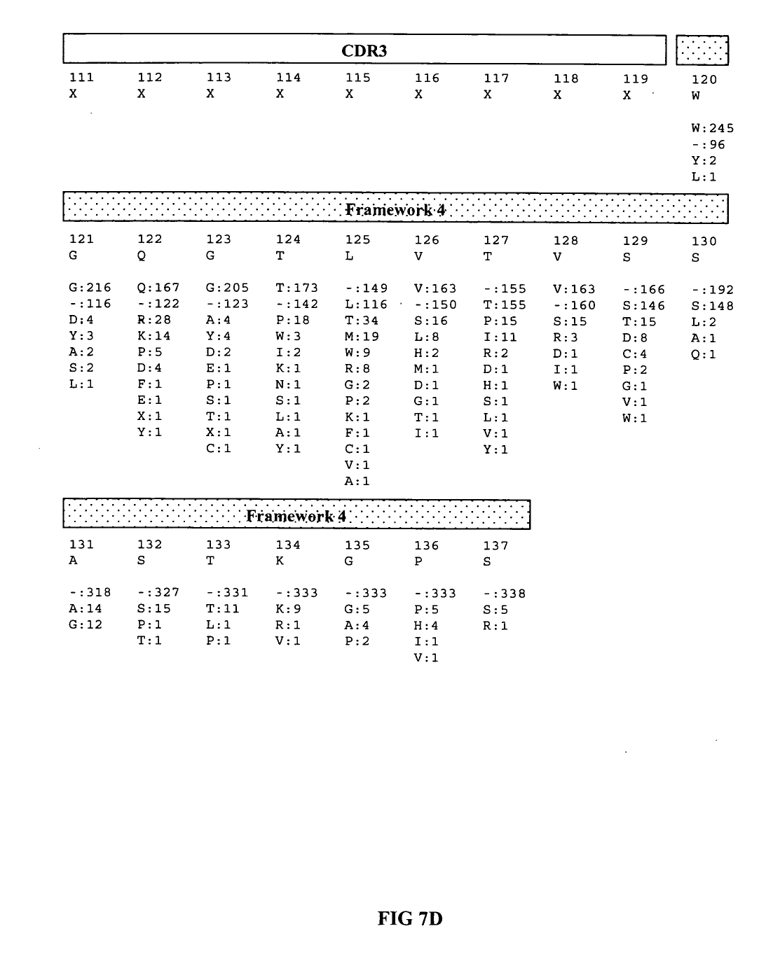

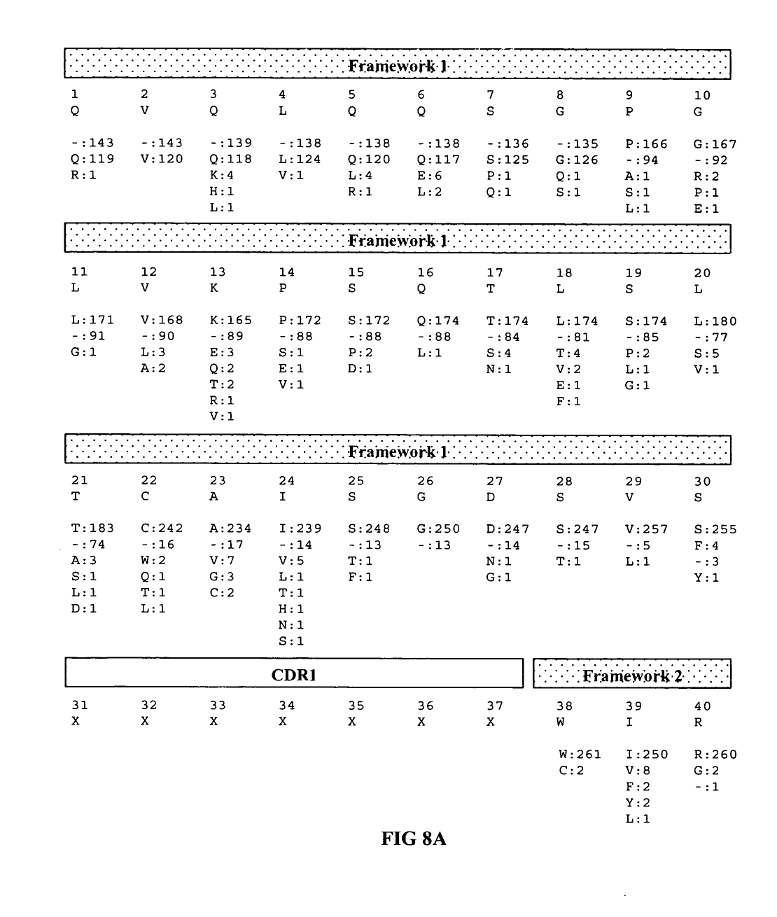

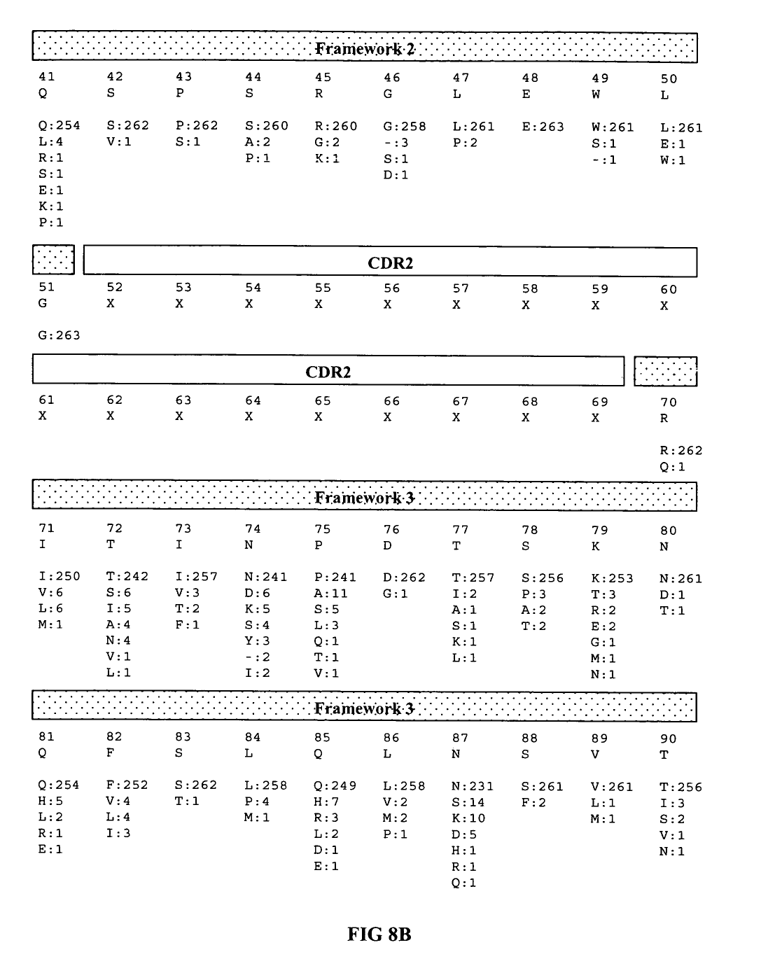

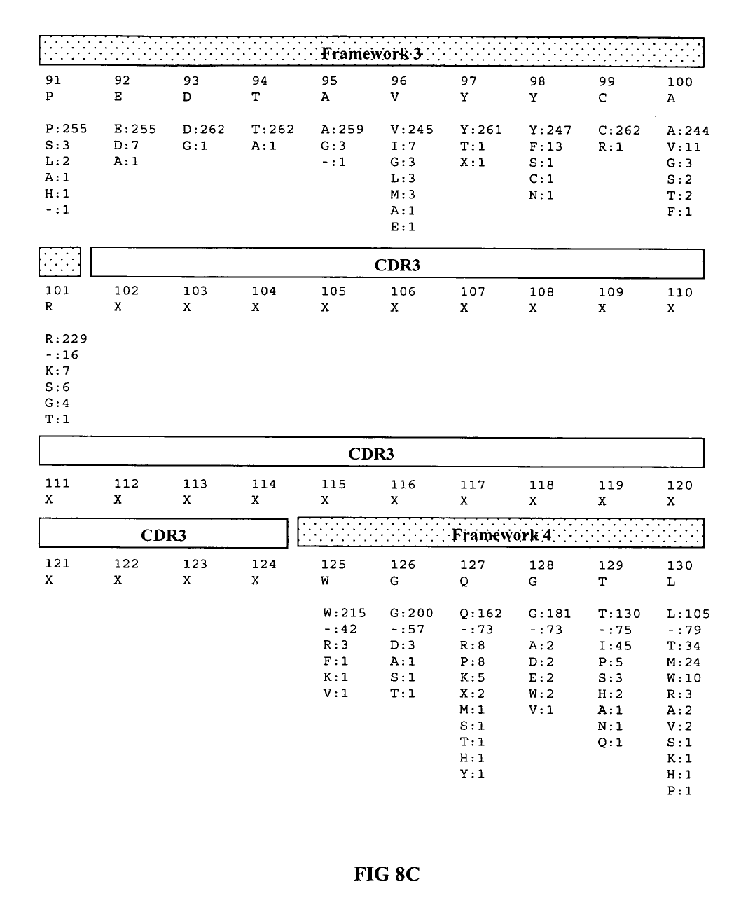

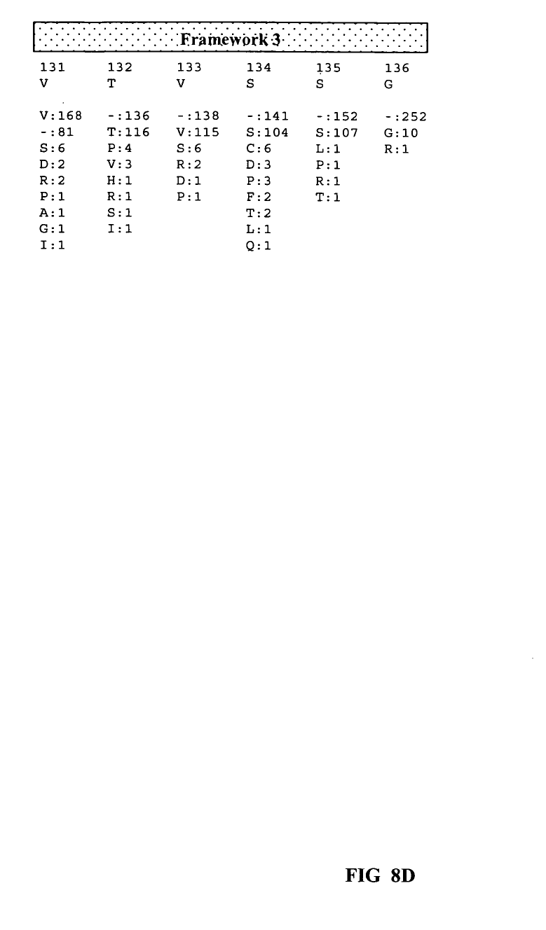

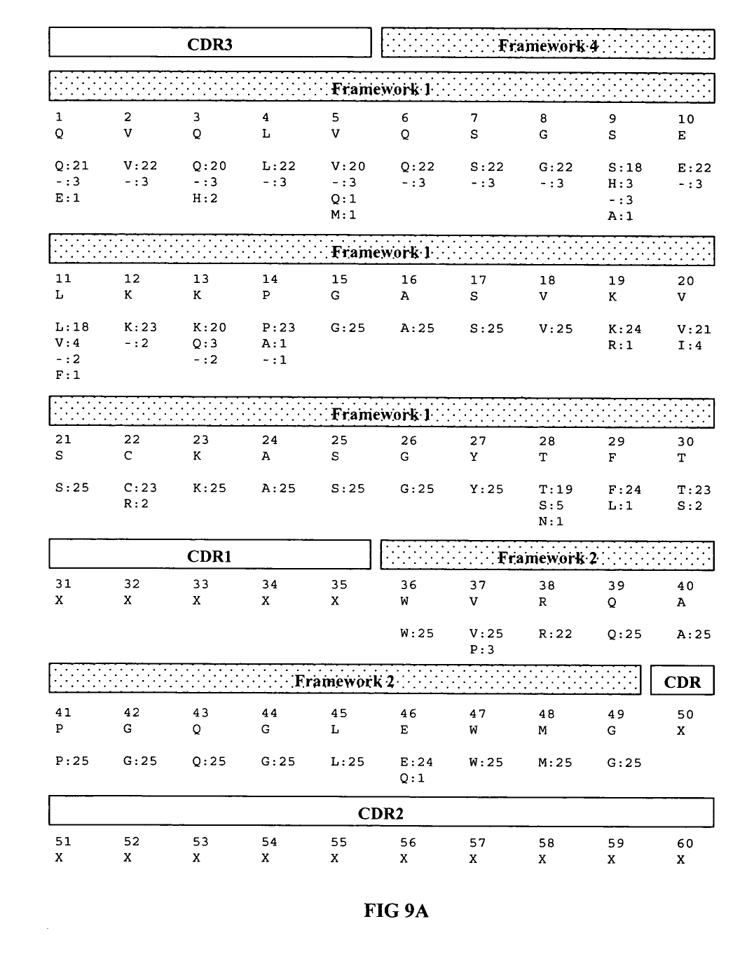

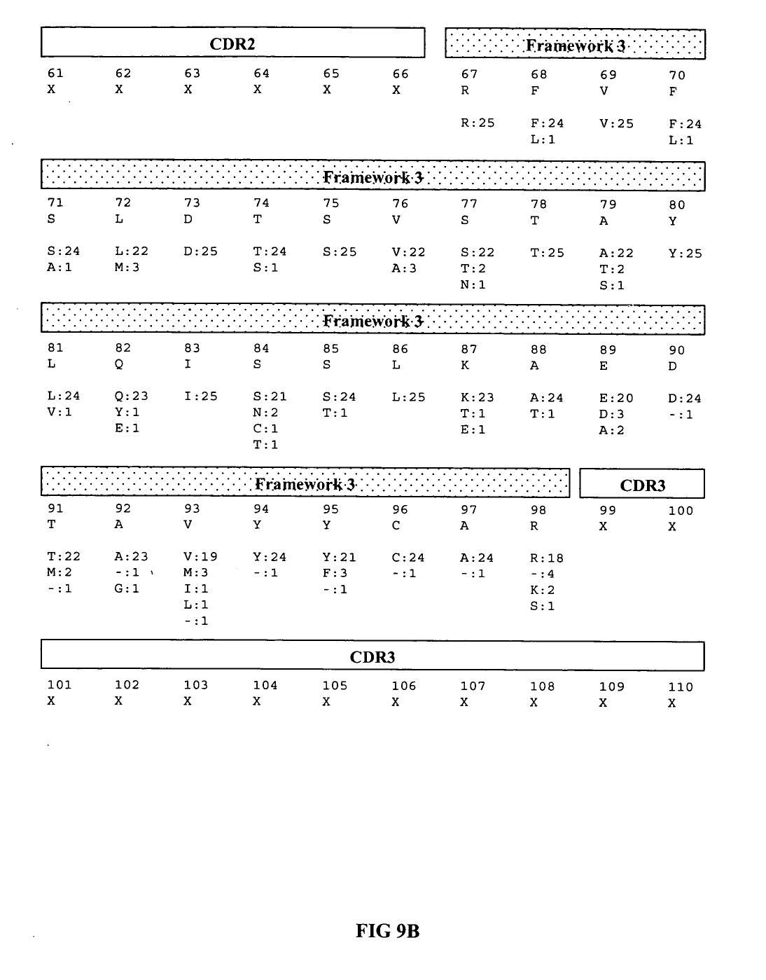

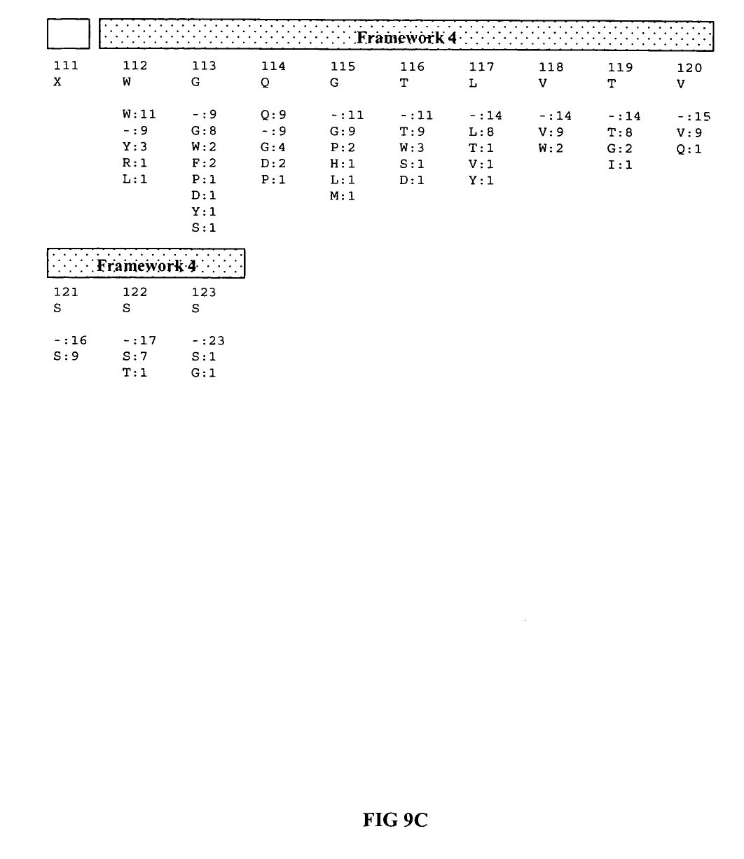

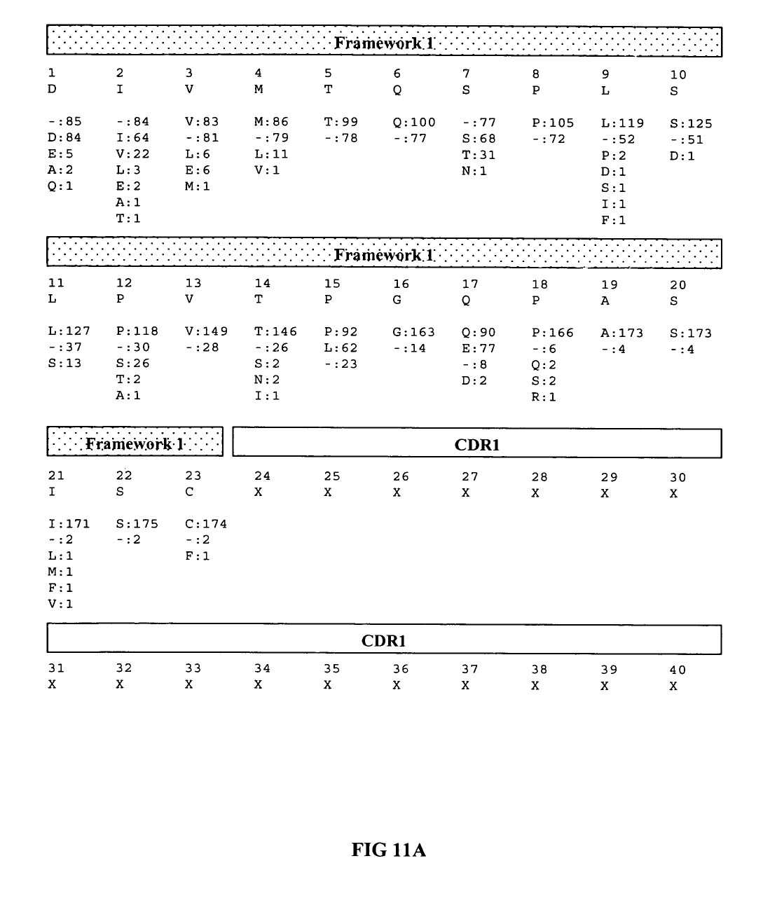

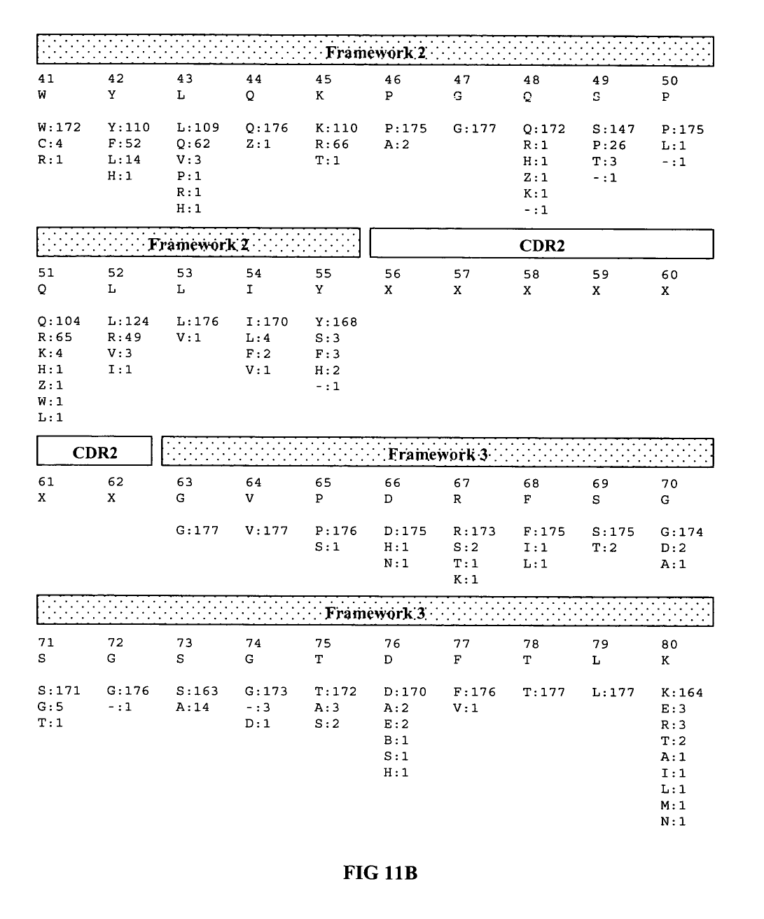

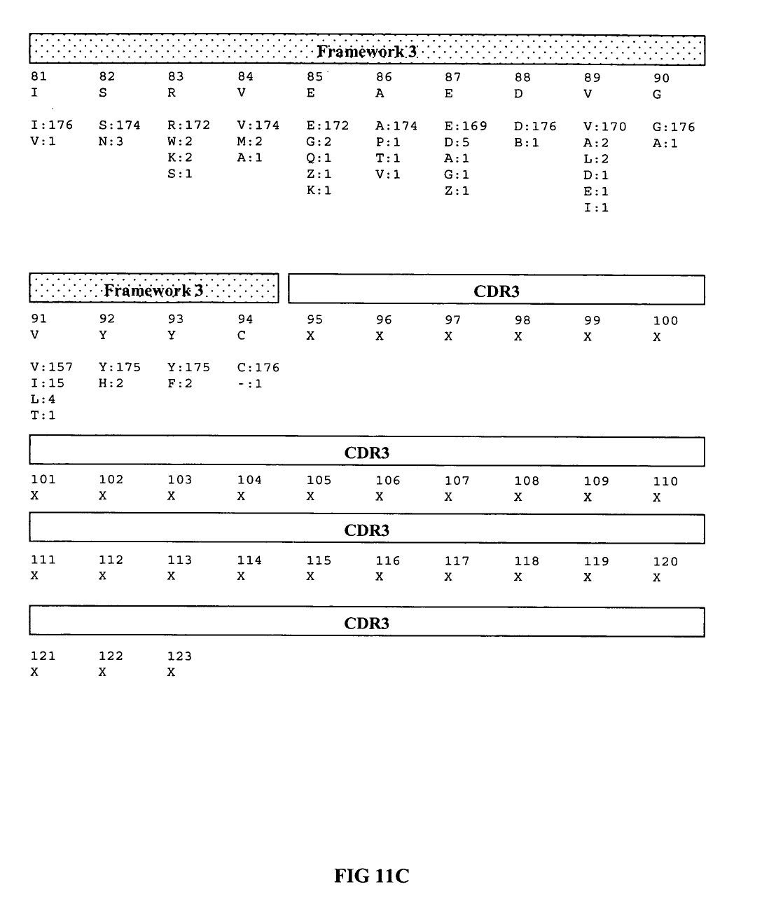

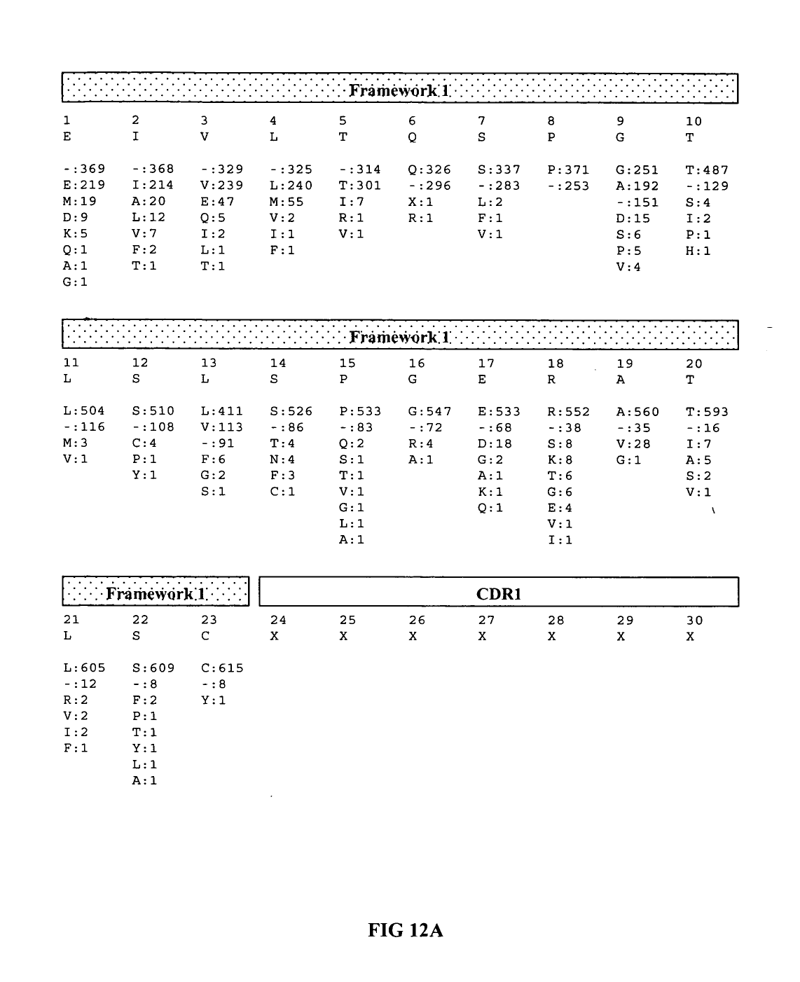

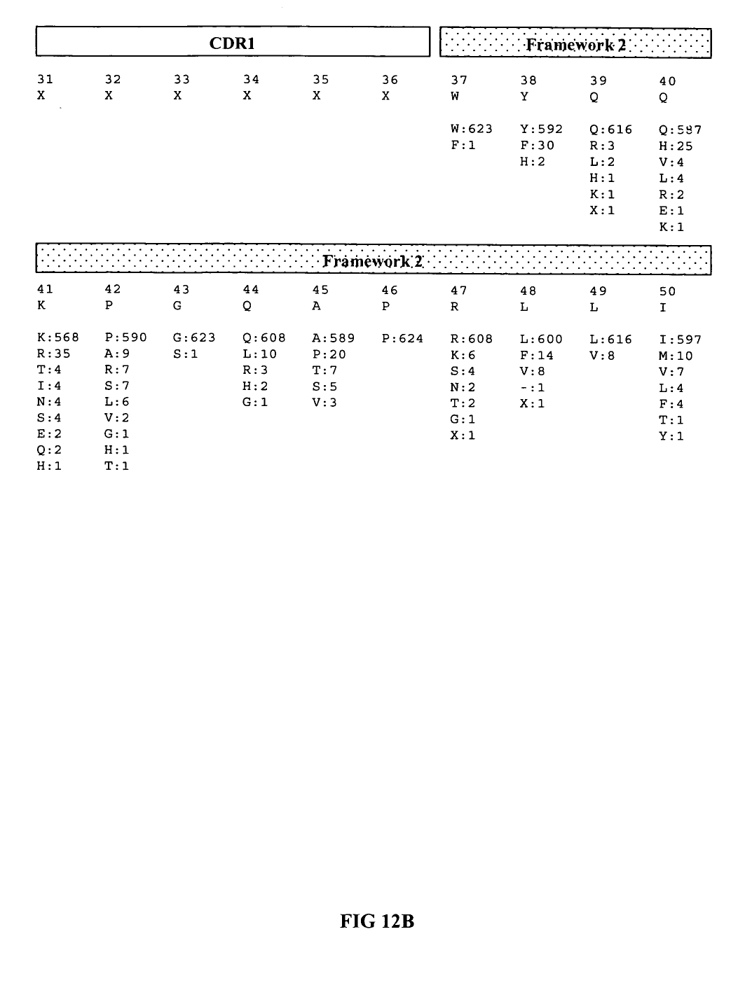

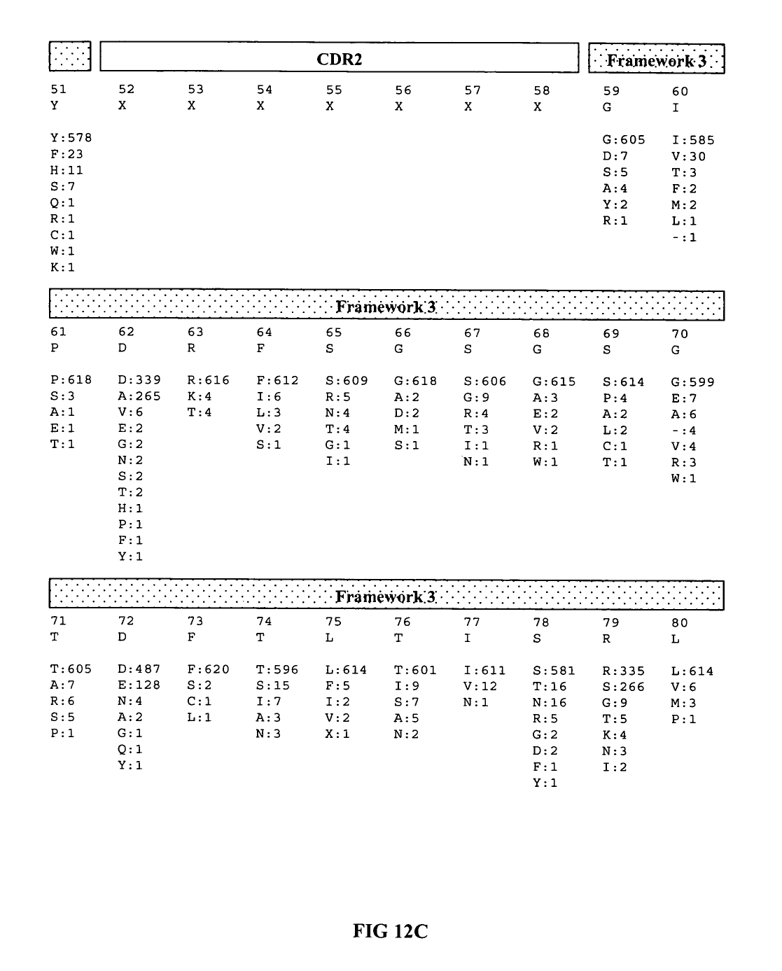

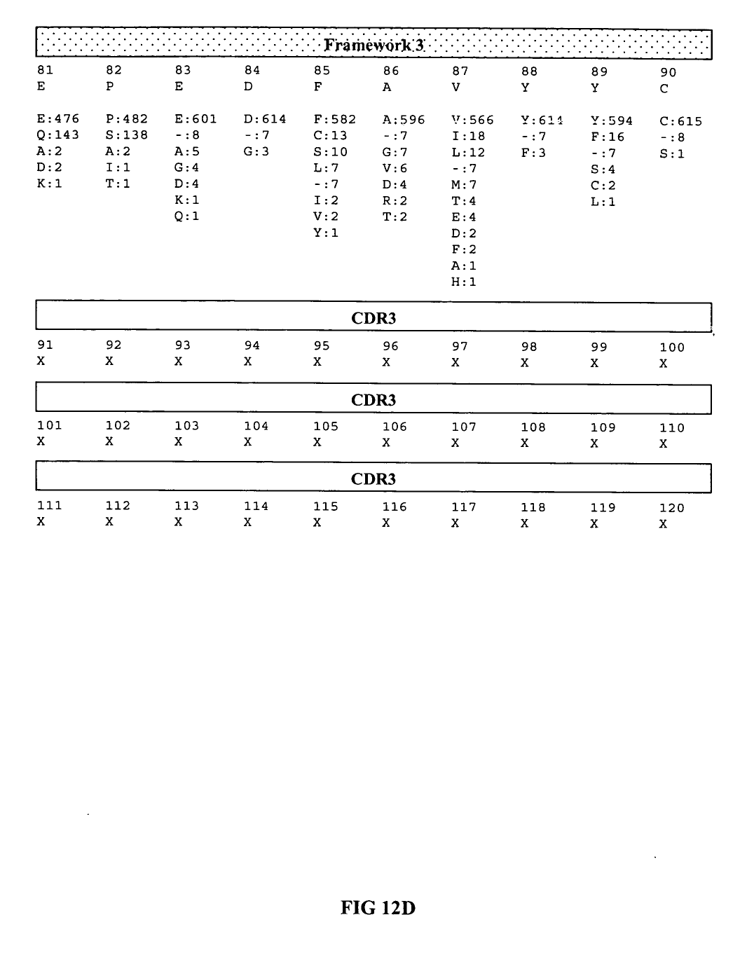

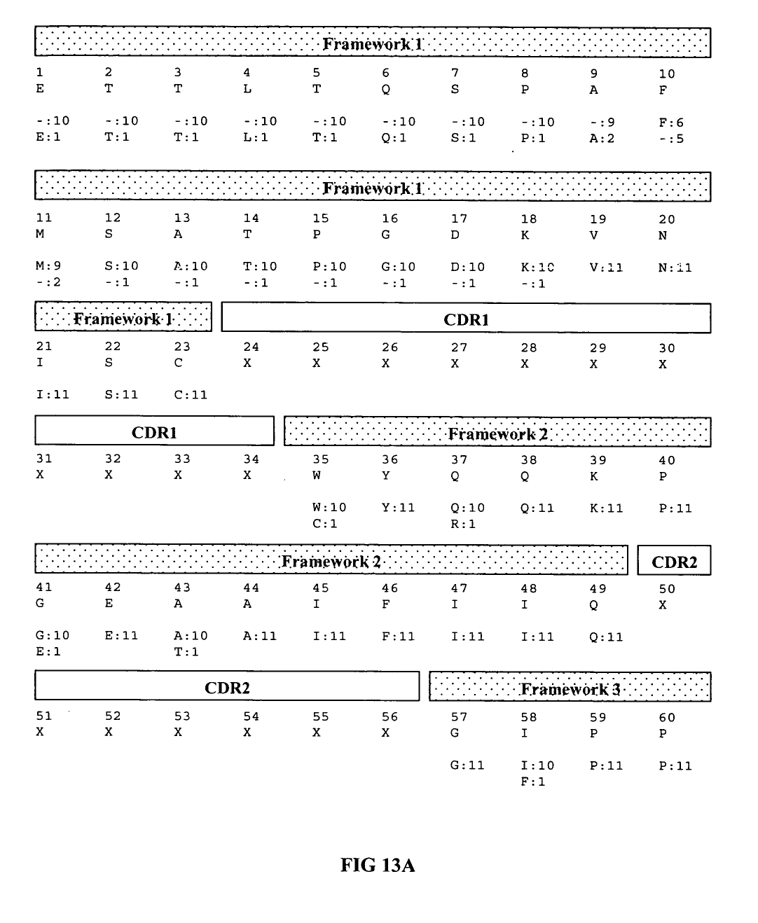

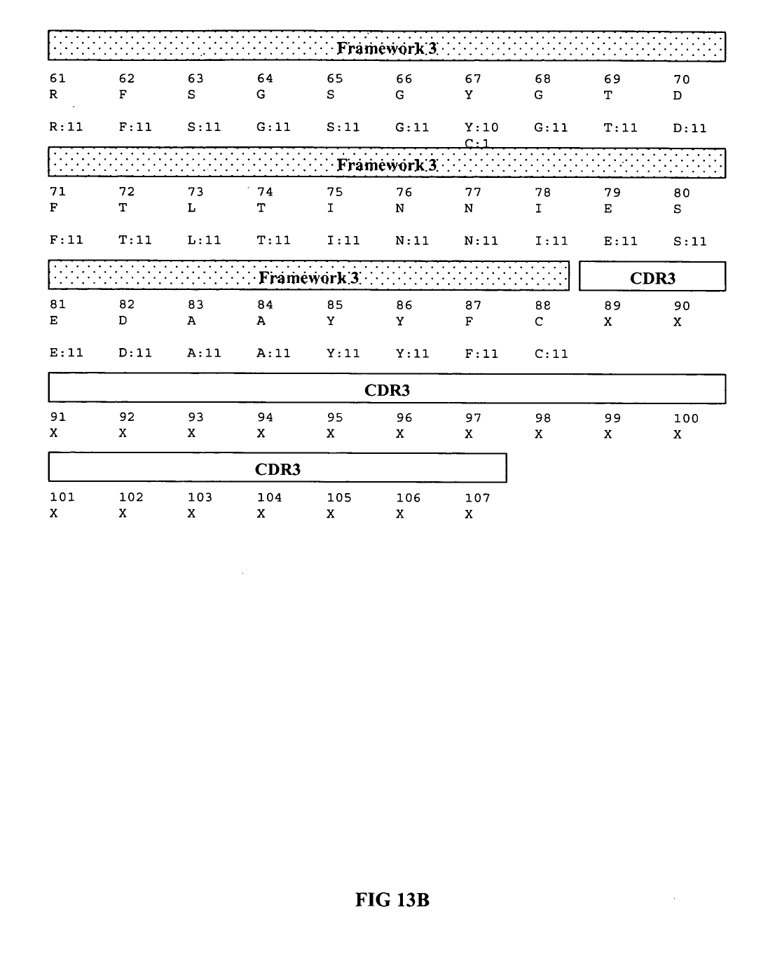

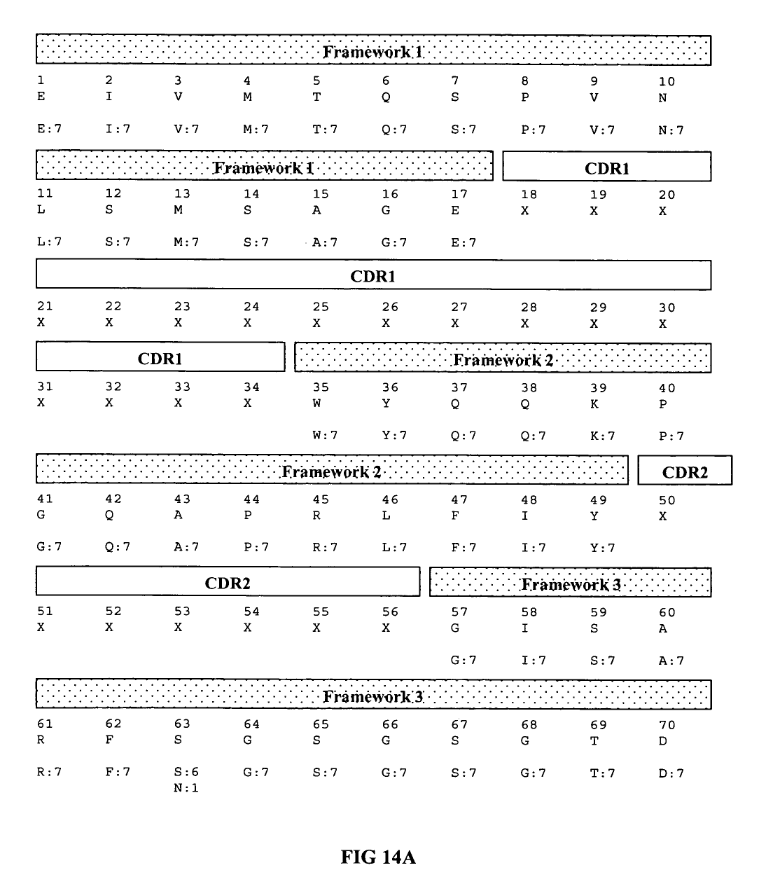

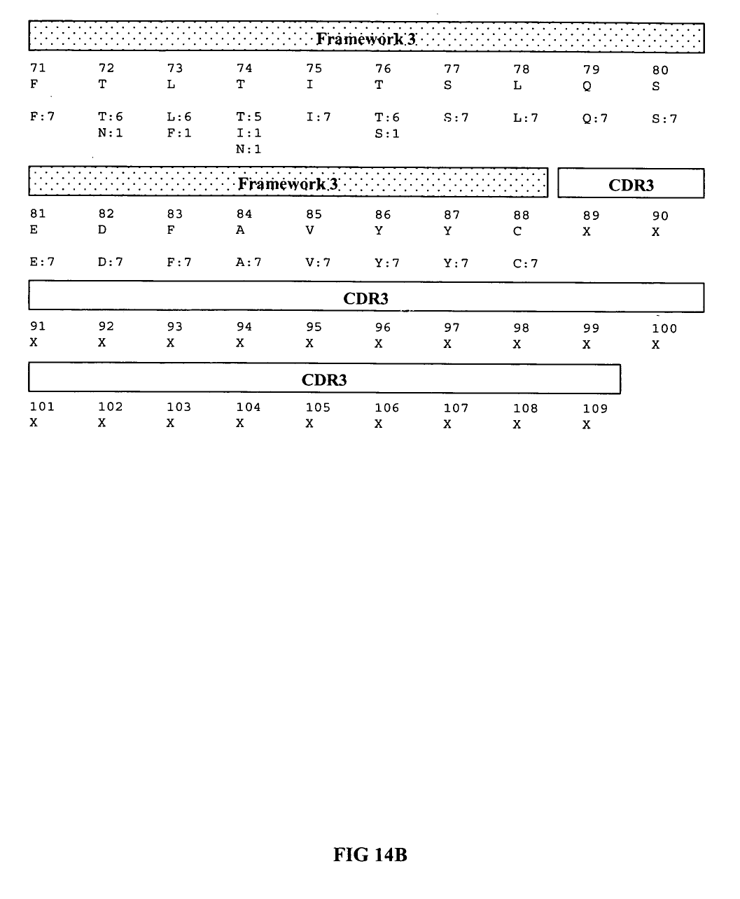

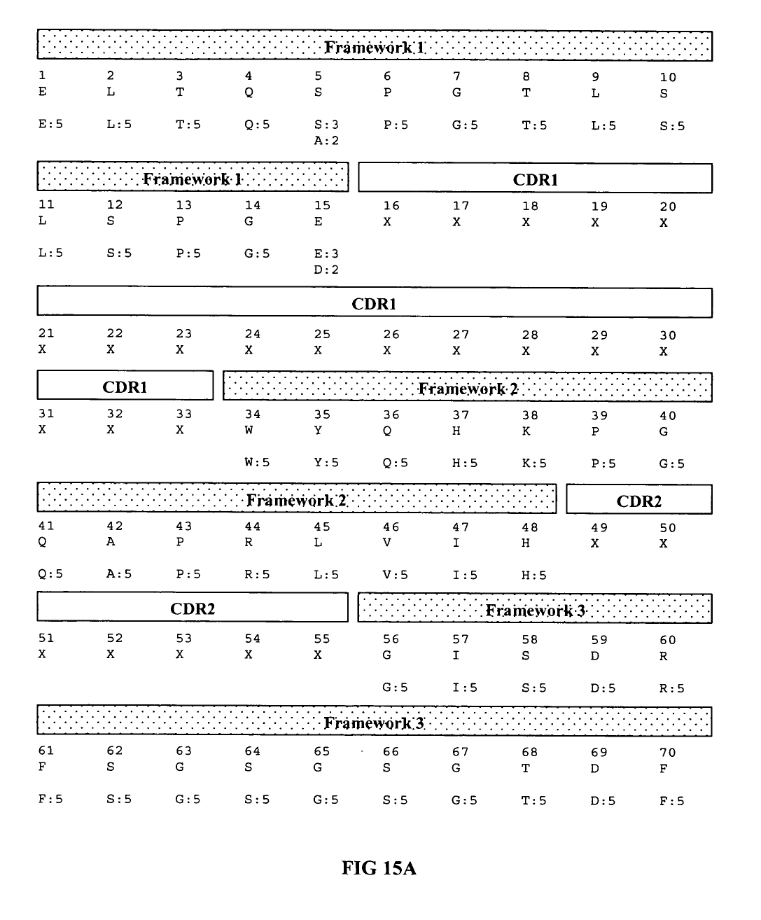

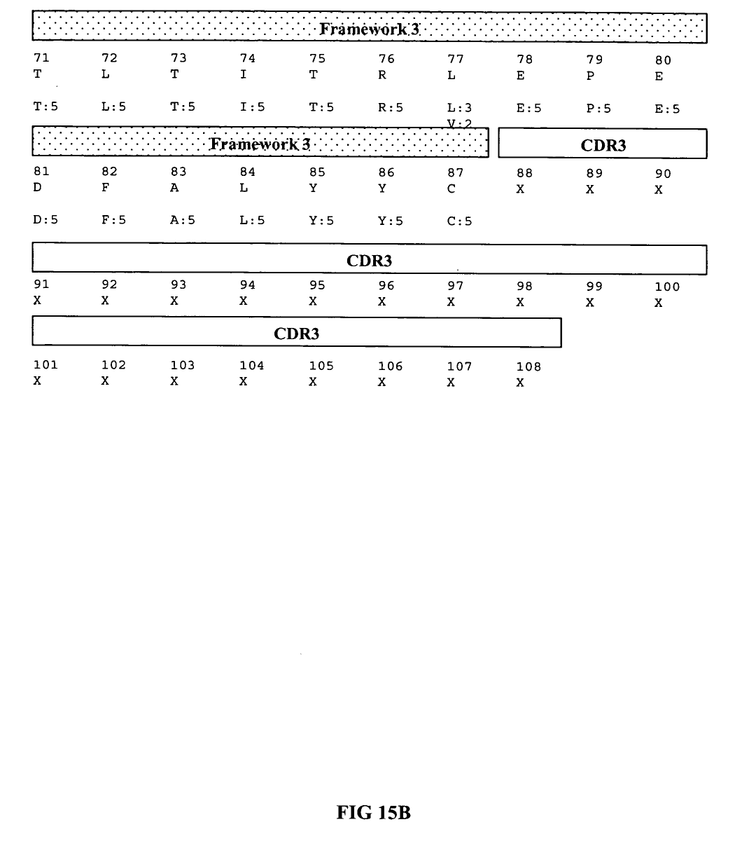

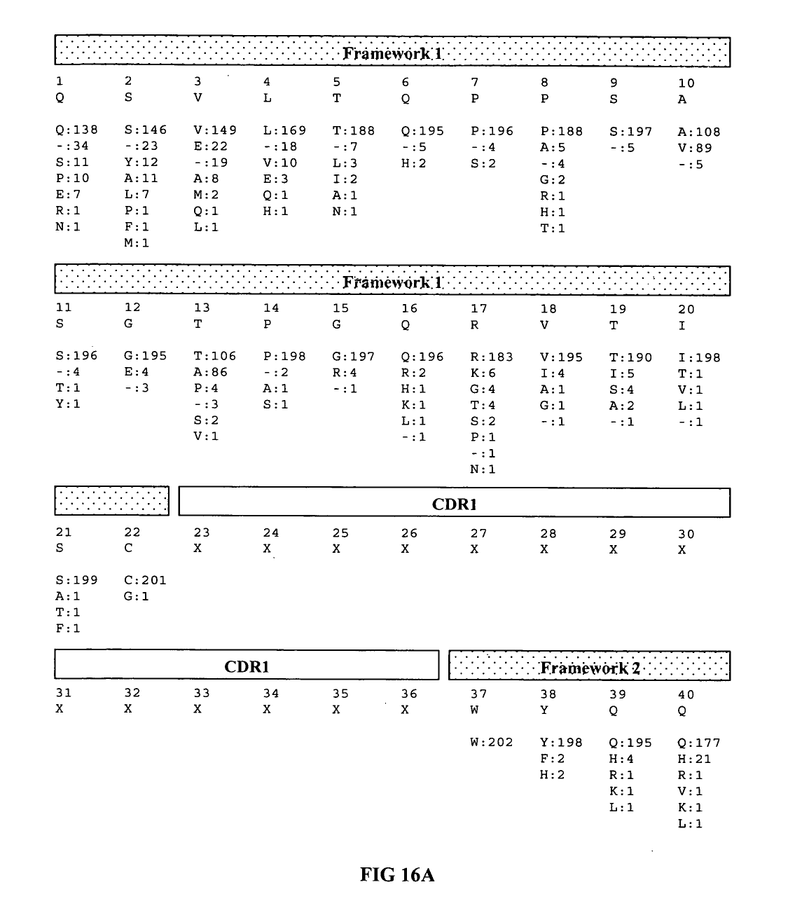

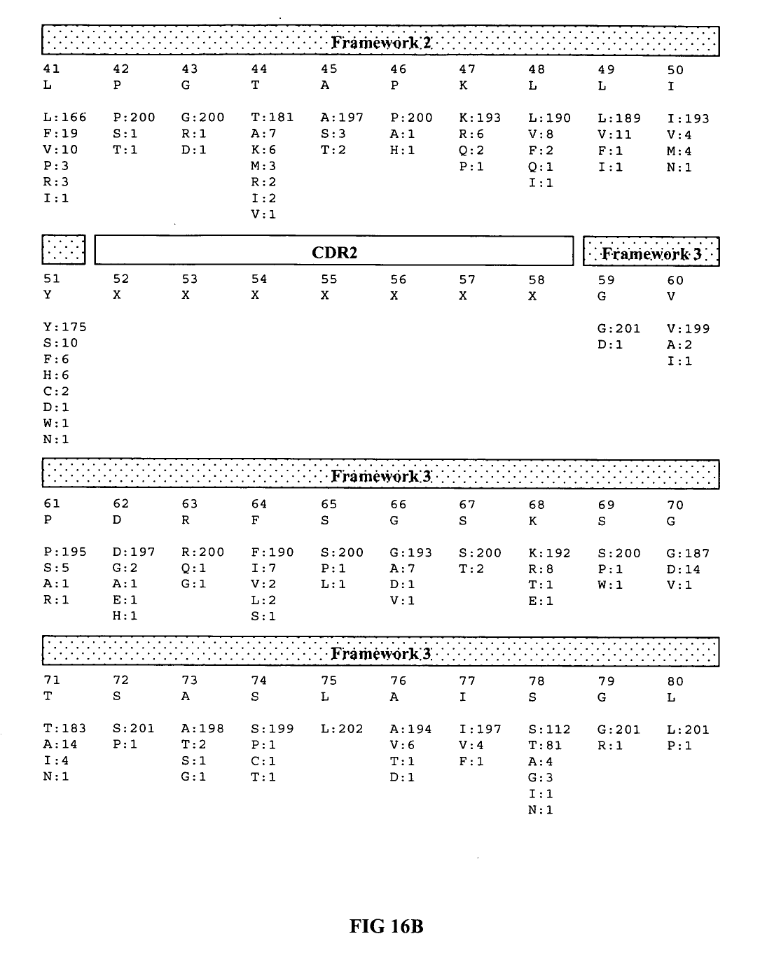

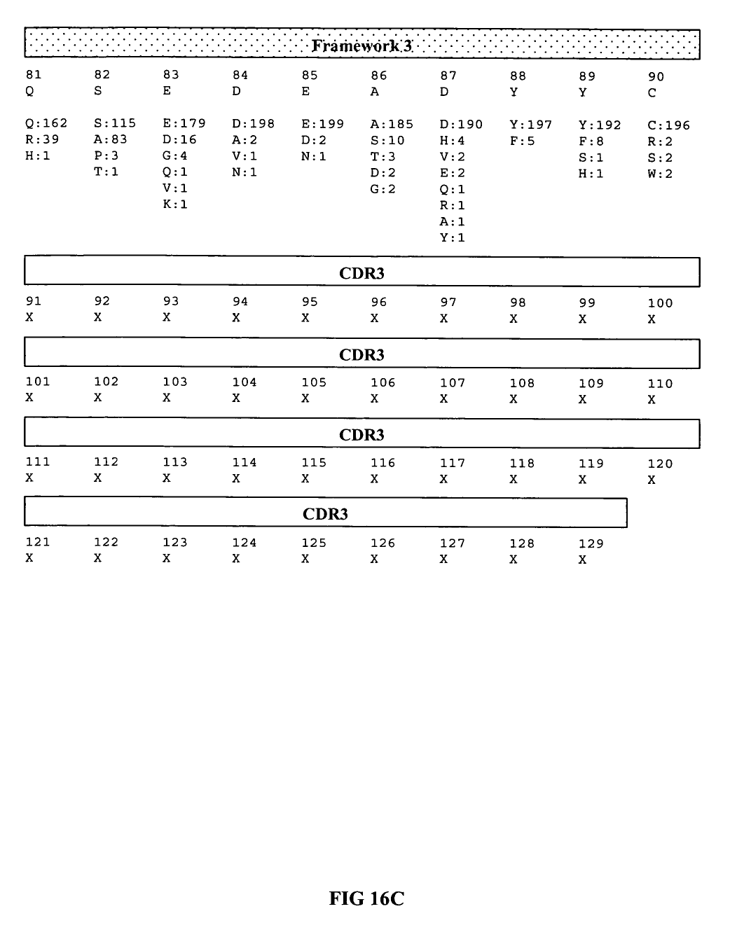

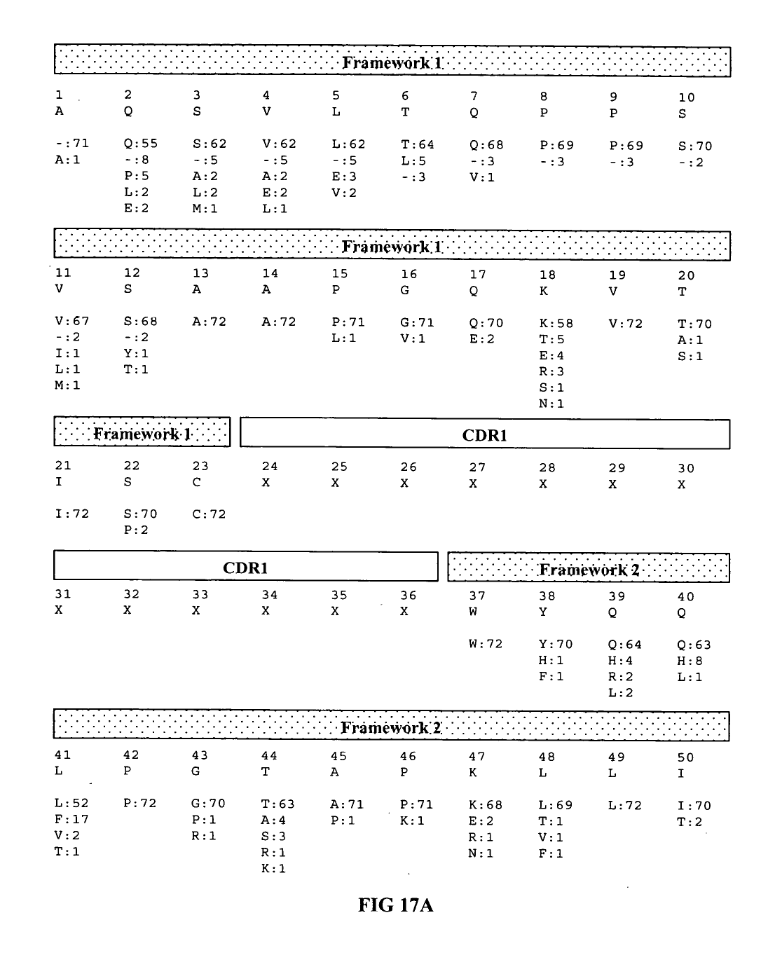

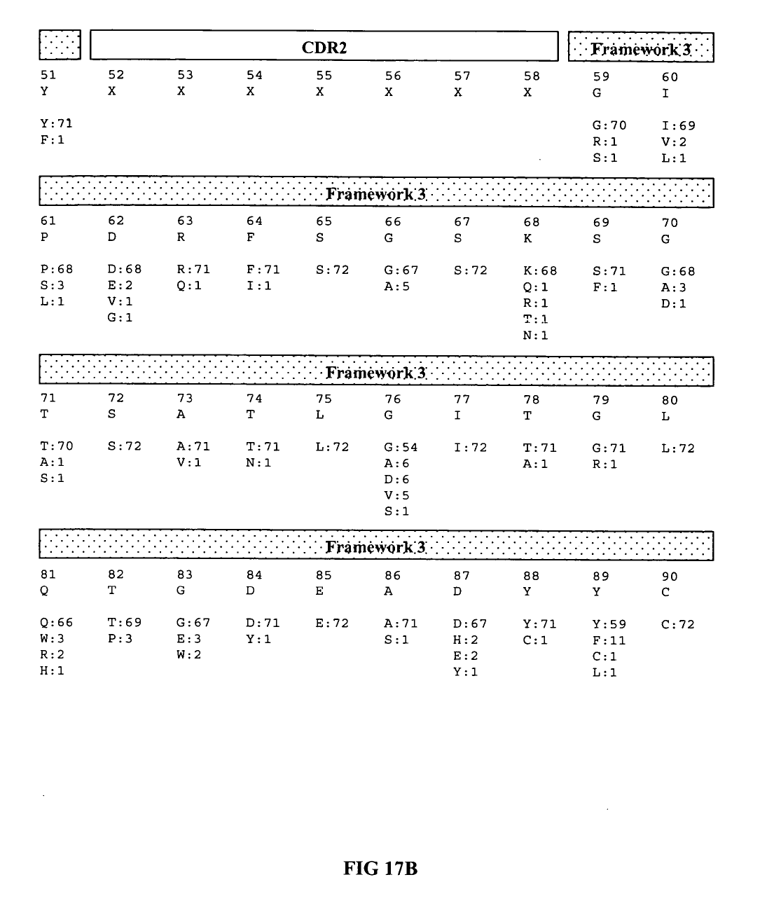

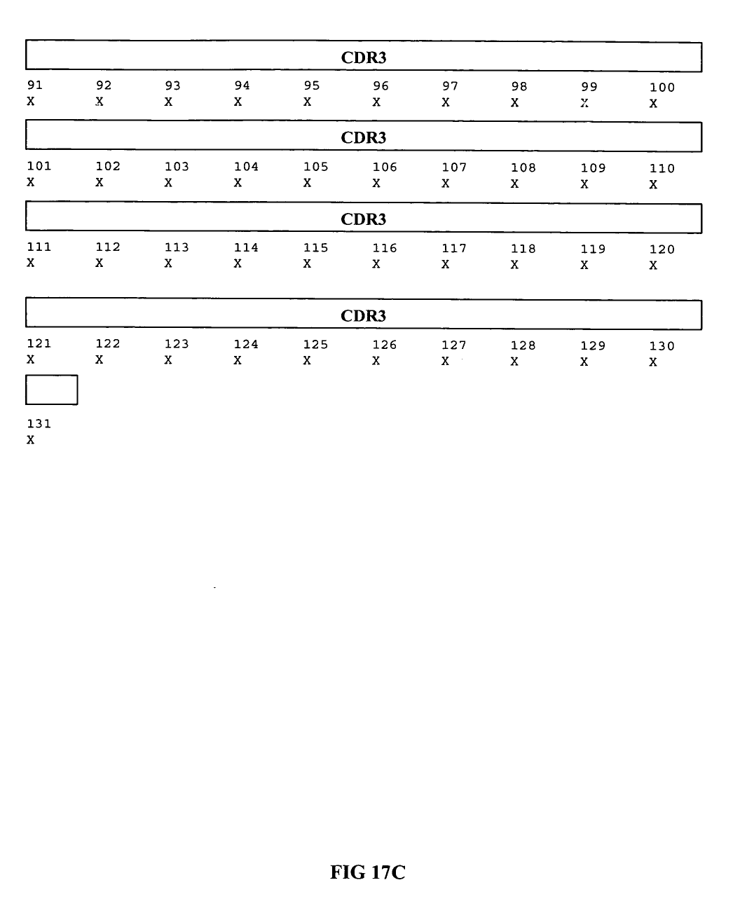

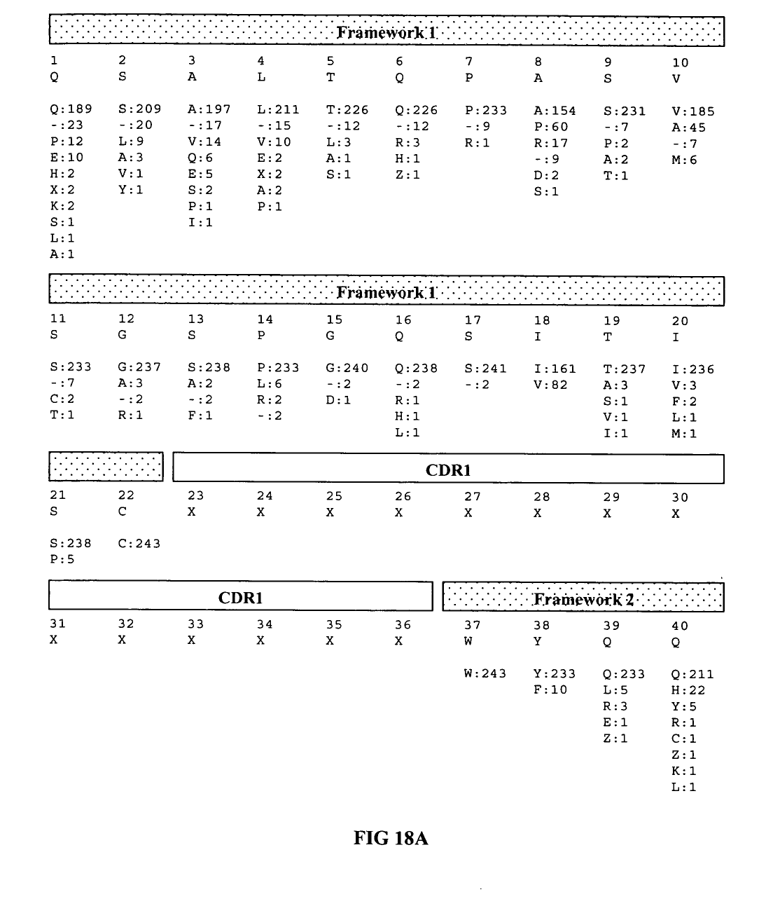

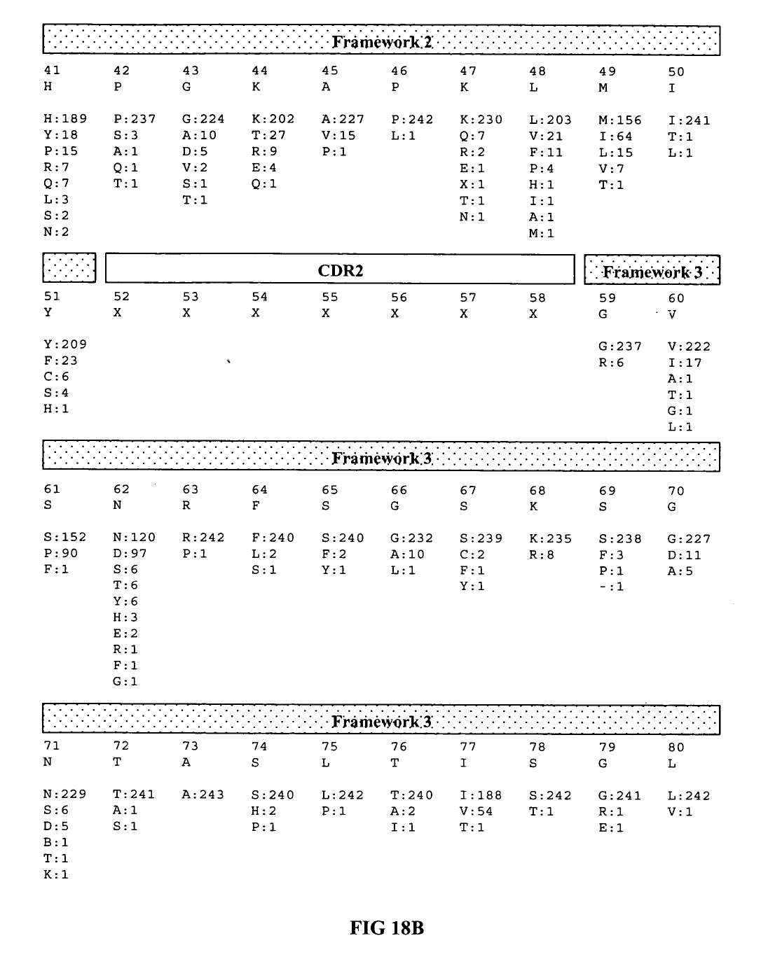

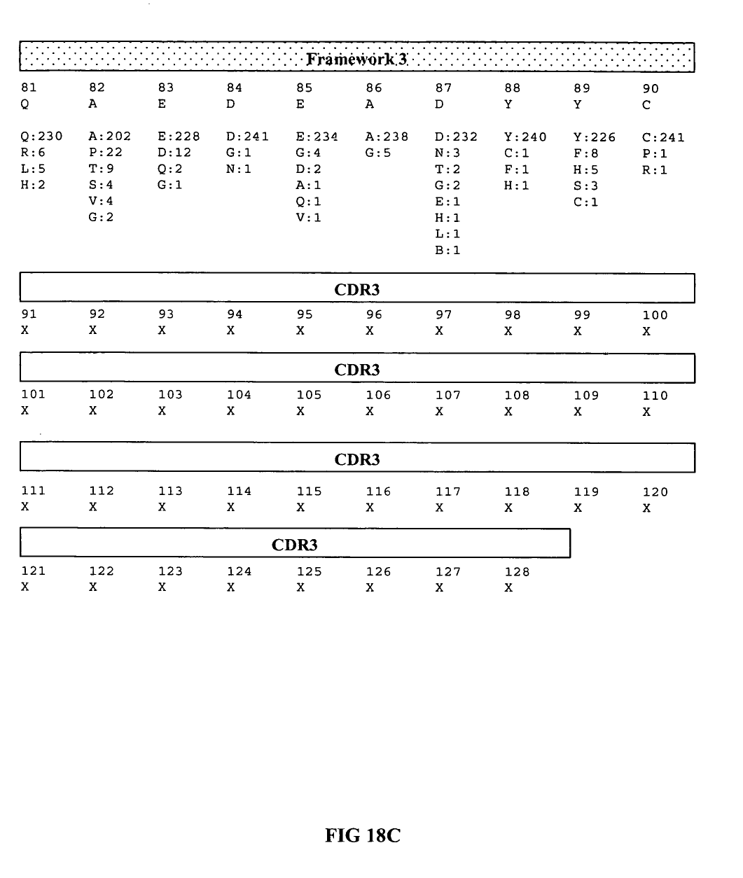

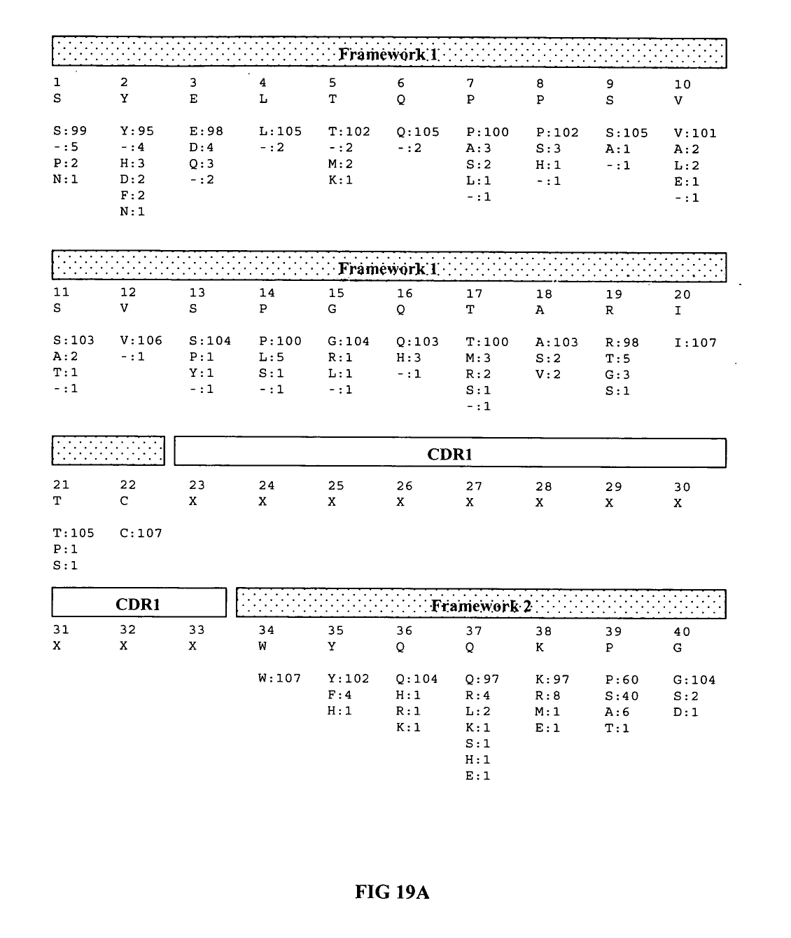

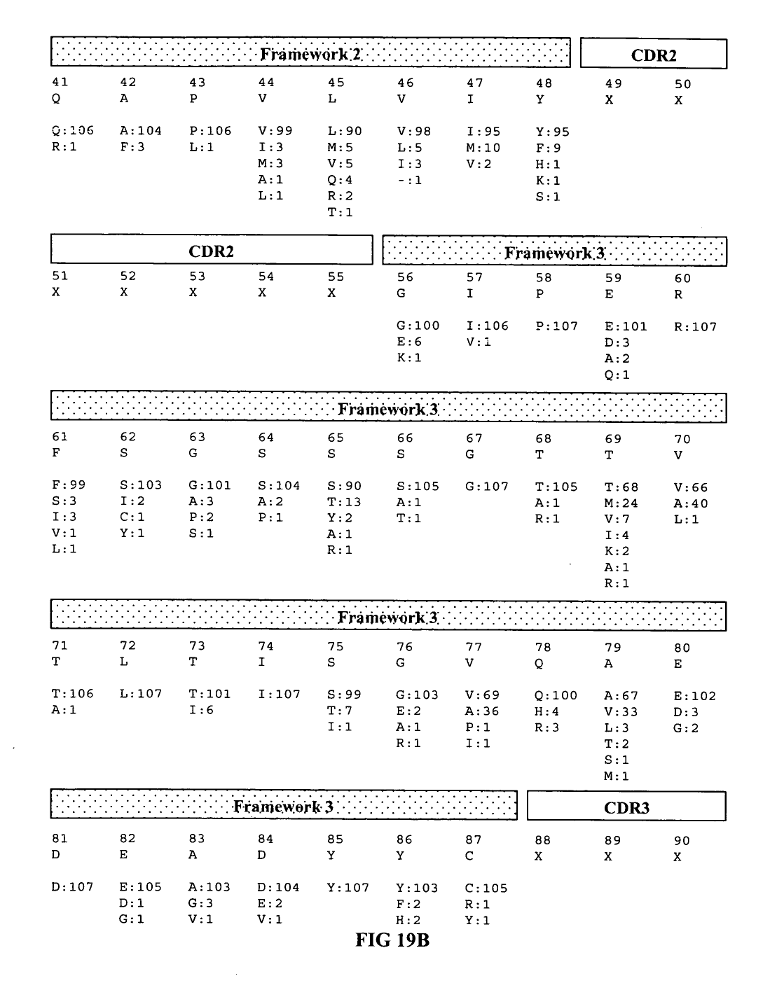

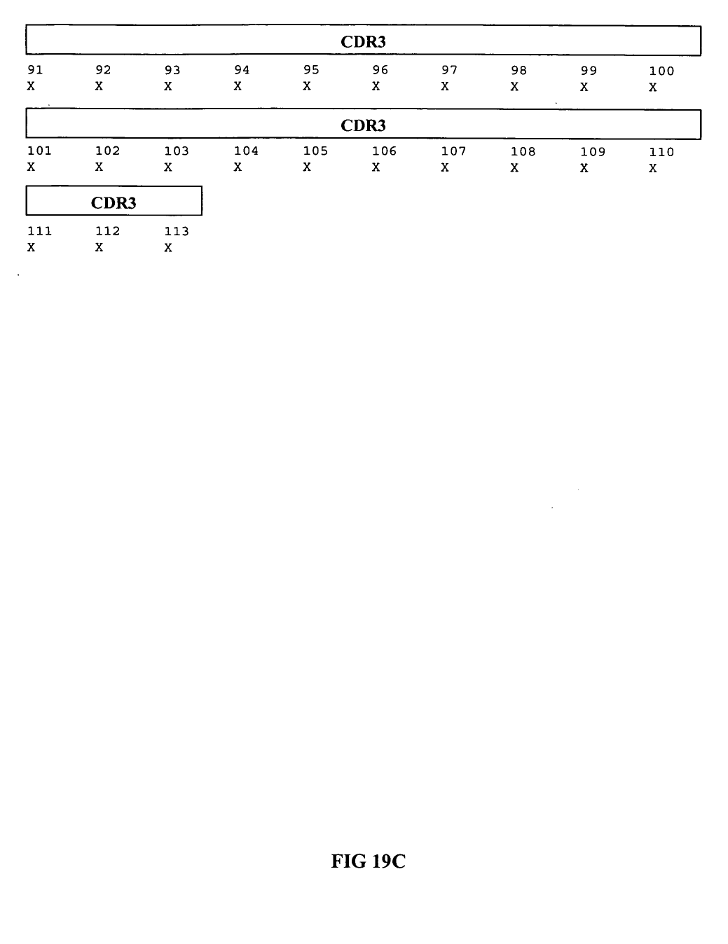

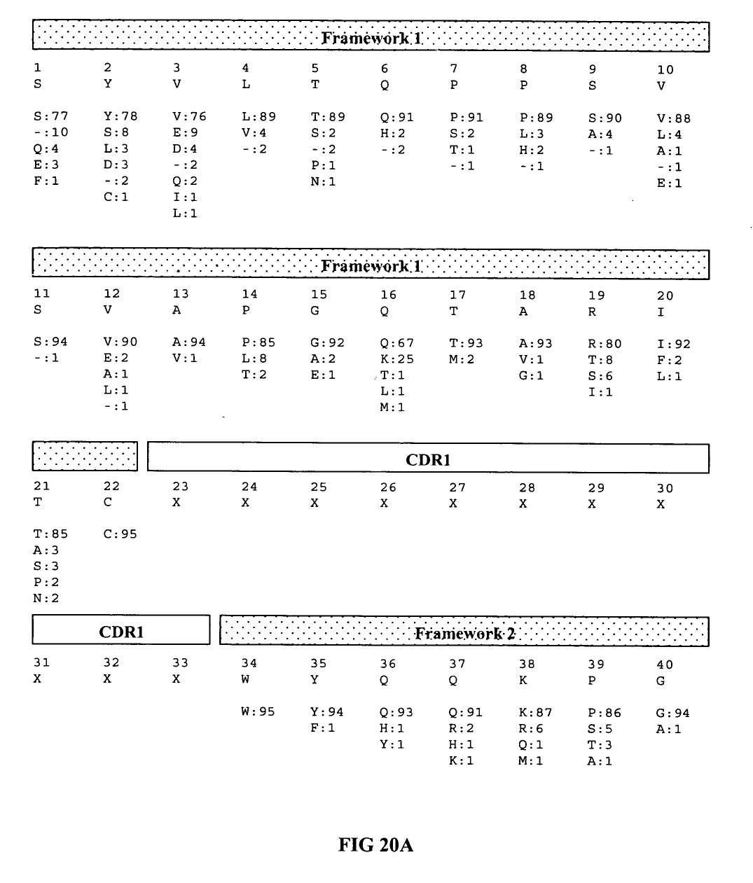

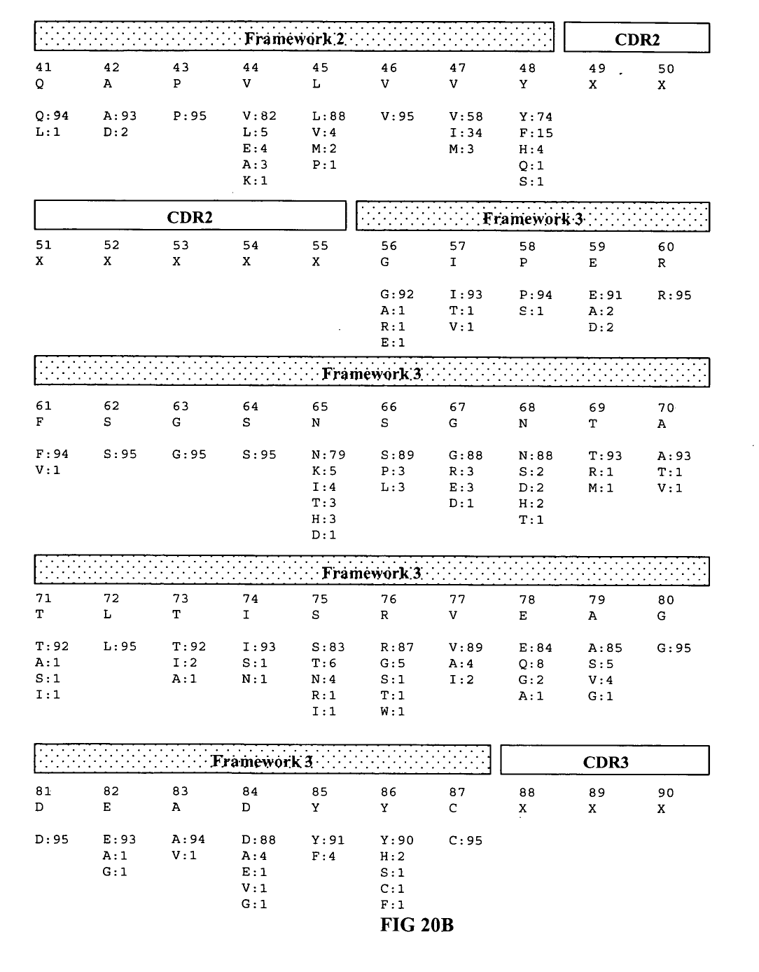

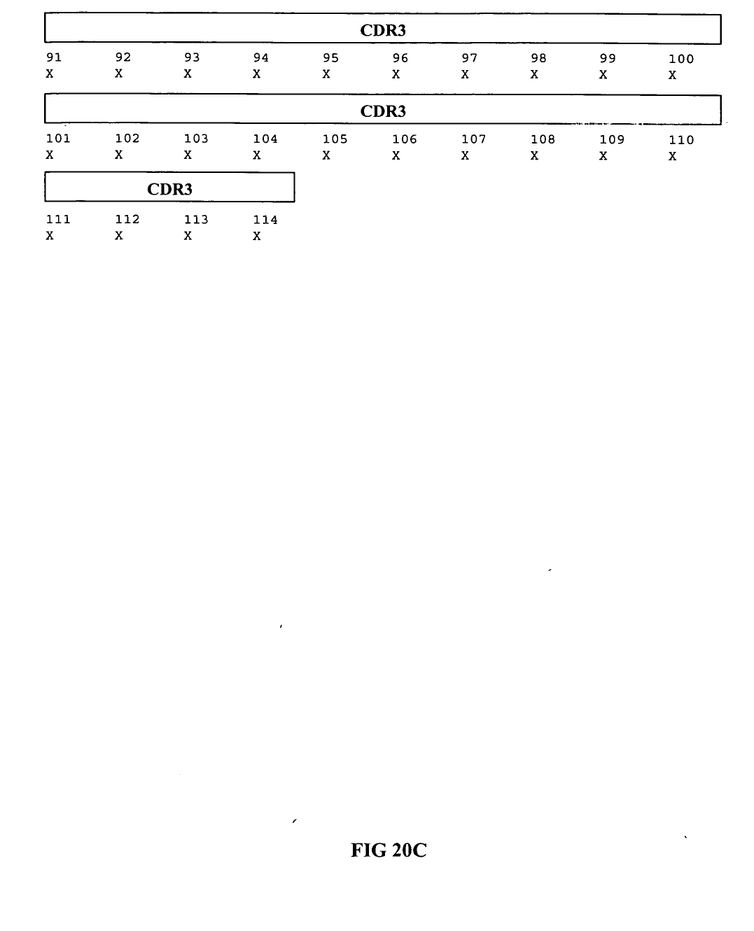

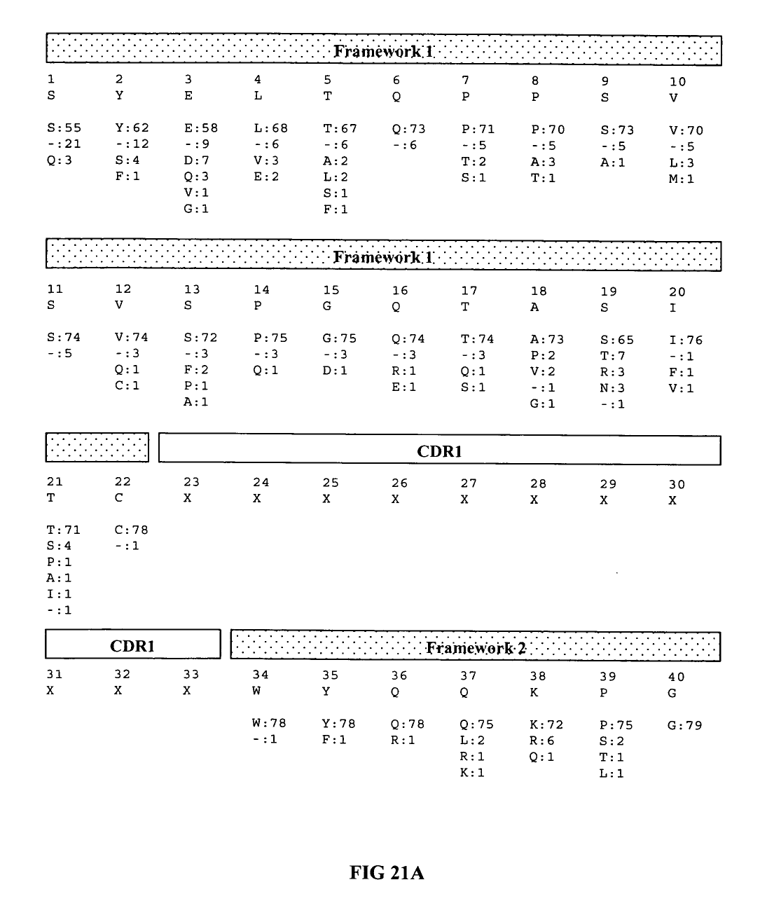

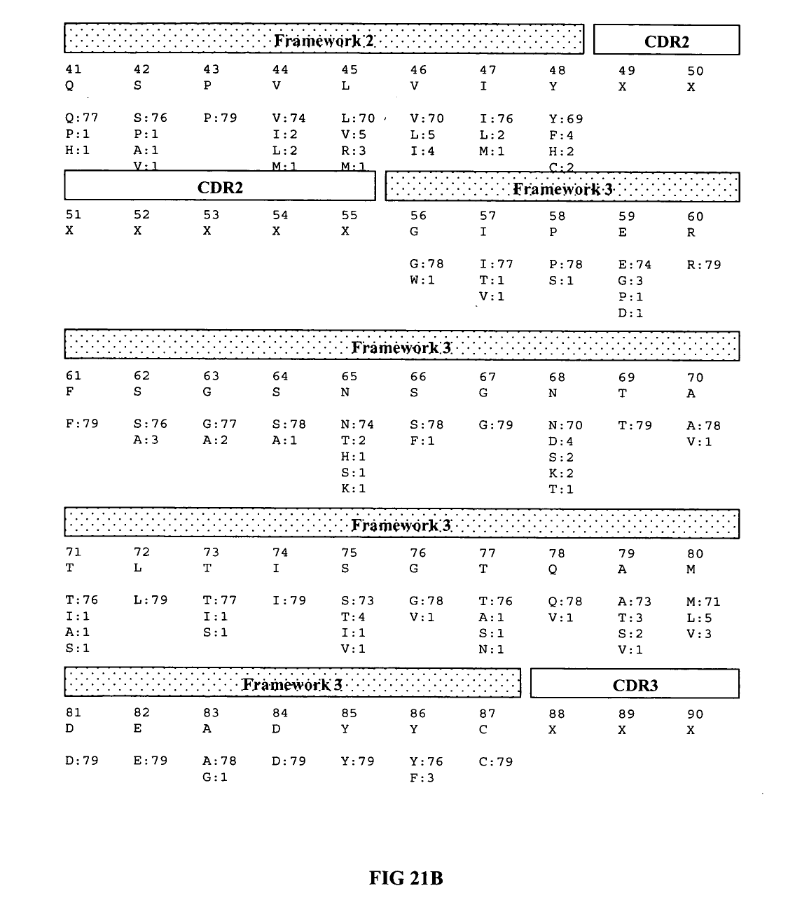

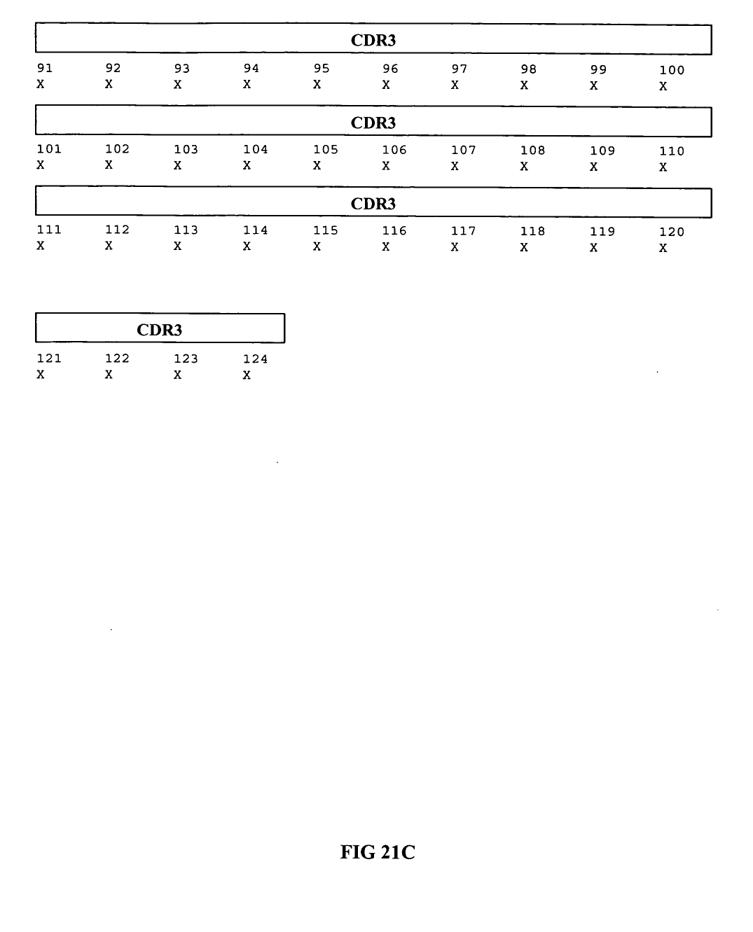

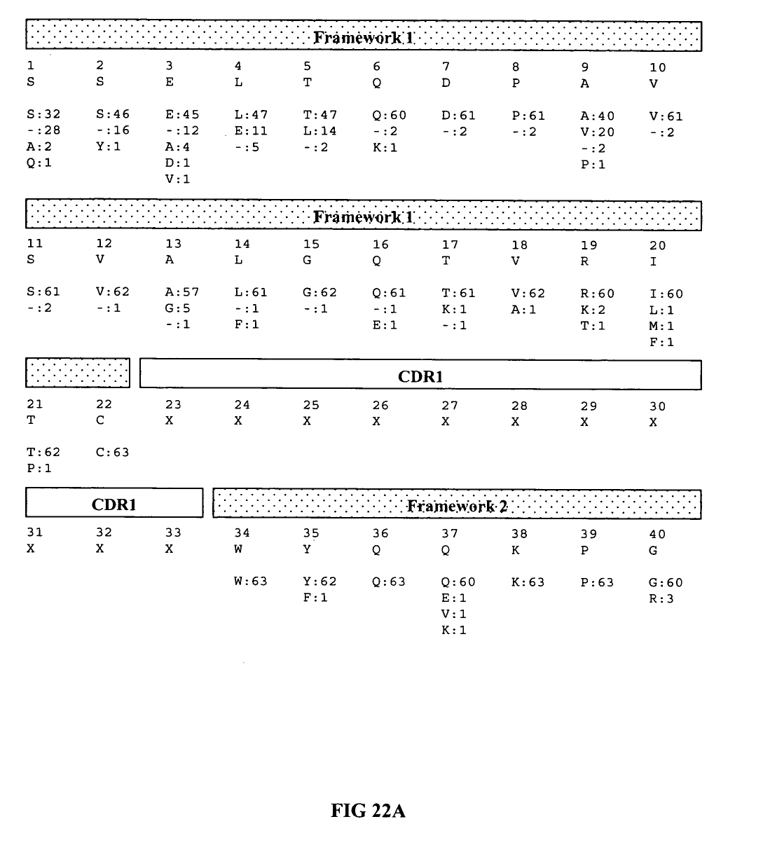

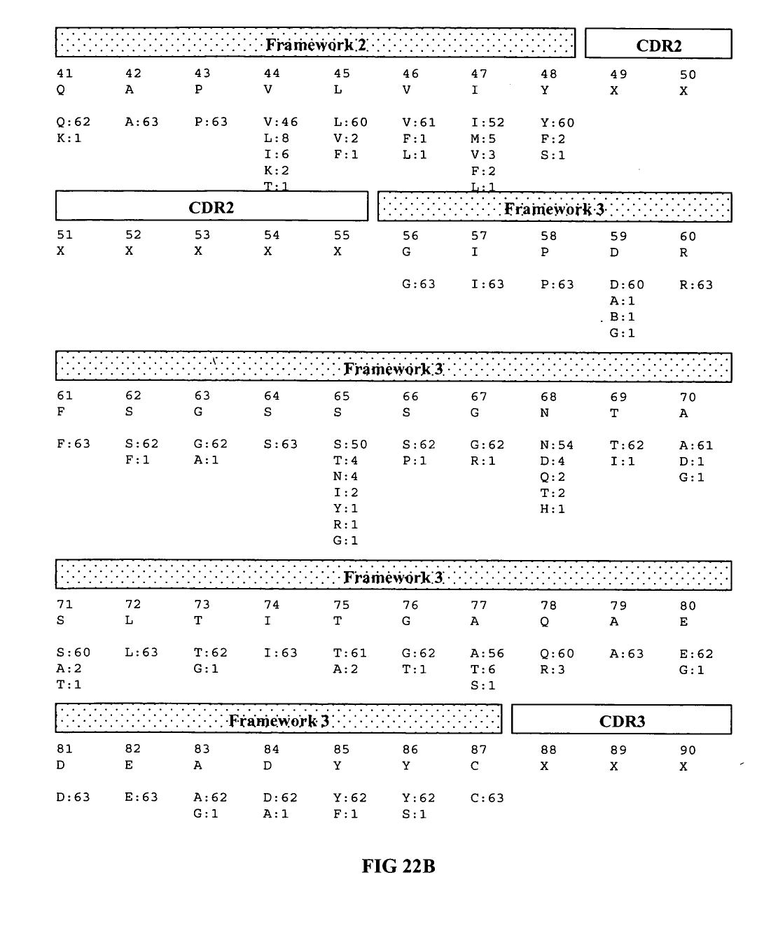

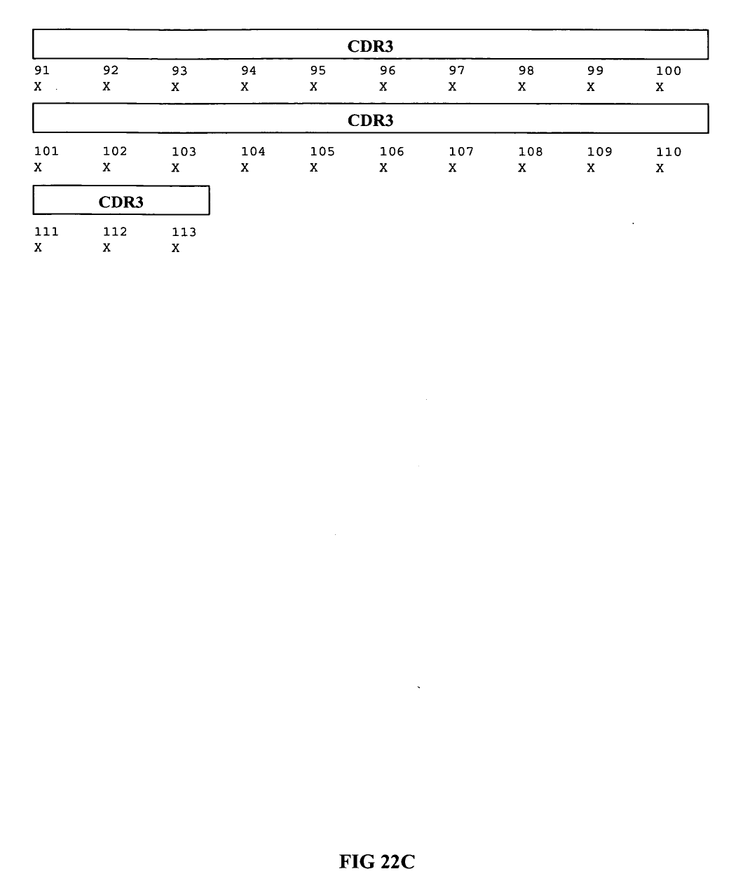

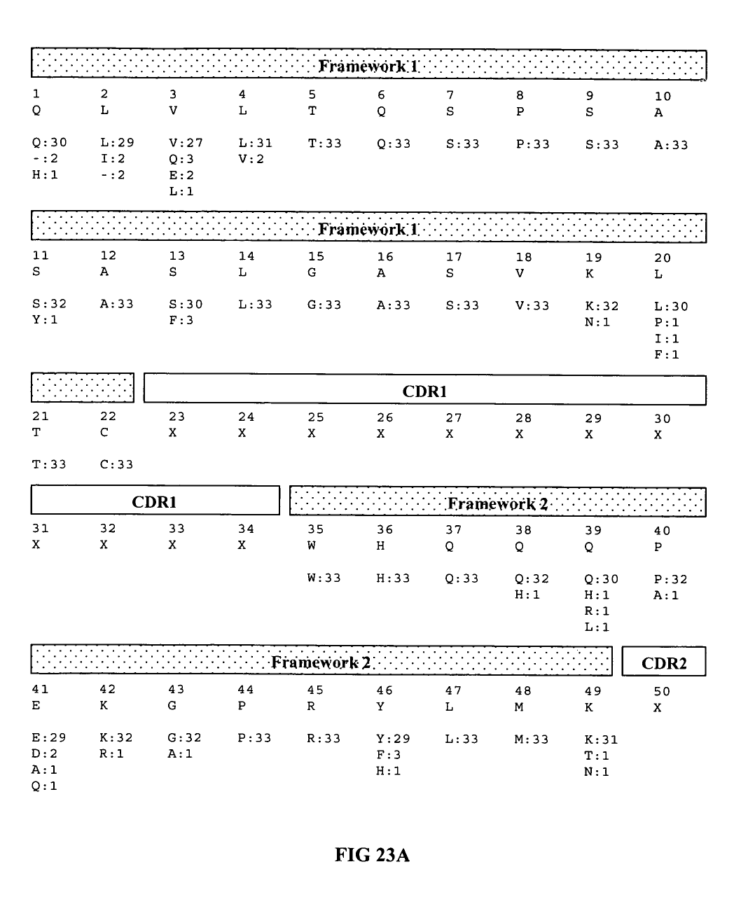

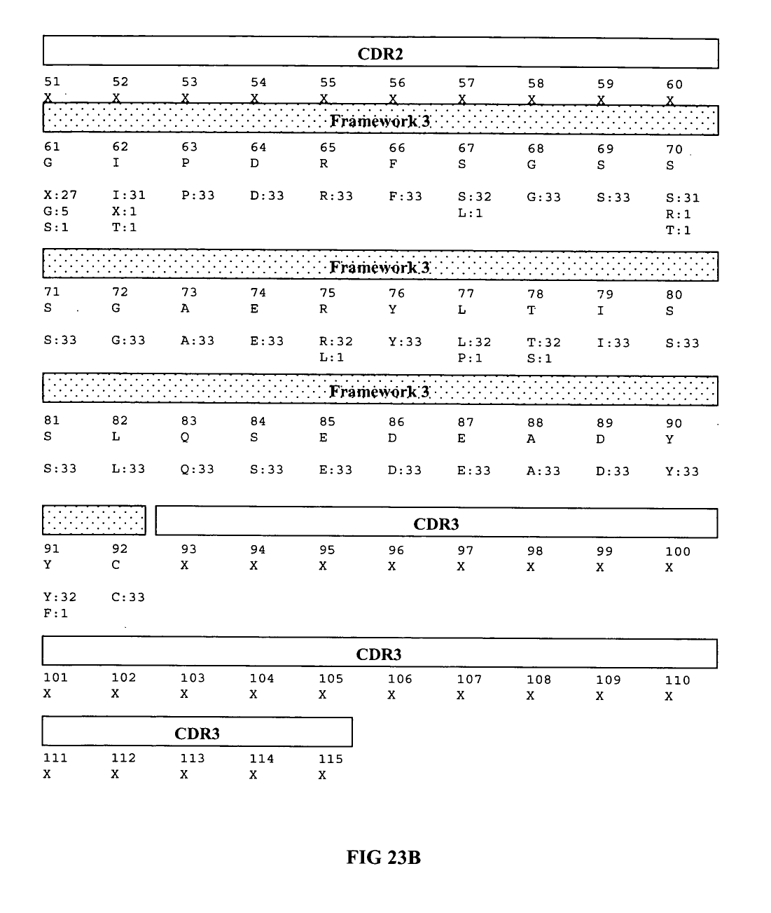

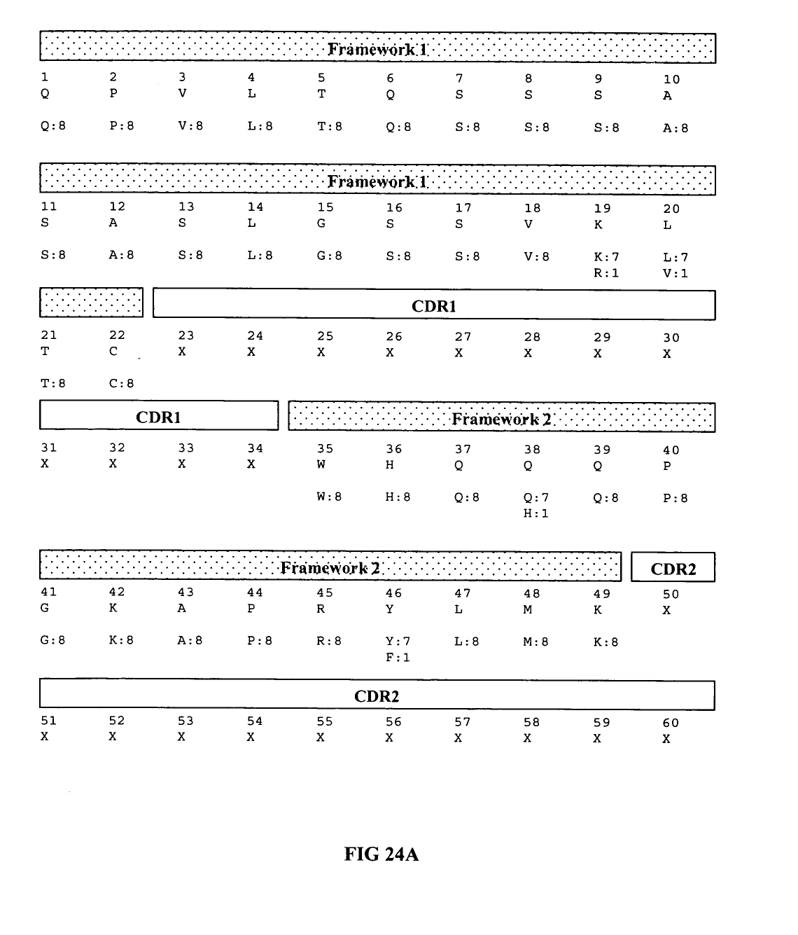

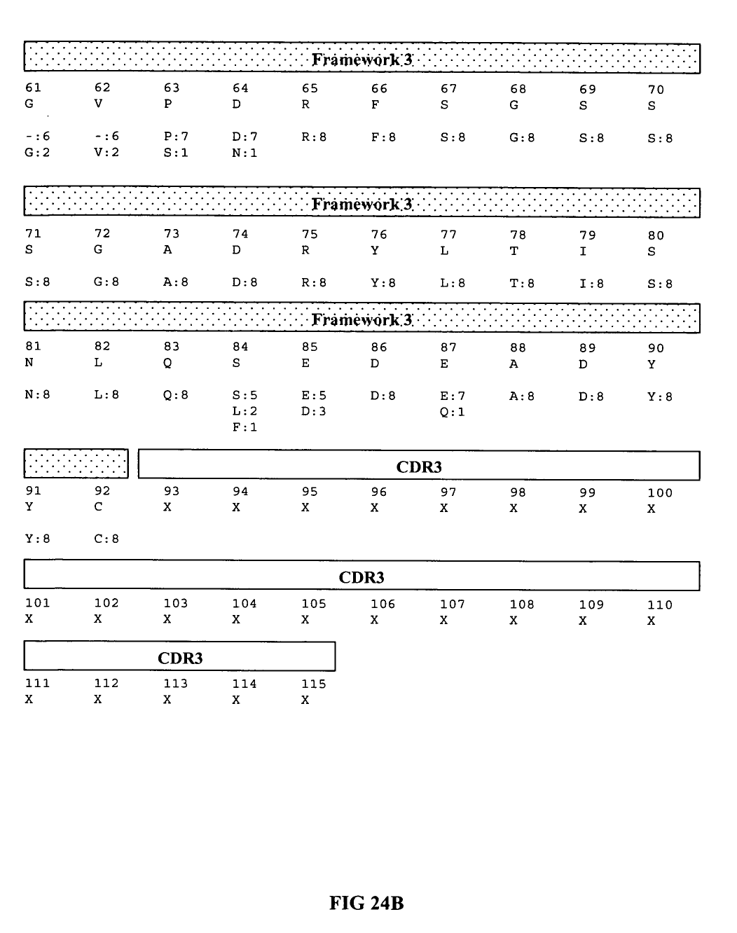

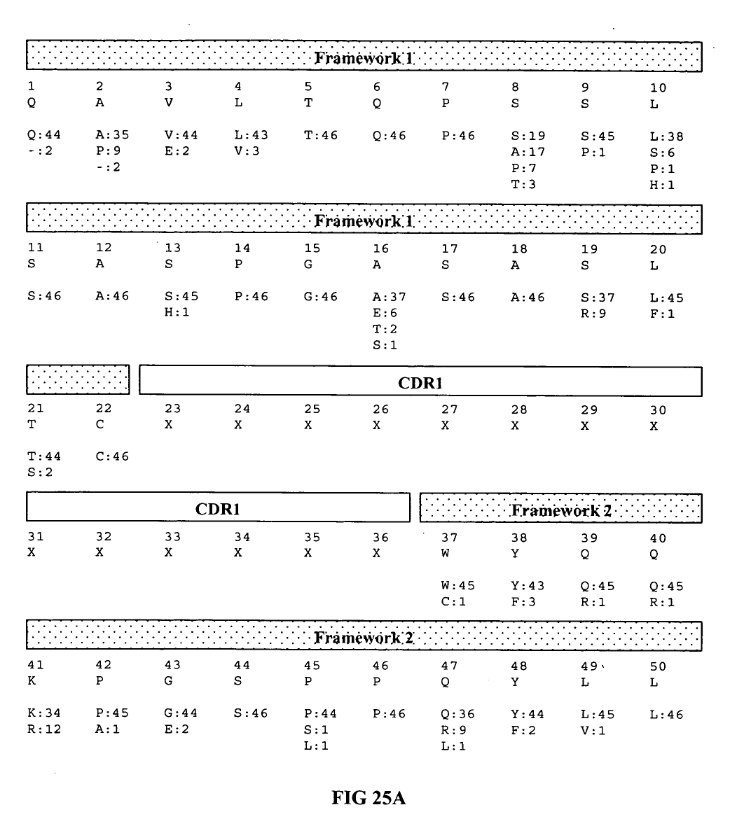

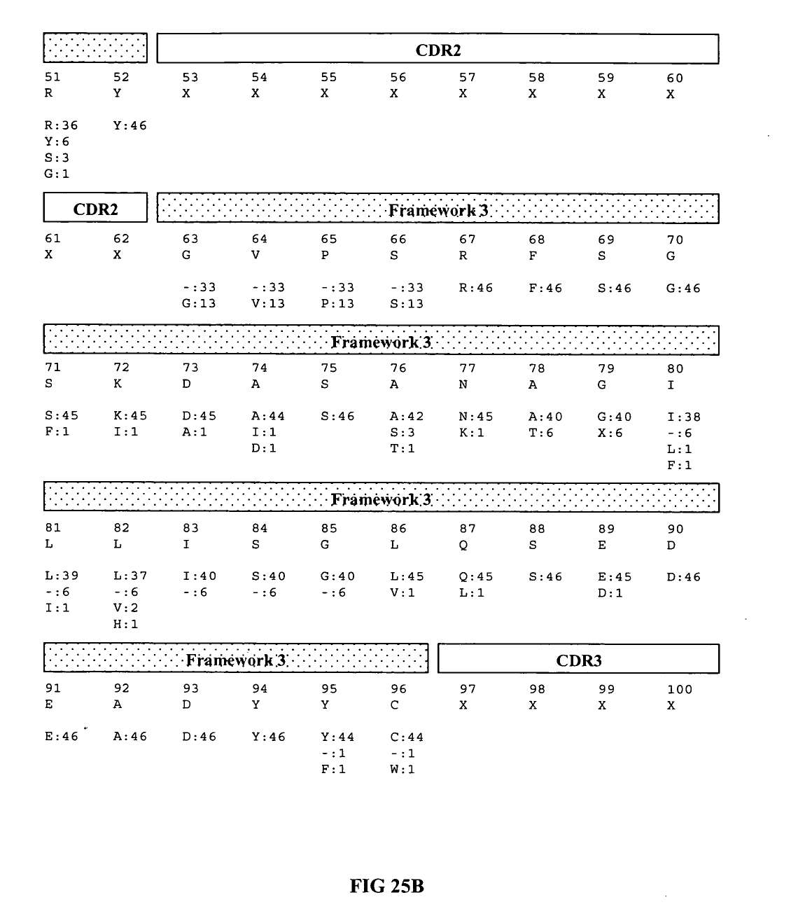

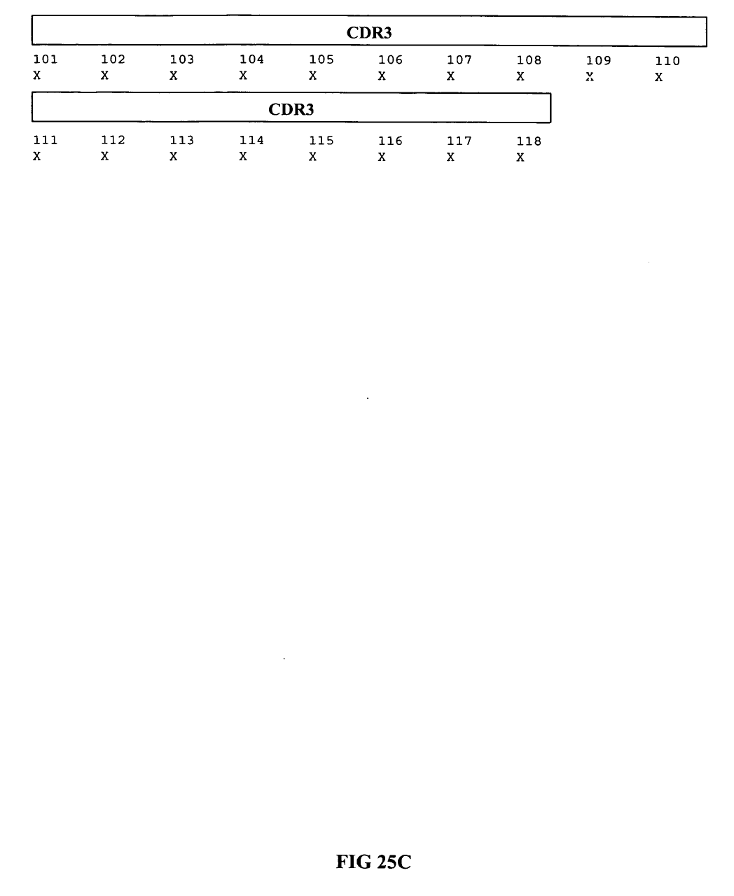

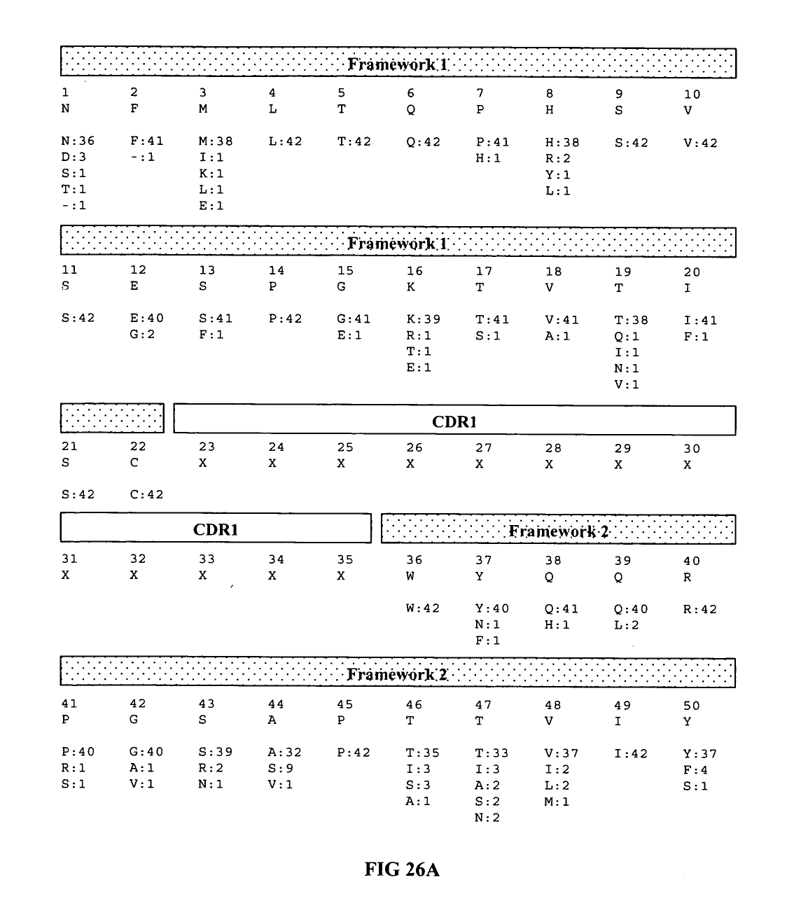

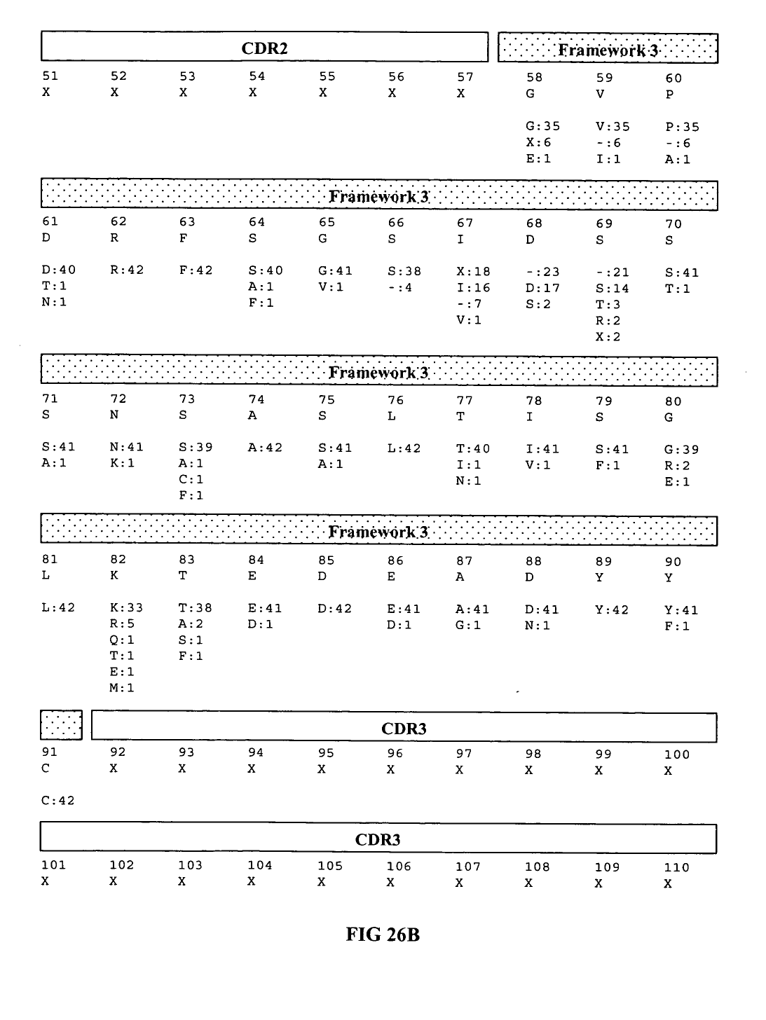

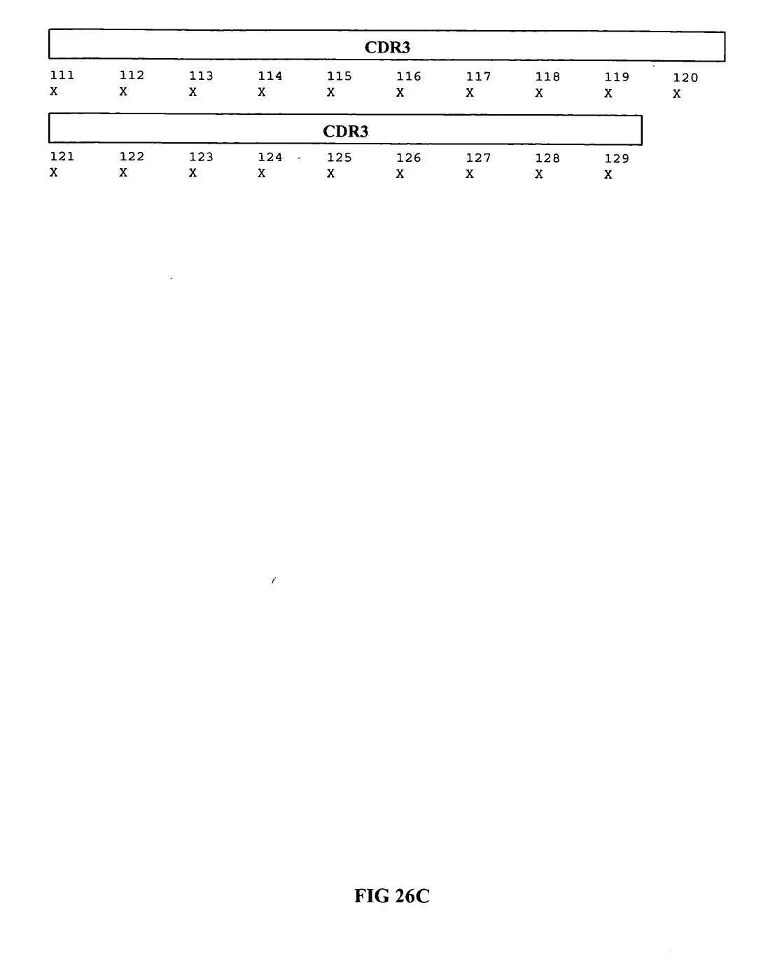

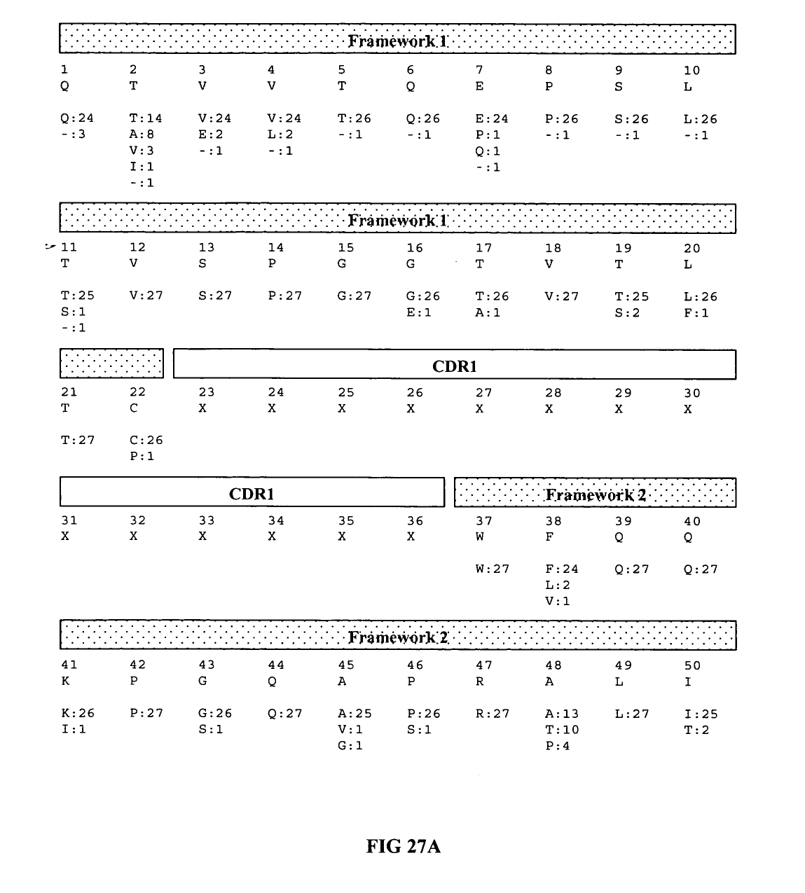

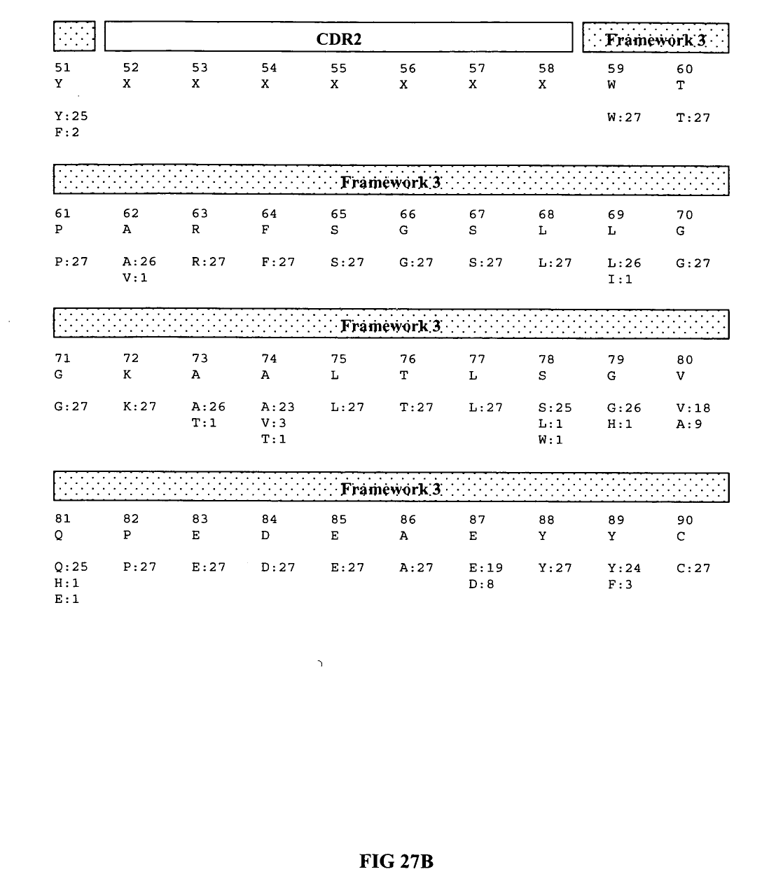

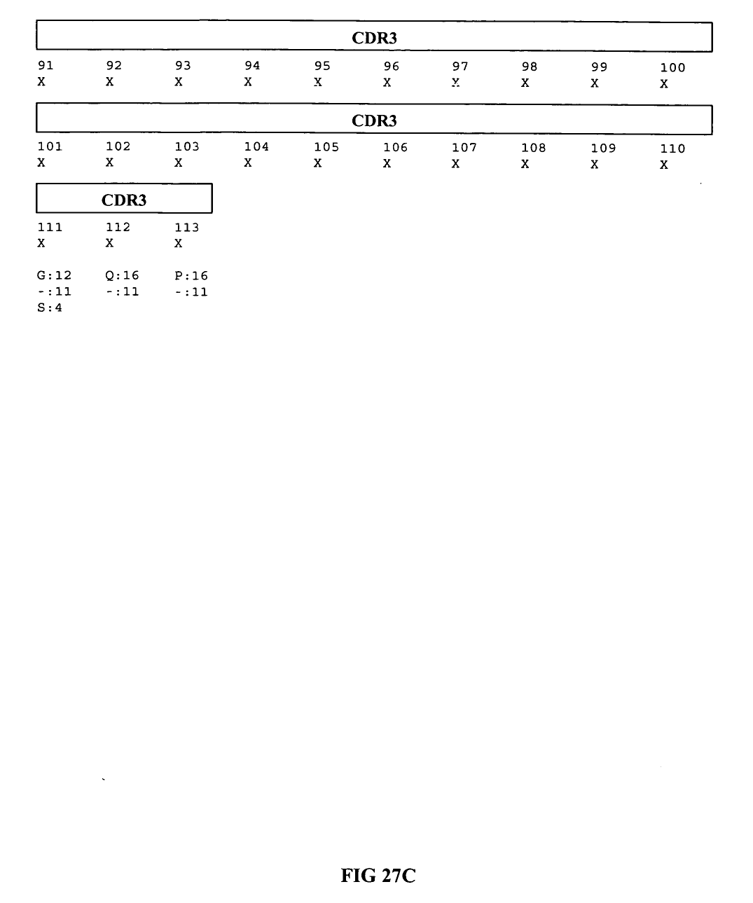

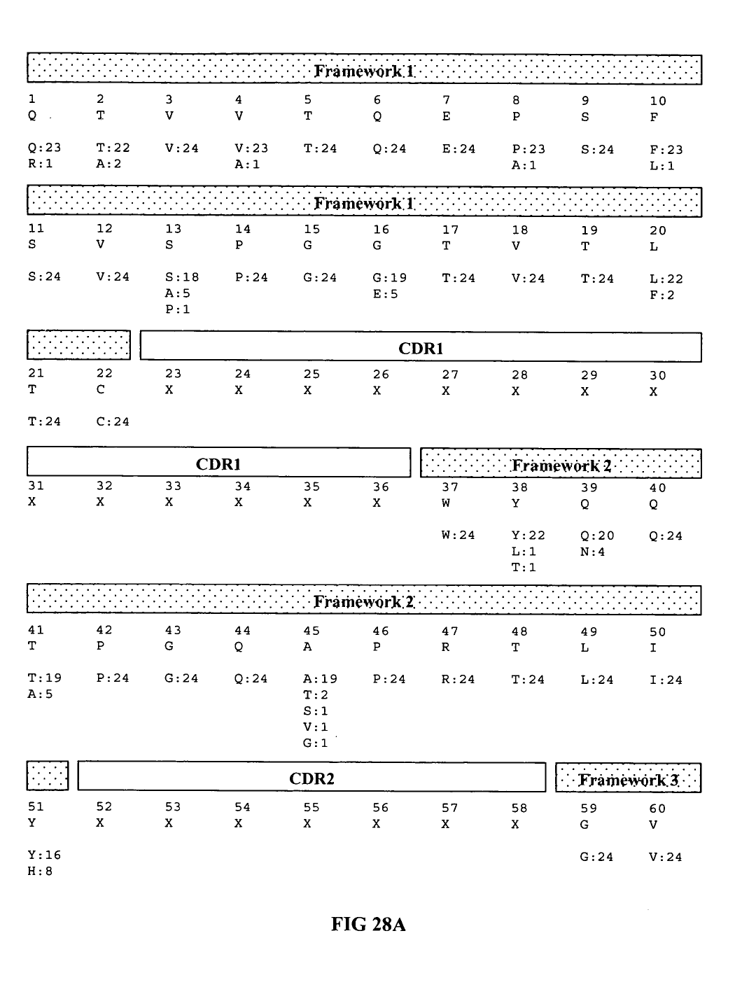

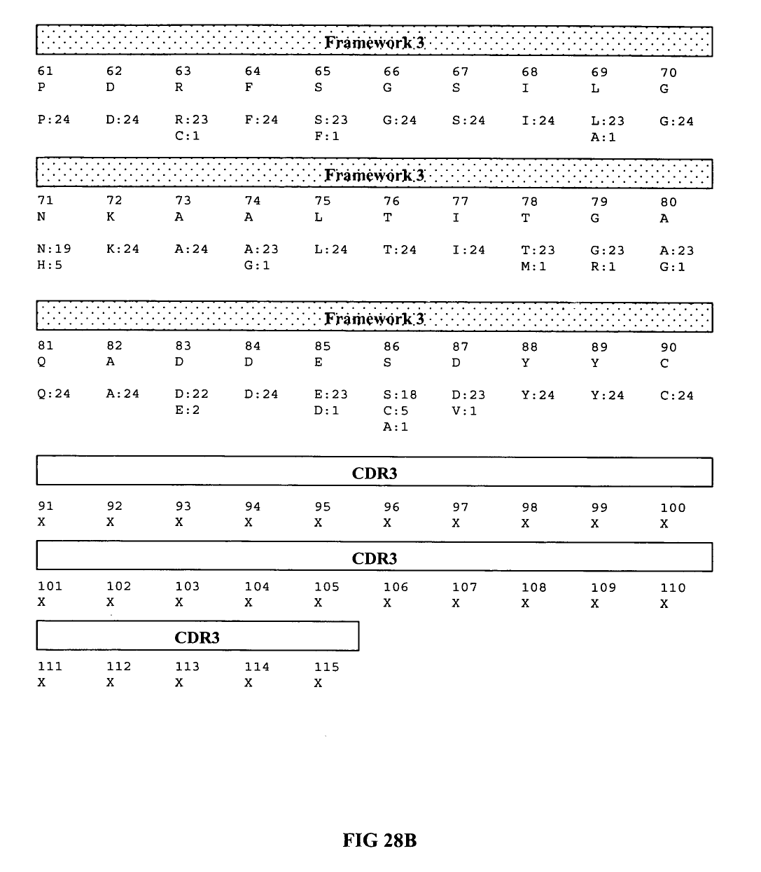

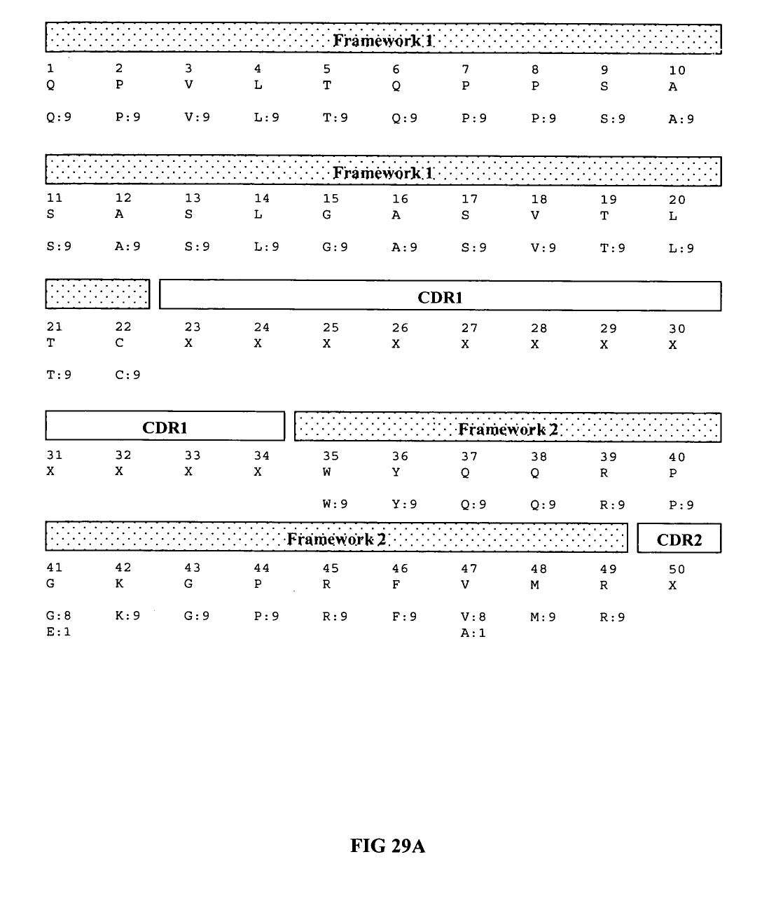

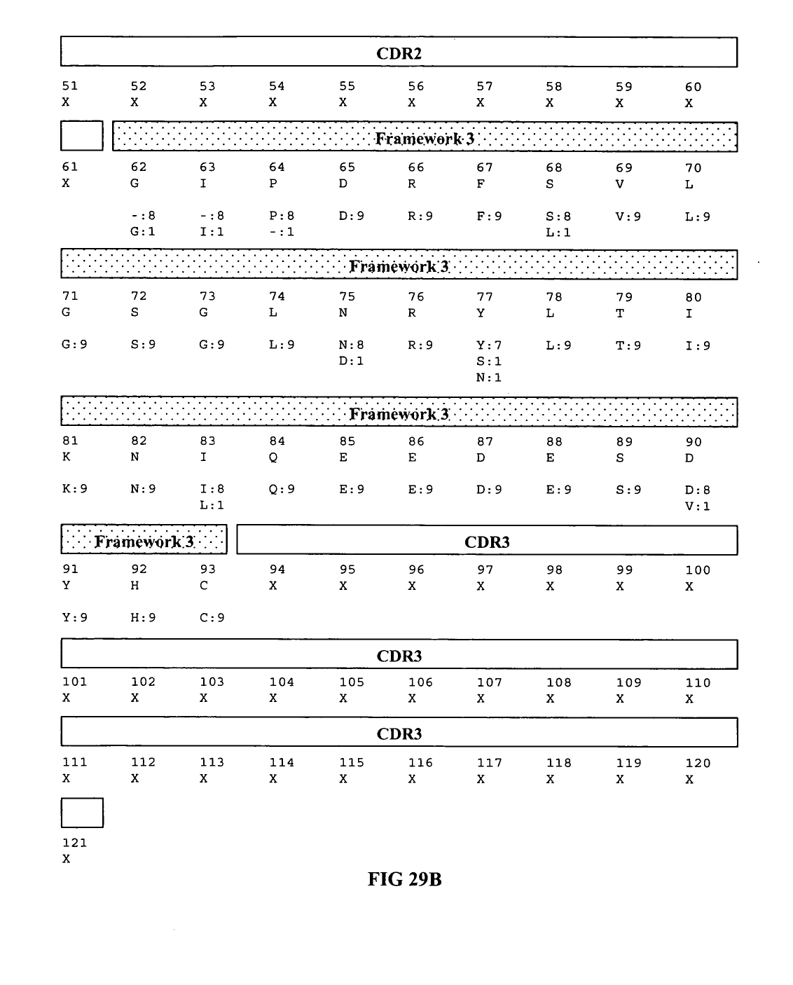

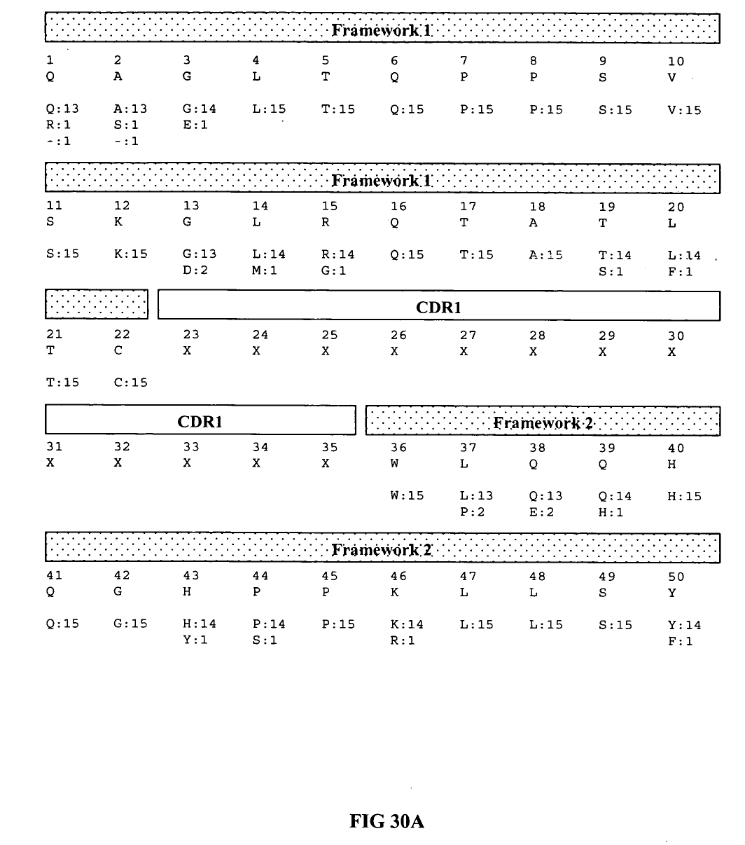

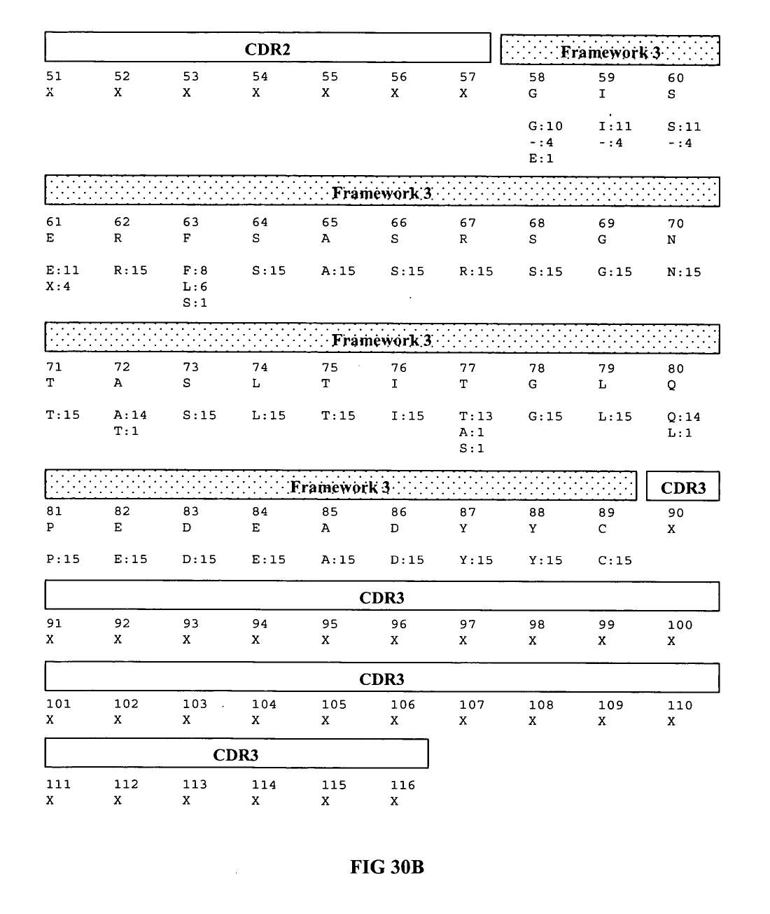

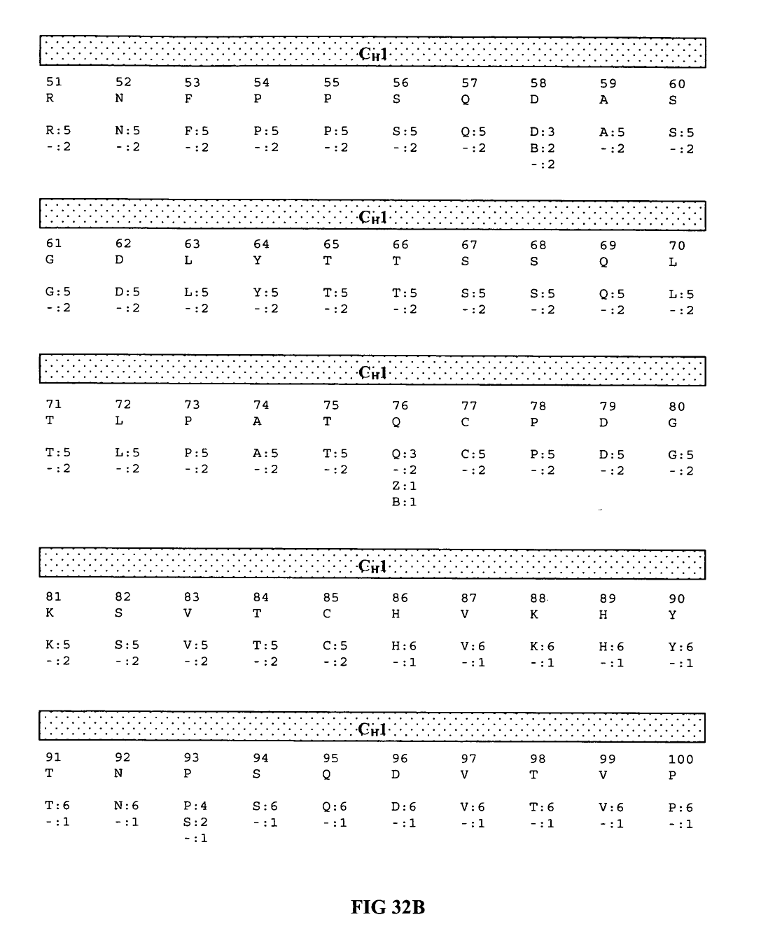

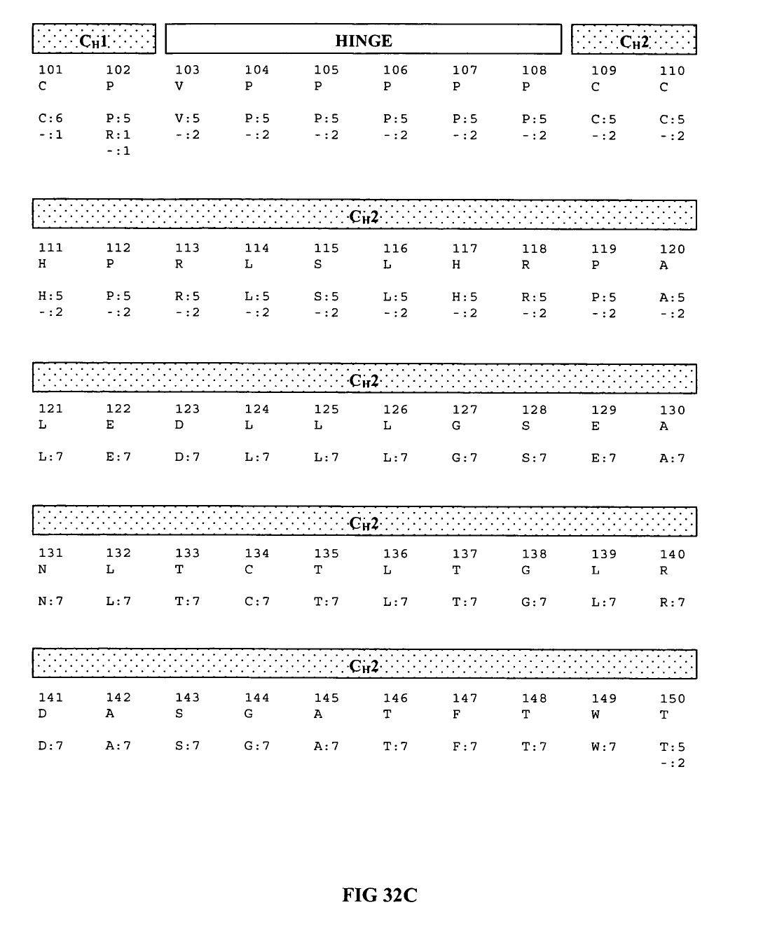

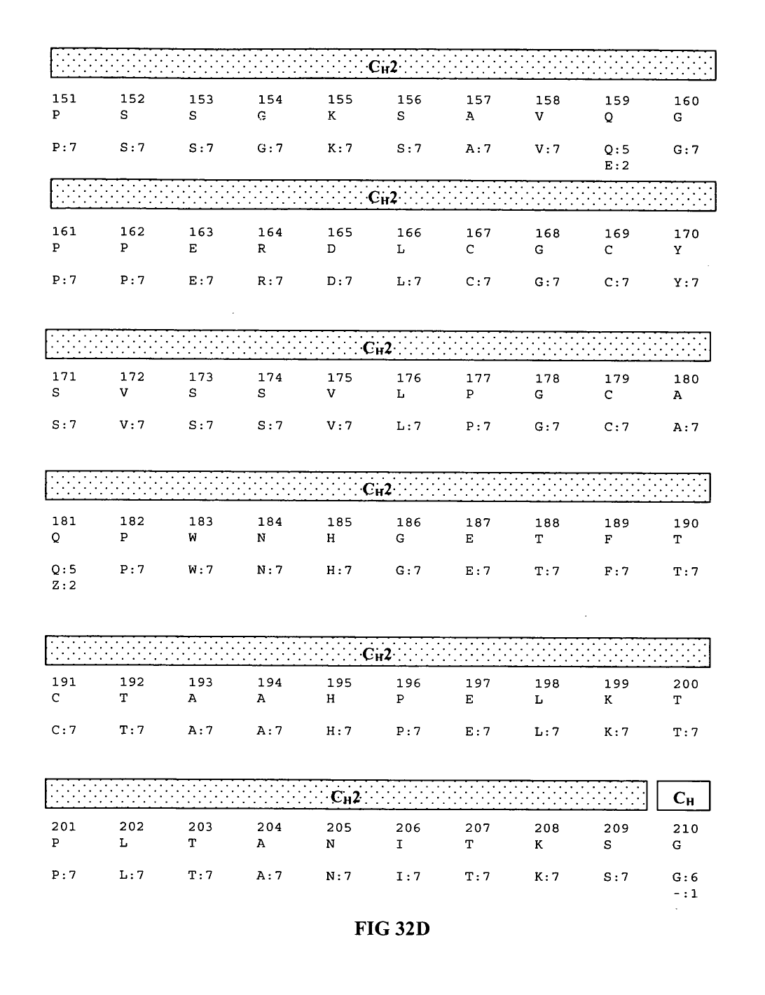

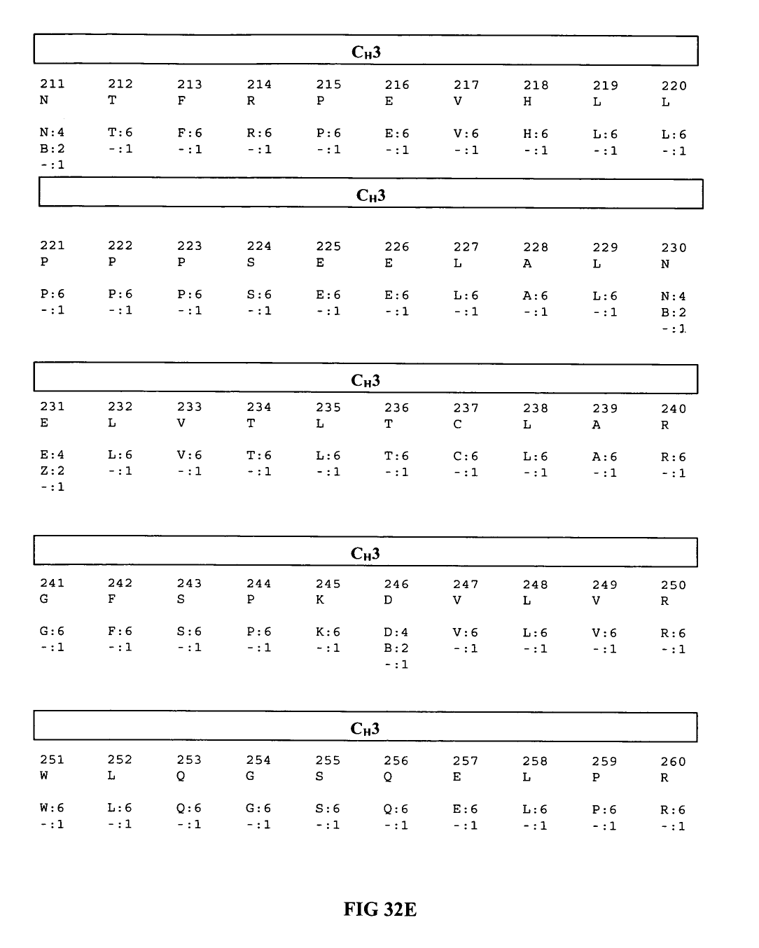

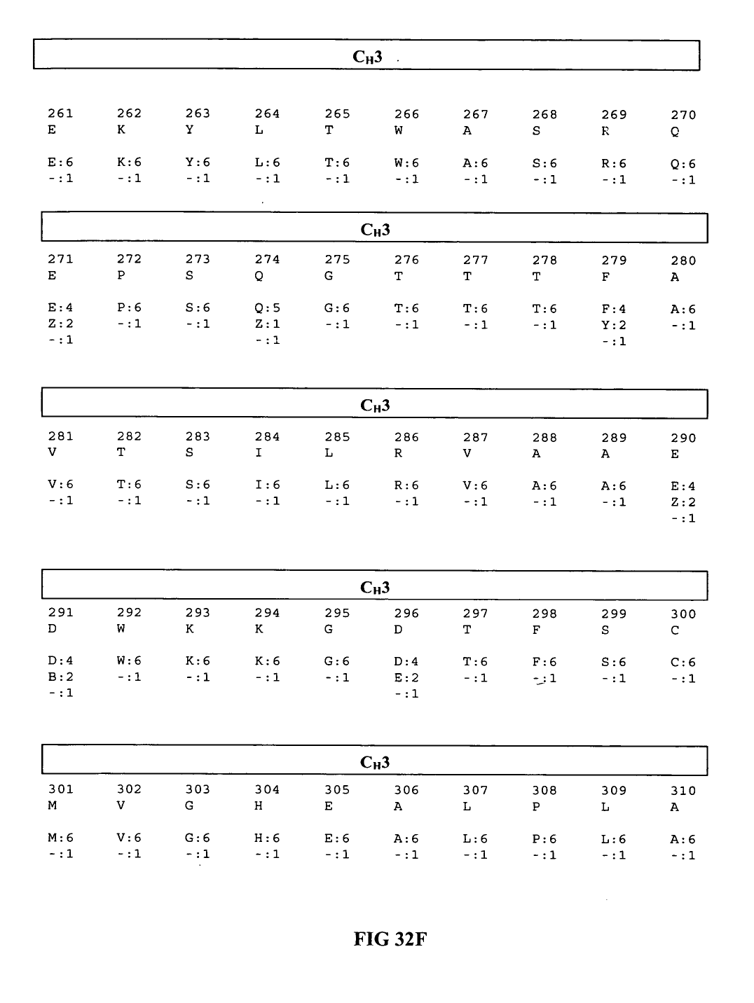

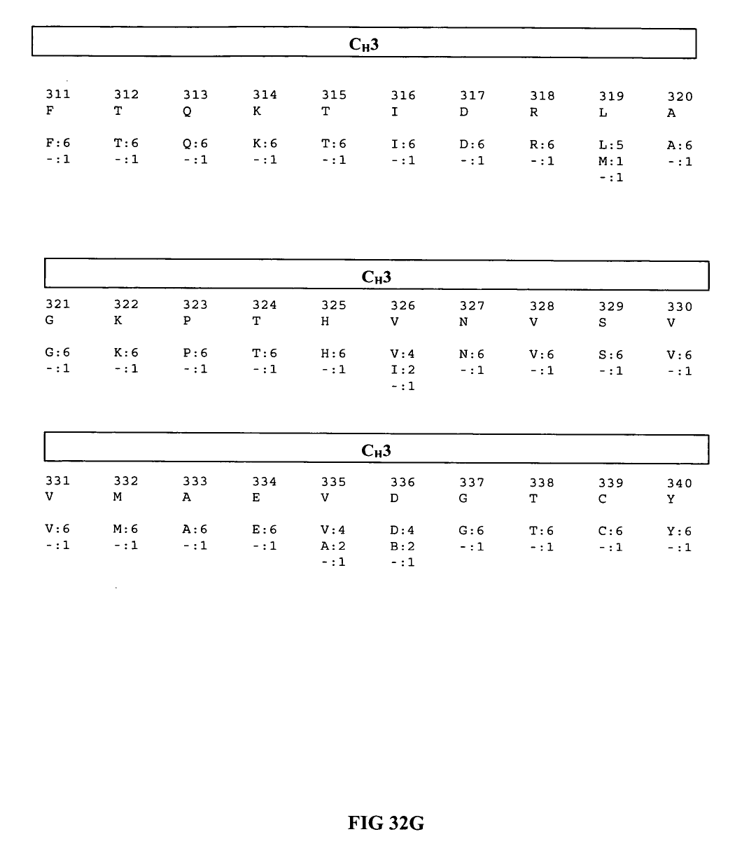

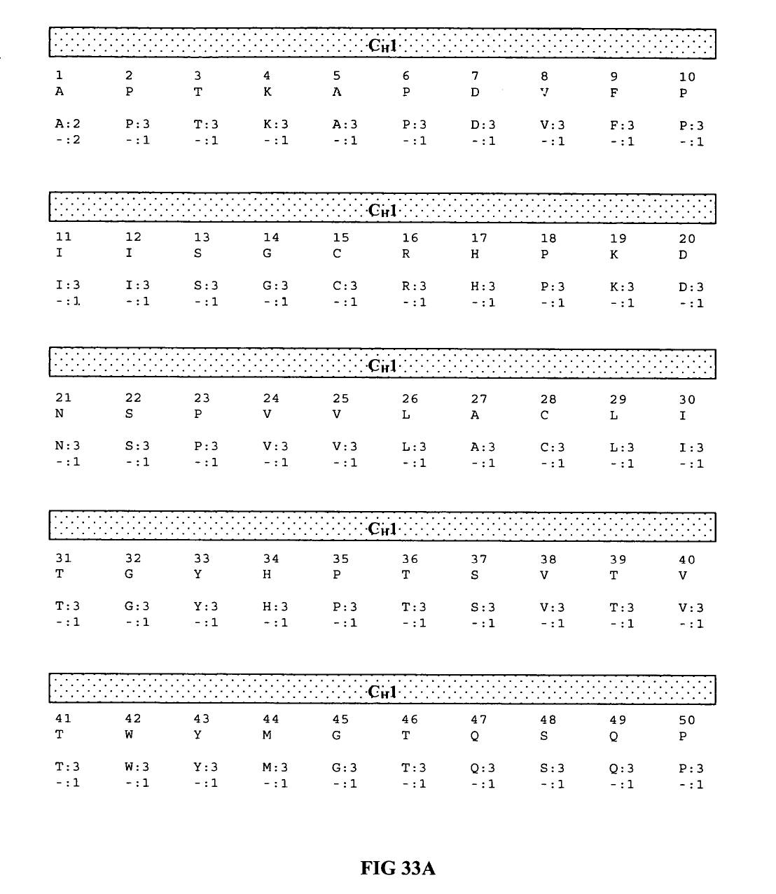

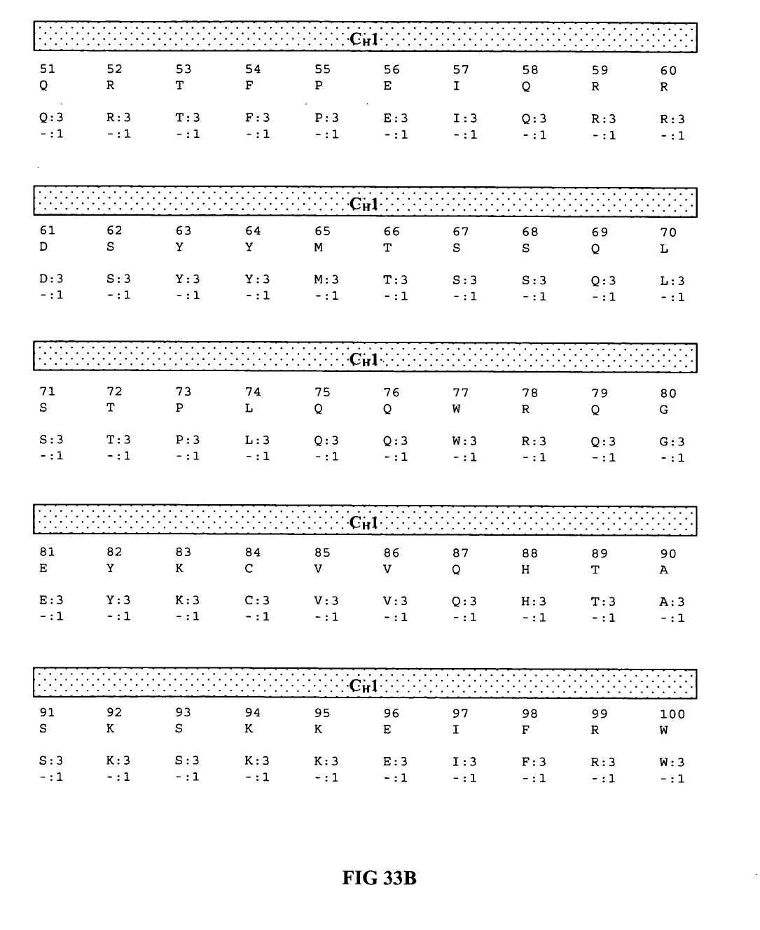

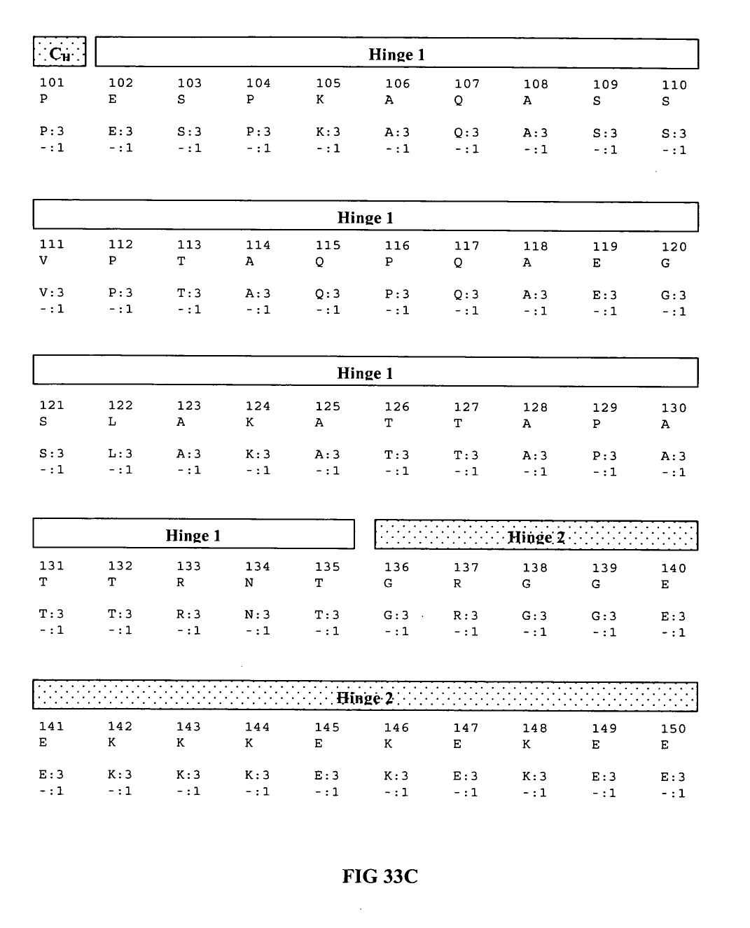

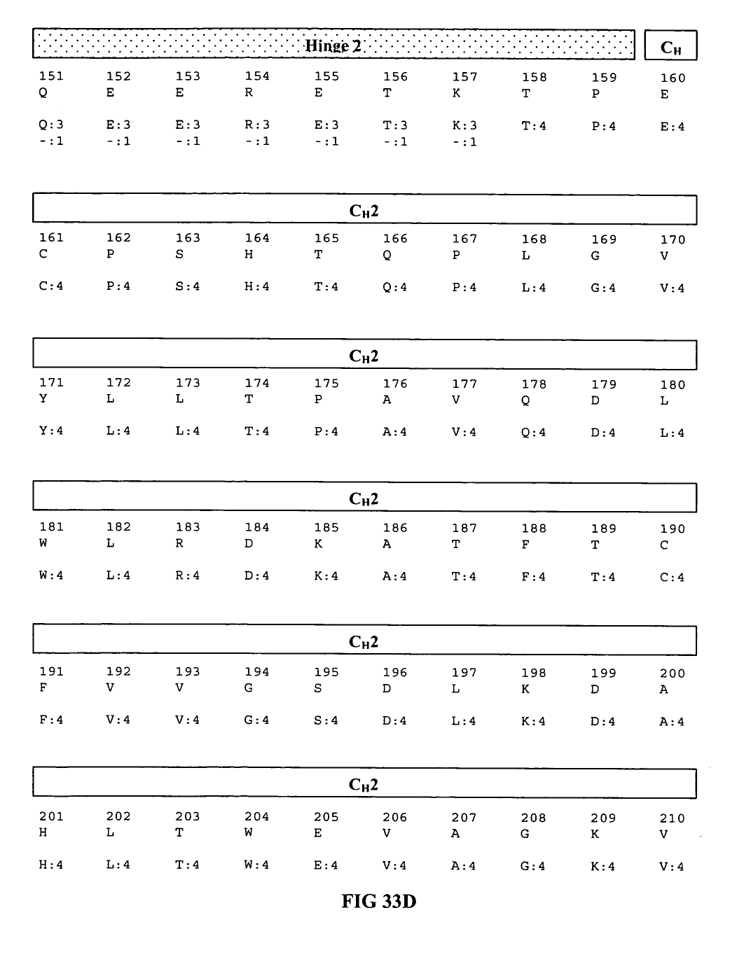

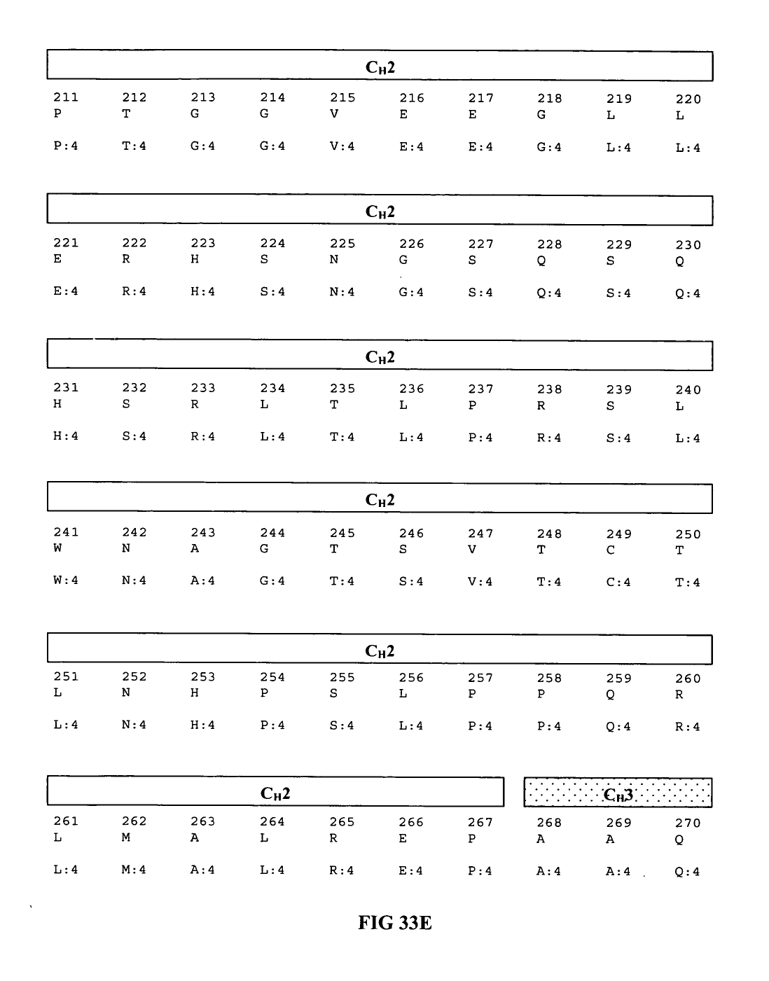

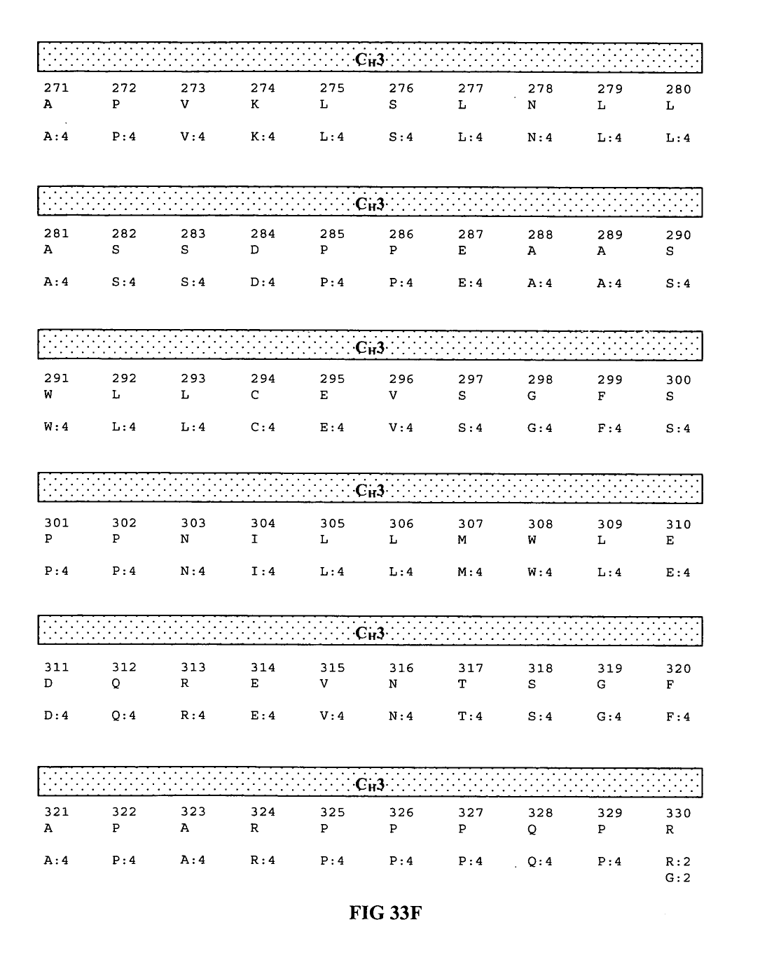

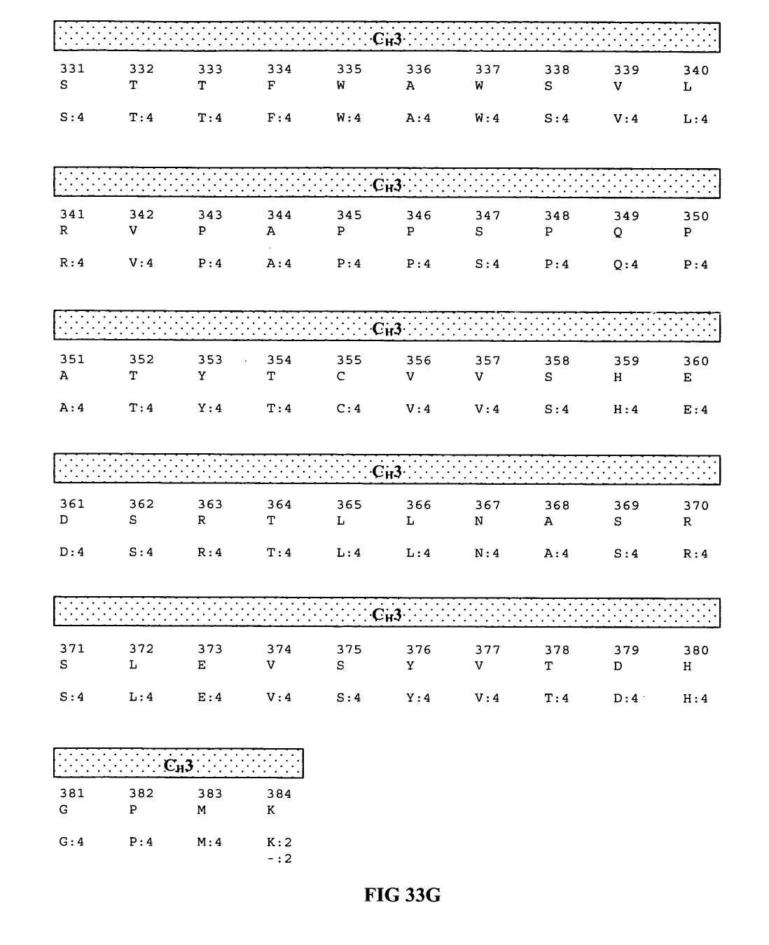

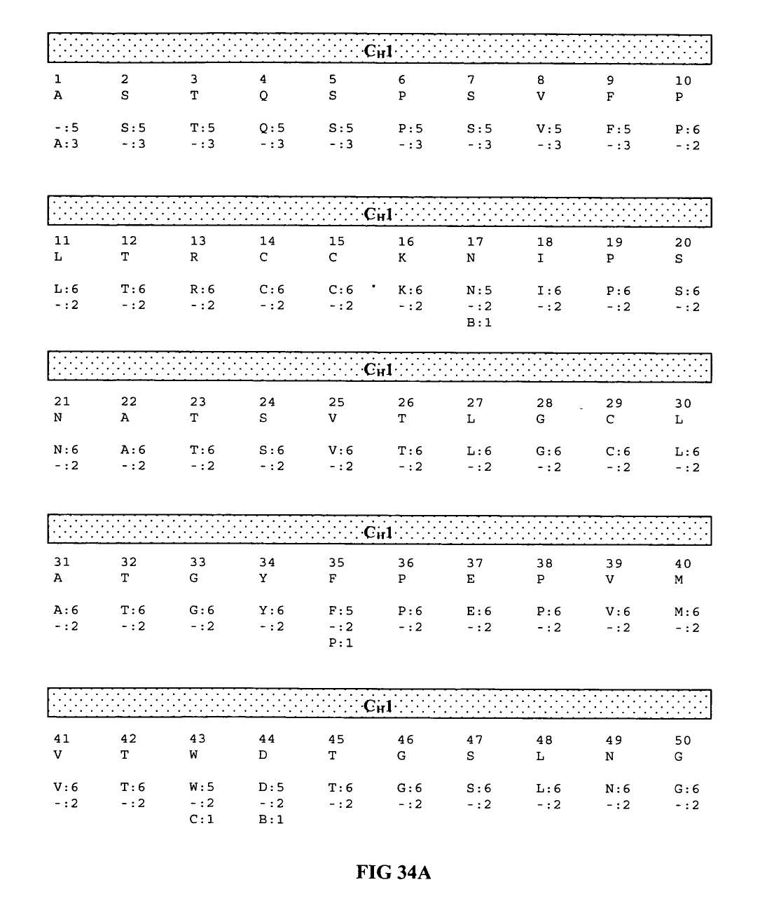

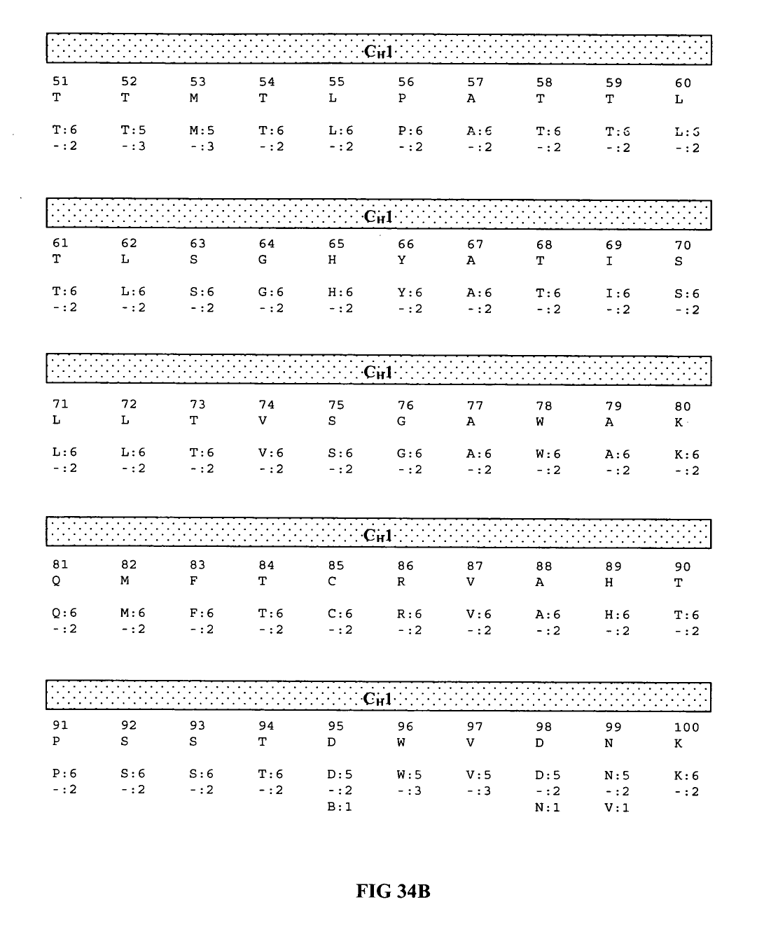

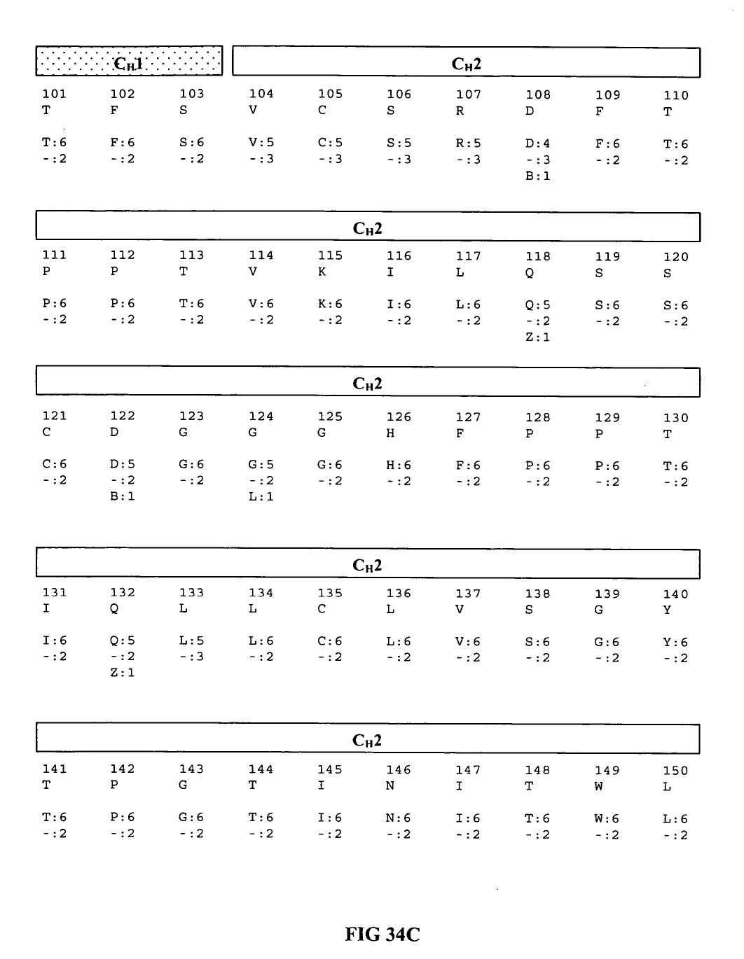

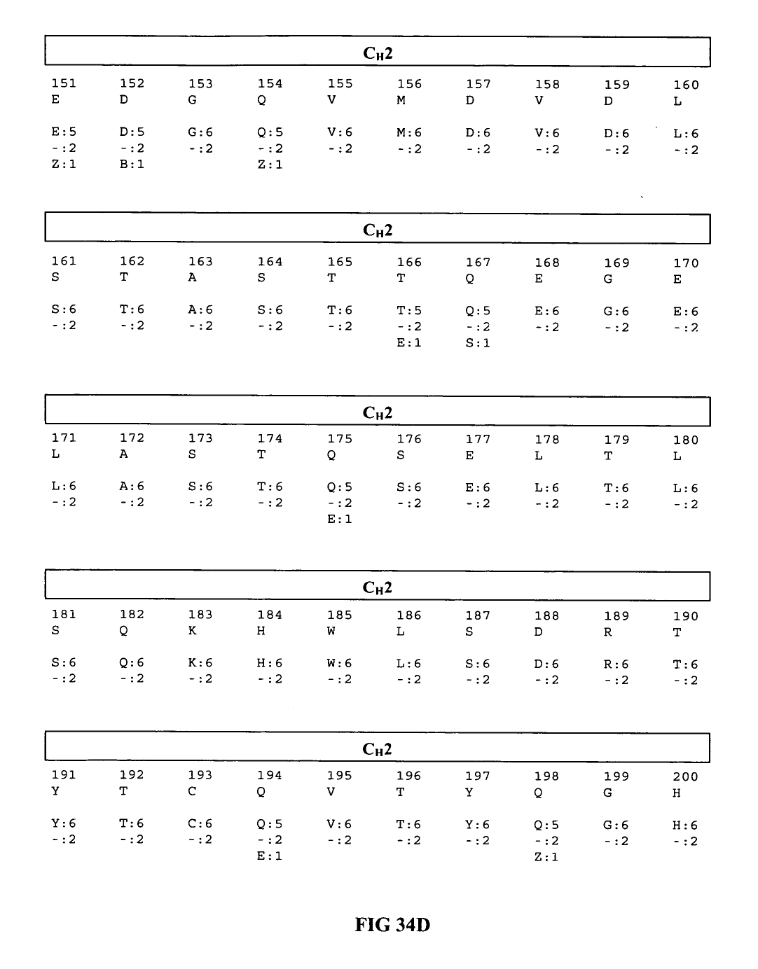

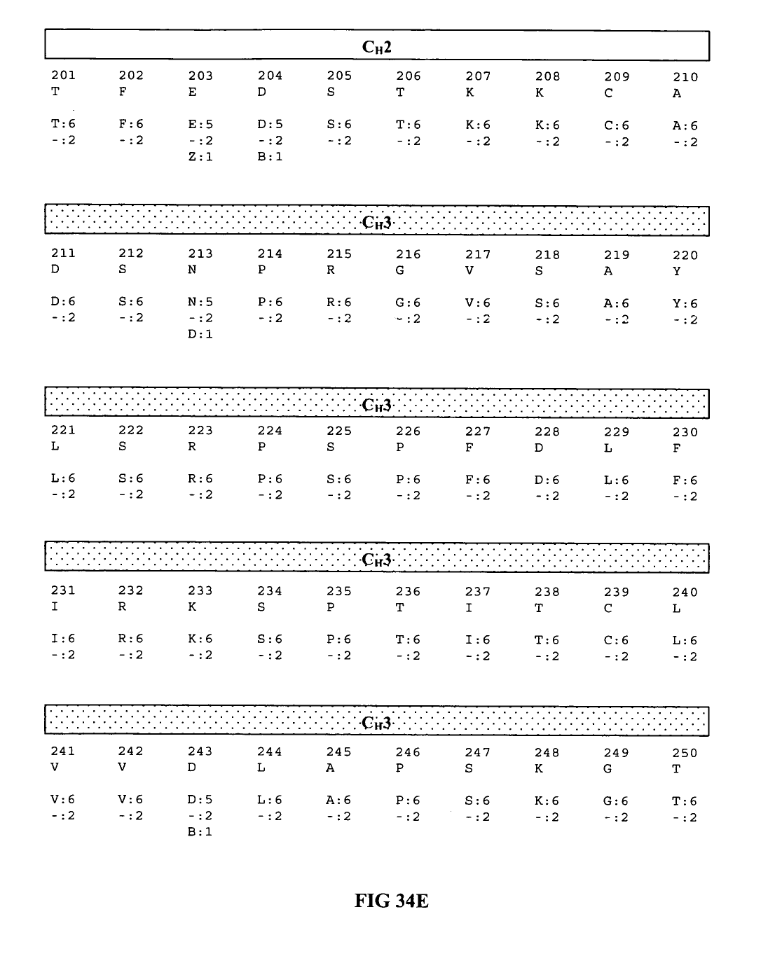

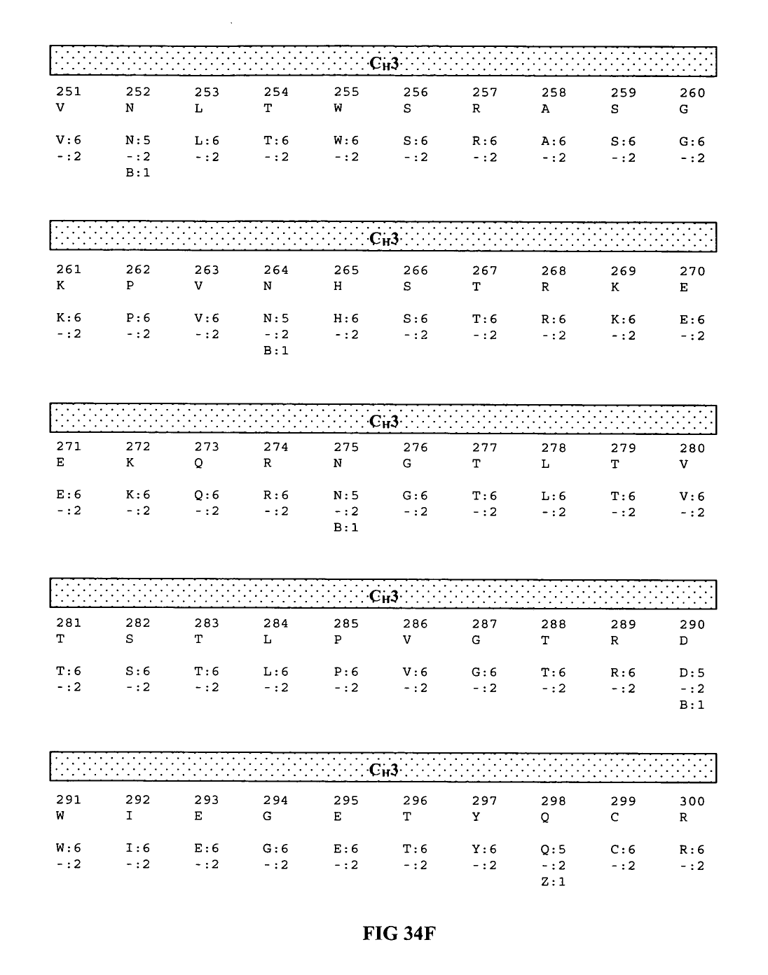

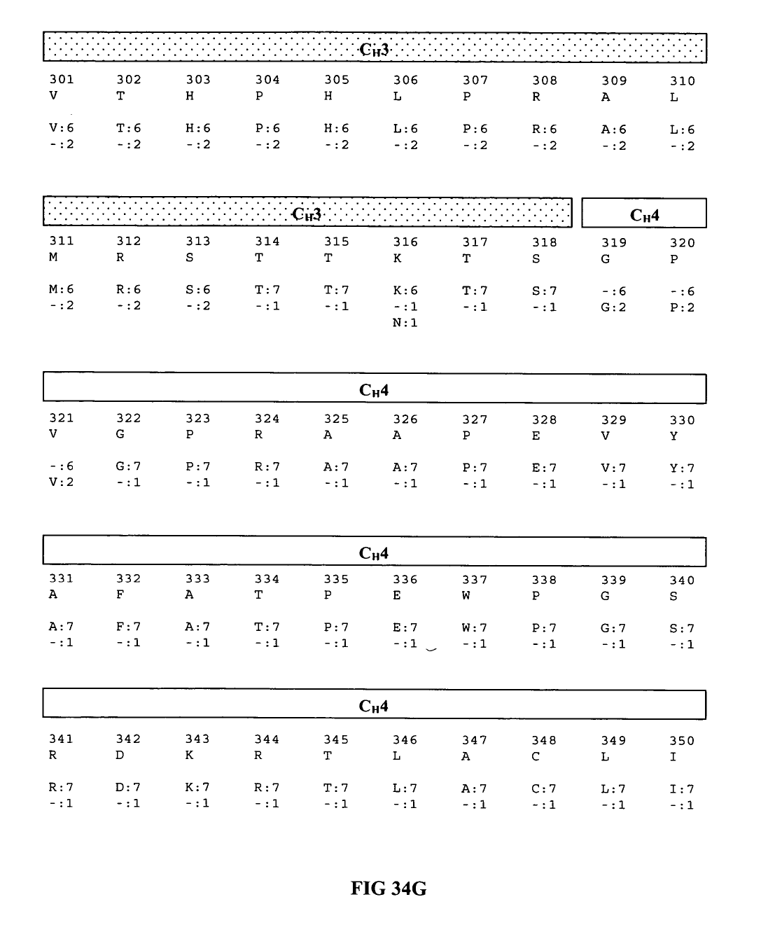

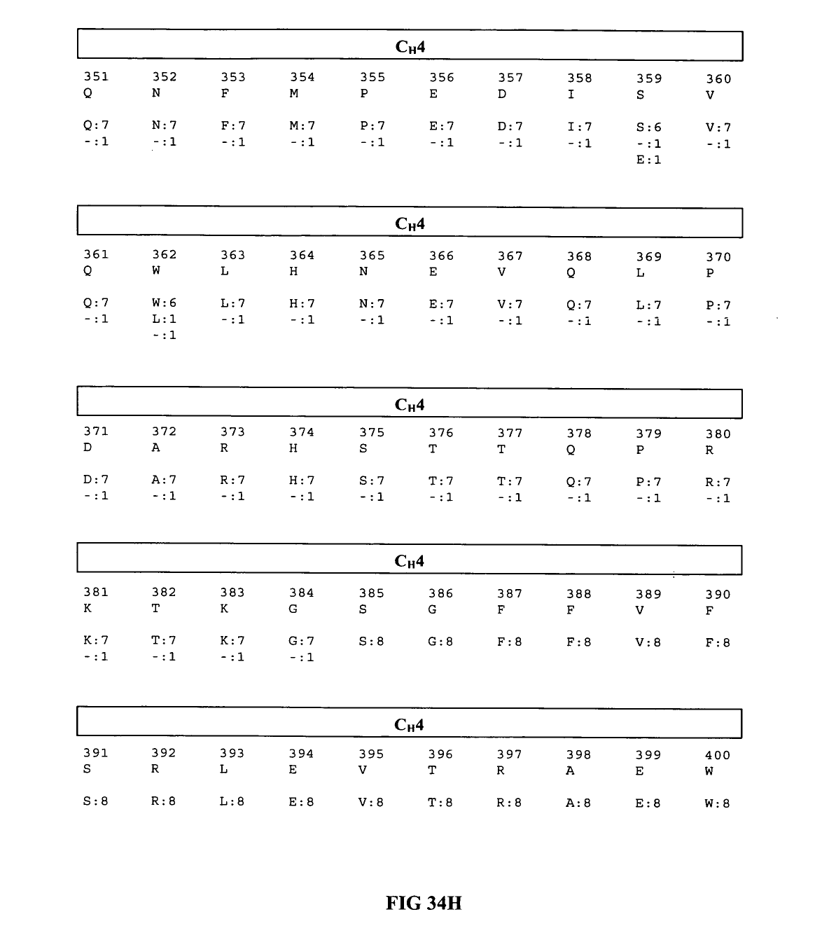

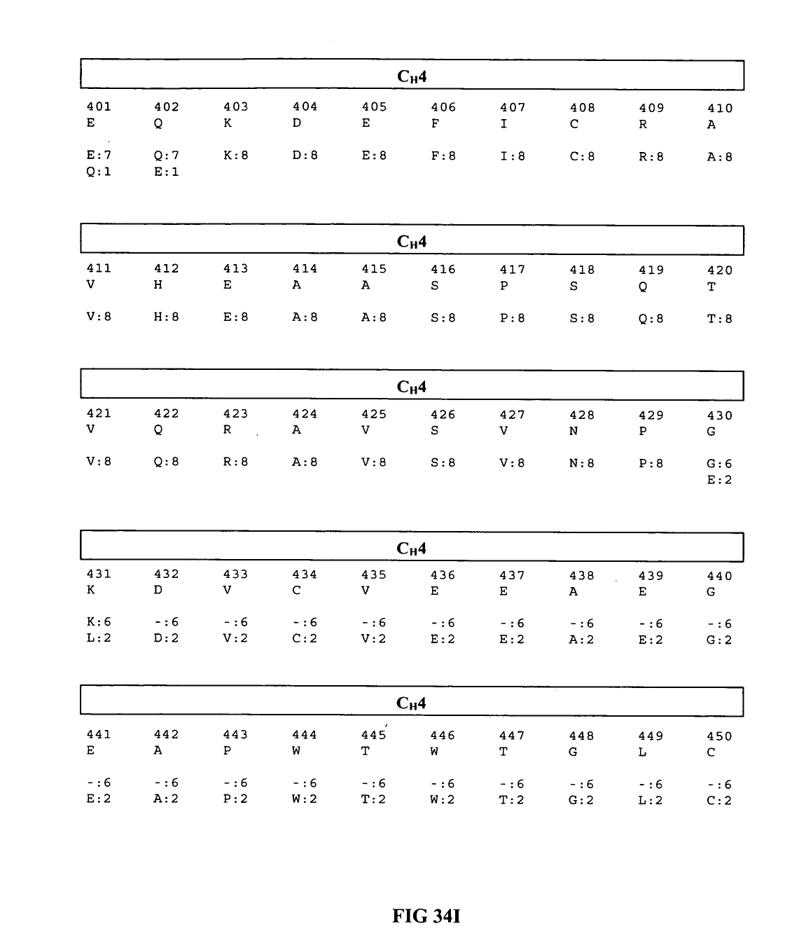

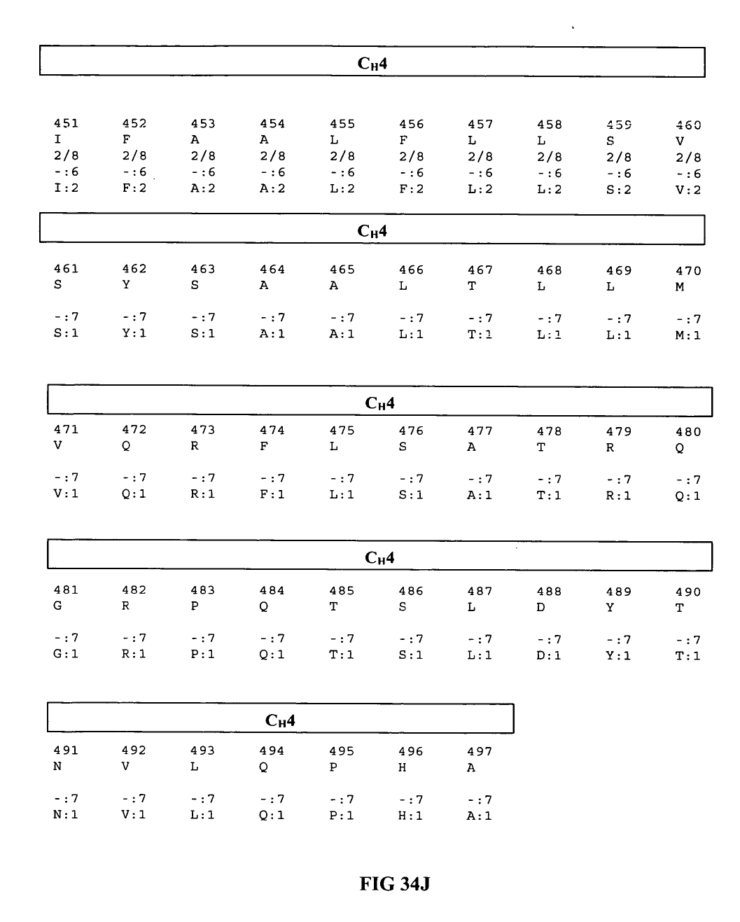

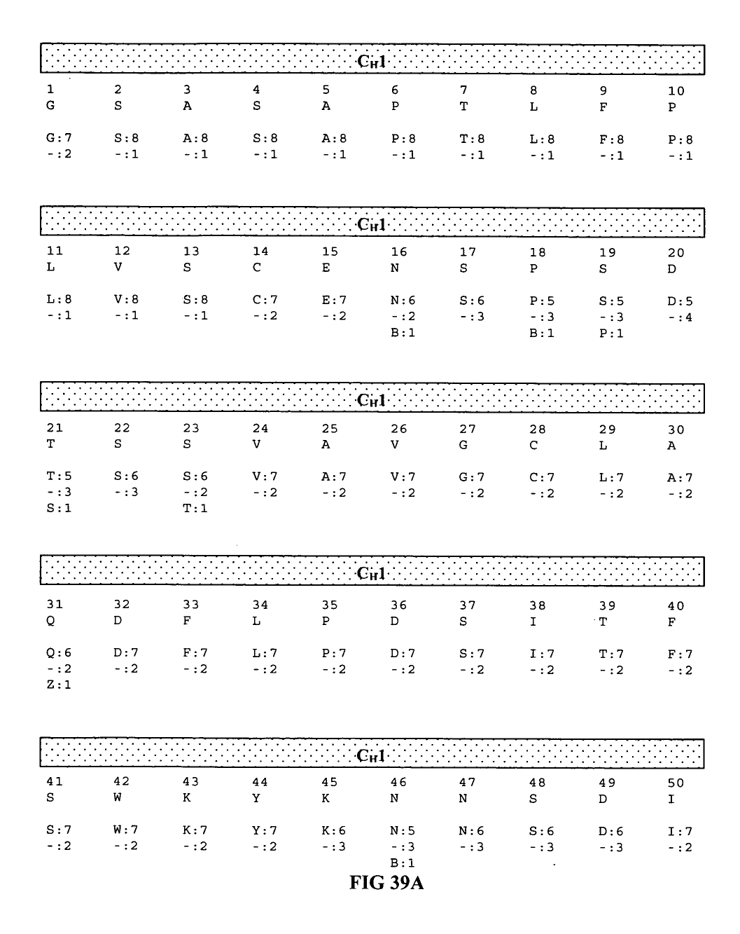

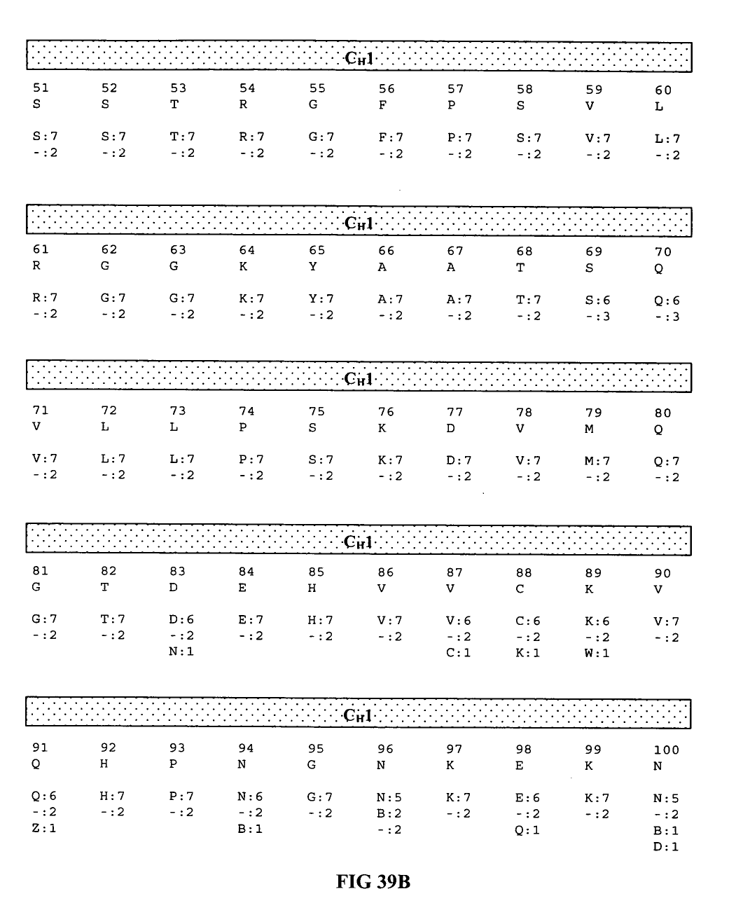

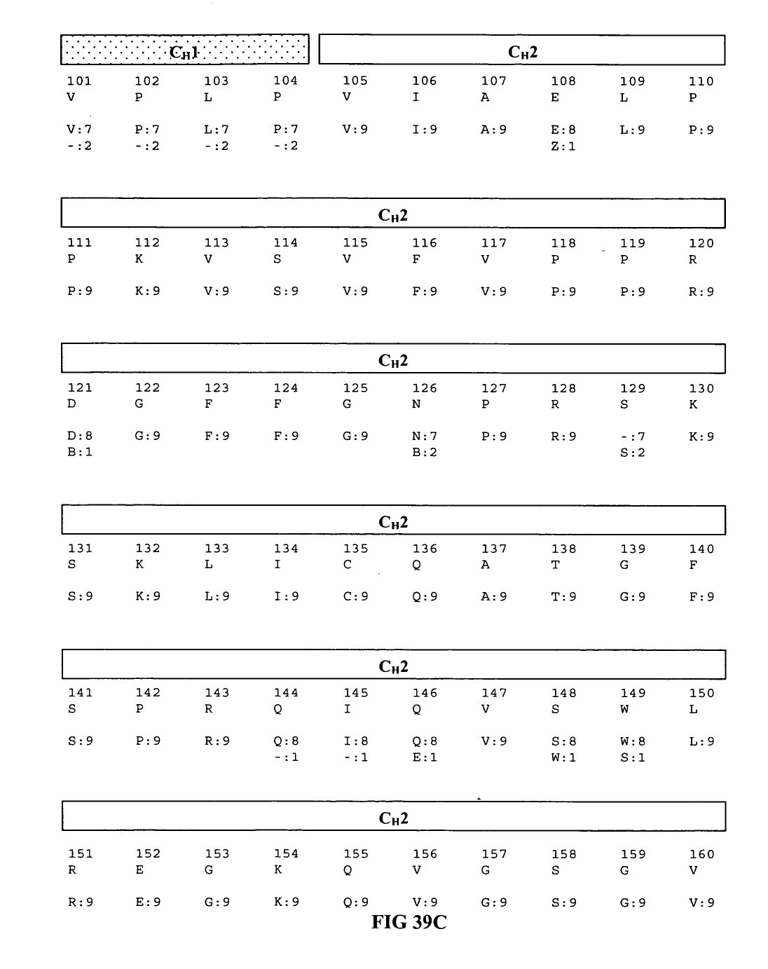

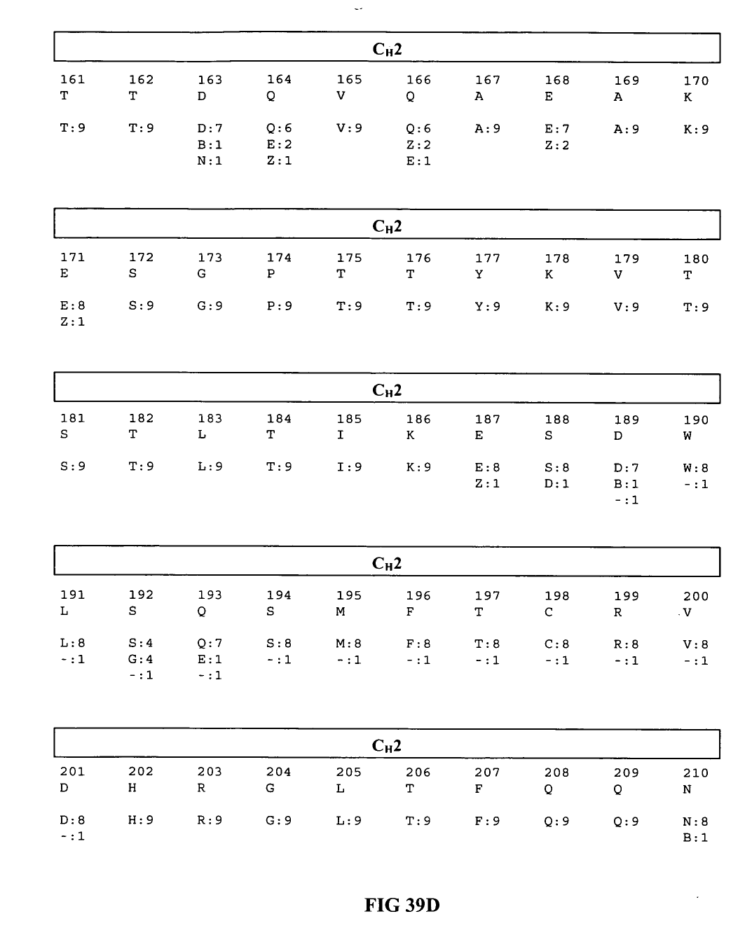

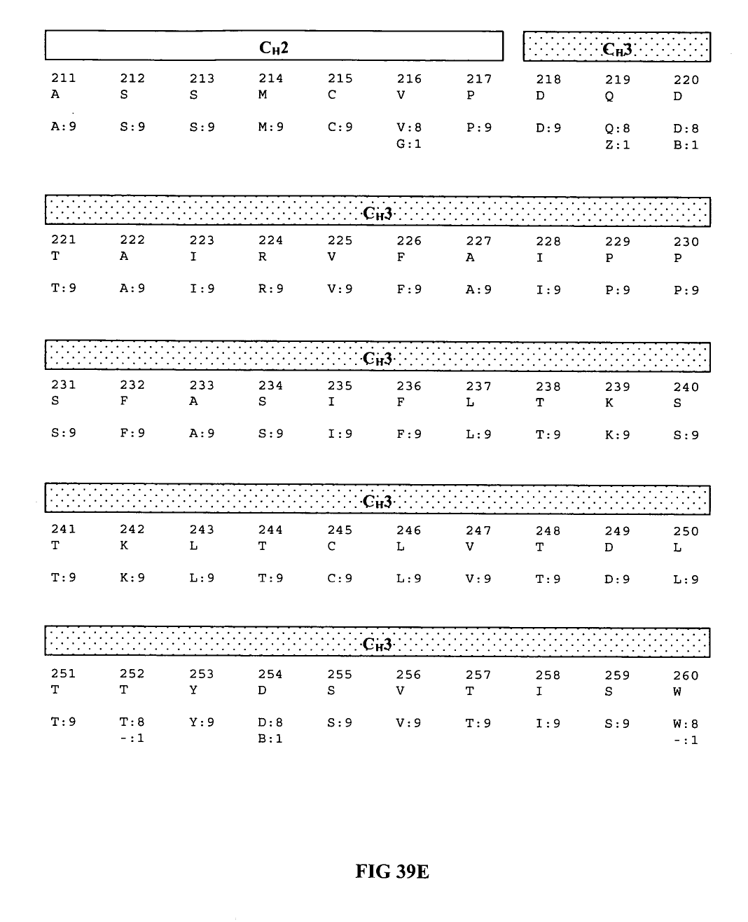

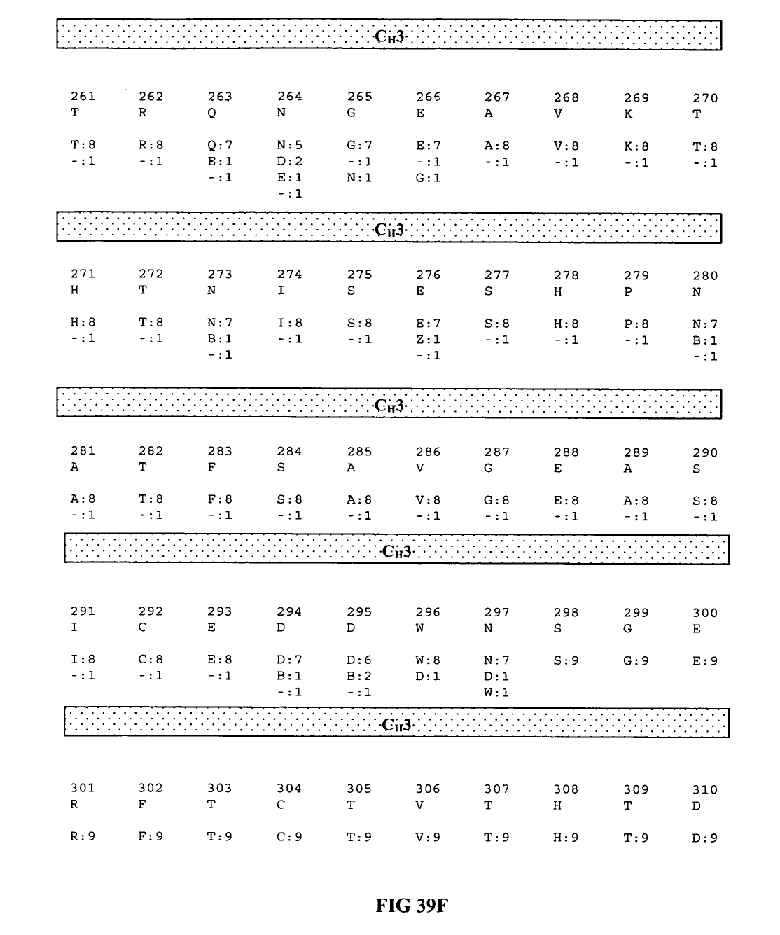

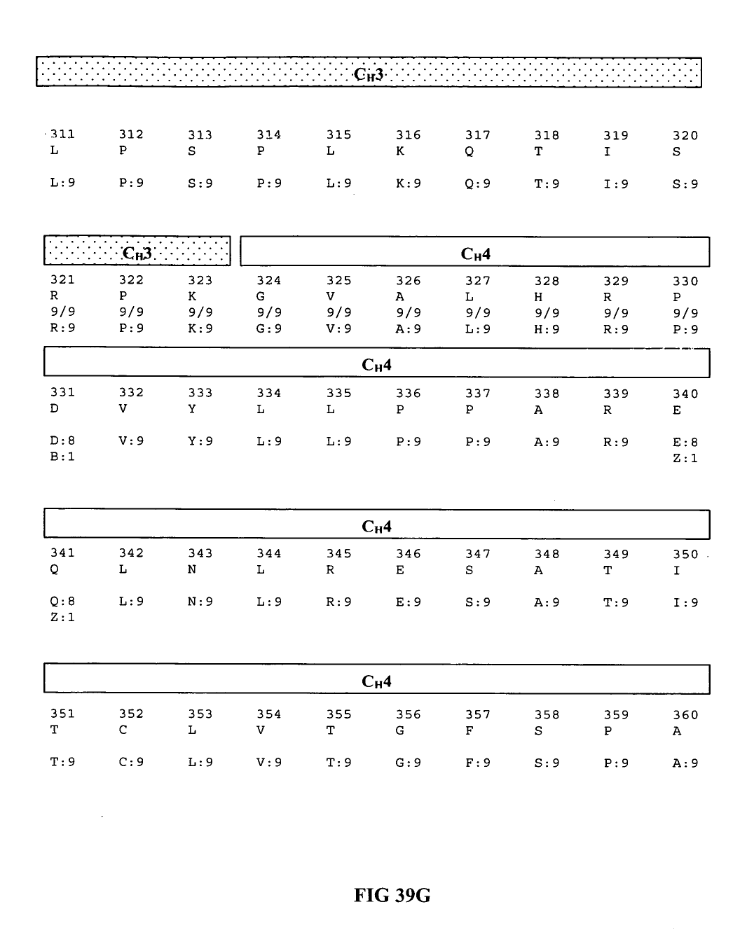

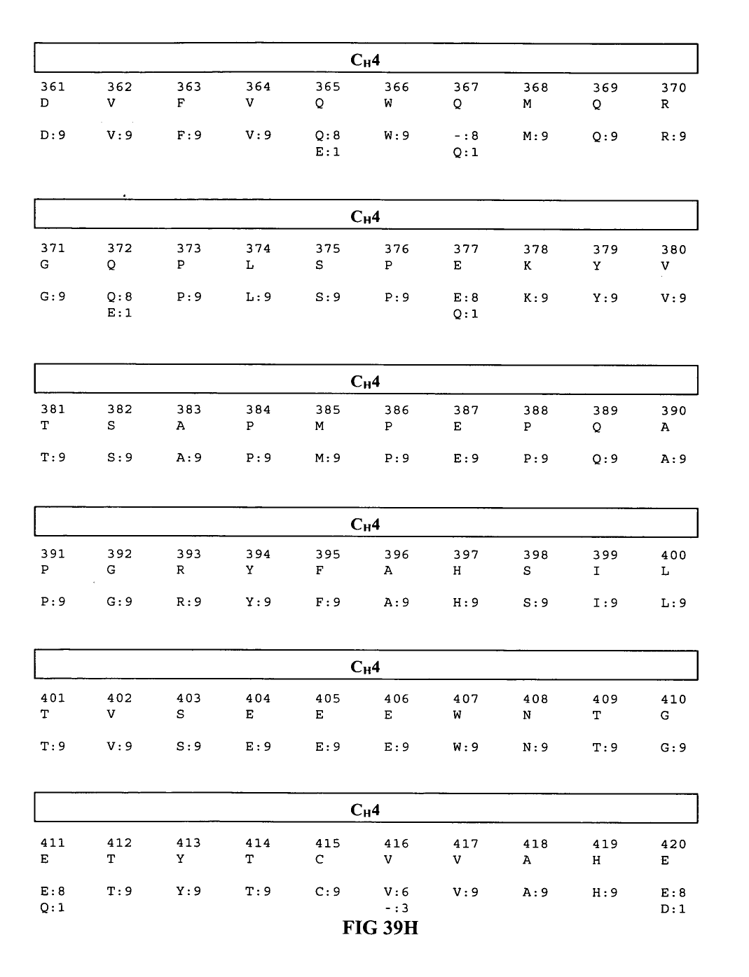

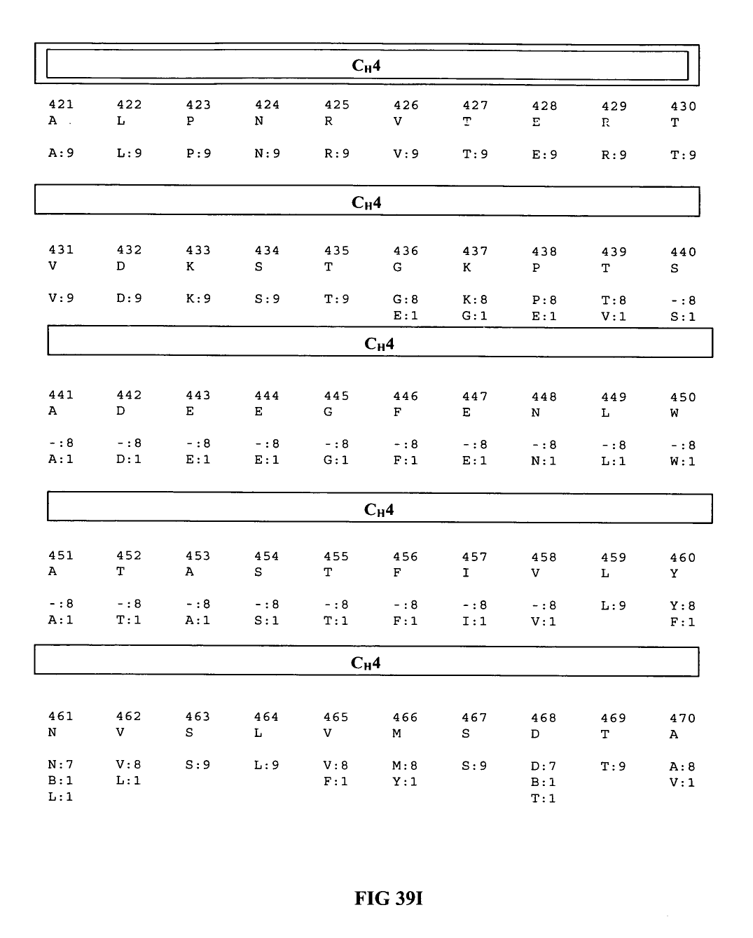

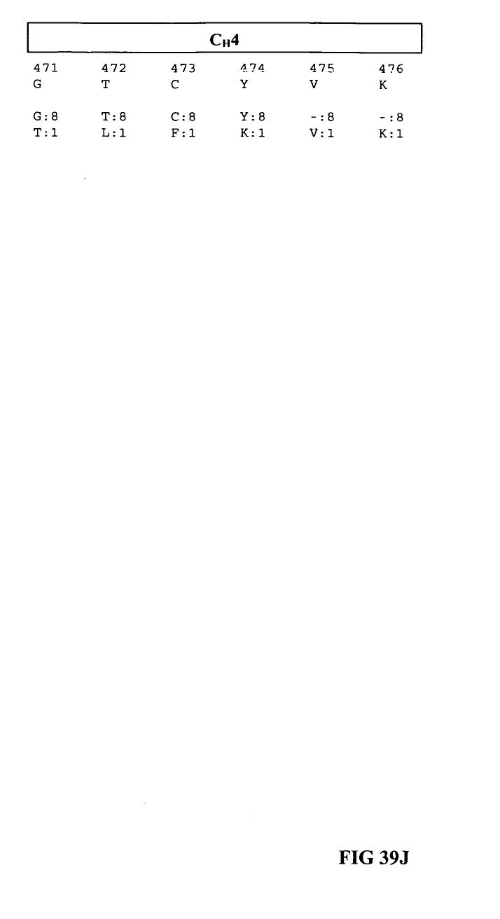

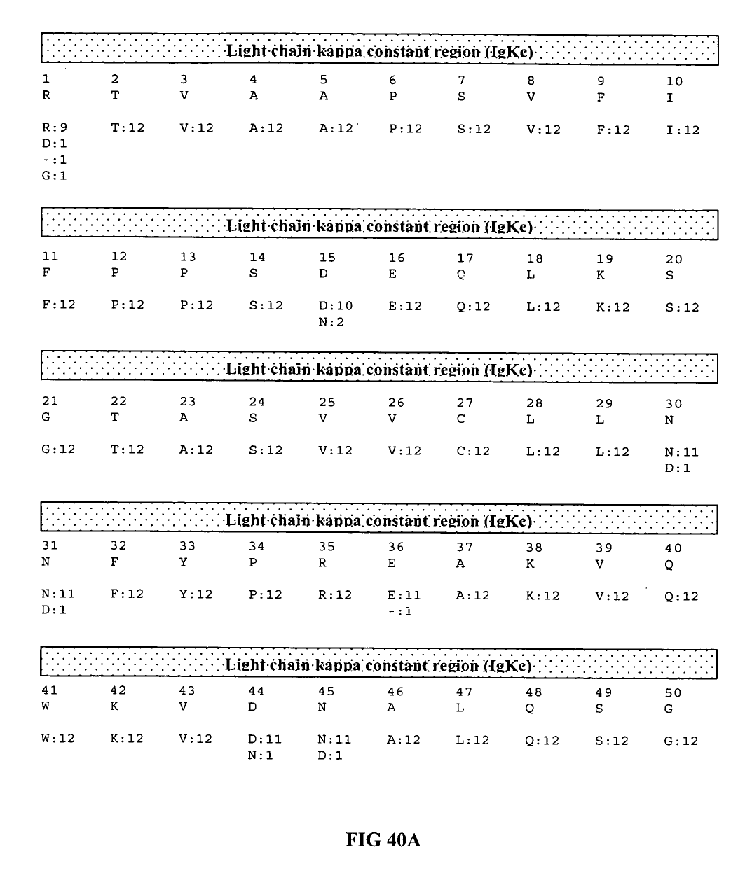

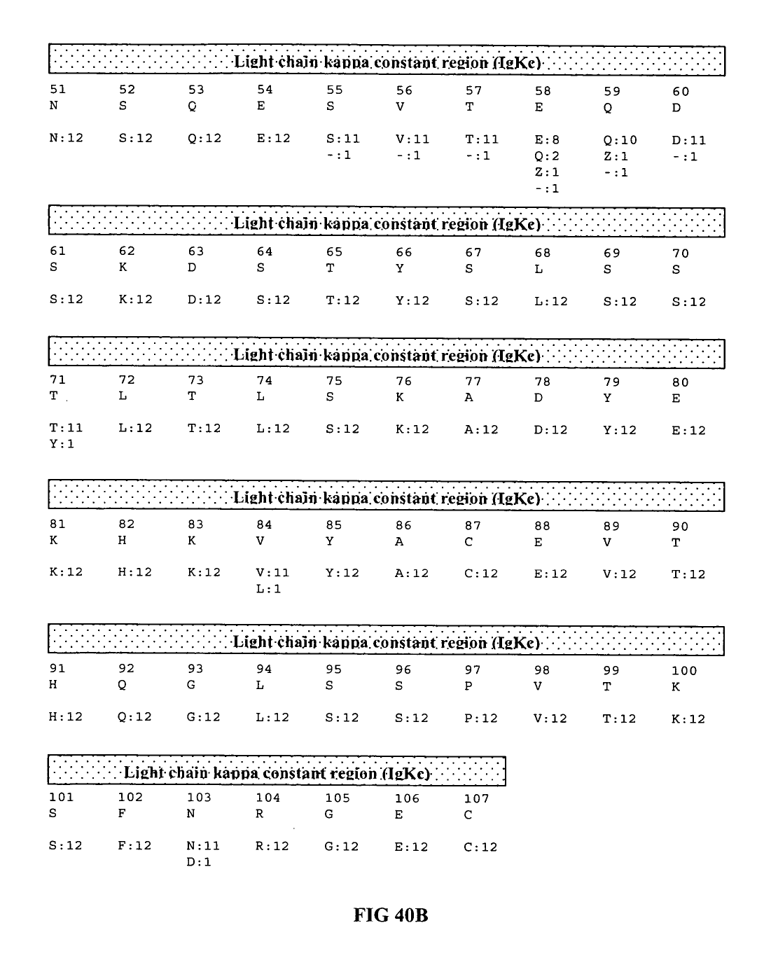

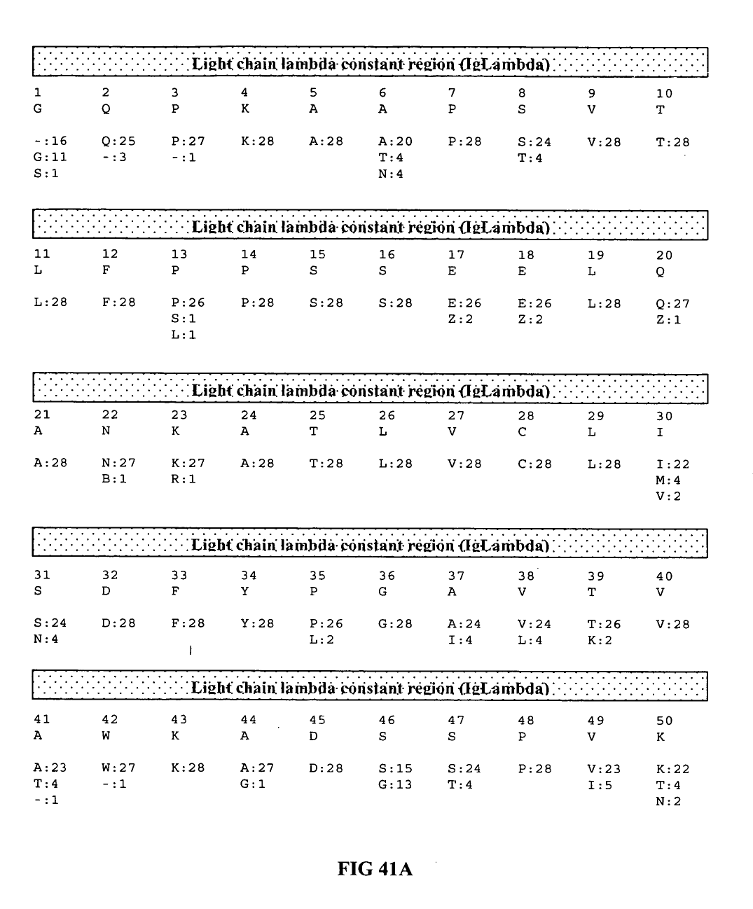

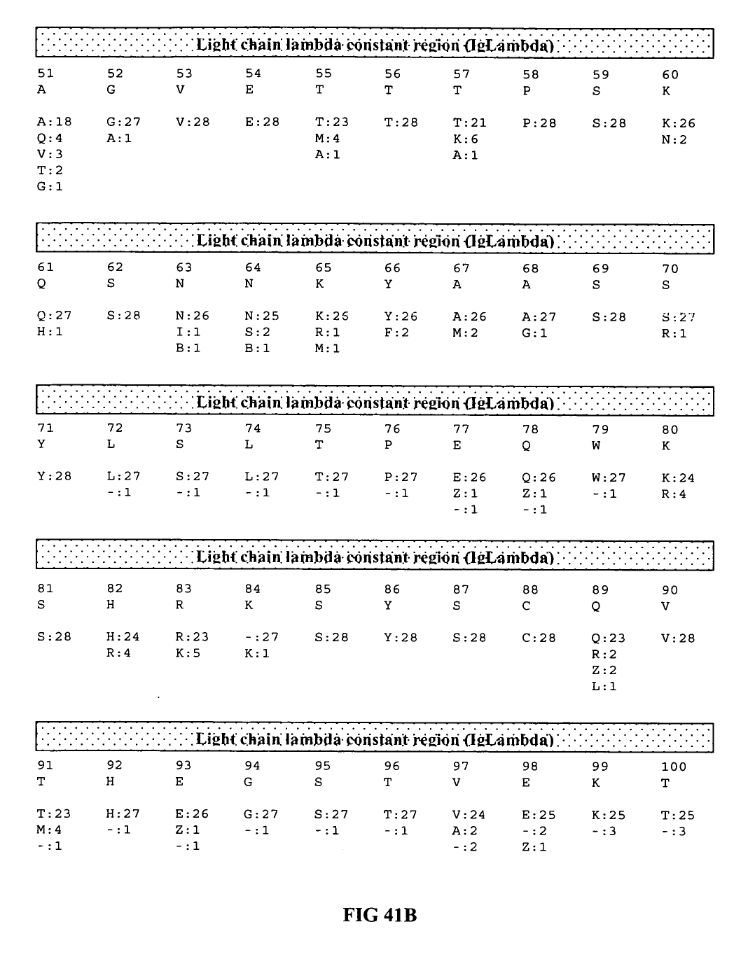

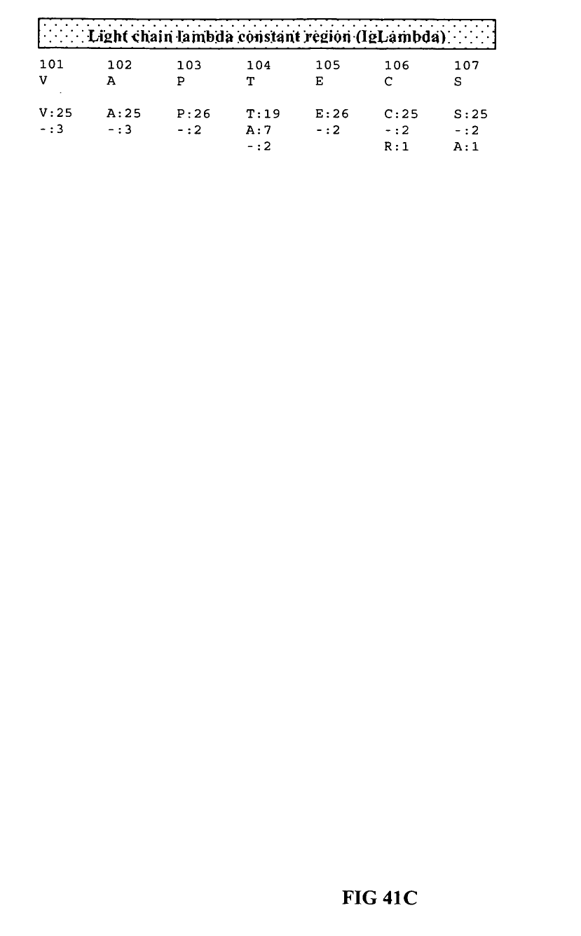

A hinge core mimetibody or specified portion or variant of the present invention can include one or more amino acid substitutions, deletions or additions, either from natural mutations or human manipulation, as specified herein. Such or other sequences that can be used in the present invention, include, but are not limited to the following sequences as presented in the following Table 23, as further detailed in FIGS. 1-41, with corresponding SEQ ID NOS 980-1020.

| SEQ | | | | | | | | | | |
|---|---|---|---|---|---|---|---|---|---|---|
| ID | | | AA | REGIONS | | | | | | |
| NO | | | NO | FR1 | CDR1 | FR2 | CDR2 | FR3 | CDR3 | FR4 |
| 980 | Heavy | Vh1 | 125 | 1-31 | 32 | 33-46 | 47 | 48-79 | 80 | 81-125 |
| 981 | chain | Vh2 | 97 | 1-30 | 31 | 32-45 | 46 | 47-78 | 79 | 80-97 |
| 982 | variable | Vh3a | 102 | 1-30 | 31 | 32-45 | 46 | 47-78 | 79 | 80-102 |
| 983 | region | Vh3b | 102 | 1-30 | 31 | 32-45 | 46 | 47-78 | 79 | 80-102 |
| 984 | | Vh3c | 94 | 1-30 | 31 | 32-45 | 46 | 47-78 | 79 | 80-94 |
| 985 | | Vh4 | 106 | 1-30 | 31 | 32-45 | 46 | 47-78 | 79 | 80-106 |
| 986 | | Vh5 | 97 | 1-30 | 31 | 32-45 | 46 | 47-78 | 79 | 80-97 |
| 987 | | Vh6 | 91 | 1-30 | 31 | 32-45 | 46 | 47-78 | 79 | 80-91 |
| 988 | | Vh7 | 91 | 1-30 | 31 | 32-45 | 46 | 47-78 | 79 | 80-91 |
| 989 | Light | κ1-4 | 73 | 1-23 | 24 | 25-39 | 40 | 41-72 | 73 | |
| 990 | chain | κ2 | 73 | 1-23 | 24 | 25-39 | 40 | 41-72 | 73 | |
| 991 | variable | κ3 | 73 | 1-23 | 24 | 25-39 | 40 | 41-72 | 73 | |
| 992 | region | κ5 | 73 | 1-23 | 24 | 25-39 | 40 | 41-72 | 73 | |
| 993 | | κ new1 | 67 | 1-17 | 18 | 19-33 | 34 | 35-66 | 67 | |
| 994 | | κ new2 | 65 | 1-15 | 16 | 17-31 | 32 | 33-64 | 65 | |
| 995 | | λ1a | 72 | 1-22 | 23 | 24-38 | 39 | 40-71 | 72 | |
| 996 | | λ1b | 73 | 1-23 | 24 | 25-39 | 40 | 41-72 | 73 | |
| 997 | | λ1c | 72 | 1-22 | 23 | 24-38 | 39 | 40-71 | 72 | |
| 998 | | λ3a | 72 | 1-22 | 23 | 24-38 | 39 | 40-71 | 72 | |
| 999 | | λ3b | 72 | 1-22 | 23 | 24-38 | 39 | 40-71 | 72 | |
| 1000 | | λ3c | 72 | 1-22 | 23 | 24-38 | 39 | 40-71 | 72 | |
| 1001 | | λ3e | 72 | 1-22 | 23 | 24-38 | 39 | 40-71 | 72 | |
| 1002 | | λ4a | 72 | 1-22 | 23 | 24-38 | 39 | 40-71 | 72 | |
| 1003 | | λ4b | 72 | 1-22 | 23 | 24-38 | 39 | 40-71 | 72 | |
| 1004 | | λ5 | 75 | 1-22 | 23 | 24-39 | 40 | 41-74 | 75 | |
| 1005 | | λ6 | 74 | 1-22 | 23 | 24-38 | 39 | 40-73 | 74 | |
| 1006 | | λ7 | 72 | 1-22 | 23 | 24-38 | 39 | 40-71 | 72 | |
| 1007 | | λ8 | 72 | 1-22 | 23 | 24-38 | 39 | 40-71 | 72 | |
| 1008 | | λ9 | 72 | 1-22 | 23 | 24-38 | 39 | 40-71 | 72 | |
| 1009 | | λ10 | 72 | 1-22 | 23 | 24-38 | 39 | 40-71 | 72 | |
| SEQ | | | | | | | | | | |
| ID | | | AA | REGIONS | | | | | | |
| NO | | | NO | CH1 | hinge1 | hinge2 | hinge3 | hinge4 | CH2 | CH3 |
| 1010 | Heavy | IgA1 | 354 | 1-102 | 103-122 | | | | 123-222 | 223-354 |
| 1011 | chain | IgA2 | 340 | 1-102 | 103-108 | | | | 109-209 | 210-340 |
| 1012 | constant | IgD | 384 | 1-101 | 102-135 | 136-159 | | | 160-267 | 268-384 |
| 1013 | region | IgE | 497 | 1-103 | | | | | 104-210 | 211-318 |
| 1014 | | IgG1 | 339 | 1-98 | 99-113 | | | | 114-223 | 224-339 |
| 1015 | | IgG2 | 326 | 1-98 | 99-110 | | | | 111-219 | 220-326 |
| 1016 | | IgG3 | 377 | 1-98 | 99-115 | 116-130 | 131-145 | 146-160 | 161-270 | 271-377 |
| 1017 | | IgG4 | 327 | 1-98 | 99-110 | | | | 111-220 | 221-327 |
| 1018 | | IgM | 476 | 1-104 | | | | | 105-217 | 218-323 |
| 1019 | Light | Igκc | 107 | | | | | | | |
| 1020 | chain | Igλc | 107 | | | | | | | |
| | constant | | | | | | | | | |
| | region | | | | | | | | | |

Of course, the number of amino acid substitutions a skilled artisan would make depends on many factors, including those described above. Generally speaking, the number of amino acid substitutions, insertions or deletions for at least one of a hinge core mimetibody or fragment, e.g., but not limited to, at least one variable, constant, light or heavy chain, or Ig will not be more than 40, 30, 20, 19, 18, 17, 16, 15, 14, 13, 12, 11, 10, 9, 8, 7, 6, 5, 4, 3, 2, 1 amino acids, such as 1-30 or any range or value therein, as specified herein.

The following description of the components of a hinge core mimetibody of the present invention is based on the use of the formula I of the present invention,
((V(m)-P(n)-L(o)-H(p)-CH2(q)-CH3(r))(s),

where V is at least one portion of an N-terminus of an immunoglobulin variable region, P is at least one bioactive peptide, L is at least one linker polypeptide H is at least one portion of at least one immunoglobulin hinge region, CH2 is at least a portion of an immunoglobulin CH2 constant region, CH3 is at least a portion of an immunoglobulin CH3 constant region, m, n, o, p, q, r and s are independently an integer between 0, 1 or 2 and 10, mimicing different types of immunoglobulin molecules, e.g., but not limited to IgG1, IgG2, IgG3, IgG4, IgA, IgM, IgD, IgE, and the like, or any subclass thereof, or any combination thereof.

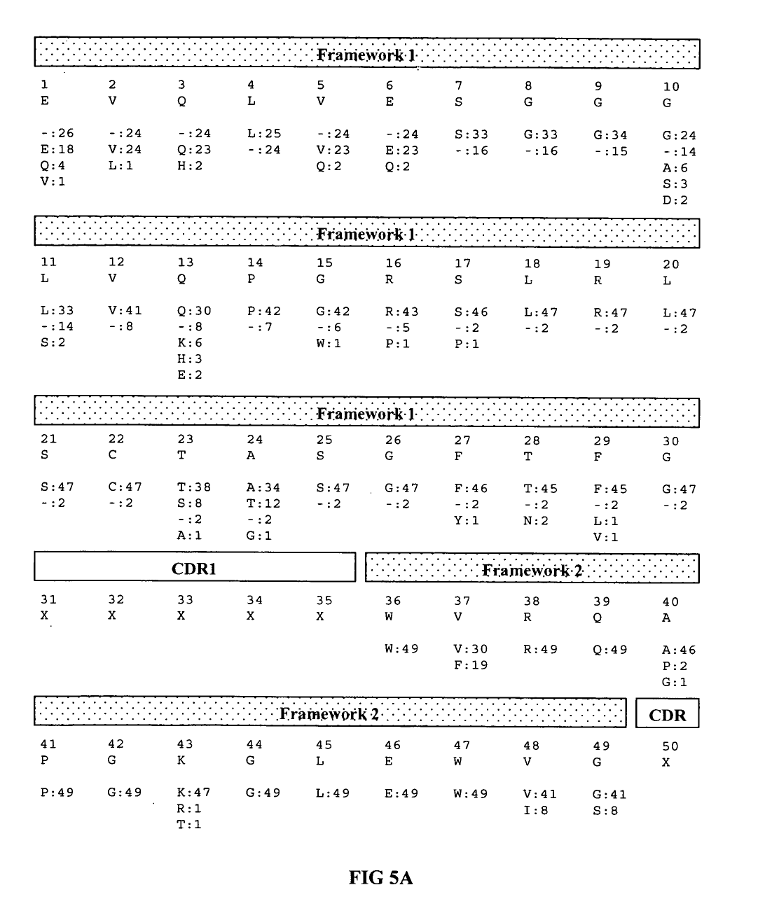

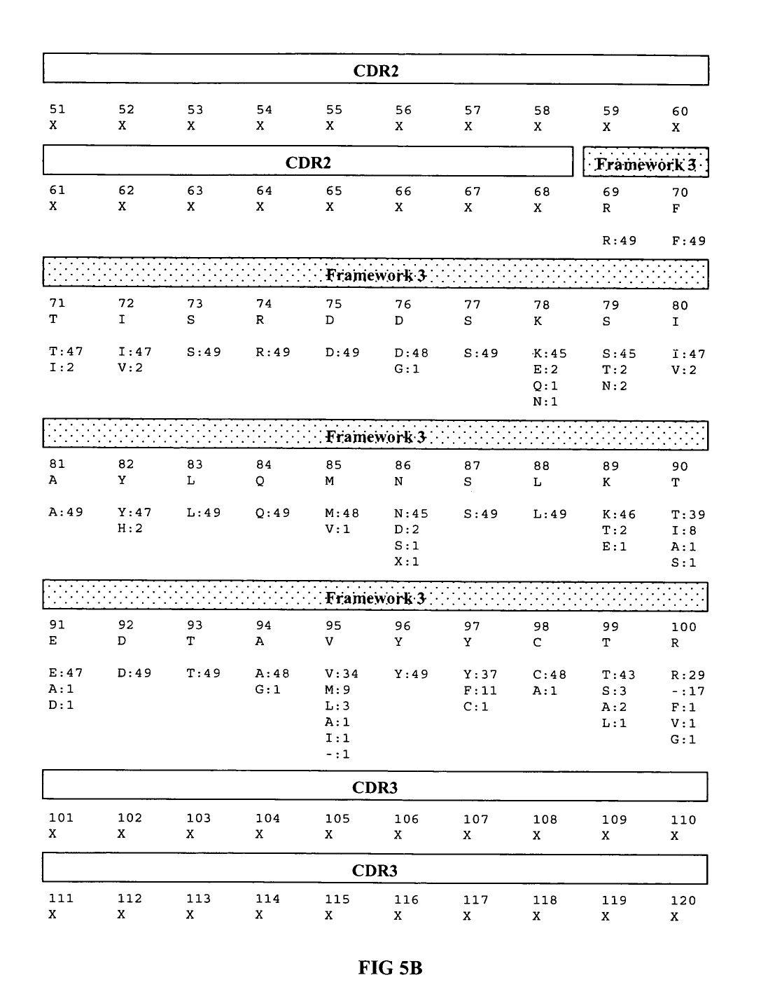

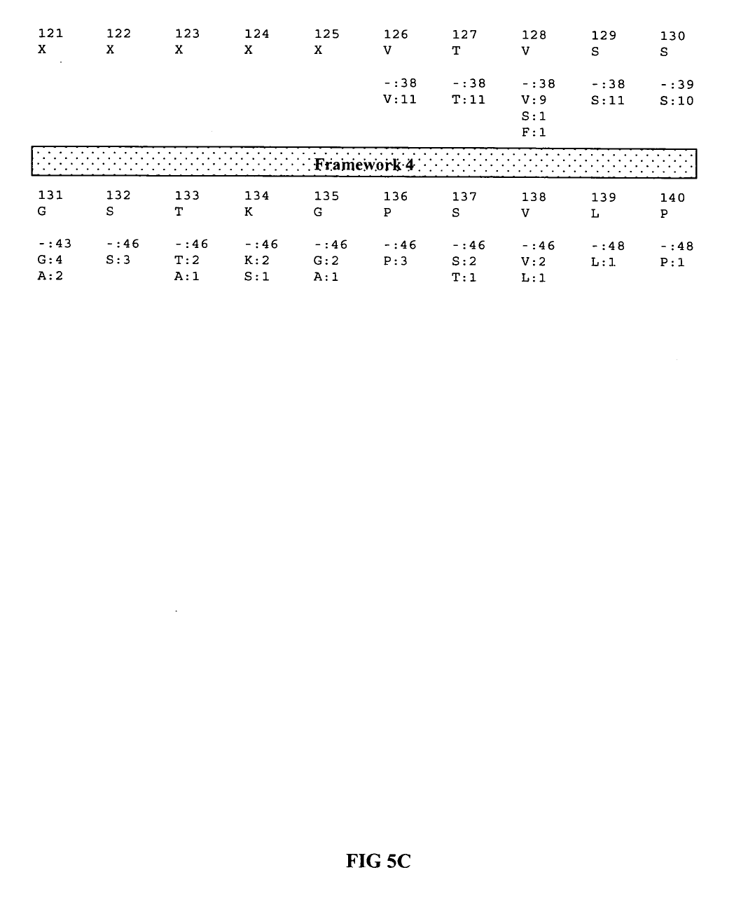

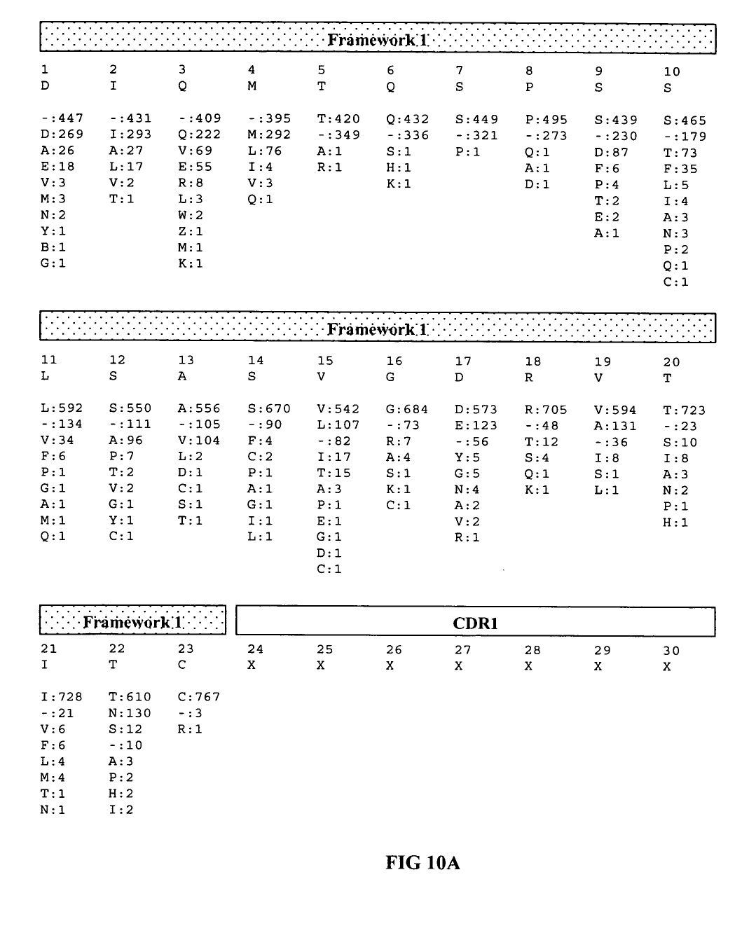

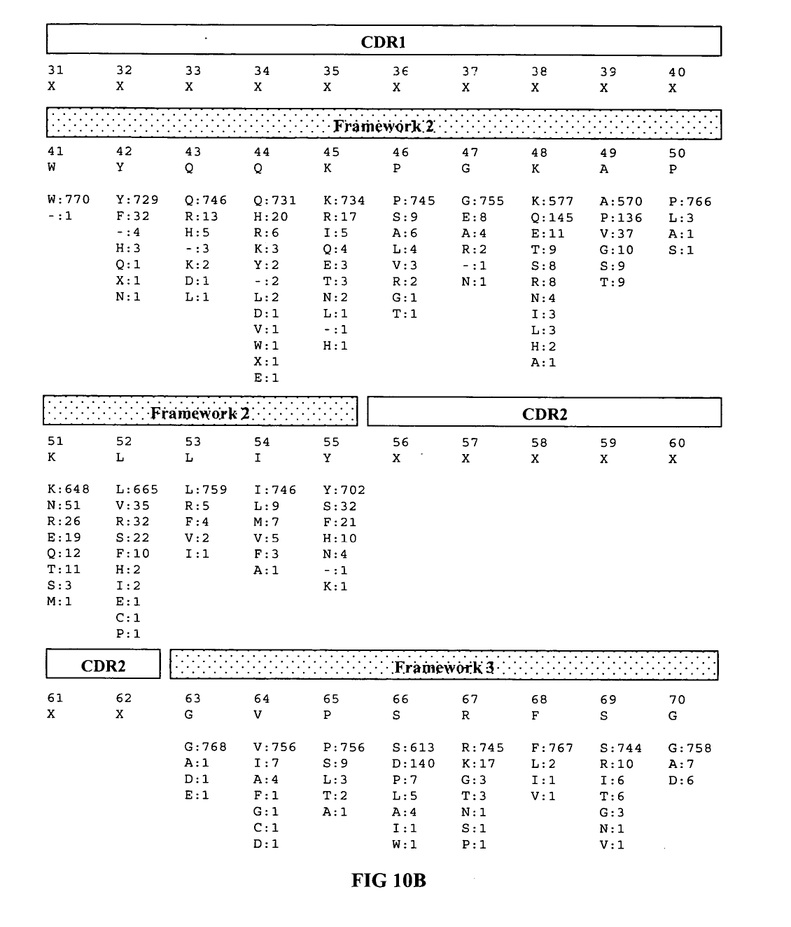

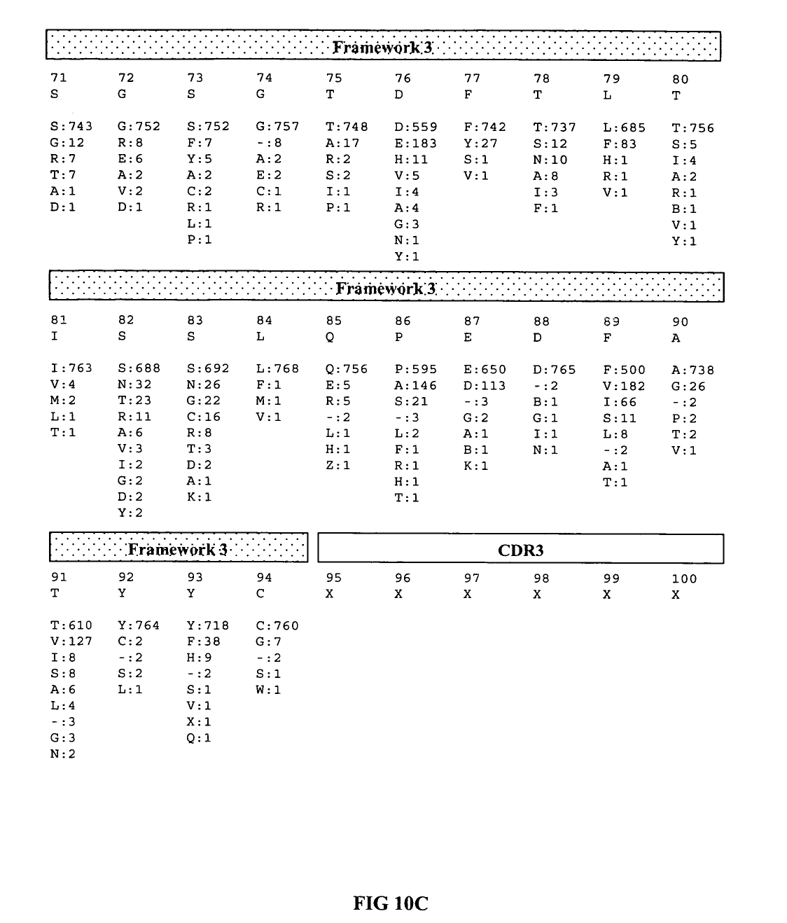

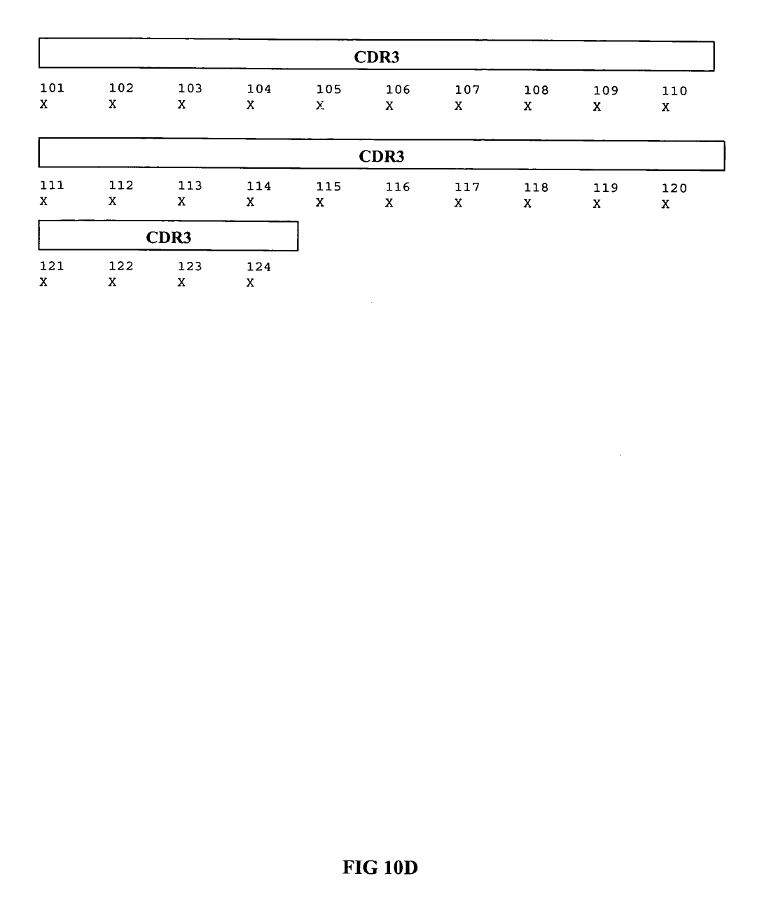

In hinge core mimetibodies of the present invention, the optional N-terminal V portion (V) can comprise 1-20 amino acids of at least one heavy chain variable framework 1 (FR1) region, e.g., as presented in FIGS. 1-9 (SEQ ID NOS:980-988) or at least one LC variable region, e.g., as presented in FIGS. 10-30 (SEQ ID NOS:989-1009), including substitutions, deletions or insertions as presented in these Figures, with those of FIGS. 5, 6, and 8 preferred. Also preferred are variable sequences that comprise the sequence Q-X-Q.

The P portion can comprise at least one any therapeutic peptide as known in the art or as described herein, such as, but not limited to those presented in Tables 1-21, SEQ ID NOS:1-979, or as known in the art, or any combination or consensus sequence thereof, or any fusion protein thereof.

The optional linker sequence can be any suitable peptide linker as known in the art. Preferred sequence include any combination of G and S, e.g., X1-X2-X3-X4-Xn, where X can be G or S, and n can be 5-30. Non-limiting examples include, GS, GGGS, GSGGGS, GSGGGSGG, and the like.

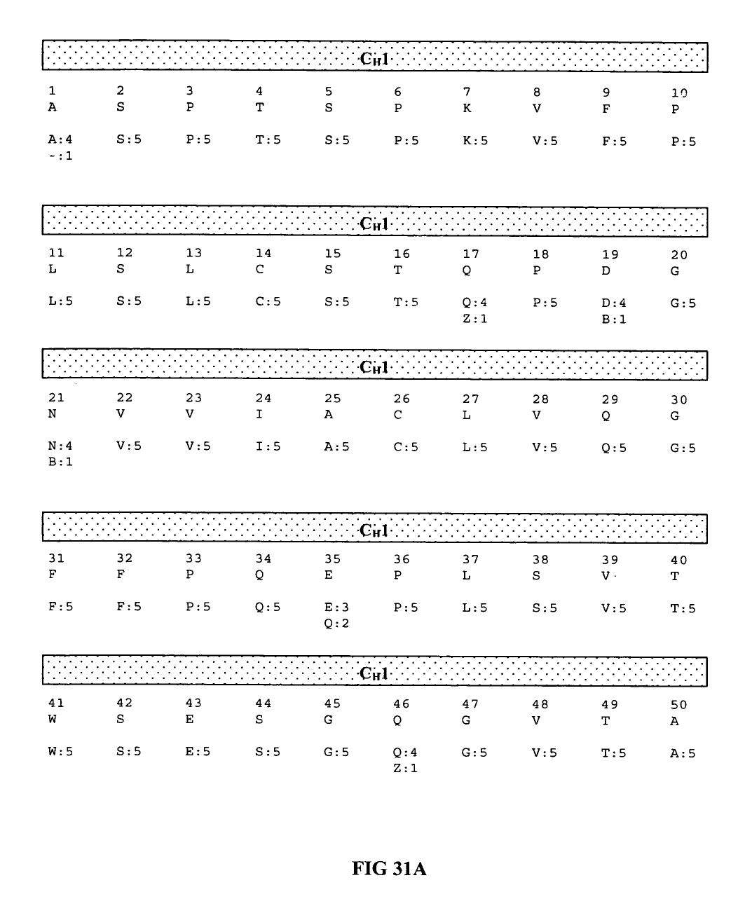

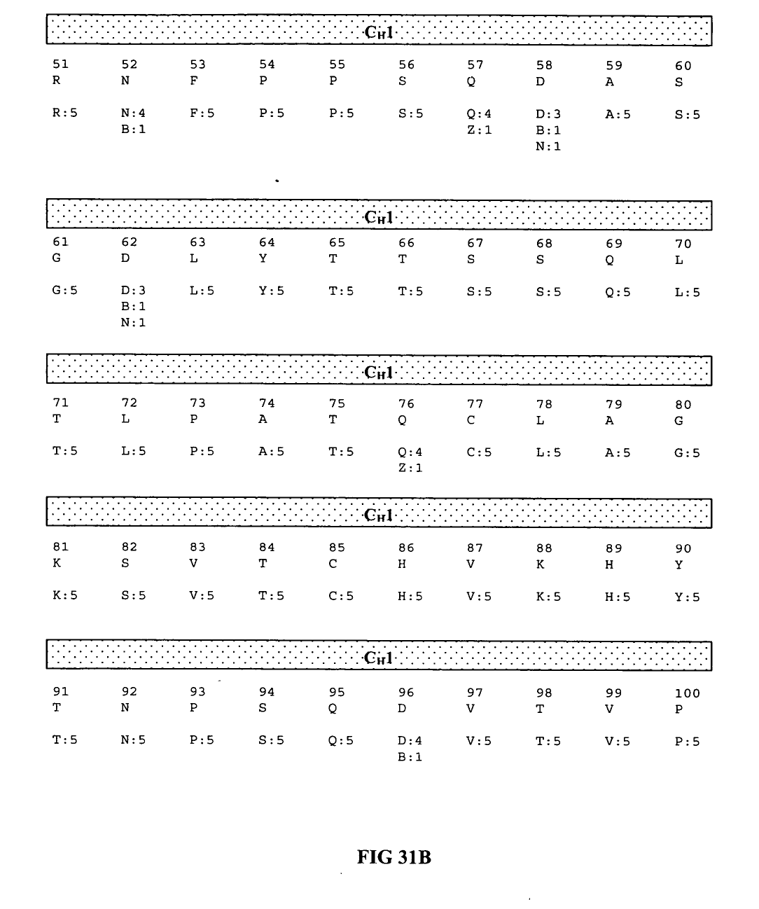

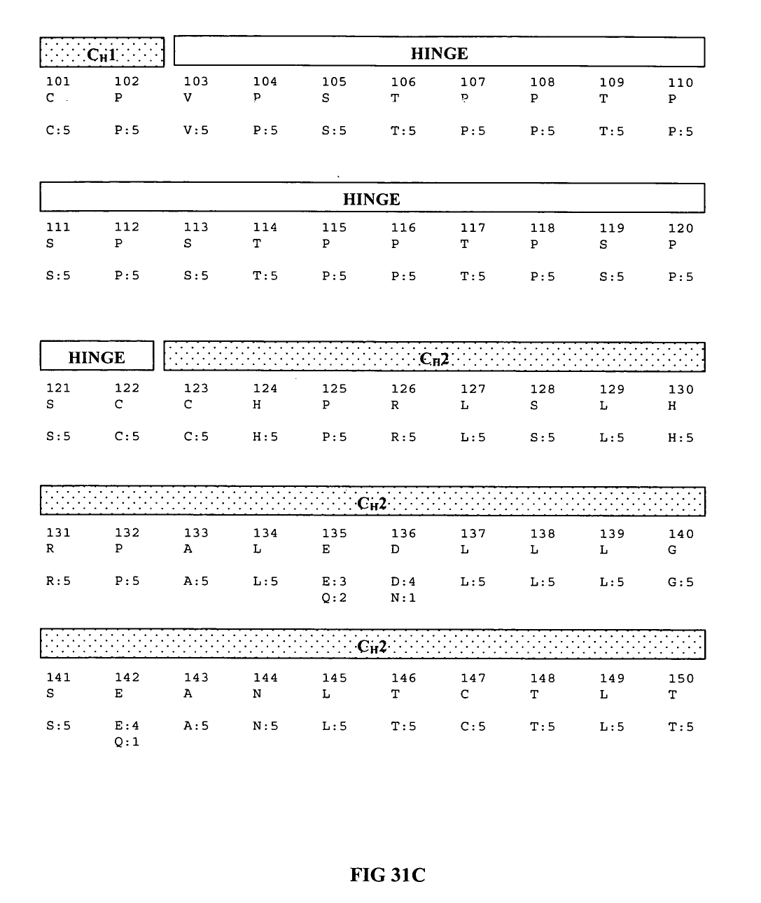

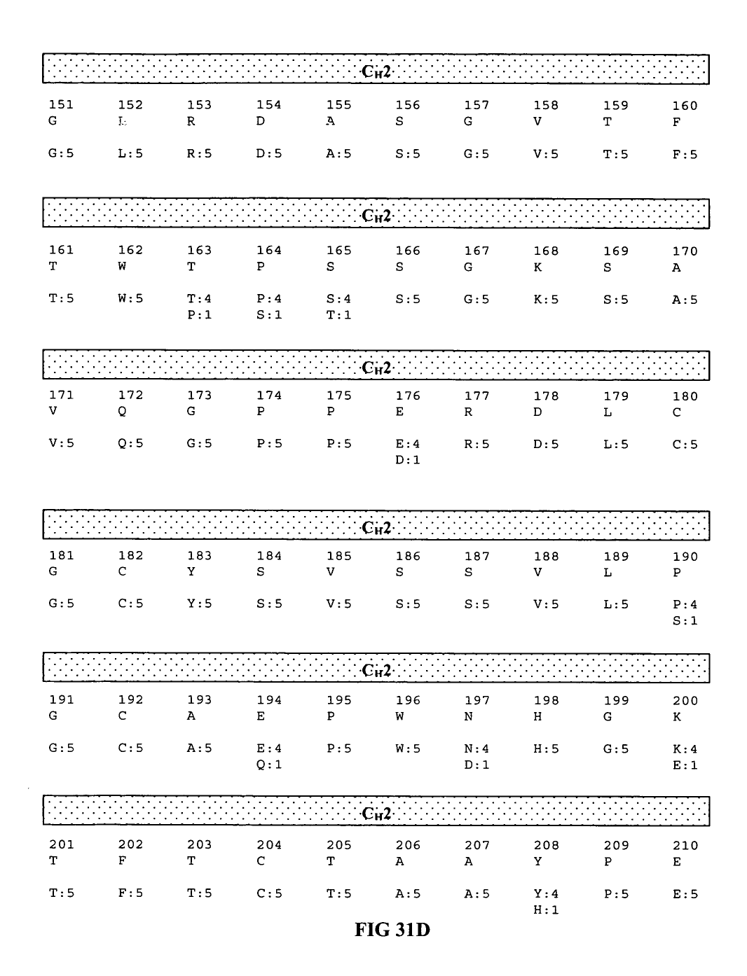

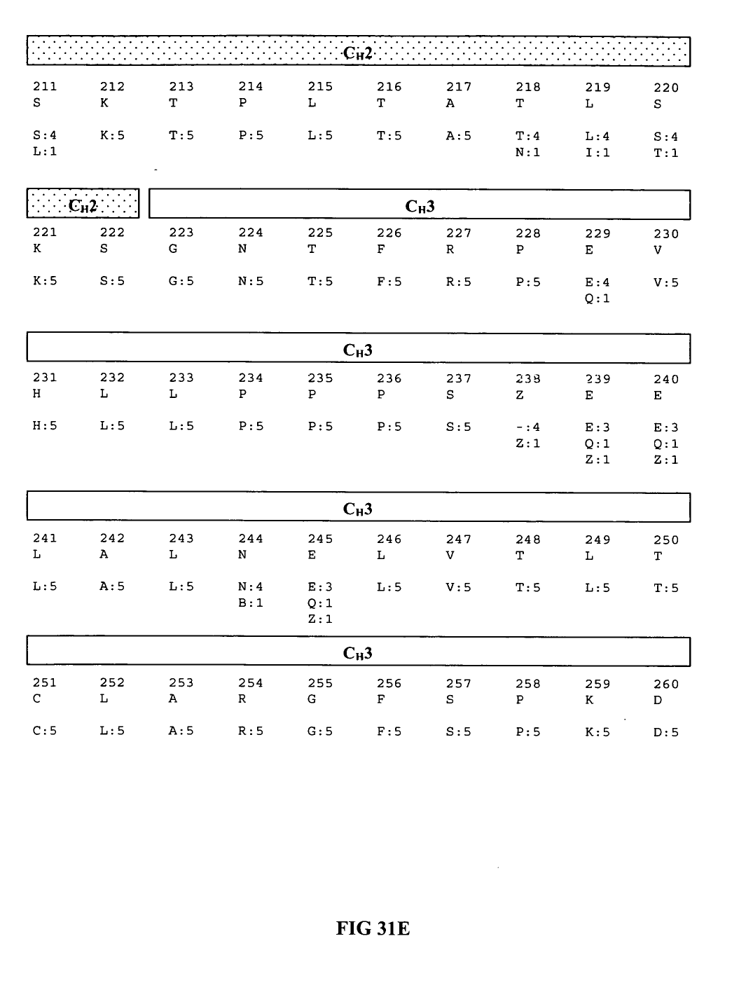

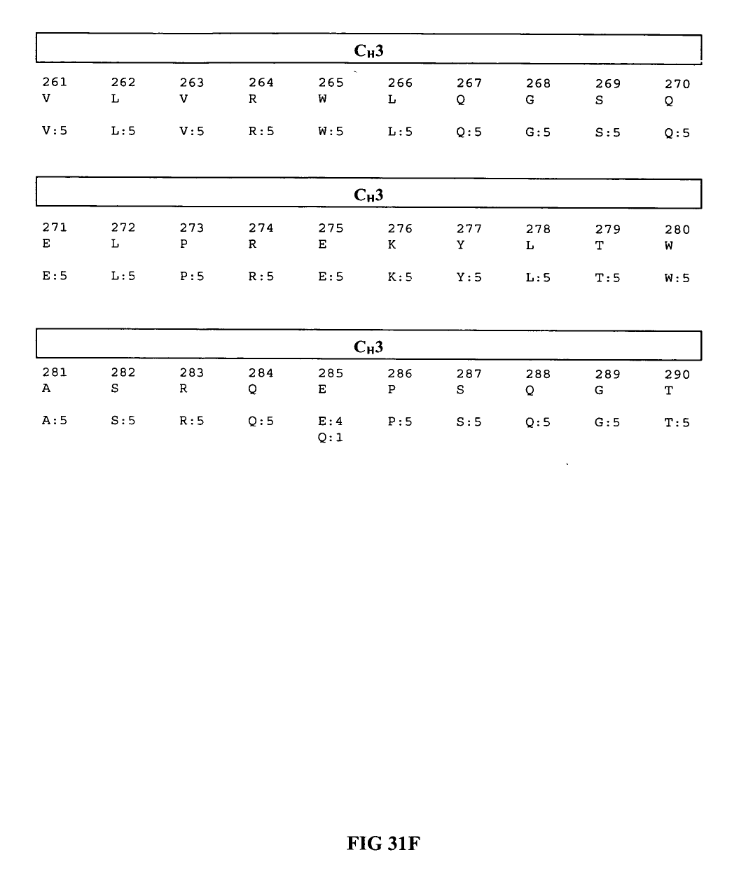

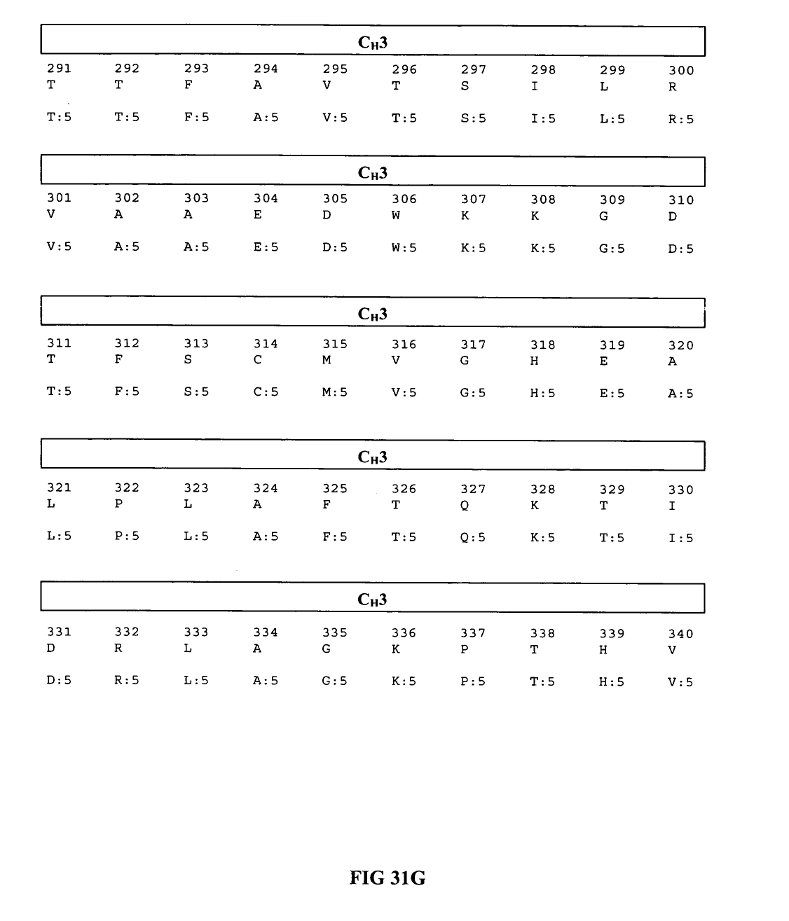

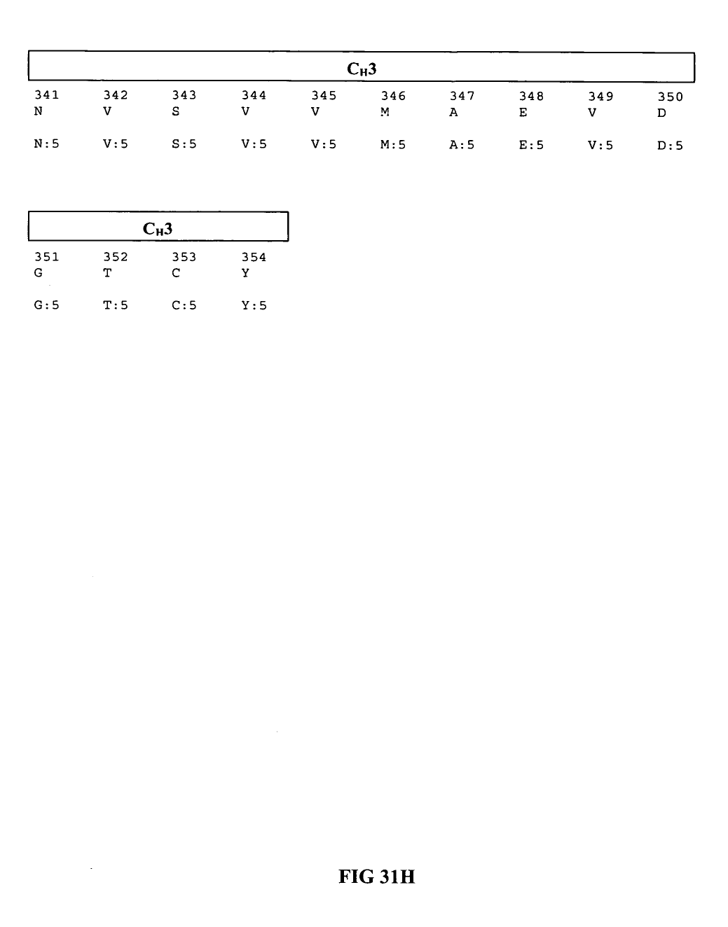

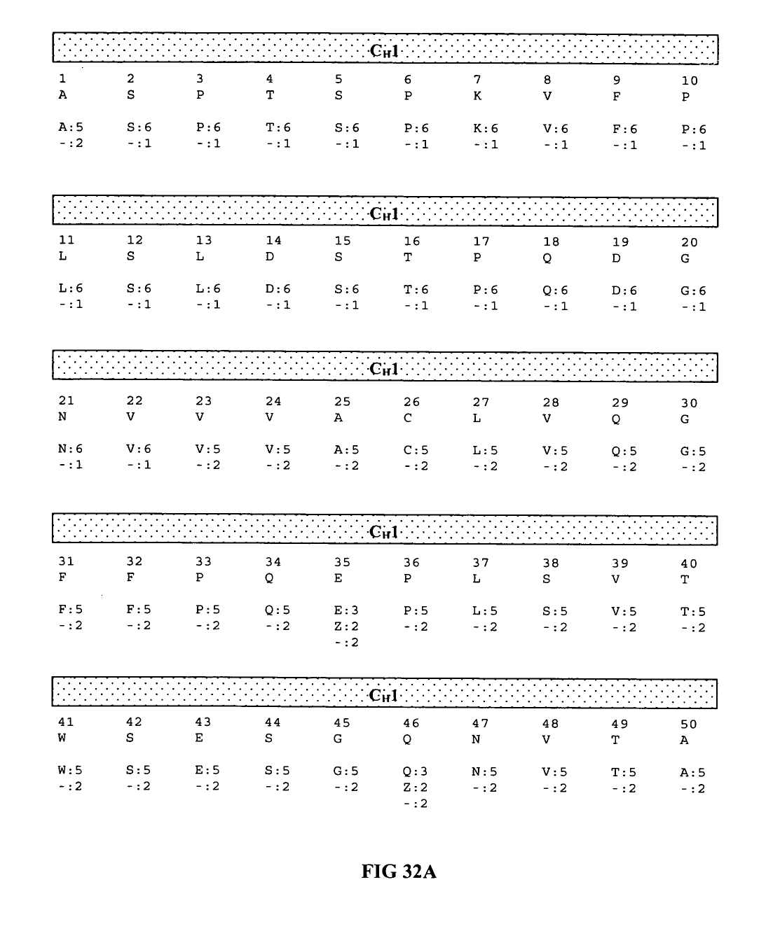

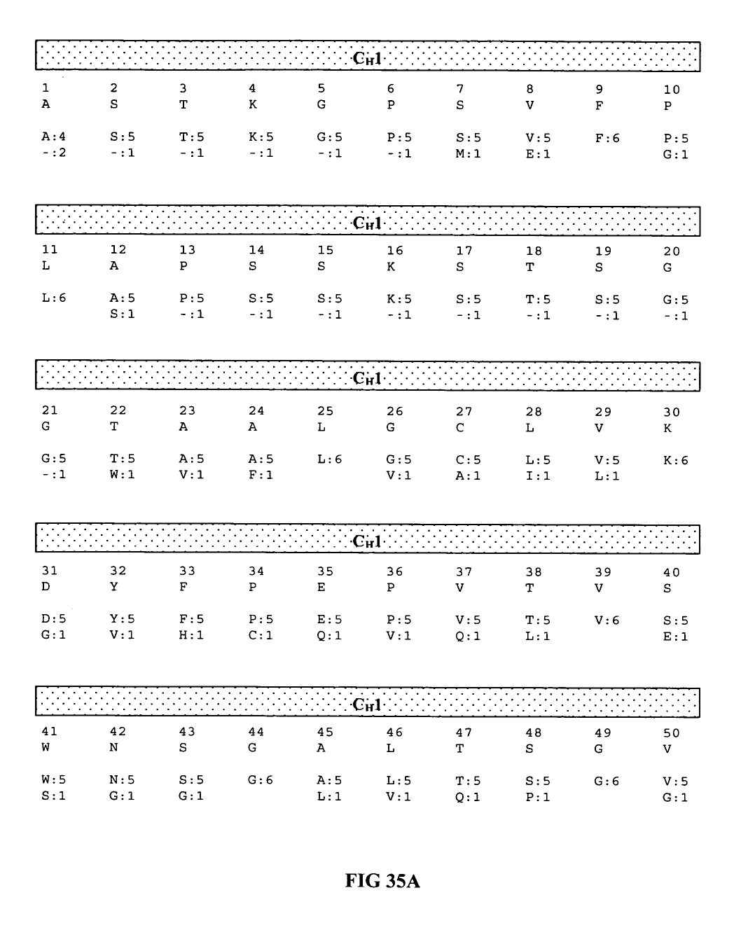

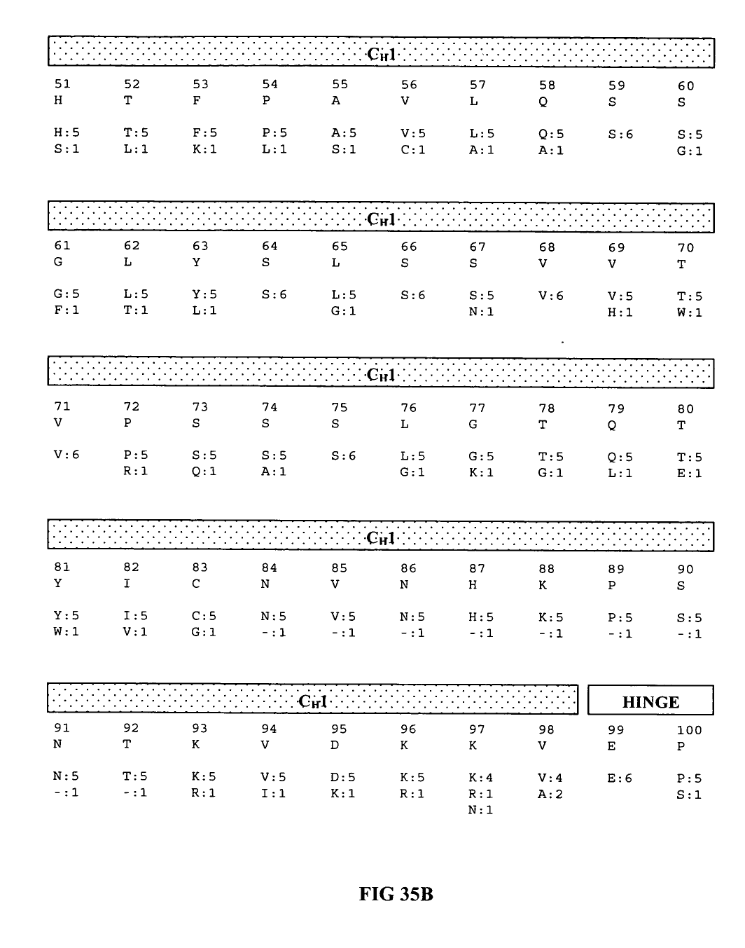

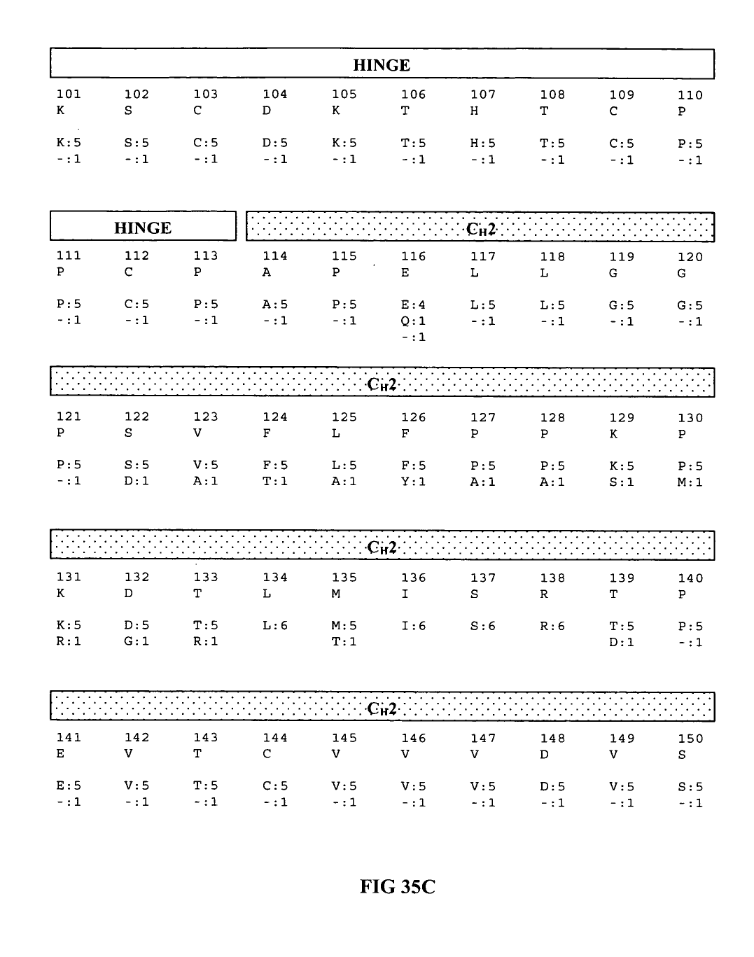

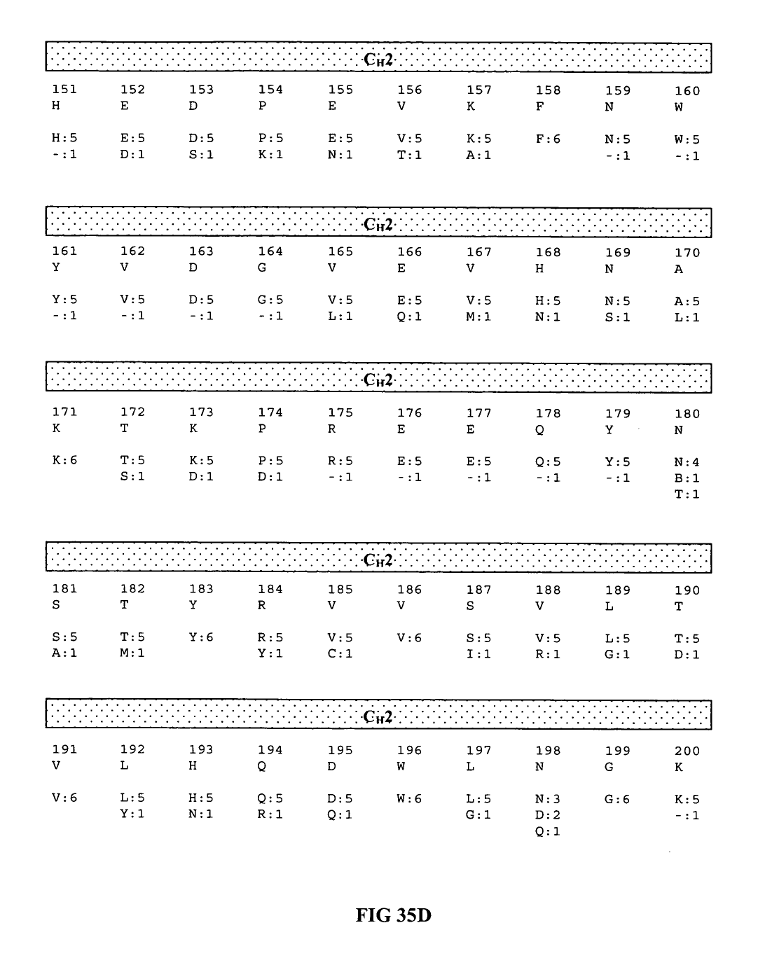

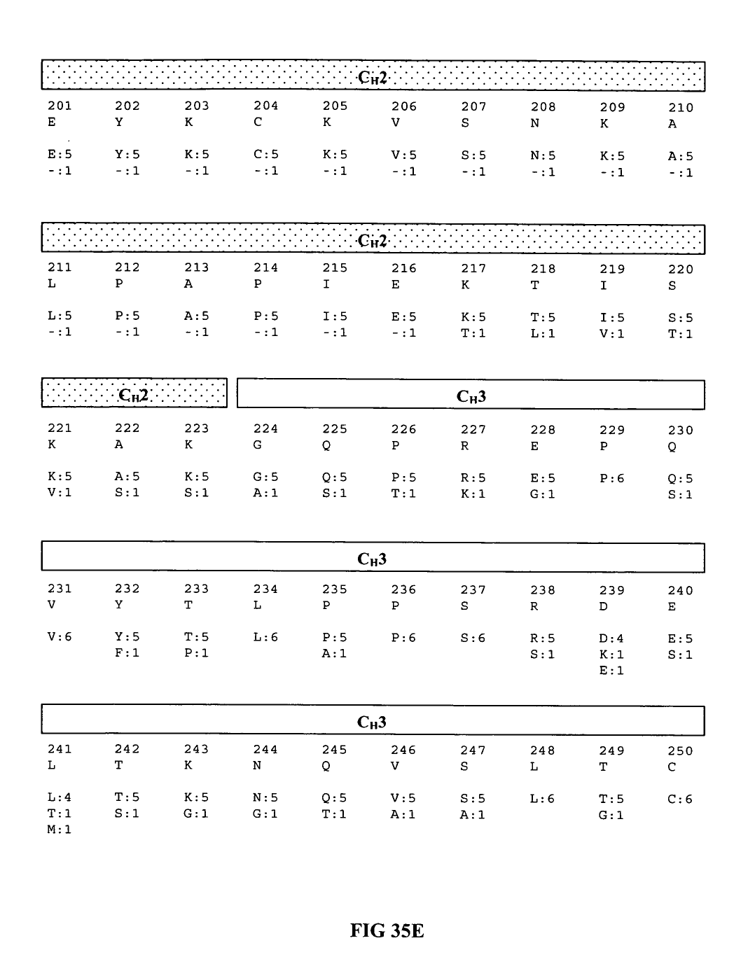

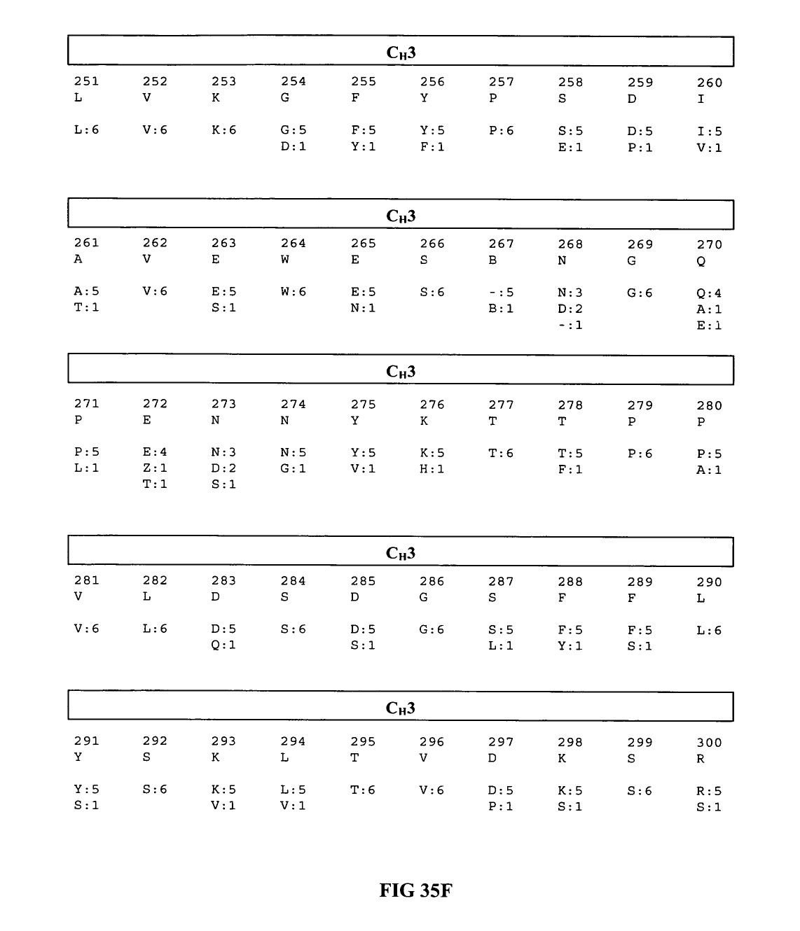

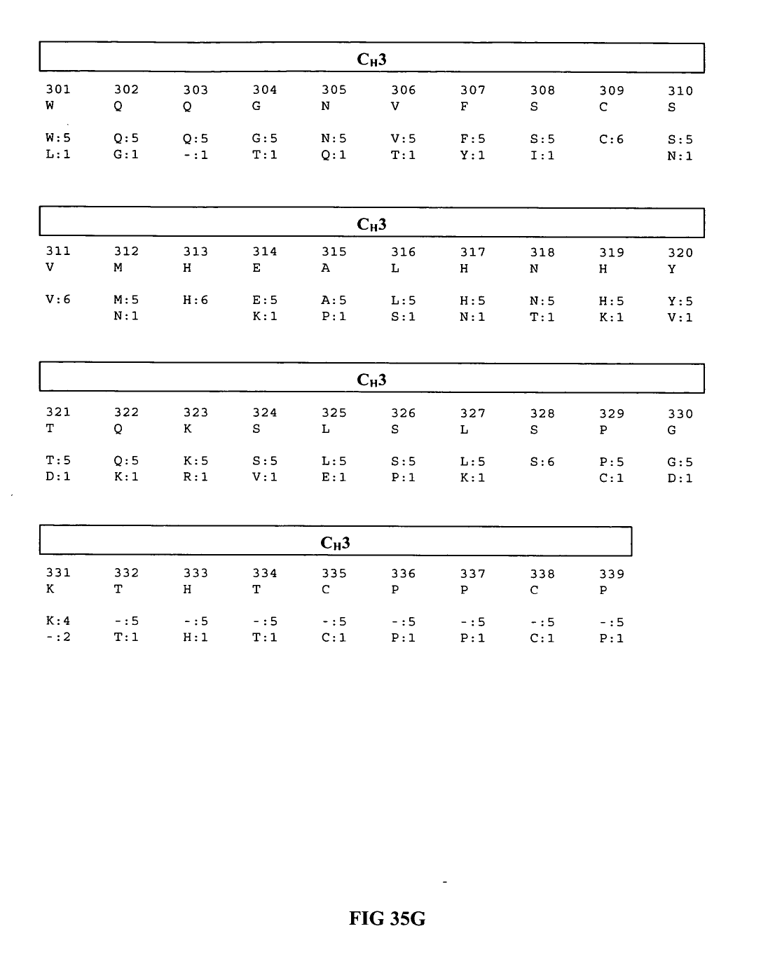

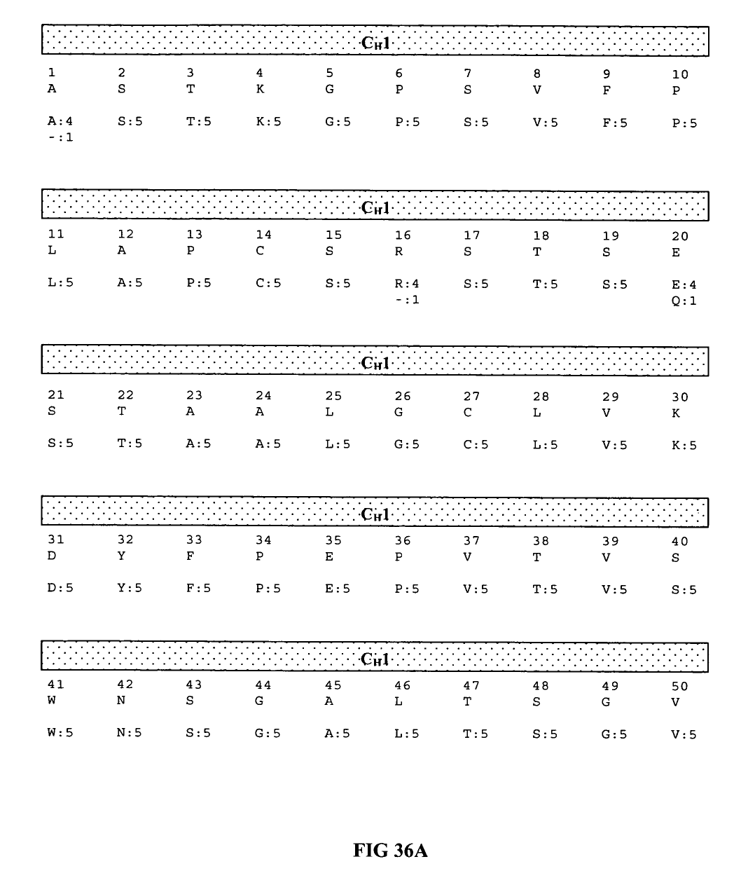

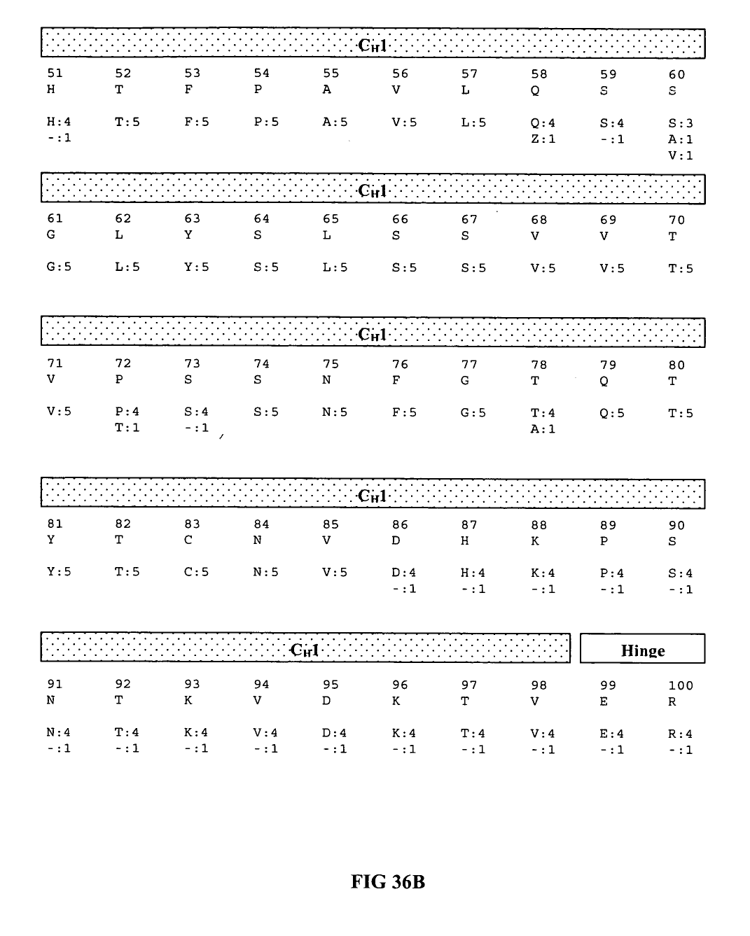

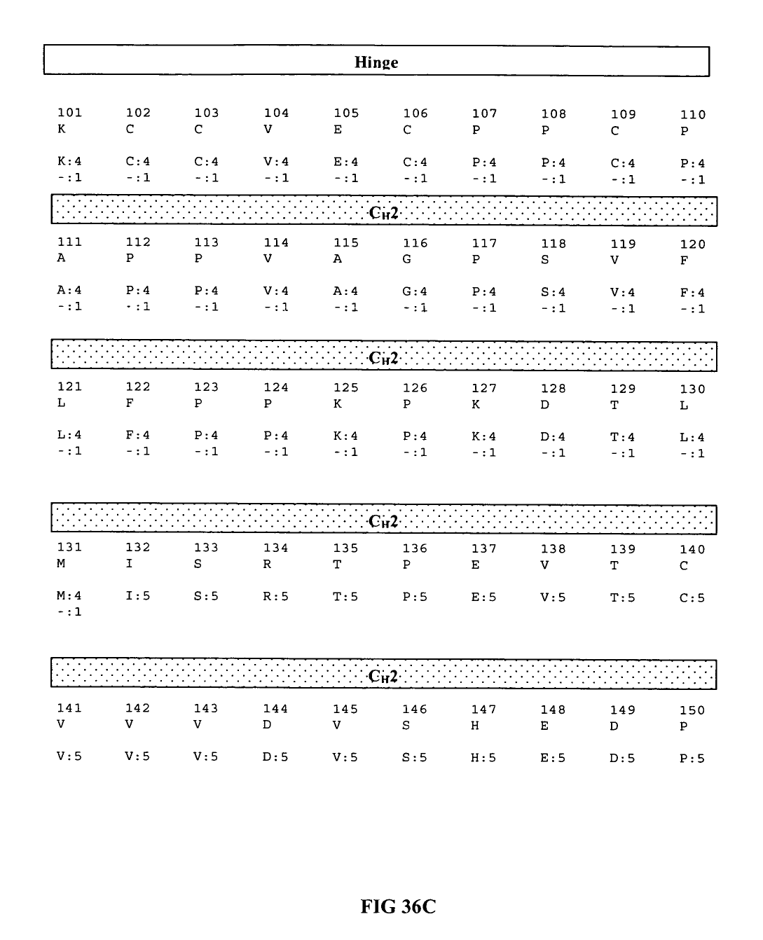

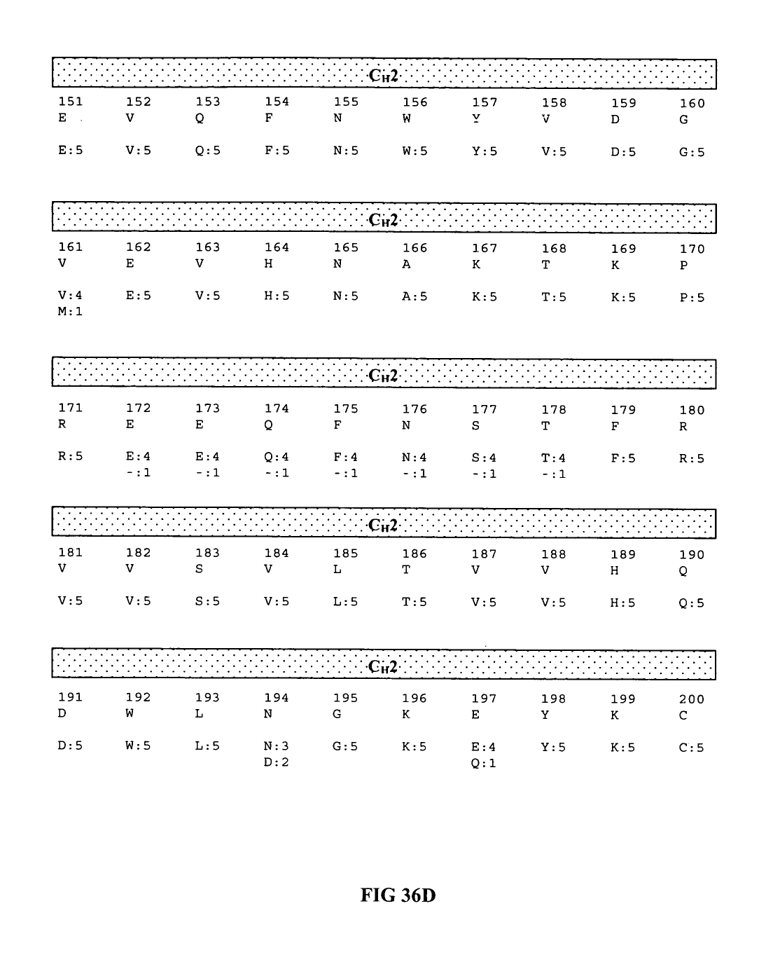

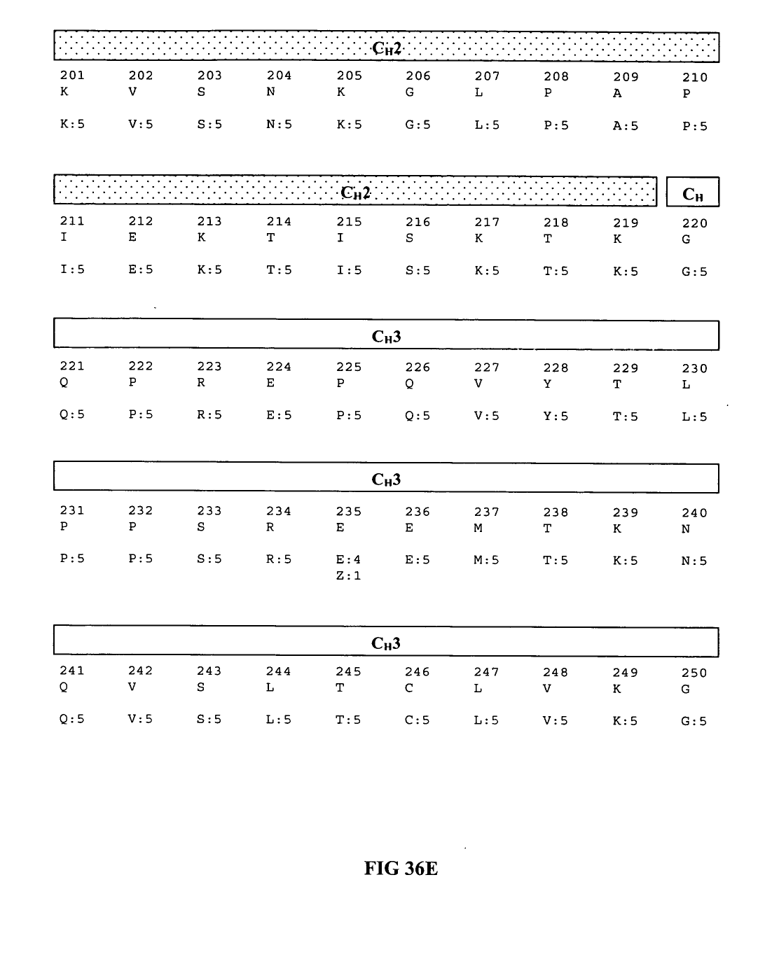

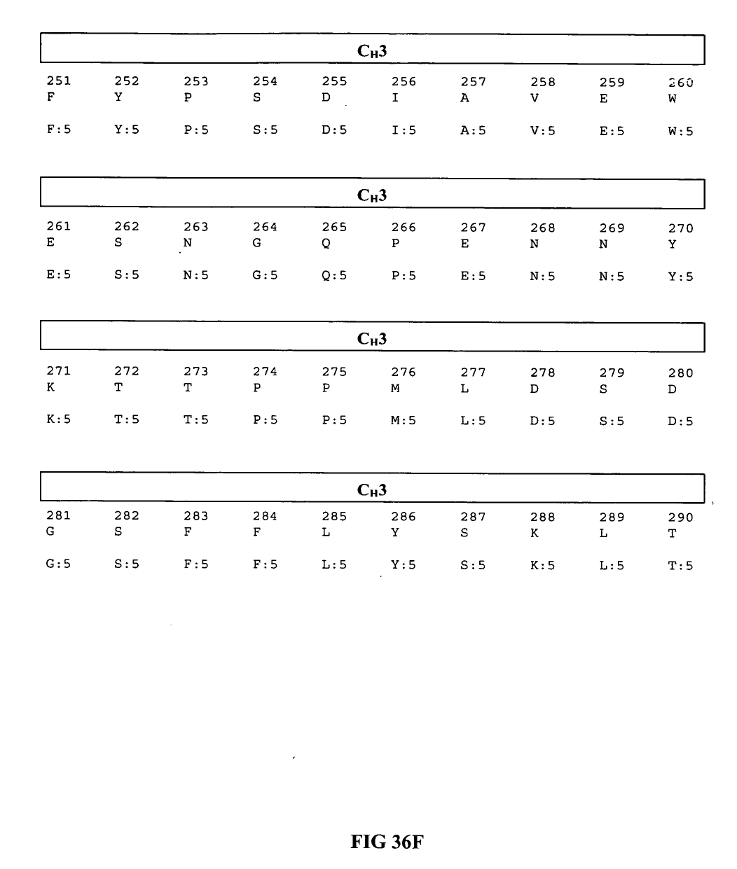

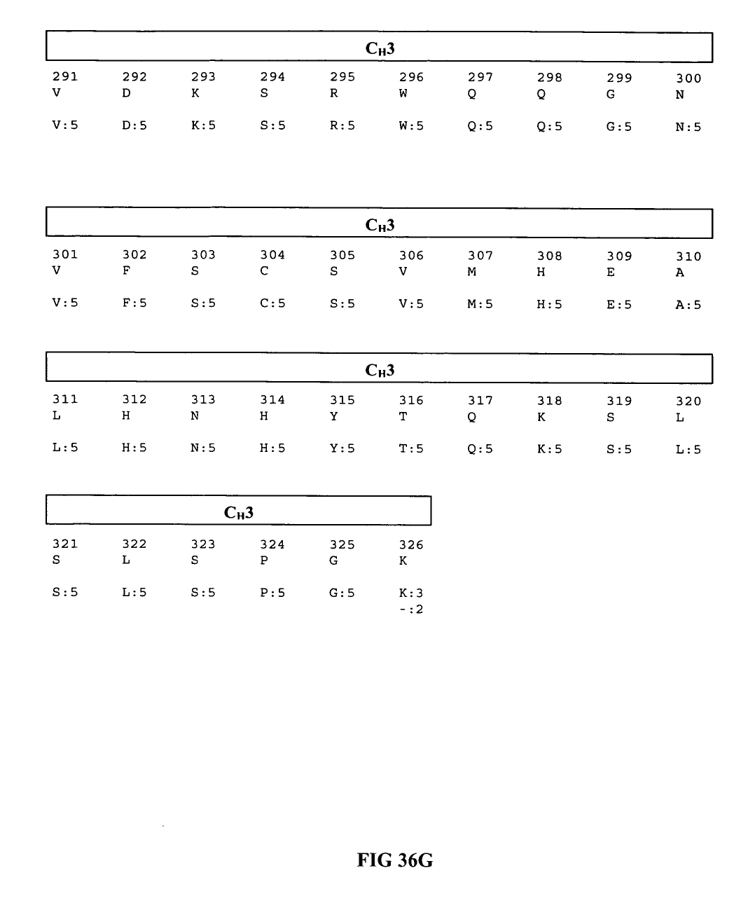

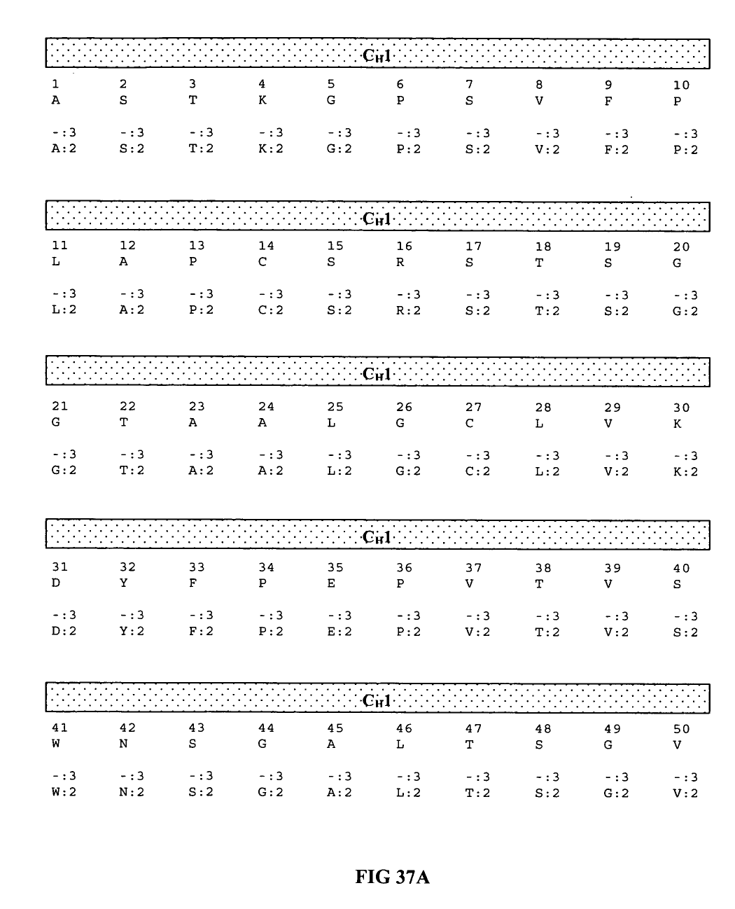

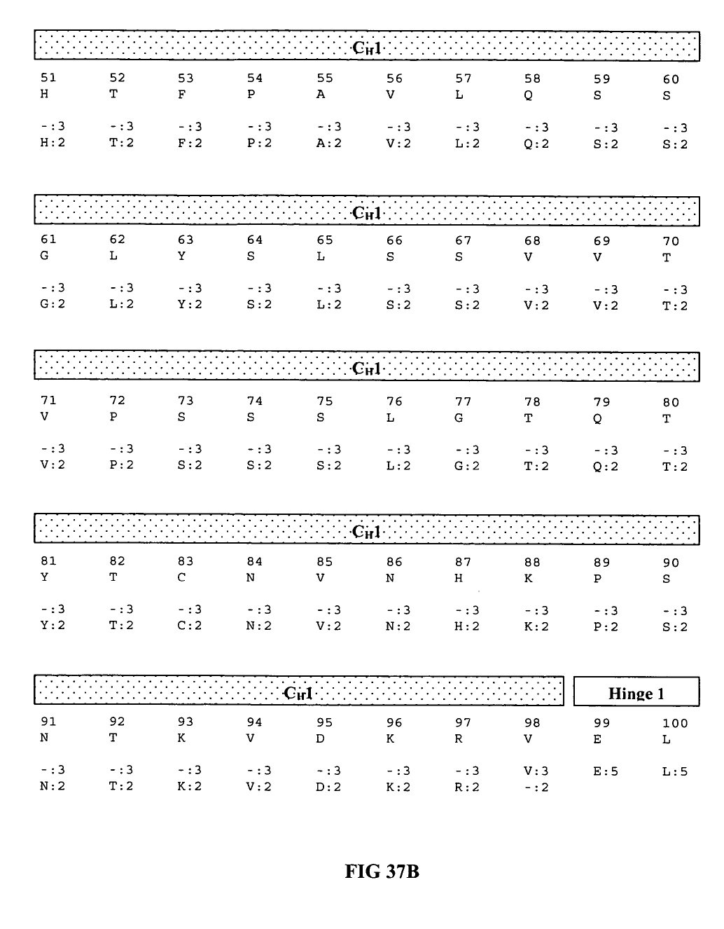

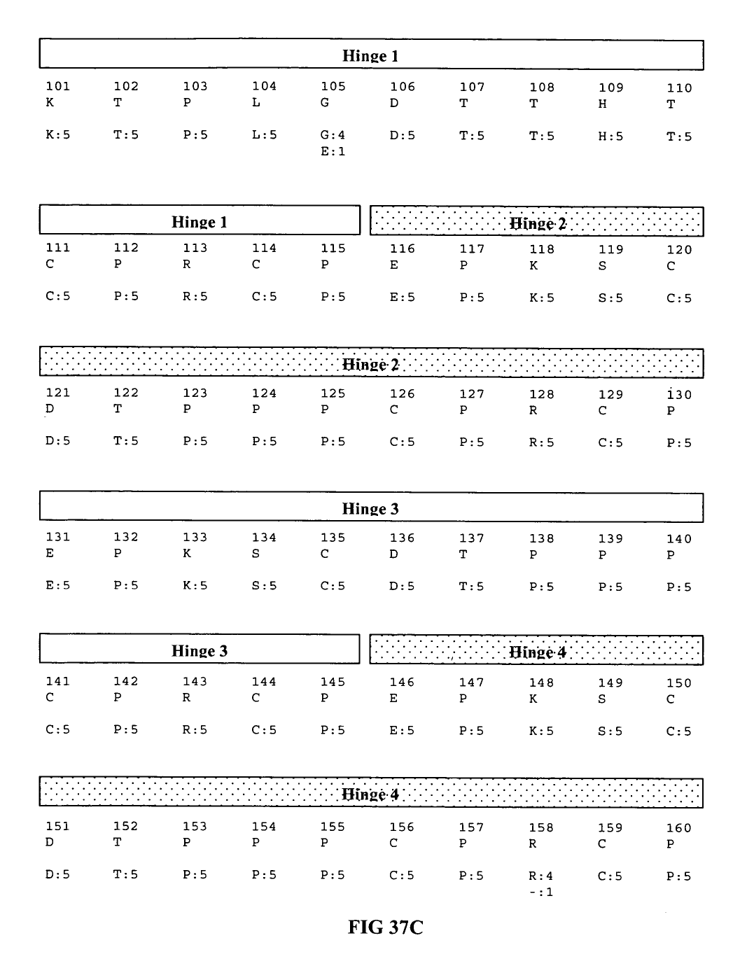

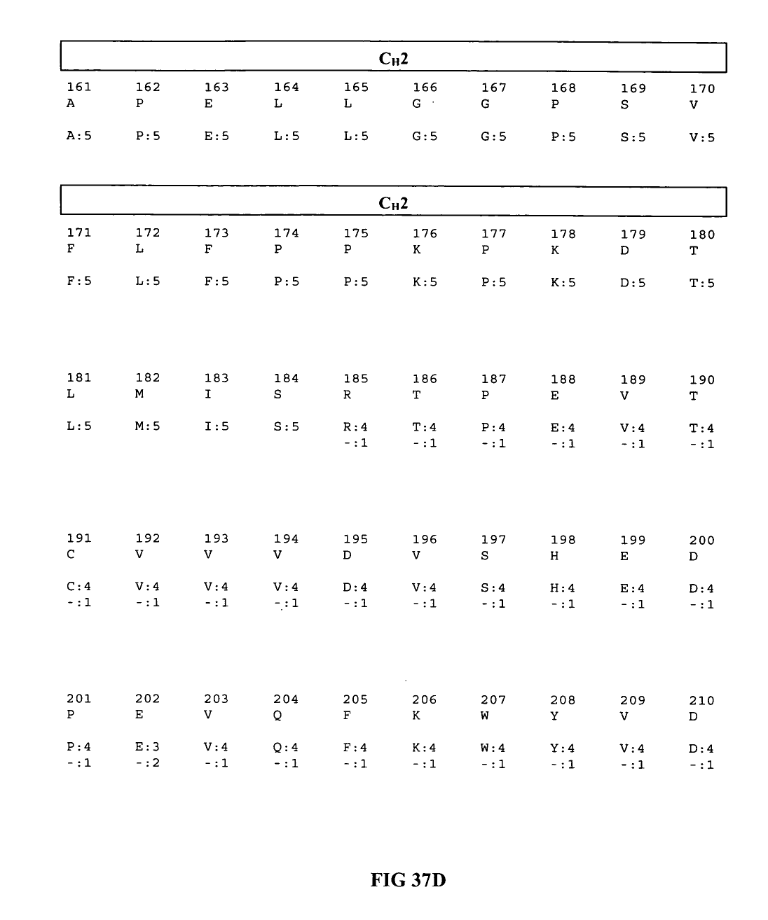

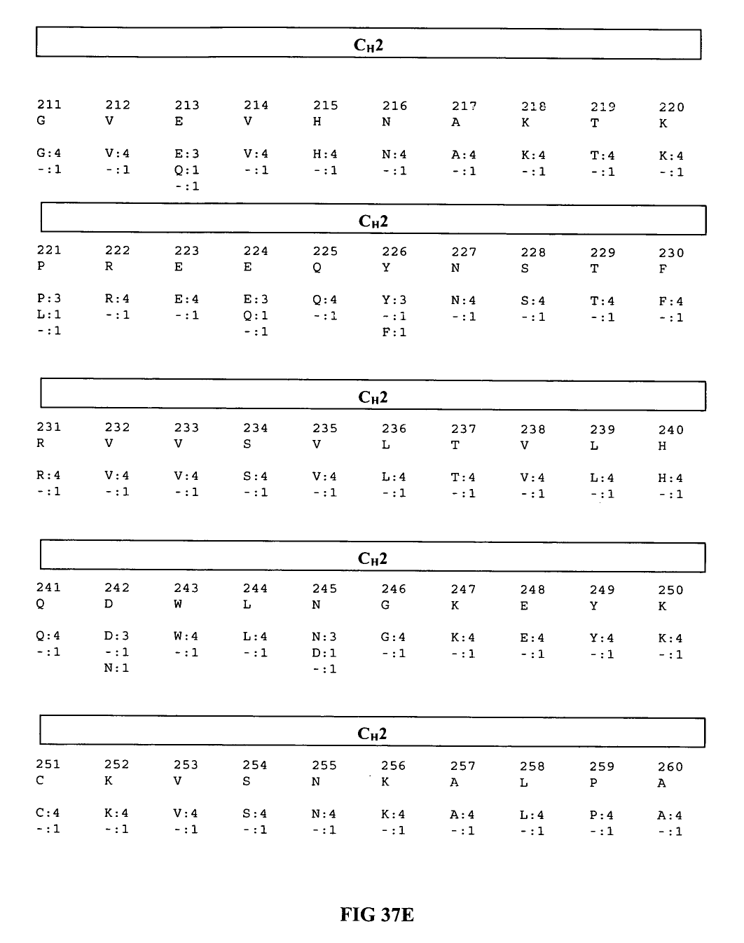

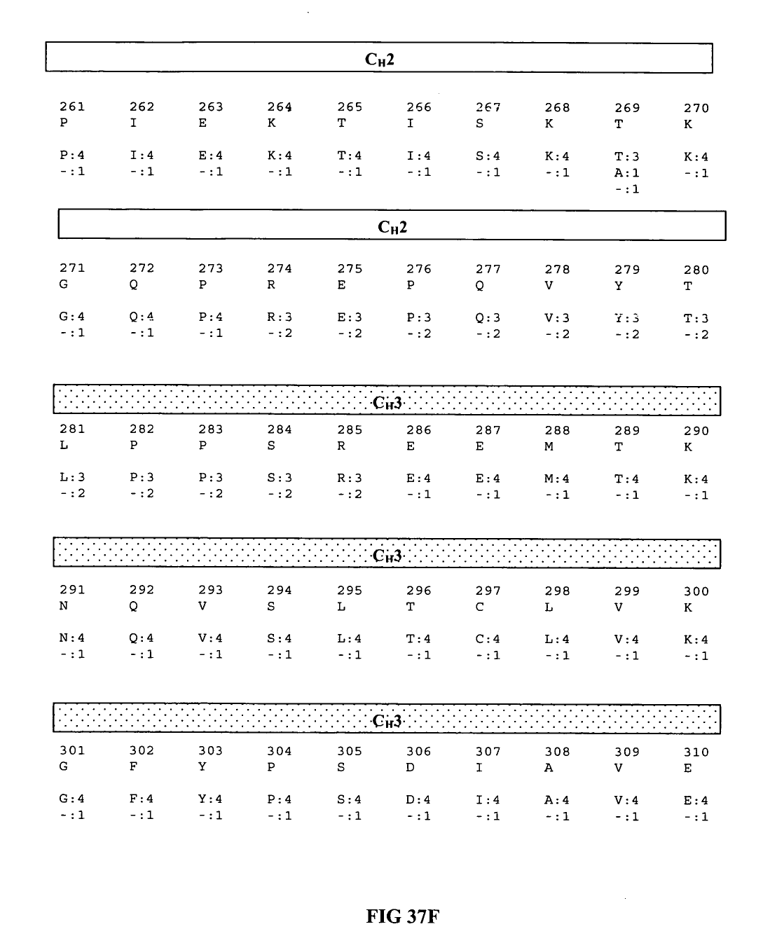

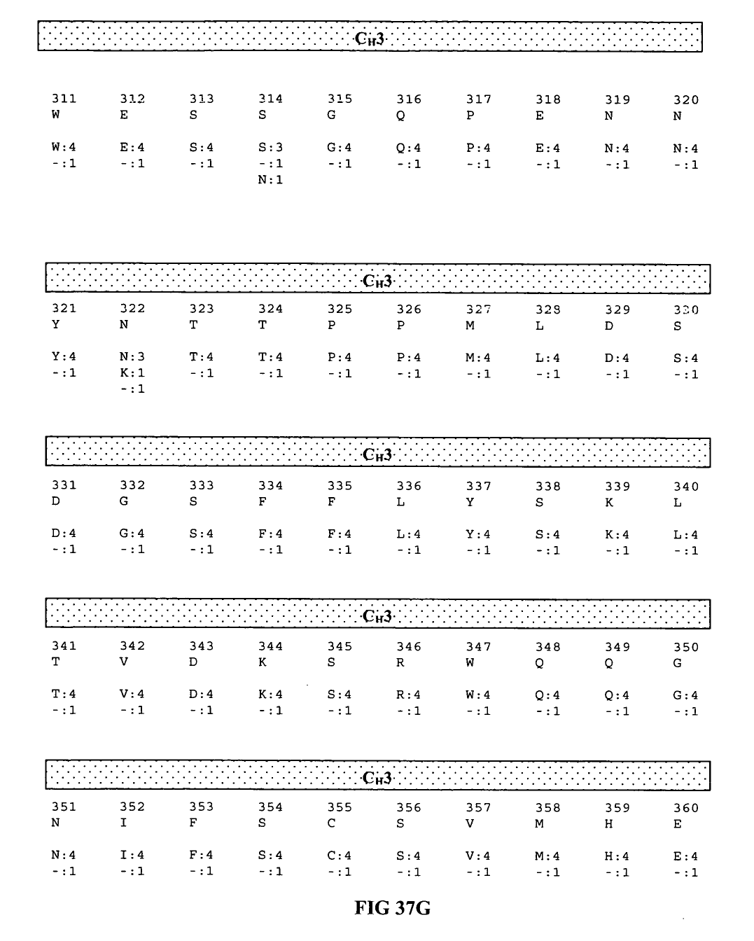

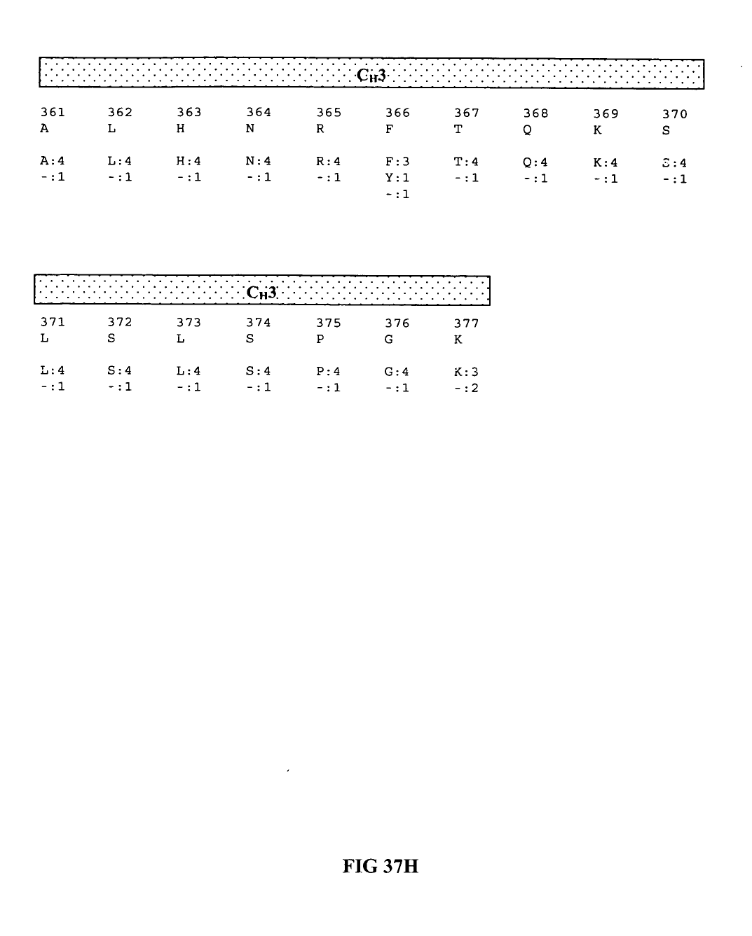

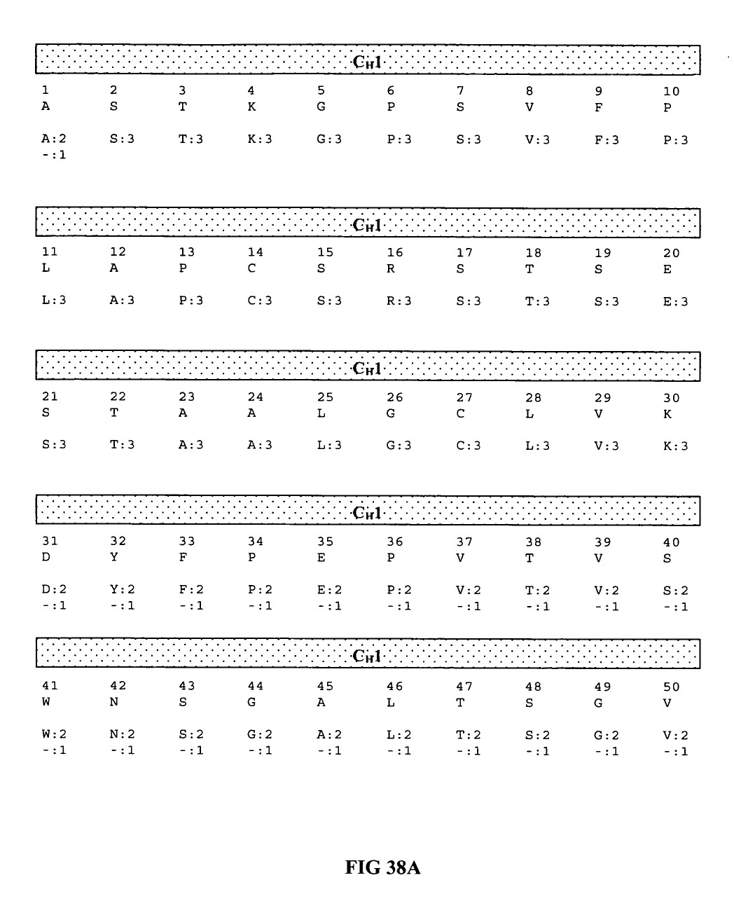

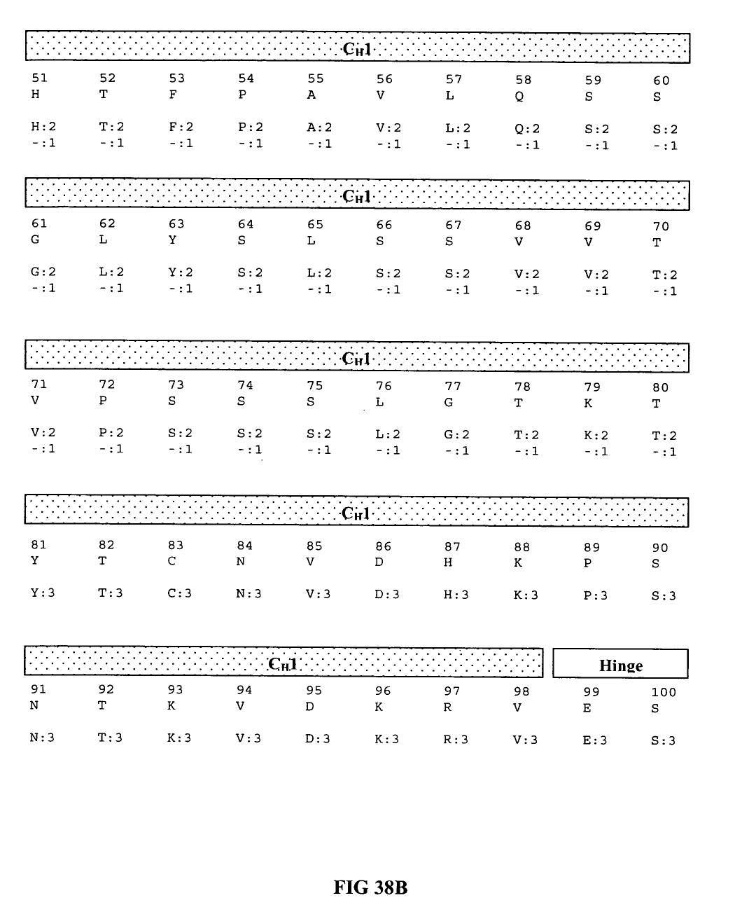

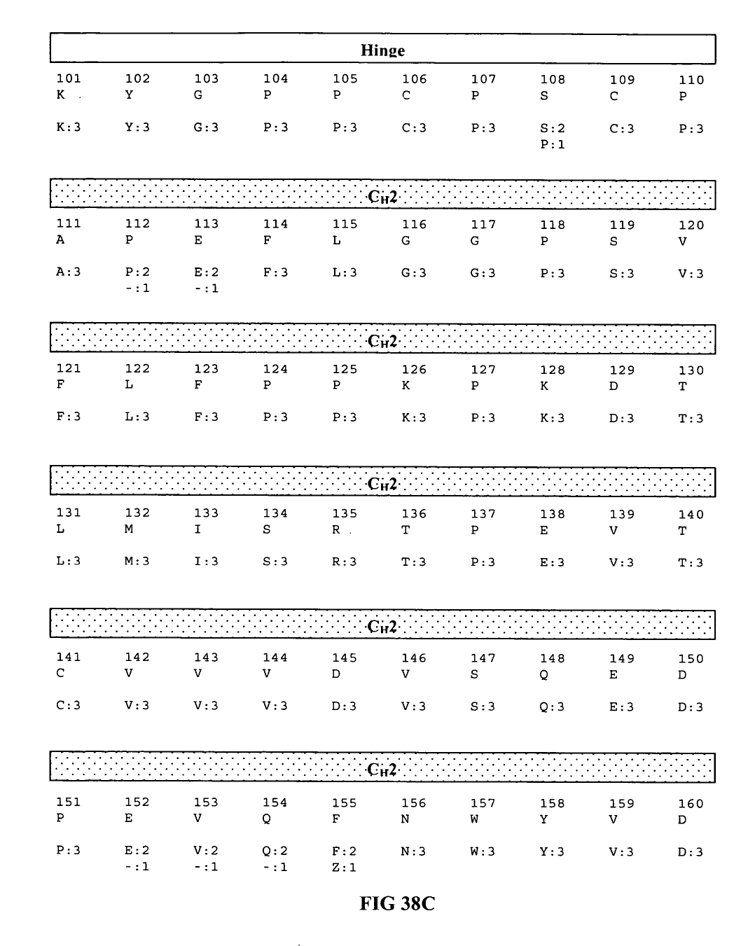

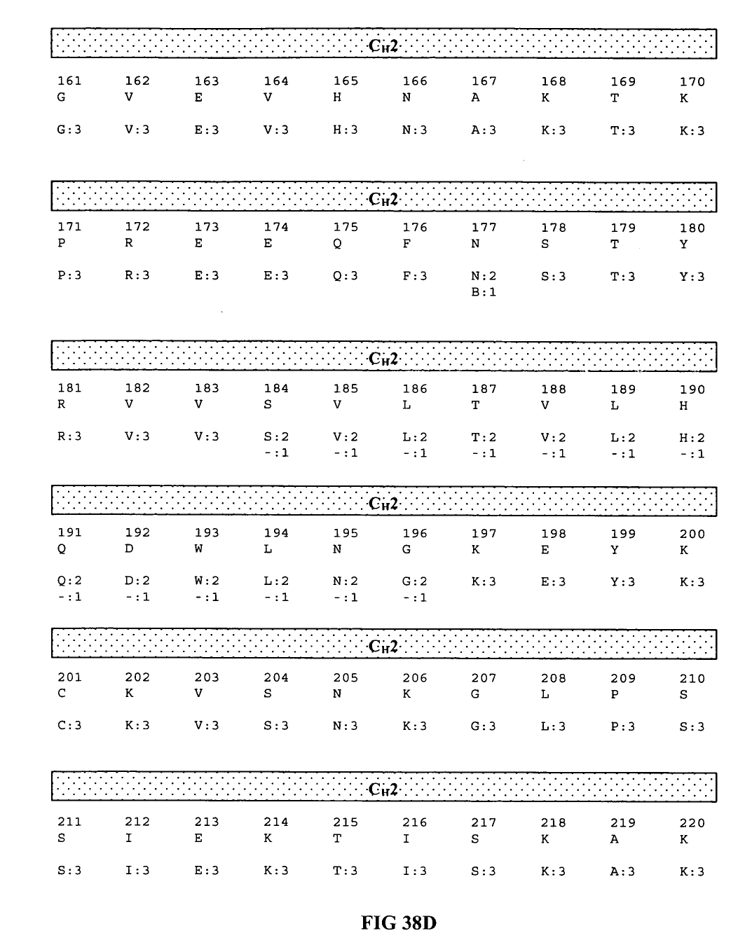

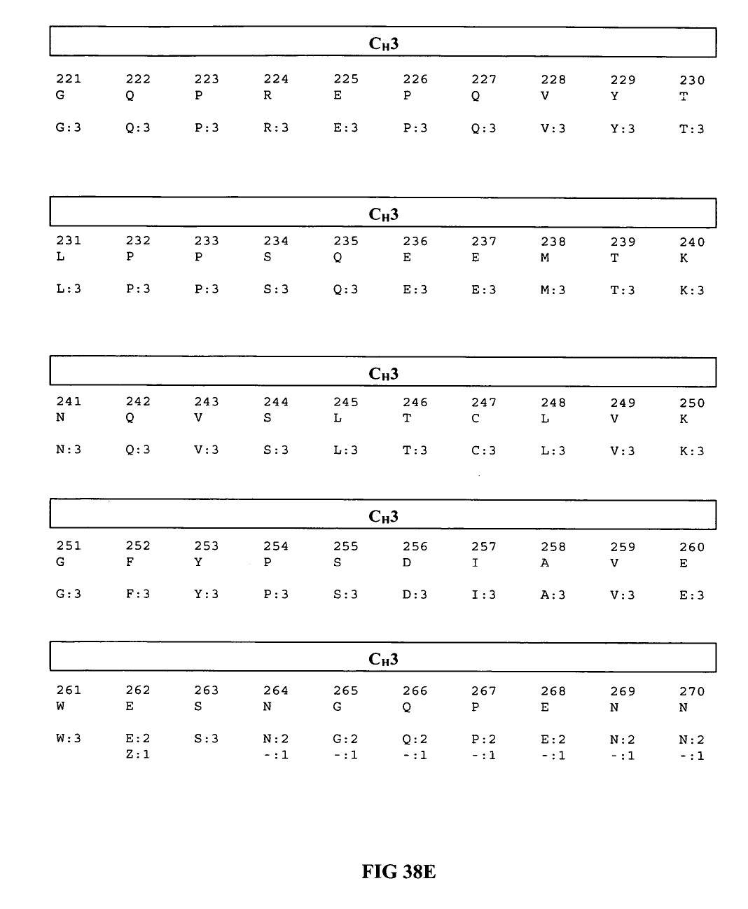

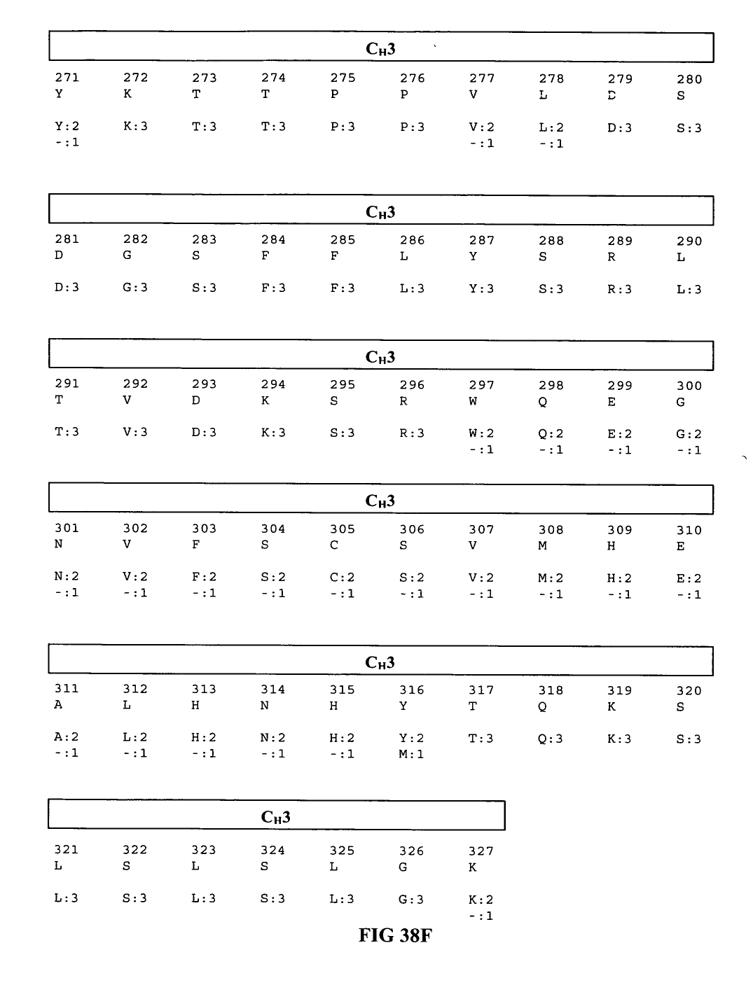

In the present invention, the CH1 portion is not included and a variable number of amino acids from the the N-terminus of the hinge region are deleted, e.g., as referenced to FIGS. 1-41 and Table 23. The variable number of amino acids used for the hinge core portion of a mimetibody of the present invention include, but are not limited to, deletion of any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 1-3, 2-5, 2-7, 2-8, 3-9, 4-10, 5-9, 5-10, 5-15, 10-20, 2-30, 20-40, 10-50, or any range or value therein, of the N-terminal amino acids of at least one hinge region, e.g., as presented in FIGS. 31-39, or Table 23 above, e.g., but not limited to, deletion of any to all of the amino acids 99-101 to 105-157 of amino acids 99-105, 99-108, 99-111, 99-112, 99-113, 99-114, 99-115, 99-119, 99-125, 99-128, 99-134, 99-140, 99-143, 99-149, 99-155 and 99-158 of FIGS. 31-39, corresponding to SEQ ID NOS:1010-1018, including the substitutions, insertions or deletions described in FIGS. 31-39. In preferred embodiments, a hinge core regions of the present invention includes a deletion of the N-terminous of the hinge region to provide a hinge core region that includes a deletion up to but not including a Cys residue or up to but not including a sequence Cys-Pro-Xaa-Cys. In further preferred embodiment, such hinge core sequences used in a hinge core mimetibody of the present invention include amino acids 109-113 or 112-113 of FIG. 35 (SEQ ID NO:1014) (IgG1); 105-110 or 109-110 of FIG. 36 (SEQ ID NO:1015) (IgG2); 111-160, 114-160, 120-160, 126-160, 129-160, 135-160, 141-160, 144-160, 150-160, 156-160 and 159-160 of FIG. 37 (SEQ ID NO:1016) (IgG3); or 106-110 or 109-110 of FIG. 38 (SEQ ID NO:1017) (IgG4).

The CH2, CH3 and optional CH4 sequence can be any suitable human or human compatable sequence, e.g., as presented in FIGS. 1-41 and Table 23, or as known in the art, or any combination or consensus sequence thereof, or any fusion protein thereof.

Amino acids in a hinge core mimetibody or specified portion or variant of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to at least one protein related activity, as specified herein or as known in the art. Sites that are critical for hinge core mimetibody or specified portion or variant binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

Mimetibodies or specified portions or variants of the present invention can comprise as P portion of Formula (I), but are not limited to, at least one portion, sequence or combination selected from 3 to all the of at least one of SEQ ID NOS:1-979. Non-limiting variants that can enhance or maintain at least one of the listed activities include, but are not limited to, any of the above polypeptides, further comprising at least one mutation corresponding to at least one substitution, insertion or deletion that does not significantly affect the suitable biological activtities or functions of said hinge core mimetibody.

A hinge core mimetibody or specified portion or variant can further optionally comprise at least one functional portion of at least one polypeptide as P portion of Formula (I), at least one of 90-100% of SEQ ID NOS:1-979. A hinge core mimetibody can further optionally comprise an amino acid sequence for the P portion of Formula (I), selected from one or more of SEQ ID NOS:1-979.

In one embodiment, the P amino acid sequence of an immunoglobulin chain, or portion thereof has about 90-100% identity (i.e., 90, 91, 92, 93, 94, 95, 96, 97, 98, 99, 100 or any range or value therein) to the corresponding amino acid sequence of the corresponding portion of at least one of SEQ ID NOS: 1-979. Preferably, 90-100% amino acid identity (i.e., 90, 91, 92, 93, 94, 95, 96, 97, 98, 99, 100 or any range or value therein) is determined using a suitable computer algorithm, as known in the art.

Mimetibodies or specified portions or variants of the present invention can comprise any number of contiguous amino acid residues from a hinge core mimetibody or specified portion or variant of the present invention, wherein that number is selected from the group of integers consisting of from 10-100% of the number of contiguous residues in a hinge core mimetibody. Optionally, this subsequence of contiguous amino acids is at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, 5, 6, 7, 8, 9, 10, 11, 12, 13, 14, 15, 16, 17, 18, 19, 20, or more.

As those of skill will appreciate, the present invention includes at least one biologically active hinge core mimetibody or specified portion or variant of the present invention. Biologically active mimetibodies or specified portions or variants have a specific activity at least 20%, 30%, or 40%, and preferably at least 50%, 60%, or 70%, and most preferably at least 80%, 90%, or 95%-1000% of that of the native (non-synthetic), endogenous or related and known inserted or fused protein or specified portion or variant. Methods of assaying and quantifying measures of enzymatic activity and substrate specificity are well known to those of skill in the art.

In another aspect, the invention relates to human mimetibodies and ligand-binding fragments, as described herein, which are modified by the covalent attachment of an organic moiety. Such modification can produce a hinge core mimetibody or ligand-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified mimetibodies and ligand-binding fragments of the invention can comprise one or more organic moieties that are covalently bonded, directly or indirectly, to the hinge core mimetibody or specified portion or variant. Each organic moiety that is bonded to a hinge core mimetibody or ligand-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, a hinge core mimetibody modified by the covalent attachment of polylysine is encompassed by the invention. Hydrophilic polymers suitable for modifying mimetibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the hinge core mimetibody of the invention has a molecular weight of about 800 to about 150,000 Daltons as a separate molecular entity. For example, PEG₂₅₀₀, PEG₅₀₀₀, PEG₇₅₀₀, PEG₉₀₀₀, PEG₁₀₀₀₀, PEG₁₂₅₀₀, PEG₅₀₀₀, and PEG_{20,000}, wherein the subscript is the average molecular weight of the polymer in Daltons, can be used.

The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying mimetibodies of the invention can be saturated or can contain one or more units of unsaturation. Fatty acids that are suitable for modifying mimetibodies of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human mimetibodies and ligand-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See, for example, Thompson, et al., WO 92/16221 the entire teachings of which are incorporated herein by reference.)

The modified mimetibodies of the invention can be produced by reacting an human hinge core mimetibody or ligand-binding fragment with a modifying agent. For example, the organic moieties can be bonded to the hinge core mimetibody in a non-site specific manner by employing an amine-reactive modifying agent, for example, an NHS ester of PEG. Modified human mimetibodies or ligand-binding fragments can also be prepared by reducing disulfide bonds (e.g., intra-chain disulfide bonds) of a hinge core mimetibody or ligand-binding fragment. The reduced hinge core mimetibody or ligand-binding fragment can then be reacted with a thiol-reactive modifying agent to produce the modified hinge core mimetibody of the invention. Modified human mimetibodies and ligand-binding fragments comprising an organic moiety that is bonded to specific sites of a hinge core mimetibody or specified portion or variant of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

Hinge Core Mimetibody Compositions

The present invention also provides at least one hinge core mimetibody or specified portion or variant composition comprising at least one, at least two, at least three, at least four, at least five, at least six or more mimetibodies or specified portions or variants thereof, as described herein and/or as known in the art that are provided in a non-naturally occurring composition, mixture or form. Such composition percentages are by weight, volume, concentration, molarity, or molality as liquid or dry solutions, mixtures, suspension, emulsions or colloids, as known in the art or as described herein.

Such compositions can comprise 0.00001-99.9999 percent by weight, volume, concentration, molarity, or molality as liquid, gas, or dry solutions, mixtures, suspension, emulsions or colloids, as known in the art or as described herein, on any range or value therein, such as but not limited to 0.00001, 0.00003, 0.00005, 0.00009, 0.0001, 0.0003, 0.0005, 0.0009, 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, 5, 6, 7, 8, 9, 10, 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9%. Such compositions of the present invention thus include but are not limited to 0.00001-100 mg/ml and/or 0.00001-100 mg/g.

The composition can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21^{st} edition, Springhouse Corp., Springhouse, Pa., 2001; Health Professional's Drug Guide 2001, ed., Shannon, Wilson, Stang, Prentice-Hall, Inc, Upper Saddle River, N.J.; Pharmcotherapy Handbook, Wells et al., ed., Appleton & Lange, Stamford, Conn., each entirely incorporated herein by reference).

The anti-infective drug can be at least one selected from amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, miscellaneous anti-infectives. The CV drug can be at least one selected from inotropics, antiarrhythmics, antianginals, antihypertensives, antilipemics, and miscellaneous cardiovascular drugs. The CNS drug can be at least one selected from nonnarcotic analgesics or at least one selected from antipyretics, nonsteroidal anti-inflammatory drugs, narcotic or at least one opiod analgesics, sedative-hypnotics, anticonvulsants, antidepressants, antianxiety drugs, antipsychotics, central nervous system stimulants, antiparkinsonians, miscellaneous central nervous system drugs. The ANS drug can be at least one selected from cholinergics (parasympathomimetics), anticholinergics, adrenergics (sympathomimetics), adrenergic blockers (sympatholytics), skeletal muscle relaxants, neuromuscular blockers. The respiratory tract drug can be at least one selected from antihistamines, bronchodilators, expectorants or at least one antitussives, miscellaneous respiratory drugs. The GI tract drug can be at least one selected from antacids or at least one adsorbents or at least one antiflatulents, digestive enzymes or at least one gallstone solubilizers, antidiarrheals, laxatives, antiemetics, antiulcer drugs. The hormonal drug can be at least one selected from corticosteroids, androgens or at least one anabolic steroids, estrogens or at least one progestins, gonadotropins, antidiabetic drugs or at least one glucagon, thyroid hormones, thyroid hormone antagonists, pituitary hormones, parathyroid-like drugs. The drug for fluid and electrolyte balance can be at least one selected from diuretics, electrolytes or at least one replacement solutions, acidifiers or at least one alkalinizers. The hematologic drug can be at least one selected from hematinics, anticoagulants, blood derivatives, thrombolytic enzymes. The antineoplastics can be at least one selected from alkylating drugs, antimetabolites, antibiotic antineoplastics, antineoplastics that alter hormone balance, miscellaneous antineoplastics. The immunomodulation drug can be at least one selected from immunosuppressants, vaccines or at least one toxoids, antitoxins or at least one antivenins, immune serums, biological response modifiers. The ophthalmic, otic, and nasal drugs can be at least one selected from ophthalmic anti-infectives, ophthalmic anti-inflammatories, miotics, mydriatics, ophthalmic vasoconstrictors, miscellaneous ophthalmics, otics, nasal drugs. The topical drug can be at least one selected from local anti-infectives, scabicides or at least one pediculicides, topical corticosteroids. The nutritional drug can be at least one selected from vitamins, minerals, or calorics. See, e.g., contents of Nursing 2001 Drug Handbook, supra.

The at least one amebicide or antiprotozoal can be at least one selected from atovaquone, chloroquine hydrochloride, chloroquine phosphate, metronidazole, metronidazole hydrochloride, pentamidine isethionate. The at least one anthelmintic can be at least one selected from mebendazole, pyrantel pamoate, thiabendazole. The at least one antifungal can be at least one selected from amphotericin B, amphotericin B cholesteryl sulfate complex, amphotericin B lipid complex, amphotericin B liposomal, fluconazole, flucytosine, griseofulvin microsize, griseofulvin ultramicrosize, itraconazole, ketoconazole, nystatin, terbinafine hydrochloride. The at least one antimalarial can be at least one selected from chloroquine hydrochloride, chloroquine phosphate, doxycycline, hydroxychloroquine sulfate, mefloquine hydrochloride, primaquine phosphate, pyrimethamine, pyrimethamine with sulfadoxine. The at least one antituberculotic or antileprotic can be at least one selected from clofazimine, cycloserine, dapsone, ethambutol hydrochloride, isoniazid, pyrazinamide, rifabutin, rifampin, rifapentine, streptomycin sulfate. The at least one aminoglycoside can be at least one selected from amikacin sulfate, gentamicin sulfate, neomycin sulfate, streptomycin sulfate, tobramycin sulfate. The at least one penicillin can be at least one selected from amoxcillin/clavulanate potassium, amoxicillin trihydrate, ampicillin, ampicillin sodium, ampicillin trihydrate, ampicillin sodium/sulbactam sodium, cloxacillin sodium, dicloxacillin sodium, mezlocillin sodium, nafcillin sodium, oxacillin sodium, penicillin G benzathine, penicillin G potassium, penicillin G procaine, penicillin G sodium, penicillin V potassium, piperacillin sodium, piperacillin sodium/tazobactam sodium, ticarcillin disodium, ticarcillin disodium/clavulanate potassium. The at least one cephalosporin can be at least one selected from at least one of cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, loracarbef. The at least one tetracycline can be at least one selected from demeclocycline hydrochloride, doxycycline calcium, doxycycline hyclate, doxycycline hydrochloride, doxycycline monohydrate, minocycline hydrochloride, tetracycline hydrochloride. The at least one sulfonamide can be at least one selected from co-trimoxazole, sulfadiazine, sulfamethoxazole, sulfisoxazole, sulfisoxazole acetyl. The at least one fluoroquinolone can be at least one selected from alatrofloxacin mesylate, ciprofloxacin, enoxacin, levofloxacin, lomefloxacin hydrochloride, nalidixic acid, norfloxacin, ofloxacin, sparfloxacin, trovafloxacin mesylate. The at least one fluoroquinolone can be at least one selected from alatrofloxacin mesylate, ciprofloxacin, enoxacin, levofloxacin, lomefloxacin hydrochloride, nalidixic acid, norfloxacin, ofloxacin, sparfloxacin, trovafloxacin mesylate. The at least one antiviral can be at least one selected from abacavir sulfate, acyclovir sodium, amantadine hydrochloride, amprenavir, cidofovir, delavirdine mesylate, didanosine, efavirenz, famciclovir, fomivirsen sodium, foscarnet sodium, ganciclovir, indinavir sulfate, lamivudine, lamivudine/zidovudine, nelfinavir mesylate, nevirapine, oseltamivir phosphate, ribavirin, rimantadine hydrochloride, ritonavir, saquinavir, saquinavir mesylate, stavudine, valacyclovir hydrochloride, zalcitabine, zanamivir, zidovudine. The at least one macroline anti-infective can be at least one selected from azithromycin, clarithromycin, dirithromycin, erythromycin base, erythromycin estolate, erythromycin ethylsuccinate, erythromycin lactobionate, erythromycin stearate. The at least one miscellaneous anti-infective can be at least one selected from aztreonam, bacitracin, chloramphenicol sodium sucinate, clindamycin hydrochloride, clindamycin palmitate hydrochloride, clindamycin phosphate, imipenem and cilastatin sodium, meropenem, nitrofurantoin macrocrystals, nitrofurantoin microcrystals, quinupristin/dalfopristin, spectinomycin hydrochloride, trimethoprim, vancomycin hydrochloride. (See, e.g., pp. 24-214 of Nursing 2001 Drug Handbook.)

The at least one inotropic can be at least one selected from amrinone lactate, digoxin, milrinone lactate. The at least one antiarrhythmic can be at least one selected from adenosine, amiodarone hydrochloride, atropine sulfate, bretylium tosylate, diltiazem hydrochloride, disopyramide, disopyramide phosphate, esmolol hydrochloride, flecainide acetate, ibutilide fumarate, lidocaine hydrochloride, mexiletine hydrochloride, moricizine hydrochloride, phenytoin, phenytoin sodium, procainamide hydrochloride, propafenone hydrochloride, propranolol hydrochloride, quinidine bisulfate, quinidine gluconate, quinidine polygalacturonate, quinidine sulfate, sotalol, tocainide hydrochloride, verapamil hydrochloride. The at least one antianginal can be at least one selected from amlodipidine besylate, amyl nitrite, bepridil hydrochloride, diltiazem hydrochloride, isosorbide dinitrate, isosorbide mononitrate, nadolol, nicardipine hydrochloride, nifedipine, nitroglycerin, propranolol hydrochloride, verapamil, verapamil hydrochloride. The at least one antihypertensive can be at least one selected from acebutolol hydrochloride, amlodipine besylate, atenolol, benazepril hydrochloride, betaxolol hydrochloride, bisoprolol fumarate, candesartan cilexetil, captopril, carteolol hydrochloride, carvedilol, clonidine, clonidine hydrochloride, diazoxide, diltiazem hydrochloride, doxazosin mesylate, enalaprilat, enalapril maleate, eprosartan mesylate, felodipine, fenoldopam mesylate, fosinopril sodium, guanabenz acetate, guanadrel sulfate, guanfacine hydrochloride, hydralazine hydrochloride, irbesartan, isradipine, labetalol hydrchloride, lisinopril, losartan potassium, methyldopa, methyldopate hydrochloride, metoprolol succinate, metoprolol tartrate, minoxidil, moexipril hydrochloride, nadolol, nicardipine hydrochloride, nifedipine, nisoldipine, nitroprusside sodium, penbutolol sulfate, perindopril erbumine, phentolamine mesylate, pindolol, prazosin hydrochloride, propranolol hydrochloride, quinapril hydrochloride, ramipril, telmisartan, terazosin hydrochloride, timolol maleate, trandolapril, valsartan, verapamil hydrochloride The at least one antilipemic can be at least one selected from atorvastatin calcium, cerivastatin sodium, cholestyramine, colestipol hydrochloride, fenofibrate (micronized), fluvastatin sodium, gemfibrozil, lovastatin, niacin, pravastatin sodium, simvastatin. The at least one miscellaneous CV drug can be at least one selected from abciximab, alprostadil, arbutamine hydrochloride, cilostazol, clopidogrel bisulfate, dipyridamole, eptifibatide, midodrine hydrochloride, pentoxifylline, ticlopidine hydrochloride, tirofiban hydrochloride. (See, e.g., pp. 215-336 of Nursing 2001 Drug Handbook.)

The at least one nonnarcotic analgesic or antipyretic can be at least one selected from acetaminophen, aspirin, choline magnesium trisalicylate, diflunisal, magnesium salicylate. The at least one nonsteroidal anti-inflammatory drug can be at least one selected from celecoxib, diclofenac potassium, diclofenac sodium, etodolac, fenoprofen calcium, flurbiprofen, ibuprofen, indomethacin, indomethacin sodium trihydrate, ketoprofen, ketorolac tromethamine, nabumetone, naproxen, naproxen sodium, oxaprozin, piroxicam, rofecoxib, sulindac. The at least one narcotic or opiod analgesic can be at least one selected from alfentanil hydrochloride, buprenorphine hydrochloride, butorphanol tartrate, codeine phosphate, codeine sulfate, fentanyl citrate, fentanyl transdermal system, fentanyl transmucosal, hydromorphone hydrochloride, meperidine hydrochloride, methadone hydrochloride, morphine hydrochloride, morphine sulfate, morphine tartrate, nalbuphine hydrochloride, oxycodone hydrochloride, oxycodone pectinate, oxymorphone hydrochloride, pentazocine hydrochloride, pentazocine hydrochloride and naloxone hydrochloride, pentazocine lactate, propoxyphene hydrochloride, propoxyphene napsylate, remifentanil hydrochloride, sufentanil citrate, tramadol hydrochloride. The at least one sedative-hypnotic can be at least one selected from chloral hydrate, estazolam, flurazepam hydrochloride, pentobarbital, pentobarbital sodium, phenobarbital sodium, secobarbital sodium, temazepam, triazolam, zaleplon, zolpidem tartrate. The at least one anticonvulsant can be at least one selected from acetazolamide sodium, carbamazepine, clonazepam, clorazepate dipotassium, diazepam, divalproex sodium, ethosuximde, fosphenytoin sodium, gabapentin, lamotrigine, magnesium sulfate, phenobarbital, phenobarbital sodium, phenytoin, phenytoin sodium, phenytoin sodium (extended), primidone, tiagabine hydrochloride, topiramate, valproate sodium, valproic acid. The at least one antidepressant can be at least one selected from amitriptyline hydrochloride, amitriptyline pamoate, amoxapine, bupropion hydrochloride, citalopram hydrobromide, clomipramine hydrochloride, desipramine hydrochloride, doxepin hydrochloride, fluoxetine hydrochloride, imipramine hydrochloride, imipramine pamoate, mirtazapine, nefazodone hydrochloride, nortriptyline hydrochloride, paroxetine hydrochloride, phenelzine sulfate, sertraline hydrochloride, tranylcypromine sulfate, trimipramine maleate, venlafaxine hydrochloride. The at least one antianxiety drug can be at least one selected from alprazolam, buspirone hydrochloride, chlordiazepoxide, chlordiazepoxide hydrochloride, clorazepate dipotassium, diazepam, doxepin hydrochloride, hydroxyzine embonate, hydroxyzine hydrochloride, hydroxyzine pamoate, lorazepam, mephrobamate, midazolam hydrochloride, oxazepam. The at least one antipsychotic drug can be at least one selected from chlorpromazine hydrochloride, clozapine, fluphenazine decanoate, fluephenazine enanthate, fluphenazine hydrochloride, haloperidol, haloperidol decanoate, haloperidol lactate, loxapine hydrochloride, loxapine succinate, mesoridazine besylate, molindone hydrochloride, olanzapine, perphenazine, pimozide, prochlorperazine, quetiapine fumarate, risperidone, thioridazine hydrochloride, thiothixene, thiothixene hydrochloride, trifluoperazine hydrochloride. The at least one central nervous system stimulant can be at least one selected from amphetamine sulfate, caffeine, dextroamphetamine sulfate, doxapram hydrochloride, methamphetamine hydrochloride, methylphenidate hydrochloride, modafinil, pemoline, phentermine hydrochloride. The at least one antiparkinsonian can be at least one selected from amantadine hydrochloride, benztropine mesylate, biperiden hydrochloride, biperiden lactate, bromocriptine mesylate, carbidopa-levodopa, entacapone, levodopa, pergolide mesylate, pramipexole dihydrochloride, ropinirole hydrochloride, selegiline hydrochloride, tolcapone, trihexyphenidyl hydrochloride. The at least one miscellaneous central nervous system drug can be at least one selected from bupropion hydrochloride, donepezil hydrochloride, droperidol, fluvoxamine maleate, lithium carbonate, lithium citrate, naratriptan hydrochloride, nicotine polacrilex, nicotine transdermal system, propofol, rizatriptan benzoate, sibutramine hydrochloride monohydrate, sumatriptan succinate, tacrine hydrochloride, zolmitriptan. (See, e.g., pp. 337-530 of Nursing 2001 Drug Handbook.)

The at least one cholinergic (e.g., parasymathomimetic) can be at least one selected from bethanechol chloride, edrophonium chloride, neostigmine bromide, neostigmine methylsulfate, physostigmine salicylate, pyridostigmine bromide. The at least one anticholinergics can be at least one selected from atropine sulfate, dicyclomine hydrochloride, glycopyrrolate, hyoscyamine, hyoscyamine sulfate, propantheline bromide, scopolamine, scopolamine butylbromide, scopolamine hydrobromide. The at least one adrenergics (sympathomimetics) can be at least one selected from dobutamine hydrochloride, dopamine hydrochloride, metaraminol bitartrate, norepinephrine bitartrate, phenylephrine hydrochloride, pseudoephedrine hydrochloride, pseudoephedrine sulfate. The at least one adrenergic blocker (sympatholytic) can be at least one selected from dihydroergotamine mesylate, ergotamine tartrate, methysergide maleate, propranolol hydrochloride. The at least one skeletal muscle relaxant can be at least one selected from baclofen, carisoprodol, chlorzoxazone, cyclobenzaprine hydrochloride, dantrolene sodium, methocarbamol, tizanidine hydrochloride. The at least one neuromuscular blockers can be at least one selected from atracurium besylate, cisatracurium besylate, doxacurium chloride, mivacurium chloride, pancuronium bromide, pipecuronium bromide, rapacuronium bromide, rocuronium bromide, succinylcholine chloride, tubocurarine chloride, vecuronium bromide. (See, e.g., pp. 531-84 of Nursing 2001 Drug Handbook.)

The at least one antihistamine can be at least one selected from brompheniramine maleate, cetirizine hydrochloride, chlorpheniramine maleate, clemastine fumarate, cyproheptadine hydrochloride, diphenhydramine hydrochloride, fexofenadine hydrochloride, loratadine, promethazine hydrochloride, promethazine theoclate, triprolidine hydrochloride. The at least one bronchodilators can be at least one selected from albuterol, albuterol sulfate, aminophylline, atropine sulfate, ephedrine sulfate, epinephrine, epinephrine bitartrate, epinephrine hydrochloride, ipratropium bromide, isoproterenol, isoproterenol hydrochloride, isoproterenol sulfate, levalbuterol hydrochloride, metaproterenol sulfate, oxtriphylline, pirbuterol acetate, salmeterol xinafoate, terbutaline sulfate, theophylline. The at least one expectorants or antitussives can be at least one selected from benzonatate, codeine phosphate, codeine sulfate, dextramethorphan hydrobromide, diphenhydramine hydrochloride, guaifenesin, hydromorphone hydrochloride. The at least one miscellaneous respiratory drug can be at least one selected from acetylcysteine, beclomethasone dipropionate, beractant, budesonide, calfactant, cromolyn sodium, dornase alfa, epoprostenol sodium, flunisolide, fluticasone propionate, montelukast sodium, nedocromil sodium, palivizumab, triamcinolone acetonide, zafirlukast, zileuton. (See, e.g., pp. 585-642 of Nursing 2001 Drug Handbook.)

The at least one antacid, adsorbents, or antiflatulents can be at least one selected from aluminum carbonate, aluminum hydroxide, calcium carbonate, magaldrate, magnesium hydroxide, magnesium oxide, simethicone, sodium bicarbonate. The at least one digestive enymes or gallstone solubilizers can be at least one selected from pancreatin, pancrelipase, ursodiol. The at least one antidiarrheal can be at least one selected from attapulgite, bismuth subsalicylate, calcium polycarbophil, diphenoxylate hydrochloride or atropine sulfate, loperamide, octreotide acetate, opium tincture, opium tincure (camphorated). The at least one laxative can be at least one selected from bisocodyl, calcium polycarbophil, cascara sagrada, cascara sagrada aromatic fluidextract, cascara sagrada fluidextract, castor oil, docusate calcium, docusate sodium, glycerin, lactulose, magnesium citrate, magnesium hydroxide, magnesium sulfate, methylcellulose, mineral oil, polyethylene glycol or electrolyte solution, psyllium, senna, sodium phosphates. The at least one antiemetic can be at least one selected from chlorpromazine hydrochloride, dimenhydrinate, dolasetron mesylate, dronabinol, granisetron hydrochloride, meclizine hydrochloride, metocloproamide hydrochloride, ondansetron hydrochloride, perphenazine, prochlorperazine, prochlorperazine edisylate, prochlorperazine maleate, promethazine hydrochloride, scopolamine, thiethylperazine maleate, trimethobenzamide hydrochloride. The at least one antiulcer drug can be at least one selected from cimetidine, cimetidine hydrochloride, famotidine, lansoprazole, misoprostol, nizatidine, omeprazole, rabeprozole sodium, rantidine bismuth citrate, ranitidine hydrochloride, sucralfate. (See, e.g., pp. 643-95 of Nursing 2001 Drug Handbook.)

The at least one coricosteroids can be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, triamcinolone diacetate. The at least one androgen or anabolic steroids can be at least one selected from danazol, fluoxymesterone, methyltestosterone, nandrol one decanoate, nandrol one phenpropionate, testosterone, testosterone cypionate, testosterone enanthate, testosterone propionate, testosterone transdermal system. The at least one estrogen or progestin can be at least one selected from esterified estrogens, estradiol, estradiol cypionate, estradiol/norethindrone acetate transdermal system, estradiol valerate, estrogens (conjugated), estropipate, ethinyl estradiol, ethinyl estradiol and desogestrel, ethinyl estradiol and ethynodiol diacetate, ethinyl estradiol and desogestrel, ethinyl estradiol and ethynodiol diacetate, ethinyl estradiol and levonorgestrel, ethinyl estradiol and norethindrone, ethinyl estradiol and norethindrone acetate, ethinyl estradiol and norgestimate, ethinyl estradiol and norgestrel, ethinyl estradiol and norethindrone and acetate and ferrous fumarate, levonorgestrel, medroxyprogesterone acetate, mestranol and norethindron, norethindrone, norethindrone acetate, norgestrel, progesterone. The at least one gonadroptropin can be at least one selected from ganirelix acetate, gonadoreline acetate, histrelin acetate, menotropins. The at least one antidiabetic or glucaon can be at least one selected from acarbose, chlorpropamide, glimepiride, glipizide, glucagon, glyburide, insulins, metformin hydrochloride, miglitol, pioglitazone hydrochloride, repaglinide, rosiglitazone maleate, troglitazone. The at least one thyroid hormone can be at least one selected from levothyroxine sodium, liothyronine sodium, liotrix, thyroid. The at least one thyroid hormone antagonist can be at least one selected from methimazole, potassium iodide, potassium iodide (saturated solution), propylthiouracil, radioactive iodine (sodium iodide ¹³¹I), strong iodine solution. The at least one pituitary hormone can be at least one selected from corticotropin, cosyntropin, desmophressin acetate. leuprolide acetate, repository corticotropin, somatrem, somatropin, vasopressin. The at least one parathyroid-like drug can be at least one selected from calcifediol, calcitonin (human), calcitonin (salmon), calcitriol, dihydrotachysterol, etidronate disodium. (See, e.g., pp. 696-796 of Nursing 2001 Drug Handbook.)

The at least one diuretic can be at least one selected from acetazolamide, acetazolamide sodium, amiloride hydrochloride, bumetanide, chlorthalidone, ethacrynate sodium, ethacrynic acid, furosemide, hydrochlorothiazide, indapamide, mannitol, metolazone, spironolactone, torsemide, triamterene, urea. The at least one electrolyte or replacement solution can be at least one selected from calcium acetate, calcium carbonate, calcium chloride, calcium citrate, calcium glubionate, calcium gluceptate, calcium gluconate, calcium lactate, calcium phosphate (dibasic), calcium phosphate (tribasic), dextran (high-molecular-weight), dextran (low-molecular-weight), hetastarch, magnesium chloride, magnesium sulfate, potassium acetate, potassium bicarbonate, potassium chloride, potassium gluconate, Ringer's injection, Ringer's injection (lactated), sodium chloride. The at least one acidifier or alkalinizer can be at least one selected from sodium bicarbonate, sodium lactate, tromethamine. (See, e.g., pp. 797-833 of Nursing 2001 Drug Handbook.)

The at least one hematinic can be at least one selected from ferrous fumarate, ferrous gluconate, ferrous sulfate, ferrous sulfate (dried), iron dextran, iron sorbitol, polysaccharide-iron complex, sodium ferric gluconate complex. The at least one anticoagulant can be at least one selected from ardeparin sodium, dalteparin sodium, danaparoid sodium, enoxaparin sodium, heparin calcium, heparin sodium, warfarin sodium. The at least one blood derivative can be at least one selected from albumin 5%, albumin 25%, antihemophilic factor, anti-inhibitor coagulant complex, antithrombin III (human), factor IX (human), factor IX complex, plasma protein fractions. The at least one thrombolytic enzyme can be at least one selected from alteplase, anistreplase, reteplase (recombinant), streptokinase, urokinase. (See, e.g., pp. 834-66 of Nursing 2001 Drug Handbook.)

The at least one alkylating drug can be at least one selected from busulfan, carboplatin, carmustine, chlorambucil, cisplatin, cyclophosphamide, ifosfamide, lomustine, mechlorethamine hydrochloride, melphalan, melphalan hydrochloride, streptozocin, temozolomide, thiotepa. The at least one antimetabolite can be at least one selected from capecitabine, cladribine, cytarabine, floxuridine, fludarabine phosphate, fluorouracil, hydroxyurea, mercaptopurine, methotrexate, methotrexate sodium, thioguanine. The at least one antibiotic antineoplastic can be at least one selected from bleomycin sulfate, dactinomycin, daunorubicin citrate liposomal, daunorubicin hydrochloride, doxorubicin hydrochloride, doxorubicin hydrochloride liposomal, epirubicin hydrochloride, idarubicin hydrochloride, mitomycin, pentostatin, plicamycin, valrubicin. The at least one antineoplastics that alter hormone balance can be at least one selected from anastrozole, bicalutamide, estramustine phosphate sodium, exemestane, flutamide, goserelin acetate, letrozole, leuprolide acetate, megestrol acetate, nilutamide, tamoxifen citrate, testolactone, toremifene citrate. The at least one miscellaneous antineoplastic can be at least one selected from asparaginase, bacillus Calmette-Guerin (BCG) (live intravesical), dacarbazine, docetaxel, etoposide, etoposide phosphate, gemcitabine hydrochloride, irinotecan hydrochloride, mitotane, mitoxantrone hydrochloride, paclitaxel, pegaspargase, porfimer sodium, procarbazine hydrochloride, rituximab, teniposide, topotecan hydrochloride, trastuzumab, tretinoin, vinblastine sulfate, vincristine sulfate, vinorelbine tartrate. (See, e.g., pp. 867-963 of Nursing 2001 Drug Handbook.)

The at least one immunosuppressant can be at least one selected from azathioprine, basiliximab, cyclosporine, daclizumab, lymphocyte immune globulin, muromonab-CD3, mycophenolate mofetil, mycophenolate mofetil hydrochloride, sirolimus, tacrolimus. The at least one vaccine or toxoid can be at least one selected from BCG vaccine, cholera vaccine, diphtheria and tetanus toxoids (adsorbed), diphtheria and tetanus toxoids and acellular pertussis vaccine adsorbed, diphtheria and tetanus toxoids and whole-cell pertussis vaccine, Haemophilius b conjugate vaccines, hepatitis A vaccine (inactivated), hepatisis B vaccine (recombinant), influenza virus vaccine 1999-2000 trivalent types A & B (purified surface antigen), influenza virus vaccine 1999-2000 trivalent types A & B (subvirion or purified subvirion), influenza virus vaccine 1999-2000 trivalent types A & B (whole virion), Japanese encephalitis virus vaccine (inactivated), Lyme disease vaccine (recombinant OspA), measles and mumps and rubella virus vaccine (live), measles and mumps and rubella virus vaccine (live attenuated), measles virus vaccine (live attenuated), meningococcal polysaccharide vaccine, mumps virus vaccine (live), plague vaccine, pneumococcal vaccine (polyvalent), poliovirus vaccine (inactivated), poliovirus vaccine (live, oral, trivalent), rabies vaccine (adsorbed), rabies vaccine (human diploid cell), rubella and mumps virus vaccine (live), rubella virus vaccine (live, attenuated), tetanus toxoid (adsorbed), tetanus toxoid (fluid), typhoid vaccine (oral), typhoid vaccine (parenteral), typhoid Vi polysaccharide vaccine, varicella virus vaccine, yellow fever vaccine. The at least one antitoxin or antivenin can be at least one selected from black widow spider antivenin, Crotalidae antivenom (polyvalent), diphtheria antitoxin (equine), Micrurus fulvius antivenin). The at least one immune serum can be at least one selected from cytomegalovirus immune globulin (intraveneous), hepatitis B immune globulin (human), immune globulin intramuscular, immune globulin intravenous, rabies immune globulin (human), respiratory syncytial virus immune globulin intravenous (human), Rh₀(D) immune globulin (human), Rh₀(D) immune globulin intravenous (human), tetanus immune globulin (human), varicella-zoster immune globulin. The at least one biological response modifiers can be at least one selected from aldesleukin, epoetin alfa, filgrastim, glatiramer acetate for injection, interferon alfacon-1, interferon alfa-2a (recombinant), interferon alfa-2b (recombinant), interferon beta-1a, interferon beta-1b (recombinant), interferon gamma-1b, ievamisole hydrochloride, oprelvekin, sargramostim. (See, e.g., pp. 964-1040 of Nursing 2001 Drug Handbook.)

The at least one ophthalmic anti-infectives can be selected form bacitracin, chloramphenicol, ciprofloxacin hydrochloride, erythromycin, gentamicin sulfate, ofloxacin 0.3%, polymyxin B sulfate, sulfacetamide sodium 10%, sulfacetamide sodium 15%, sulfacetamide sodium 30%, tobramycin, vidarabine. The at least one ophthalmic anti-inflammatories can be at least one selected from dexamethasone, dexamethasone sodium phosphate, diclofenac sodium 0.1%, fluorometholone, flurbiprofen sodium, ketorolac tromethamine, prednisolone acetate (suspension) prednisolone sodium phosphate (solution). The at least one miotic can be at least one selected from acetylocholine chloride, carbachol (intraocular), carbachol (topical), echothiophate iodide, pilocarpine, pilocarpine hydrochloride, pilocarpine nitrate. The at least one mydriatic can be at least one selected from atropine sulfate, cyclopentolate hydrochloride, epinephrine hydrochloride, epinephryl borate, homatropine hydrobromide, phenylephrine hydrochloride, scopolamine hydrobromide, tropicamide. The at least one ophthalmic vasoconstrictors can be at least one selected from naphazoline hydrochloride, oxymetazoline hydrochloride, tetrahydrozoline hydrochloride. The at least one miscellaneous ophthalmics can be at least one selected from apraclonidine hydrochloride, betaxolol hydrochloride, brimonidine tartrate, carteolol hydrochloride, dipivefrin hydrochloride, dorzolamide hydrochloride, emedastine difumarate, fluorescein sodium, ketotifen fumarate, latanoprost, levobunolol hydrochloride, metipranolol hydrochloride, sodium chloride (hypertonic), timolol maleate. The at least one otic can be at least one selected from boric acid, carbamide peroxide, chloramphenicol, triethanolamine polypeptide oleate-condensate. The at least one nasal drug can be at least one selected from beclomethasone dipropionate, budesonide, ephedrine sulfate, epinephrine hydrochloride, flunisolide, fluticasone propionate, naphazoline hydrochloride, oxymetazoline hydrochloride, phenylephrine hydrochloride, tetrahydrozoline hydrochloride, triamcinolone acetonide, xylometazoline hydrochloride. (See, e.g., pp. 1041-97 of Nursing 2001 Drug Handbook.)

The at least one local anti-infectives can be at least one selected from acyclovir, amphotericin B, azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, tolnaftate. The at least one scabicide or pediculicide can be at least one selected from crotamiton, lindane, permethrin, pyrethrins. The at least one topical corticosteroid can be at least one selected from betamethasone dipropionate, betamethasone valerate, clobetasol propionate, desonide, desoximetasone, dexamethasone, dexamethasone sodium phosphate, diflorasone diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, triamcinolone acetonide. (See, e.g., pp. 1098-1136 of Nursing 2001 Drug Handbook.)

The at least one vitamin or mineral can be at least one selected from vitamin A, vitamin B complex, cyanocobalamin, folic acid, hydroxocobalamin, leucovorin calcium, niacin, niacinamide, pyridoxine hydrochloride, riboflavin, thiamine hydrochloride, vitamin C, vitamin D, cholecalciferol, ergocalciferol, vitamin D analogue, doxercalciferol, paricalcitol, vitamin E, vitamin K analogue, phytonadione, sodium fluoride, sodium fluoride (topical), trace elements, chromium, copper, iodine, manganese, selenium, zinc. The at least one calorics can be at least one selected from amino acid infusions (crystalline), amino acid infusions in dextrose, amino acid infusions with electrolytes, amino acid infusions with electrolytes in dextrose, amino acid infusions for hepatic failure, amino acid infusions for high metabolic stress, amino acid infusions for renal failure, dextrose, fat emulsions, medium-chain triglycerides. (See, e.g., pp. 1137-63 of Nursing 2001 Drug Handbook.)

Hinge core mimetibody antibody or polypeptide compositions of the present invention can further comprise at least one of any suitable and/or effective amount of a composition or pharmaceutical composition comprising at least one hinge core mimetibody protein or antibody to a cell, tissue, organ, animal or patient in need of such modulation, treatment or therapy, optionally further comprising at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, enteracept, CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Non-limiting examples of such cytokines include, but are not limted to, any of IL-1 to IL-23. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Such compositions can also include toxin molecules that are associated, bound, co-formulated or co-administered with at least one antibody or polypeptide of the present invention. The toxin can optionally act to selectively kill the pathologic cell or tissue. The pathologic cell can be a cancer or other cell. Such toxins can be, but are not limited to, purified or recombinant toxin or toxin fragment comprising at least one functional cytotoxic domain of toxin, e.g., selected from at least one of ricin, diphtheria toxin, a venom toxin, or a bacterial toxin. The term toxin also includes both endotoxins and exotoxins produced by any naturally occurring, mutant or recombinant bacteria or viruses which may cause any pathological condition in humans and other mammals, including toxin shock, which can result in death. Such toxins may include, but are not limited to, enterotoxigenic E. coli heat-labile enterotoxin (LT), heat-stable enterotoxin (ST), Shigella cytotoxin, Aeromonas enterotoxins, toxic shock syndrome toxin-1 (TSST-1), Staphylococcal enterotoxin A (SEA), B (SEB), or C (SEC), Streptococcal enterotoxins and the like. Such bacteria include, but are not limited to, strains of a species of enterotoxigenic E. coli (ETEC), enterohemorrhagic E. coli (e.g., strains of serotype 0157:H7), Staphylococcus species (e.g., Staphylococcus aureus, Staphylococcus pyogenes), Shigella species (e.g., Shigella dysenteriae, Shigella flexneri, Shigella boydii, and Shigella sonnei), Salmonella species (e.g., Salmonella typhi, Salmonella cholera-suis, Salmonella enteritidis), Clostridium species (e.g., Clostridium perfringens, Clostridium dificile, Clostridium botulinum), Camphlobacter species (e.g., Camphlobacter jejuni, Camphlobacter fetus), Heliobacter species, (e.g., Heliobacter pylori), Aeromonas species (e.g., Aeromonas sobria, Aeromonas hydrophila, Aeromonas caviae), Pleisomonas shigelloides, Yersina enterocolitica, Vibrios species (e.g., Vibrios cholerae, Vibrios parahemolyticus), Klebsiella species, Pseudomonas aeruginosa, and Streptococci. See, e.g., Stein, ed., INTERNAL MEDICINE, 3rd ed., pp 1-13, Little, Brown and Co., Boston, (1990); Evans et al., eds., Bacterial Infections of Humans: Epidemiology and Control, 2d. Ed., pp 239-254, Plenum Medical Book Co., New York (1991); Mandell et al, Principles and Practice of Infectious Diseases, 3d. Ed., Churchill Livingstone, New York (1990); Berkow et al, eds., The Merck Manual, 16th edition, Merck and Co., Rahway, N.J., 1992; Wood et al, FEMS Microbiology Immunology, 76:121-134 (1991); Marrack et al, Science, 248:705-711 (1990), the contents of which references are incorporated entirely herein by reference.

hinge core mimetibody or specified portion or variant compositions of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the hinge core mimetibody composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/hinge core mimetibody or specified portion or variant components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

hinge core mimetibody compositions can also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts such as citrate.

Additionally, the hinge core mimetibody or specified portion or variant compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the hinge core mimetibody compositions according to the invention are known in the art, e.g., as listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), the disclosures of which are entirely incorporated herein by reference. Preferrred carrier or excipient materials are carbohydrates (e.g., saccharides and alditols) and buffers (e.g., citrate) or polymeric agents.

Formulations

As noted above, the invention provides for stable formulations, which can preferably include a suitable buffer with saline or a chosen salt, as well as optional preserved solutions and formulations containing a preservative as well as multi-use preserved formulations suitable for pharmaceutical or veterinary use, comprising at least one hinge core mimetibody or specified portion or variant in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1., 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one hinge core mimetibody or specified portion or variant with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one hinge core mimetibody or specified portion or variant, and a second vial comprising an aqueous diluent of prescribed buffer or preservative, wherein said packaging material comprises a label that instructs a patient to reconstitute the at least one hinge core mimetibody or specified portion or variant in the aqueous diluent to form a solution that can be held over a period of twenty-four hours or greater.

The at least one hinge core mimetibody or specified portion or variant used in accordance with the present invention can be produced by recombinant means, including from mammalian cell or transgenic preparations, or can be purified from other biological sources, as described herein or as known in the art.

The range of amounts of at least one hinge core mimetibody or specified portion or variant in the product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g. isotonicity agents, buffers, antioxidants, preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably the formulations of the present invention have pH between about 6.8 and about 7.8. Preferred buffers include phosphate buffers, most preferably sodium phosphate, particularly phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one hinge core mimetibody or specified portion or variant and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one hinge core mimetibody or specified portion or variant and preservative in an aqueous diluent is carried out using conventional dissolution and mixing procedures. To prepare a suitable formulation, for example, a measured amount of at least one hinge core mimetibody or specified portion or variant in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that may be optimized for the concentration and means of administration used.

The claimed formulations can be provided to patients as clear solutions or as dual vials comprising a vial of lyophilized at least one hinge core mimetibody or specified portion or variant that is reconstituted with a second vial containing water, a preservative and/or excipients, preferably a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period of immediately to twenty-four hours or greater. Accordingly, the presently claimed articles of manufacture offer significant advantages to the patient. Formulations of the invention can optionally be safely stored at temperatures of from about 2 to about 40° C. and retain the biologically activity of the protein for extended periods of time, thus, allowing a package label indicating that the solution can be held and/or used over a period of 6, 12, 18, 24, 36, 48, 72, or 96 hours or greater. If preserved diluent is used, such label can include use up to at least one of 1-12 months, one-half, one and a half, and/or two years.

The solutions of at least one hinge core mimetibody or specified portion or variant in the invention can be prepared by a process that comprises mixing at least one hinge core mimetibody or specified portion or variant in an aqueous diluent. Mixing is carried out using conventional dissolution and mixing procedures. To prepare a suitable diluent, for example, a measured amount of at least one hinge core mimetibody or specified portion or variant in water or buffer is combined in quantities sufficient to provide the protein and optionally a preservative or buffer at the desired concentrations. Variations of this process would be recognized by one of ordinary skill in the art. For example the order the components are added, whether additional additives are used, the temperature and pH at which the formulation is prepared, are all factors that may be optimized for the concentration and means of administration used.

The claimed products can be provided to patients as clear solutions or as dual vials comprising a vial of lyophilized at least one hinge core mimetibody or specified portion or variant that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one hinge core mimetibody or specified portion or variant that is reconstituted with a second vial containing the aqueous diluent. The clear solution in this case can be up to one liter or even larger in size, providing a large reservoir from which smaller portions of the at least one hinge core mimetibody or specified portion or variant solution can be retrieved one or multiple times for transfer into smaller vials and provided by the pharmacy or clinic to their customers and/or patients.

Recognized devices comprising these single vial systems include those pen-injector devices for delivery of a solution such as Humaject®, NovoPen®, B-D®Pen, AutoPen®, and OptiPen®. Recognized devices comprising a dual vial system include those pen-injector systems for reconstituting a lyophilized drug in a cartridge for delivery of the reconstituted solution such as the HumatroPen®.

The products presently claimed include packaging material. The packaging material provides, in addition to the information required by the regulatory agencies, the conditions under which the product can be used. The packaging material of the present invention provides instructions to the patient to reconstitute the at least one hinge core mimetibody or specified portion or variant in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one hinge core mimetibody or specified portion or variant and a selected buffer, preferably a phosphate buffer containing saline or a chosen salt. Mixing the at least one hinge core mimetibody or specified portion or variant and buffer in an aqueous diluent is carried out using conventional dissolution and mixing procedures. To prepare a suitable formulation, for example, a measured amount of at least one hinge core mimetibody or specified portion or variant in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized at least one hinge core mimetibody or specified portion or variant that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

At least one hinge core mimetibody or specified portion or variant in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

Therapeutic Applications

The present invention for mimetibodies also provides a method for modulating or treating anemia, in a cell, tissue, organ, animal, or patient including, but not limited to, at least one of any anemia, cancer treatment related anemia, radiotherapy or chemotherapy related anemia, viral or bacterial infection treatment related anemia, renal anemia, anemia of prematurity, pediatric and/or adult cancer-associated anemia, anemia associated with lymphoma, myeloma, multple myeloma, AIDS-associated anemia, concomitant treatment for patients with or without autologous blood donation awaiting elective surgery, preoperatve and post operative for surgery, autologous blood donation or transfusion, perioperative management, cyclic neutropenia or Kostmann syndrome (congenital agranulocytosis), end-stage renal disease, anemia associated with dialysis, chronic renal insufficiency, primary hemopoietic diseases, such as congenital hypoplastic anemia, thalassemia major, or sickle cell disease, vaso-occlusive complications of sickle cell disease. Furman et al., Pediatrics 1992; 90: 716-728, Goldberg Science. 1988;242:1412-1415; Paul et al., Exp Hematol. 1984;12:825-830; Erslev et al., Arch Intern Med. 1968;122:230-235; Ersley et al., Ann Clin Lab Sci. 1980;10:250-257; Jacobs et al., Nature. 1985;313:806-810; Lin et al., Proc Natl Acad Sci USA. 1985;82:7580-7584; Law et al., Proc Natl Acad Sci USA. 1986;83:6920-6924; Goldwasser et al., J Biol Chem. 1974;249:4202-4206; Eaves et a., Blood. 1978;52:1196-1210; Sawyer et al., Blood. 1989;74:103-109; Winearls et al., Lancet. 1986;2:1175-1178; Eschbach et al., N Engl J Med. 1987;316:73-78; Eschbach et al., Ann Intern Med. 1989; 111:992-1000, each reference entirely incoporated herein by reference.

Mimetibodies of the present invention can also be used for non-renal forms of anemia induced, for example, by chronic infections, inflammatory processes, radiation therapy, and cytostatic drug treatment, and encouraging results in patients with non-renal anemia have been reported. See, e.g., Abels R I and Rudnick S A Erythropoietin: evolving clinical applications. Experimental Hematology 19: 842-50 (1991); Graber S E and Krantz S B Erythropoietin: biology and clinical use. Hematology/Oncol. Clin. North Amer. 3: 369-400 (1989); Jelkman W and Gross A J (eds) Erythropoietin. Springer, Berlin 1989; Koury M J and Bondurant M C The molecular mechanism of erythropoietin action. European Journal of Biochemistry 210: 649-63 (1992); Krantz S B Erythropoietin. Blood 77: 419-34 (1991); Tabbara I A Erythropoietin. Biology and clinical applications. Archives of Internal Medicine 153: 298-304 (1993), each entirely incorporated herein by reference.

The present invention also provides a method for modulating or treating an anemia or blood cell related condition, in a cell, tissue, organ, animal, or patient, wherein said anemia or blood cell related condition is associated with at least one including, but not limited to, at least one of immune related disease, cardiovascular disease, infectious, malignant and/or neurologic disease. Such a method can optionally comprise administering an effective amount of at least one composition or pharmaceutical composition comprising at least one hinge core mimetibody or specified portion or variant to a cell, tissue, organ, animal or patient in need of such modulation, treatment or therapy.

The present invention also provides a method for modulating or treating cancer/infecteous disease in a cell, tissue, organ, animal or patient, including, but not limited to, at least one of acute or chronic bacterial infection, acute and chronic parasitic or infectious processes, including bacterial, viral and fungal infections, HIV infection/HIV neuropathy, meningitis, hepatitis, septic arthritis, peritonitis, pneumonia, epiglottitis, e. coli 0157:h7, hemolytic uremic syndrome/thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis/epidydimitis, legionella, lyme disease, influenza a, epstein-barr virus, vital-associated hemaphagocytic syndrome, vital encephalitis/aseptic meningitis, and the like; (ii) leukemia, acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia, myelodyplastic syndrome (MDS), a lymphoma, Hodgkin's disease, a malignamt lymphoma, non-hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, adenocarcinomas, sarcomas, malignant melanoma, and the like; or (iii) neurodegenerative diseases, multiple sclerosis, migraine headache, AIDS dementia complex, demyelinating diseases, such as multiple sclerosis and acute transverse myelitis; extrapyramidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supranucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi.system disorder); demyelinating core disorders, such as multiple sclerosis, acute transverse myelitis; and disorders of the motor unit' such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Hallerrorden-Spatz disease; and Dementia pugilistica, and the like. Such a method can optionally comprise administering an effective amount of a composition or pharmaceutical composition comprising at least one TNF antibody or specified portion or variant to a cell, tissue, organ, animal or patient in need of such modulation, treatment or therapy. See, e.g., the Merck Manual, 16^{th} Edition, Merck & Company, Rahway, N.J. (1992)

Such a method can optionally comprise administering an effective amount of at least one composition or pharmaceutical composition comprising at least one hinge core mimetibody or specified portion or variant to a cell, tissue, organ, animal or patient in need of such modulation, treatment or therapy.

The present invention also provides a method for modulating or treating at least one cardiovascular disease in a cell, tissue, organ, animal, or patient, including, but not limited to, at least one of cardiac stun syndrome, myocardial infarction, congestive heart failure, stroke, ischemic stroke, hemorrhage, arteriosclerosis, atherosclerosis, diabetic ateriosclerotic disease, hypertension, arterial hypertension, renovascular hypertension, syncope, shock, syphilis of the cardiovascular system, heart failure, cor pulmonale, primary pulmonary hypertension, cardiac arrhythmias, atrial ectopic beats, atrial flutter, atrial fibrillation (sustained or paroxysmal), chaotic or multifocal atrial tachycardia, regular narrow QRS tachycardia, specific arrythmias, ventricular fibrillation, His bundle arrythmias, atrioventricular block, bundle branch block, myocardial ischemic disorders, coronary artery disease, angina pectoris, myocardial infarction, cardiomyopathy, dilated congestive cardiomyopathy, restrictive cardiomyopathy, valvular heart diseases, endocarditis, pericardial disease, cardiac tumors, aordic and peripheral aneuryisms, aortic dissection, inflammation of the aorta, occulsion of the abdominal aorta and its branches, peripheral vascular disorders, occulsive arterial disorders, peripheral atherlosclerotic disease, thromboangitis obliterans, functional peripheral arterial disorders, Raynaud's phenomenon and disease, acrocyanosis, erythromelalgia, venous diseases, venous thrombosis, varicose veins, arteriovenous fistula, lymphederma, lipedema, unstable angina, reperfusion injury, post pump syndrome, ischemia-reperfusion injury, and the like. Such a method can optionally comprise administering an effective amount of a composition or pharmaceutical composition comprising at least one hinge core mimetibody or specified portion or variant to a cell, tissue, organ, animal or patient in need of such modulation, treatment or therapy.

Any method of the present invention can comprise administering an effective amount of a composition or pharmaceutical composition comprising at least one hinge core mimetibody or specified portion or variant to a cell, tissue, organ, animal or patient in need of such modulation, treatment or therapy. Such a method can optionally further comprise co-administration or combination therapy for treating such immune diseases, wherein the administering of said at least one hinge core mimetibody, specified portion or variant thereof, further comprises administering, before concurrently, and/or after, at least one selected from at least one TNF antagonist (e.g., but not limited to a TNF antibody or fragment, a soluble TNF receptor or fragment, fusion proteins thereof, or a small molecule TNF antagonist), an antirheumatic, a muscle relaxant, a narcotic, a non-steroid anti-inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Mimetibodies can also be used ex vivo, such as in autologous marrow culture. Briefly, bone marrow is removed from a patient prior to chemotherapy and treated with TPO and/or EPO, optionally in combination with mimetibodies, optionally in combination with one or more additional cytokines. The treated marrow is then returned to the patient after chemotherapy to speed the recovery of the marrow. In addition, TPO, alone and in combination with EPO mimetibodies and/or EPO, can also be used for the ex vivo expansion of marrow or peripheral blood progenitor (PBPC) cells. Prior to chemotherapy treatment, marrow can be stimulated with stem cell factor (SCF) or G-CSF to release early progenitor cells into peripheral circulation. These progenitors are optionally collected and concentrated from peripheral blood and then treated in culture with TPO and mimetibodies, optionally in combination with one or more other cytokines, including but not limited to SCF, G-CSF, IL-3, GM-CSF, IL-6 or IL-11, to differentiate and proliferate into high-density megakaryocyte cultures, which are optionally then be returned to the patient following high-dose chemotherapy. Doses of TPO for ex vivo treatment of bone marrow will be in the range of 100 pg/ml to 10 ng/ml, preferably 500 pg/ml to 3 ng/ml. Doses of mimetibodies will be equivalent in activity to EPO which can be used from 0.1 units/ml to 20 units/ml, preferably from 0.5 units/ml to 2 units/ml, or any range or value therein.

TNF antagonists suitable for compositions, combination therapy, co-administration, devices and/or methods of the present invention (further comprising at least one anti body, specified portion and variant thereof, of the present invention), include, but are not limited to, anti-TNF antibodies, ligand-binding fragments thereof, and receptor molecules which bind specifically to TNF; compounds which prevent and/or inhibit TNF synthesis, TNF release or its action on target cells, such as thalidomide, tenidap, phosphodiesterase inhibitors (e.g, pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

As used herein, a “tumor necrosis factor antibody,” “TNF antibody,” “TNFα antibody,” or fragment and the like decreases, blocks, inhibits, abrogates or interferes with TNFα activity in vitro, in situ and/or preferably in vivo. For example, a suitable TNF human antibody of the present invention can bind TNFα and includes anti-TNF antibodies, antigen-binding fragments thereof, and specified mutants or domains thereof that bind specifically to TNFα. A suitable TNF antibody or fragment can also decrease block, abrogate, interfere, prevent and/or inhibit TNF RNA, DNA or protein synthesis, TNF release, TNF receptor signaling, membrane TNF cleavage, TNF activity, TNF production and/or synthesis.

Chimeric antibody cA2 consists of the antigen binding variable region of the high-affinity neutralizing mouse anti-human TNFα IgG1 antibody, designated A2, and the constant regions of a human IgG1, kappa immunoglobulin. The human IgG1 Fc region improves allogeneic antibody effector function, increases the circulating serum half-life and decreases the immunogenicity of the antibody. The avidity and epitope specificity of the chimeric antibody cA2 is derived from the variable region of the murine antibody A2. In a particular embodiment, a preferred source for nucleic acids encoding the variable region of the murine antibody A2 is the A2 hybridoma cell line.

Chimeric A2 (cA2) neutralizes the cytotoxic effect of both natural and recombinant human TNFα in a dose dependent manner. From binding assays of chimeric antibody cA2 and recombinant human TNFα, the affinity constant of chimeric antibody cA2 was calculated to be 1.04×10¹⁰M⁻¹. Preferred methods for determining monoclonal antibody specificity and affinity by competitive inhibition can be found in Harlow, et al., Antibodies: A Laboratory Manual, Cold Spring Harbor Laboratory Press, Cold Spring Harbor, N.Y., 1988; Colligan et al., eds., Current Protocols in Immunology, Greene Publishing Assoc. and Wiley Interscience, New York, (1992-2003); Kozbor et al., Immunol. Today, 4:72-79 (1983); Ausubel et al., eds. Current Protocols in Molecular Biology, Wiley Interscience, New York (1987-2003); and Muller, Meth. Enzymol., 92:589-601 (1983), which references are entirely incorporated herein by reference.

In a particular embodiment, murine monoclonal antibody A2 is produced by a cell line designated c134A. Chimeric antibody cA2 is produced by a cell line designated c168A.

Additional examples of monoclonal anti-TNF antibodies that can be used in the present invention are described in the art (see, e.g., U.S. Pat. No. 5,231,024; Möller, A. et al., Cytokine 2(3):162-169 (1990); U.S. application Ser. No. 07/943,852 (filed Sep. 11, 1992); Rathjen et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); and Hirai, et al., J. Immunol. Meth. 96:57-62 (1987), which references are entirely incorporated herein by reference).

TNF Receptor Molecules

Preferred TNF receptor molecules useful in the present invention are those that bind TNFα with high affinity (see, e.g., Feldmann et al., International Publication No. WO 92/07076 (published Apr. 30, 1992); Schall et al., Cell 61:361-370 (1990); and Loetscher et al., Cell 61:351-359 (1990), which references are entirely incorporated herein by reference) and optionally possess low immunogenicity. In particular, the 55 kDa (p55 TNF-R) and the 75 kDa (p75 TNF-R) TNF cell surface receptors are useful in the present invention. Truncated forms of these receptors, comprising the extracellular domains (ECD) of the receptors or functional portions thereof (see, e.g., Corcoran et al., Eur. J. Biochem. 223:831-840 (1994)), are also useful in the present invention. Truncated forms of the TNF receptors, comprising the ECD, have been detected in urine and serum as 30 kDa and 40 kDa TNFα inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof, are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high affinity, as well as other undefined properties, may contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. An example of such a TNF immunoreceptor fusion molecule is TNF receptor/IgG fusion protein. TNF immunoreceptor fusion molecules and methods for their production have been described in the art (Lesslauer et al., Eur. J. Immunol. 21:2883-2886 (1991); Ashkenazi et al., Proc. Natl. Acad. Sci. USA 88:10535-10539 (1991); Peppel et al., J. Exp. Med. 174:1483-1489 (1991); Kolls et al., Proc. Natl. Acad. Sci. USA 91:215-219 (1994); Butler et al., Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (filed May 16, 1995), each of which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

A functional equivalent, derivative, fragment or region of TNF receptor molecule refers to the portion of the TNF receptor molecule, or the portion of the TNF receptor molecule sequence which encodes TNF receptor molecule, that is of sufficient size and sequences to functionally resemble TNF receptor molecules that can be used in the present invention (e.g., bind TNFα with high affinity and possess low immunogenicity). A functional equivalent of TNF receptor molecule also includes modified TNF receptor molecules that functionally resemble TNF receptor molecules that can be used in the present invention (e.g., bind TNFα with high affinity and possess low immunogenicity). For example, a functional equivalent of TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1987-2003).

Cytokines include, but are not limited to all known cytokines. See, e.g., CopewithCytokines.com. Cytokine antagonists include, but are not limited to, any antibody, fragment or mimetic, any soluble receptor, fragment or mimetic, any small molecule antagonist, or any combination thereof.

Any method of the present invention can comprise a method for treating a protein mediated disorder, comprising administering an effective amount of a composition or pharmaceutical composition comprising at least one hinge core mimetibody or specified portion or variant to a cell, tissue, organ, animal or patient in need of such modulation, treatment or therapy. Such a method can optionally further comprise co-administration or combination therapy for treating such immune diseases, wherein the administering of said at least one hinge core mimetibody, specified portion or variant thereof, further comprises administering, before concurrently, and/or after, at least one selected from at least one other cytokines such as IL-3, IL -6 and IL-11; stem cell factor; G-CSF and GM-CSF. Combination therapy with GM-CSF, for example, is indicated in patients with low neutrophil levels.

Typically, treatment of pathologic conditions is effected by administering an effective amount or dosage of at least one hinge core mimetibody composition that total, on average, a range from at least about 0.01 to 500 milligrams of at least one hinge core mimetibody or specified portion or variant/kilogram of patient per dose, and preferably from at least about 0.1 to 100 milligrams hinge core mimetibody or specified portion or variant/kilogram of patient per single or multiple administration, depending upon the specific activity of contained in the composition. Alternatively, the effective serum concentration can comprise 0.1-5000 μg/ml serum concentration per single or multiple adminstration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.01, 0.02, 0.03, 0.04, 0.05, 0.06, 0.07, 0.08, 009, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, and/or 30 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5., 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably 0.1 to 10 milligrams per kilogram per administration or in sustained release form is effective to obtain desired results.

As a non-limiting example, treatment of humans or animals can be provided as a one-time or periodic dosage of at least one hinge core mimetibody or specified portion or variant of the present invention 0.01 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20, or any combination thereof, using single, infusion or repeated doses.

Dosage forms (composition) suitable for internal administration generally contain from about 0.0001 milligram to about 500 milligrams of active ingredient per unit or container. In these pharmaceutical compositions the active ingredient will ordinarily be present in an amount of about 0.5-95% by weight based on the total weight of the composition.

For parenteral administration, the hinge core mimetibody or specified portion or variant can be formulated as a solution, suspension, emulsion or lyophilized powder in association, or separately provided, with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Therapeutic Administration

Many known and developed modes of can be used according to the present invention for administering pharmaceutically effective amounts of at least one hinge core mimetibody or specified portion or variant according to the present invention. While pulmonary administration is used in the following description, other modes of administration can be used according to the present invention with suitable results.

A hinge core mimetibody of the present invention can be delivered in a carrier, as a solution, emulsion, colloid, or suspension, or as a powder, using any of a variety of devices and methods suitable for administration by inhalation or other modes described here within or known in the art.

Parenteral Formulations and Administration

Formulations for parenteral administration can contain as common excipients sterile water or saline, polyalkylene glycols such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent such as aquous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

Alternative Delivery

The invention further relates to the administration of at least one hinge core mimetibody or specified portion or variant by parenteral, subcutaneous, intramuscular, intravenous, bolus, vaginal, rectal, buccal, sublingual, intranasal, or transdermal means. Protein, hinge core mimetibody or specified portion or variant compositions can be prepared for use for parenteral (subcutaneous, intramuscular or intravenous) administration particularly in the form of liquid solutions or suspensions; for use in vaginal or rectal administration particularly in semisolid forms such as creams and suppositories; for buccal, or sublingual administration particularly in the form of tablets or capsules; or intranasally particularly in the form of powders, nasal drops or aerosols or certain agents; or transdermally particularly in the form of a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement”; Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways such as electroporation, or to increase the mobility of charged drugs through the skin such as iontophoresis, or application of ultrasound such as sonophoresis (U.S. Pat. Nos. 4,309,989 and 4,767,402) (the above publications and patents being entirely incorporated herein by reference).

Pulmonary/Nasal Administration

For pulmonary administration, preferably at least one hinge core mimetibody or specified portion or variant composition is delivered in a particle size effective for reaching the lower airways of the lung or sinuses. According to the invention, at least one hinge core mimetibody or specified portion or variant can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of hinge core mimetibody or specified portion or variants are also known in the art. All such devices can use of formulations suitable for the administration for the dispensing of hinge core mimetibody or specified portion or variant in an aerosol. Such aerosols can be comprised of either solutions (both aqueous and non aqueous) or solid particles. Metered dose inhalers like the Ventolin® metered dose inhaler, typically use a propellent gas and require actuation during inspiration (See, e.g., WO 94/16970, WO 98/35888). Dry powder inhalers like Turbuhaler™ (Astra), Rotahaler® (Glaxo), Diskus® (Glaxo), Spiros™ inhaler (Dura), devices marketed by Inhale Therapeutics, and the Spinhaler® powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent® nebulizer (Mallinckrodt), and the Acorn II® nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention. Preferably, a composition comprising at least one hinge core mimetibody or specified portion or variant is delivered by a dry powder inhaler or a sprayer. There are a several desirable features of an inhalation device for administering at least one hinge core mimetibody or specified portion or variant of the present invention. For example, delivery by the inhalation device is advantageously reliable, reproducible, and accurate. The inhalation device can optionally deliver small dry particles, e.g. less than about 10 μm, preferably about 1-5 μm, for good respirability.

Administration of Hinge Core Mimetibody or Specified Portion or Variant Compositions as a Spray

A spray including hinge core mimetibody or specified portion or variant composition protein can be produced by forcing a suspension or solution of at least one hinge core mimetibody or specified portion or variant through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one hinge core mimetibody or specified portion or variant composition protein delivered by a sprayer have a particle size less than about 10 μm, preferably in the range of about 1 μm to about 5 μm, and most preferably about 2 μm to about 3 μm.

Formulations of at least one hinge core mimetibody or specified portion or variant composition protein suitable for use with a sprayer typically include hinge core mimetibody or specified portion or variant composition protein in an aqueous solution at a concentration of about 1 mg to about 20 mg of at least one hinge core mimetibody or specified portion or variant composition protein per ml of solution. The formulation can include agents such as an excipient, a buffer, an isotonicity agent, a preservative, a surfactant, and, preferably, zinc. The formulation can also include an excipient or agent for stabilization of the hinge core mimetibody or specified portion or variant composition protein, such as a buffer, a reducing agent, a bulk protein, or a carbohydrate. Bulk proteins useful in formulating hinge core mimetibody or specified portion or variant composition proteins include albumin, protamine, or the like. Typical carbohydrates useful in formulating hinge core mimetibody or specified portion or variant composition proteins include sucrose, mannitol, lactose, trehalose, glucose, or the like. The hinge core mimetibody or specified portion or variant composition protein formulation can also include a surfactant, which can reduce or prevent surface-induced aggregation of the hinge core mimetibody or specified portion or variant composition protein caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of a protein such as mimetibodies, or specified portions or variants, can also be included in the formulation.

Administration of Hinge Core Mimetibody or Specified Portion or Variant Compositions by a Nebulizer

hinge core mimetibody or specified portion or variant composition protein can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of hinge core mimetibody or specified portion or variant composition protein through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of hinge core mimetibody or specified portion or variant composition protein either directly or through a coupling fluid, creating an aerosol including the hinge core mimetibody or specified portion or variant composition protein. Advantageously, particles of hinge core mimetibody or specified portion or variant composition protein delivered by a nebulizer have a particle size less than about 10 μm, preferably in the range of about 1 μm to about 5 μm, and most preferably about 2 μm to about 3 μm.

Formulations of at least one hinge core mimetibody or specified portion or variant suitable for use with a nebulizer, either jet or ultrasonic, typically include hinge core mimetibody or specified portion or variant composition protein in an aqueous solution at a concentration of about 1 mg to about 20 mg of at least one hinge core mimetibody or specified portion or variant protein per ml of solution. The formulation can include agents such as an excipient, a buffer, an isotonicity agent, a preservative, a surfactant, and, preferably, zinc. The formulation can also include an excipient or agent for stabilization of the at least one hinge core mimetibody or specified portion or variant composition protein, such as a buffer, a reducing agent, a bulk protein, or a carbohydrate. Bulk proteins useful in formulating at least one hinge core mimetibody or specified portion or variant composition proteins include albumin, protamine, or the like. Typical carbohydrates useful in formulating at least one hinge core mimetibody or specified portion or variant include sucrose, mannitol, lactose, trehalose, glucose, or the like. The at least one hinge core mimetibody or specified portion or variant formulation can also include a surfactant, which can reduce or prevent surface-induced aggregation of the at least one hinge core mimetibody or specified portion or variant caused by atomization of the solution in forming an aerosol. Various conventional surfactants can be employed, such as polyoxyethylene fatty acid esters and alcohols, and polyoxyethylene sorbital fatty acid esters. Amounts will generally range between 0.001 and 4% by weight of the formulation. Especially preferred surfactants for purposes of this invention are polyoxyethylene sorbitan mono-oleate, polysorbate 80, polysorbate 20, or the like. Additional agents known in the art for formulation of a protein such as hinge core mimetibody or specified portion or variant protein can also be included in the formulation.

Administration of Hinge Core Mimetibody or Specified Portion or Variant Compositions by a Metered Dose Inhaler

In a metered dose inhaler (MDI), a propellant, at least one hinge core mimetibody or specified portion or variant, and any excipients or other additives are contained in a canister as a mixture including a liquefied compressed gas. Actuation of the metering valve releases the mixture as an aerosol, preferably containing particles in the size range of less than about 10 μm, preferably about 1 μm to about 5 μm, and most preferably about 2 μm to about 3 μm. The desired aerosol particle size can be obtained by employing a formulation of hinge core mimetibody or specified portion or variant composition protein produced by various methods known to those of skill in the art, including jet-milling, spray drying, critical point condensation, or the like. Preferred metered dose inhalers include those manufactured by 3M or Glaxo and employing a hydrofluorocarbon propellant.

Formulations of at least one hinge core mimetibody or specified portion or variant for use with a metered-dose inhaler device will generally include a finely divided powder containing at least one hinge core mimetibody or specified portion or variant as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluroalkane-134a), HFA-227 (hydrofluroalkane-227), or the like. Preferably the propellant is a hydrofluorocarbon. The surfactant can be chosen to stabilize the at least one hinge core mimetibody or specified portion or variant as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol. Additional agents known in the art for formulation of a protein such as protein can also be included in the formulation.

One of ordinary skill in the art will recognize that the methods of the current invention can be achieved by pulmonary administration of at least one hinge core mimetibody or specified portion or variant compositions via devices not described herein.

Mucosal Formulations and Administration

For absorption through mucosal surfaces, compositions and methods of administering at least one hinge core mimetibody or specified portion or variant include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. Nos. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g. suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration excipients include sugars, calcium stearate, magnesium stearate, pregelinatined starch, and the like (U.S. Pat. No. 5,849,695).

Oral Formulations and Administration

Formulations for oral rely on the co-administration of adjuvants (e.g., resorcinols and nonionic surfactants such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant such as magnesium stearate, paraben, preserving agent such as sorbic acid, ascorbic acid, alpha-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations may contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. No. 5,879,681 and U.S. Pat. No. 5,5,871,753 are used to deliver biologically active agents orally are known in the art.

Transdermal Formulations and Administration

For transdermal administration, the at least one hinge core mimetibody or specified portion or variant is encapsulated in a delivery device such as a liposome or polymeric nanoparticles, microparticle, microcapsule, or microspheres (referred to collectively as microparticles unless otherwise stated). A number of suitable devices are known, including microparticles made of synthetic polymers such as polyhydroxy acids such as polylactic acid, polyglycolic acid and copolymers thereof, polyorthoesters, polyanhydrides, and polyphosphazenes, and natural polymers such as collagen, polyamino acids, albumin and other proteins, alginate and other polysaccharides, and combinations thereof (U.S. Pat. No. 5,814,599).

Prolonged Administration and Formulations

It can be sometimes desirable to deliver the compounds of the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b) e.g. a zinc tannate salt. Additionally, the compounds of the present invention or, preferably, a relatively insoluble salt such as those just described, can be formulated in a gel, for example, an aluminum monostearate gel with, e.g. sesame oil, suitable for injection. Particularly preferred salts are zinc salts, zinc tannate salts, pamoate salts, and the like. Another type of slow release depot formulation for injection would contain the compound or salt dispersed for encapsulated in a slow degrading, non-toxic, non-antigenic polymer such as a polylactic acid/polyglycolic acid polymer for example as described in U.S. Pat. No. 3,773,919. The compounds or, preferably, relatively insoluble salts such as those described above can also be formulated in cholesterol matrix silastic pellets, particularly for use in animals. Additional slow release, depot or implant formulations, e.g. gas or liquid liposomes are known in the literature (U.S. Pat. No. 5,770,222 and “Sustained and Controlled Release Drug Delivery Systems”, J. R. Robinson ed., Marcel Dekker, Inc., N.Y., 1978).

Having generally described the invention, the same will be more readily understood by reference to the following examples, which are provided by way of illustration and are not intended as limiting.

### EXAMPLE 1

### Cloning and Expression of Hinge Core Mimetibody in Mammalian Cells

A typical mammalian expression vector contains at least one promoter element, which mediates the initiation of transcription of mRNA, the hinge core mimetibody or specified portion or variant coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or pLNCX (Clonetech Labs, Palo Alto, Calif.), pcDNA3.1 (±), pcDNA/Zeo (±) or pcDNA3.1/Hygro (±) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded hinge core mimetibody or specified portion or variant. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of hinge core mimetibody or specified portion or variants.

The expression vectors pC1 and pC4 contain the strong promoter (LTR) of the Rous Sarcoma Virus (Cullen, et al., Molec. Cell. Biol. 5:438-447 (1985)) plus a fragment of the CMV-enhancer (Boshart, et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

Cloning and Expression in CHO Cells

The vector pC4 is used for the expression of hinge core mimetibody or specified portion or variant. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (e.g., alpha minus MEM, Life Technologies, Gaithersburg, Md.) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained that contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the EPO in a regulated way in mammalian cells (M. Gossen, and H. Bujard, Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete hinge core mimetibody or specified portion or variant is used, corresponding to HC and LC variable regions of a hinge core mimetibody of the present invention, according to known method steps. Isolated nucleic acid encoding a suitable human constant region (i.e., HC and LC regions) is also used in this construct.

The isolated variable and constant region encoding DNA and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained that grow at a concentration of 100-200 mM. Expression of the desired gene product is analyzed, for instance, by SDS-PAGE and Western blot or by reverse phase HPLC analysis.

Additional constructs can be expressed with single or multiple amino acid changes in order to avoid undesirable activities. These changes may be expressed alone or multiple changes combined in a single construct. The cysteine normally involved in a disulfide bridge between the HC and LC will be mutated to an alanine. While this cysteine may be involved in stabilizing the construct by forming a third disulfide bridge, it is possible that it may aberrantly form a disulfide bond with other cyseines within the construct, or it could form a disulfide linkage between two constructs. By removing the cysteine, proper folding and assembly could be enhanced and the possibility of self-association diminished.

It has been shown that mutation of two lysine (L) residues, L234 & L235, in the IgG1 lower hinge region to alanine (A) will abrogate the ability of the immunoglobulin to mediate complement dependent cytotoxicity (CDC) and antibody dependant cellular cytotoxicity (ADCC) (Hezereh et al., 2001, J. Virol., vol. 75 (24), 12161-68). Similar changes can be made in the hinge region of other immunoglobulin classes and subclasses.

Another modification that would result in a decrease in mediation of immune effector functions is the removal of the glycosylation attachment site. This can be accomplished by mutation of the asparagine to glutamine (Q). Aglycosylated versions of the IgG1 subclass are known to be poor mediators of immune effector function (Jefferis et al. 1998, Immol. Rev., vol. 163, 50-76).

An additional modification that is currently being pursued is the replacement of the IgG1 CH2 and CH3 regions with the same regions of the IgG4 subtype while retaining the G1 hinge region. As discussed previously, the ability of the IgG4 subclass to mediate immune effector functions is much lower than that of the G1 subclass. So this construct is expected to retain activity without the concerns of potential immune effector functions.

Other envisioned modifications are those that would decrease the potential immunogenicity of the constructs. One important determinant of immunogenicity is the ability of peptides derived from a protein to be efficiently bound and presented by MHC molecules to T cells and to elicit a cell based immune response or T cell help for an antibody response. Using publicly available web based algorithms for MHC binding (SYFPETHI, Ramensee et al., 1999, Immunogenetics, vol. 50, 213-19 and BIMAS) potential MHC binding epitopes within the mimetibody were analyzed. Mutations that would decrease the predicted immunogenicity of one or more peptides are evaluated for in vivo effect on immunogenicity.

Advantages: The mimetibody constructs described above offers an alternative way of displaying bioactive peptides. In addition, proposed modifications are expected to, in combination and in addition to the novel features of mimetibodies of the present invention, enhance their utility.

It will be clear that the invention can be practiced otherwise than as particularly described in the foregoing description and examples.

Numerous modifications and variations of the present invention are possible in light of the above teachings and, therefore, are within the scope of the present invention.

What is claimed is:

1. At least one hinge core mimetibody nucleic acid, comprising at least one polynucleotide encoding a polypeptide according to Formula (I):
((V(m)-P(n)-L(o)-H(p)-CH2(q)-CH3(r))(s),
where V is at least one portion of an N-terminus of an immunoglobulin variable region, P is at least one bioactive peptide, L is linker sequence, H is at least a portion of an immunoglobulin variable hinge region, CH2 is at least one portion of an immunoglobulin CH2 constant region, CH3 is at least one portion of an immunoglobulin CH3 constant region, and m, n, o, p, q, r and s, can independently be any integer between 0, 1 or 2 and 10.

2. At least one hinge core mimetibody polypeptide, comprising a polypeptide according to Formula (I):
((V(m)-P(n)-L(o)-H(p)-CH2(q)-CH3(r))(s),
where V is at least one portion of an N-terminus of an immunoglobulin variable region, P is at least one bioactive peptide selected from SEQ ID NOS:43-518, L is linker sequence, H is at least a portion of an immunoglobulin variable hinge region, CH2 is at least a portion of an immunoglobulin CH2 constant region, CH3 is at least a portion of an immunoglobulin CH3 constant region, n and m can be independently an integer between 0, 1 or 2 and 10.

3. At least one hinge core mimetibody polypeptide, comprising a polypeptide according to Formula (I):
((V(m)-P(n)-L(o)-H(p)-CH2(q)-CH3(r))(s),
where V is at least one portion of an N-terminus of an immunoglobulin variable region, P is at least one bioactive peptide selected from SEQ ID NOS:519-979, L is linker sequence, H is at least a portion of an immunoglobulin variable hinge region, CH2 is at least a portion of an immunoglobulin CH2 constant region, CH3 is at least a portion of an immunoglobulin CH3 constant region, n and m can be independently an integer between 0, 1 or 2 and 10.

4. A(n) hinge core mimetibody nucleic acid or hinge core mimetibody polypeptide according to claim 1, wherein said polypeptide has at least one activity of at least one P polypeptide.

5. A hinge core mimetibody antibody, comprising a monoclonal or polyclonal antibody, fusion protein, or fragment thereof, that specifically binds at least one hinge core mimetibody polypeptide according to claim 1.

6. A hinge core mimetibody nucleic acid encoding at least one hinge core mimetibody polypeptide or hinge core mimetibody antibody according to claim 1.

7. A hinge core mimetibody vector comprising at least one isolated nucleic acid according to claim 6.

8. A hinge core mimetibody host cell comprising an isolated nucleic acid according to claim 7.

9. A hinge core mimetibody host cell according to claim 8, wherein said host cell is at least one selected from COS-1, COS-7, HEK293, BHK21, CHO, BSC-1, Hep G2, 653, SP2/0, 293, NSO, DG44 CHO, CHO K1, HeLa, myeloma, or lymphoma cells, or any derivative, immortalized or transformed cell thereof.

10. A method for producing at least one hinge core mimetibody polypeptide or hinge core mimetibody antibody, comprising translating a nucleic acid according to claim 6 under conditions in vitro, in vivo or in situ, such that the hinge core mimetibody or antibody is expressed in detectable or recoverable amounts.

11. A composition comprising at least one hinge core mimetibody nucleic acid, hinge core mimetibody polypeptide, or hinge core mimetibody antibody according to claim 1.

12. A composition according to claim 11, wherein said composition further comprises at least one pharmaceutically acceptable carrier or diluent.

13. A composition according to claim 11, further comprising at least one composition comprising an therapeutically effective amount of at least one compound, composition or polypeptide selected from at least one of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an opthalmic, otic or nasal drug, a topical drug, a nutritional drug, a cytokine, or a cytokine antagonist.

14. A composition according to claim 11, in a form of at least one selected from a liquid, gas, or dry, solution, mixture, suspension, emulsion or colloid, a lyophilized preparation, or a powder.

15. A method for diagnosing or treating a hinge core mimetibody ligand related condition in a cell, tissue, organ or animal, comprising
(a) contacting or administering a composition comprising an effective amount of at least one hinge core mimetibody nucleic acid, polypeptide or antibody according to claim 1, with, or to, said cell, tissue, organ or animal.

16. A method according to claim 15, wherein said effective amount is 0.001-50 mg of hinge core mimetibody antibody; 0.000001-500 mg of said hinge core mimetibody; or 0.0001-100 μg of said hinge core mimetibody nucleic acid per kilogram of said cells, tissue, organ or animal.

17. A method according to claim 15, wherein said contacting or said administrating is by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

18. A method according to claim 15, further comprising administering, prior, concurrently or after said (a) contacting or administering, at least one composition comprising an effective amount of at least one compound or polypeptide selected from at least one of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an opthalmic, otic or nasal drug, a topical drug, a nutritional drug, a cytokine, or a cytokine antagonist.

19. A device, comprising at least one isolated hinge core mimetibody polypeptide, antibody or nucleic acid according to claim 1, wherein said device is suitable for contacting or administerting said at least one of said hinge core mimetibody polypeptide, antibody or nucleic acid, by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

20. An article of manufacture for human pharmaceutical or diagnostic use, comprising packaging material and a container comprising at least one isolated hinge core mimetibody polypeptide, antibody or nucleic acid according to claim 1.

21. The article of manufacture of claim 20, wherein said container is a component of a parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal intraspinal, intrasynovial, intrathoracic, intrauterine, intravesical, intralesional, bolus, vaginal, rectal, buccal, sublingual, intranasal, or transdermal delivery device or system.

22. A method for producing at least one isolated hinge core mimetibody polypeptide, antibody or nucleic acid according to claim 1, comprising providing at least one host cell, transgenic animal, transgenic plant, plant cell capable of expressing in detectable or recoverable amounts said polypeptide, antibody or nucleic acid.

23. At least one hinge core mimetibody polypeptide, antibody or nucleic acid, produced by a method according to claim 22.
